(19) United States
(12) Patent Application Publication
(10) Pub. No.: US 2024/0102104 A1
(43) Pub. Date: Mar. 28, 2024

(54) COMPOSITIONS AND METHODS FOR DETECTING AND TREATING ORAL CAVITY SQUAMOUS CELL CARCINOMA

(71) Applicant: THE UNIVERSITY OF CHICAGO, Chicago, IL (US)

(72) Inventors: Nishant AGRAWAL, Chicago, IL (US); Rifat HASINA, Chicago, IL (US); Evgeny IZUMCHENKO, Chicago, IL (US)

(73) Assignee: The University of Chicago, Chicago, IL (US)

(21) Appl. No.: 18/266,404

(22) PCT Filed: Dec. 8, 2021
(86) PCT No.: PCT/US21/72803

§ 371 Date: Jun. 10, 2023

Related U.S. Application Data
(60) Provisional application No. 63/123,360, filed on Dec. 9, 2020.

(51) Int. Cl. C12Q 1/6886

(52) U.S. Cl. CPC C12Q 1/6886; C12Q 2600/112; C12Q 2600/118; C12Q 2600/156

(57) ABSTRACT

Described herein is a method for evaluating a subject comprising detecting genetic mutation(s) in the DNA sequence of one or more oral cavity squamous cell carcinoma (OCSCC) biomarker(s) in a biological sample from the subject comprising DNA, wherein the OCSCC biomarker(s) comprise TP53, CDKN2A, FAT1, CASP8, NOTCH1, PIK3CA, and/or ERAS.

### CROSS-REFERENCE TO RELATED APPLICATIONS

This application claims the benefit of priority of U.S. Provisional Patent Application No. 63/123,360 filed Dec. 9, 2020, which is hereby incorporated by reference in its entirety.

### STATEMENT OF GOVERNMENT SUPPORT

This invention was made with government support under grant number CA230691 awarded by the National Institutes of Health. The government has certain rights in the invention.

### BACKGROUND

### I. Field of the Invention

This invention relates to the field of medicine and molecular biology.

### II. Background

Oral cavity squamous cell carcinoma (OCSCC) accounts for nearly 50% of all head and neck cancers (1). In 2018 alone, there was an estimated incidence of 355,000 oral cancer cases and 177,000 deaths worldwide (2), with India having the highest case burden of 120,000. OCSCC is notorious for poor prognosis, which reflects its propensity to present as clinically advanced disease upon diagnosis (1,3,4). Despite numerous therapeutic advances, the long-term survival for patients with HPV-negative OCSCC has remained −55%, and earlier detection is critical (5-10). If caught early, a patient has a survival rate of 80%, sharply in contrast to survival of 20-60% when diagnosed in the later stages. Consumption of alcohol, tobacco products, and betel quid and areca nut increase the risk of OCSCC. While prominent in oropharyngeal cancer, the prevalence of human papilloma virus (HPV) infection in OCSCC is low (˜2.2%) and its significance remains debatable (11-14).

Current standard detection methods for OCSCC include the conventional visual and tactile exam (CVTE) followed by tissue biopsy and histologic evaluation. However, its use as a modality for large-scale population-based screening has recognized limitations. First, a sampling bias may lead to underdiagnosis or misdiagnosis, particularly in diffuse and/or multifocal lesions. And second, these procedures are invasive, require specialized expertise, and are associated with pain/discomfort, sometimes leading to treatment delay (15-17). As early diagnosis is crucial for reducing mortality rate of OSCC patients, several adjunctive screening devices/tests (such as hand-held light-based devices for assessing autofluorescence/tissue reflectance) have recently emerged with claims of enhancing the identification and prognostication of oral lesions (18-22). Recently, the Council on Scientific Affairs of the American Dental Association (ADA) conducted a comprehensive systematic review of the published literature with a goal of providing primary care clinicians with practical, real-world recommendations regarding the clinical utility of the commercially available adjuncts/tests in the context of screening for oral potentially malignant disorders (23,24). The conclusion of the meta-analysis was that there is insufficient evidence to support the contention that any of the current devices/tests demonstrated sufficient diagnostic accuracy to be used in conjunction with the CVTE, underscoring the need for molecular-based biomarkers.

### SUMMARY

Aspects of the disclosure provide for more efficient ways to detect and treat oral cavity squamous cell carcinoma and premalignant lesions in subjects. Aspects relate to a method for evaluating a subject comprising detecting genetic mutation(s) in the DNA sequence of one or more oral cavity squamous cell carcinoma (OCSCC) biomarker(s) in a biological sample from the subject, wherein the biological sample consists of an oral rinse sample comprising saliva DNA, wherein the OCSCC biomarker(s) consist of TP53, CDKN2A, FAT1, CASP8, NOTCH1, PIK3CA, and HRAS, and wherein the genetic mutations are detected by next generation sequencing (NGS). Further aspects relate to a method for evaluating a subject comprising detecting genetic mutation(s) in the DNA sequence of one or more biomarker(s) in a biological sample from the subject, wherein the biological sample consists of an oral rinse sample comprising saliva DNA, wherein the biomarker(s) consist of TP53, CDKN2A, FAT1, CASP8, NOTCH1, PIK3CA, and HRAS, and wherein the genetic mutations are detected by next generation sequencing (NGS). Further aspects relate to a method for evaluating a subject comprising detecting genetic mutation(s) in the DNA sequence of one or more oral cavity squamous cell carcinoma (OCSCC) biomarker(s) in a biological sample from the subject comprising DNA, wherein the OCSCC biomarker(s) comprise TP53, CDKN2A, FAT1, CASP8, NOTCH1, PIK3CA, and/or HRAS. Further aspects relate to a method for evaluating a subject comprising detecting genetic mutation(s) in the DNA sequence of one or more head and neck cancer biomarker(s) in a biological sample from the subject comprising DNA, wherein the biomarker(s) comprise TP53, CDKN2A, FAT1, CASP8, NOTCH1, PIK3CA, and/or HRAS. Also provided is a method for treating a subject with OCSCC or premalignant oral cavity lesion, the method comprising administering a treatment for OCSCC to a subject that has, or has been determined to have, at least one genetic mutation in the DNA sequence of one or more OCSCC biomarker(s) in a biological sample from the subject comprising DNA, wherein the OCSCC biomarker(s) comprise TP53, CDKN2A, FAT1, CASP8, NOTCH1, PIK3CA, and/or HRAS. Also provided is a method for treating a subject with head and neck cancer or premalignant lesions related thereto, the method comprising administering a treatment for the cancer or lesion to a subject that has, or has been determined to have, at least one genetic mutation in the DNA sequence of one or more biomarker(s) in a biological sample from the subject comprising DNA, wherein the biomarker(s) comprise TP53, CDKN2A, FAT1, CASP8, NOTCH1, PIK3CA, and/or HRAS. Aspects provide for a method of diagnosing or screening a subject with OCSCC or premalignant oral cavity lesion comprising a) detecting genetic mutations in the DNA sequence of one or more OCSCC biomarker(s) in a biological sample from the subject comprising DNA, wherein the biomarker(s) comprise TP53, CDKN2A, FAT1, CASP8, NOTCH1, PIK3CA, and/or HRAS; b) determining that the subject has or is at high risk of having OCSCC when at least one genetic mutation in a OCSCC biomarker gene is detected or determining that the subject does not have or is at low risk of having OCSCC when no genetic mutation in a OCSCC biomarker gene is detected. Further aspects provide for a method of diagnosing or screening a subject with head and neck cancer or premalignant lesions thereof comprising a) detecting genetic mutations in the DNA sequence of one or more biomarker(s) in a biological sample from the subject comprising DNA, wherein the biomarker(s) comprise TP53, CDKN2A, FAT1, CASP8, NOTCH1, PIK3CA, and/or HRAS; b) determining that the subject has or is at high risk of having head and neck cancer or a premalignant lesion when at least one genetic mutation in a biomarker gene is detected or determining that the subject does not have or is at low risk of having head and neck cancer or premalignant lesion when no genetic mutation in a biomarker gene is detected. Further aspects relate to a kit or composition comprising primers or probes for sequencing one or more biomarker(s), wherein the biomarker(s) comprise TP53, CDKN2A, FAT1, CASP8, NOTCH1, PIK3CA, and/or HRAS. Also described is a method comprising: (i) isolating saliva DNA from an oral rinse sample from a subject; and (ii) sequencing TP53, CDKN2A, FAT1, CASP8, NOTCH1, PIK3CA, and HRAS genes in the DNA isolated from (i). Aspects relate to a method of making a nucleic acid comprising: isolating saliva DNA from an oral rinse sample from a subject; annealing primers to the isolated DNA, wherein the primers amplify and/or sequence the TP53, CDKN2A, FAT1, CASP8, NOTCH1, PIK3CA, and HRAS genes in the isolated DNA. In some aspects, the methods are for differentiating between an inflammatory and premalignant oral cavity lesion.

The premalignant lesion in aspects of the disclosure may further be classified as mild, moderate, or severe dysplasia. In some aspects, the premalignant lesion is further classified as leukoplakia, erythroplakia, or proliferative leukoplakia.

In aspects of the disclosure, the biological sample may comprise saliva DNA. The biological sample may comprise an oral rinse sample. In some aspects, the biological sample comprises cells or an extract thereof. In some aspects, the biological sample excludes serum or plasma. The method may exclude detecting genetic mutations in the DNA sequence of one or more biomarkers in serum or plasma or the subject excludes one that has had detection of genetic mutations in the DNA sequence of one or more biomarkers in a serum or plasma sample from the subject. In some aspects, the method excludes detecting genetic mutation(s) or analysis of DNA in a non-saliva sample or the subject excludes one that has had a non-saliva biological sample evaluated for genetic mutations in an biomarker gene. In some aspects, the method excludes centrifugation of the biological sample from the subject. In some aspects, the method excludes centrifugation of the biological sample from the subject prior to DNA isolation. The methods may comprise or further comprise isolating DNA from a cellular fraction of the biological sample.

The methods may comprise or further comprise ligation of an adaptor to the DNA. The adaptor may comprise at least one barcode. In some aspects, the adaptor comprises or further comprises a 5′ and/or 3′ primer binding site. The methods may comprise or further comprise enrichment of the DNA in the biological sample for the biomarker genes. Enrichment may comprise contacting the sample with a nucleic acid probe complimentary to the biomarker gene under conditions that allow for the hybridization of the probe and DNA in the biological sample that is at least partially complimentary to the probe. The enrichment may comprise or further comprise isolating the DNA hybridized to the probe. The methods may comprise or further comprise sequencing the DNA hybridized to the probe. The methods may comprise or further comprise sequencing DNA comprising all or part of the biomarker genes to provide the sequence of all or part of the biomarker genes. In some aspects, sequencing comprising contacting the biomarker gene with a polymerase and primer(s)s that hybridize to the biomarker gene or adjacent regions and using polymerase chain reaction (PCR) to amplify DNA sequences comprising the gene. In some aspects, sequencing comprises next generation sequencing. In some aspects, the coding exon regions of the gene are sequenced. In some aspects, all of the coding exon regions of the gene are sequenced. The methods may comprise or further comprise comparing the sequence of the biomarker genes to a control. The control may comprise the wild-type sequence of the gene. In some aspects, the method excludes whole exome sequencing methods. In some aspects, the method excludes droplet digital PCR.

The number of biomarkers evaluated in the biological sample is 1-7 biomarkers. In some aspects, the number of biomarkers evaluated comprises or consists of, comprises at least, or comprises at most 1, 2, 3, 4, 5, 6, or 7 biomarker genes. The biomarker may comprise TP53. The biomarker may comprise CDKN2A. The biomarker may comprise FAT1. The biomarker may comprise CASP8. The biomarker may comprise NOTCH1. The biomarker may comprise HRAS. The biomarker may comprise PIK3CA. The biomarker may comprise or consist of TP53, CDKN2A, FAT1, CASP8, and Notch1. The biomarker may comprise or consist of TP53, CDKN2A, FAT1, CASP8, Notch1, PIK3CA, and HRAS. In some aspects, the biomarkers consist of TP53, CDKN2A, FAT1, CASP8, Notch1, PIK3CA, and HRAS. In some aspects, at least one genetic mutation in the biomarker is detected. In some aspects, at least 2, 3, 4, 5, 6, 7, 8, 8, 9, or 10 genetic mutations in at least 1, 2, 3, 4, 5, 6, or 7 biomarker gene(s) are detected.

The methods may comprise or further comprise performing one or more diagnostic tests for OCSCC or head and neck cancer. The diagnostic test may comprise a conventional visual and tactile exam, tissue biopsy, and/or histological evaluation of a tissue biopsy. In some aspects, the method comprises or further comprises treating the subject for OCSCC or head and neck cancer. In some aspects, no genetic mutations were detected. In some aspects, the method excludes performing one or more diagnostic tests for OCSCC or head and neck cancer.

The subject may be a human subject. In some aspects, the subject is greater than 50 years old. In some aspects, the subject is at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 years in age (or any derivable range therein). The subject may be one that does not have any symptoms of OCSCC or head and neck cancer. In some aspects, the subject has one or more symptoms of OCSCC or head and neck cancer. The subject may be one that has not been treated with therapeutic levels of chemotherapy or radiation. The method may comprise or further comprise diagnosing the subject with head and neck cancer, OCSCC, premalignant oral cavity lesions, or premalignant head and neck lesions based on the evaluation. The OCSCC may comprise carcinoma of the tongue, buccal mucosa, alveolus, gingivobuccal sulcus, hard palate, lip, retromolar trigone, maxilla, or gum. The subject may be diagnosed with, or the cancer may comprise premalignant lesion, stage I, II, III, or IV cancer. In some aspects, the subject has a history of smoking or using tobacco products orally. In some aspects, the subject is not a current smoker or user of oral tobacco products, but has smoked or used tobacco products orally in the past. In some aspects, the subject is a current smoker or user of oral tobacco products. In some aspects, the subject is a non-smoker or non-user of oral tobacco products, and/or has no history of past smoking or use of oral tobacco products. The term “smoker” refers to one that smokes tobacco products.

The methods may include treating the subject for OCSCC or head and neck cancer. Treatments may include, for example, radiotherapy and chemotherapy, or surgery. The treatment may also include monoclonal antibody therapy, such as cetuximab. In some aspects, the treatment includes cisplatin. In some aspects, the treatment includes an immunotherapy. The immunotherapy, may comprise, for example, a PD-1 inhibitor. The PD-1 inhibitor may be an anti-PD-1 antibody. In some aspects, the immunotherapy comprises nivolumab or pembrolizumab. In further aspects, the immunotherapy comprises an immunotherapy described herein. In some aspects, the treatment comprises the combination of chemotherapy and an anti-PD-1 antibody. In some aspects, the treatment comprises the combination of i) pembrolizumab, ii) 5-FU, and iii) cisplatin or carboplatin. In some aspects, the treatment comprises the combination of i) an anti-PD-1 antibody, ii) 5-FU, and iii) cisplatin or carboplatin. In some aspects, the treatment comprises the combination of i) an anti-PD-1 and ii) cisplatin or carboplatin. In some aspects, the treatment comprises the combination of i) pembrolizumab and ii) cisplatin or carboplatin. In some aspects, the treatment comprises the combination of i) nivolumab and ii) cisplatin or carboplatin.

In some aspects, the OCSCC comprises HPV-negative OCSCC. In some aspects, the mutation is further defined as a somatic mutation. In some aspects, the variant allele frequency (VAF) of the mutation is less than 1%. In some aspects, the variant allele frequency (VAF) of the mutation is 0.1-0.25%. In some aspects, the VAF is less than 0.3%. In some aspects, the VAF is, or the VAF is less than 1, 0.9. 0.8, 0.7, 0.6, 0.5, 0.4, 0.3, 0.2, or 0.1% or any derivable range therein. In some aspects, the DNA excludes cfDNA.

In some aspects in the methods of the disclosure, the methods are for treating, diagnosing, screening, or evaluating OCSCC or premalignant oral cavity lesions in a subject. In some aspects, the methods exclude treatment, diagnosis, screening, or evaluation of head and neck cancer in a subject or premalignant lesions related thereto.

The kits of the disclosure may comprise or further comprise saliva collection vessels. In some aspects, the saliva collection vessel comprises a preservative. In some aspects, the kit or compositions comprises or further comprises DNA adaptors comprising a barcode. In some aspects, the DNA adaptors further comprise a 5′ and/or 3′ primer binding site. In some aspects, the kit or compositions further comprise one or more nucleic acid probes complimentary to the biomarker gene. The probes may be attached to a capture moiety. The capture moiety may comprise biotin. The kit may comprise or further comprise streptavidin bound to a solid support. The kit or compositions may comprise or further comprise primers that hybridize with the adaptor. The kit may comprise or further comprise one or more negative or positive control samples.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B: Selection of genes for the targeted OCSCC panel. A. A minimal set of genes where mutations would represent >85% distinct samples in the dataset were identified from three different WES studies on OCSCC patients, namely the TCGA-HNSC dataset restricted to OCSCC data (n=329), the ICGC (n=50) and the MD Anderson dataset (n=40). The intersection on the Venn diagram represents the core set of genes (TP53, FAT1 and CASP8) that captures the highest number of OCSCC samples across the three datasets. PIK3CA, HRAS, NOTCH1, and CDKN2A were manually curated for their clinical and biological significance in OCSCC, after evaluating the top 20 genes in each dataset. B. Bar chart shows the proportion of distinct samples (in each dataset and all three datasets combined) carrying at least one mutation in the 7 genes panel.

FIG. 2A-B: Targeted sequencing of primary OCSCC malignancies. A. Heatmap shows a sample-wise mutation distribution across the sequenced primary tumor specimens (106 subjects where at least one mutation was detected by the targeted 7 gene panel). Top panel: number of mutations per patient; Right panel: mutational frequency for each gene included in the targeted sequencing panel. The gender and histopathological stage classification are indicated in the strip chart below the heat map. B. Histogram comparing frequency distribution of mutations per gene in the study cohort and combined (TCGA, MD Anderson and ICGC) public datasets (n=419).

FIG. 3A-B: Analytical validation of the assay performance for low frequency variant detection. A. A positive control containing synthetic loci with 7 known mutations in TP53 and PIK3CA genes (at 0.25% VAF) was sequenced across 8 independent sequencing runs (solid colored lines). The same input material was also assessed by ddPCR assay (dashed black line). All mutations were detected in each one of the sequencing runs with expected VAF and remarkable concordance between ddPCR and NGS generated VAF values. B. Mutation free genomic loci containing a region of 665 bases in HRAS gene from a well-characterized contemporary normal NA12878 cell line was sequenced across 9 independent sequencing runs. Table summarizes the number of false positive calls detected in each sequencing run at >0.1% VAF.

FIG. 4: Concordance between primary tumor and oral rinse specimens. Percentage of OCSCC samples with functional mutations identified in primary tumor biopsies that were also detected in paired pre-treatment oral rinse specimens. Bar chart indicates concordance seen in patients with early stage (I and II) disease, late stage (III and IV) disease, and combined concordance across the entire cohort. Samples with more than one functional mutation in the primary tumor were considered to be concordant if any one of the mutations was detected in the paired oral rinse specimen.

FIG. 5A-H. Mutations distribution in primary tumor biopsies and matched oral rinse specimens. A-G. Lollipop plots show the landscape of genetic aberrations detected in primary tumors (top) and matched oral rinses (bottom) in each gene included in the sequencing panel. The variants are color coded by the mutation type (red—nonsense, green—missense, blue—deletion, violet—insertion). Gene domains are indicated in the bottom of each panel. H. Table depicts a dynamic increase in cumulative detection in the oral rinse with addition of each gene to the sequencing panel.

FIG. 6A-B: NGS summary data. Boxplots showing quality control metrics for all reads and final filtered reads across FFPE-derived primary OCSCC tumors (A) and oral rinse specimens (B). Each boxplot depicts the 25th quartile, the median and the 75th quartile of each metric.

FIG. 7. Squamous Cell Carcinoma Progression Model. Mucosal lesions do not follow a linear progression pattern to squamous cell carcinoma (SCC). While a small percentage of dysplastic lesions will progress to SCC, the majority will either remain quiescent or regress.

FIG. 8. Significantly mutated genes in HNSCC. Significantly mutated genes (rows) ordered by q value; additional genes with trends towards significance are also shown. Left, mutation percentage in TCGA. Right, mutation percentage in COSMIC.

FIG. 9. Most frequently mutated genes in OCSCC. Sequencing data and clinical information was downloaded from TCGA portal. Samples from oral cavity (alveolar ridge, buccal mucosa, floor of mouth, tongue, lip, oral cavity and hard palate) were selected, while other anatomical sites (larynx hypopharynx, oropharynx and tonsil) were excluded. 329 OCSCC samples were included for analysis.

FIG. 10A-B. Targeted sequencing of primary OSCC malignancies. A. Left bottom: Sample-wise mutation distribution across the sequenced specimens; Left top: number of mutations per patient; Right panel: Mutational frequency for each gene included in the sequencing panel. B. Number of mutations per patient (sorted from high to low). Red dashed line represents medium mutation number across the analyzed cohort.

FIG. 11A-B. Sequencing of paired premalignant and OCSCC neoplasms. A. Mutations detected in premalignant and invasive neoplasms. B. Fractional abundance of mutations shared between matched dysplasia/OSCC samples.

FIG. 12. Schema demonstrating the release of tumor DNA. Tumor-associated DNA mutations were detectable in the saliva of 46 out of 46 patients with OCSCC.

FIG. 13. Essential elements of Safe-SeqS. Step 1: each DNA fragment to be analyzed is assigned a unique identification (UID) DNA sequence (green or blue bars). Step 2: uniquely tagged fragments are amplified, producing UID families, each member of which has the same UID and sequenced, allowing for differentiation between real mutations and errors. A real mutant is defined as a UID family in which ≥90% of family members have the same mutation. Adapted from Kinde 2011.

FIG. 14A-B. A. A tDNA reference panel of synthetic loci containing 7 known mutations in TP53 and PIK3CA genes with allele frequency of 0.25% was ordered from Seraseq. Sequencing was performed independently 7 times. The same input material was also assessed by ddPCR assay. B. 92 primary OCSCCs and matched saliva samples were sequenced using ultra-deep targeted sequencing. Tumor associated mutations were detected in 85.7% and 92.5% of saliva collected from patients with early (blue bar) and late (red bar) stage disease respectively.

FIG. 15A-B. The landscape of mutations distribution across two most muted gene (A) TP53 and (B) FAT1 in either primary malignancies (top) or matched saliva samples collected from the same patients.

### DETAILED DESCRIPTION OF THE INVENTION

### I. Library Construction

The current disclosure may include detection of mutations in genetic biomarkers. The methods may include methods for constructing a cDNA library from the subject's DNA, such as saliva DNA. The terms “oligonucleotide,” “polynucleotide,” and “nucleic acid are used interchangeable and include linear oligomers of natural or modified monomers or linkages, including deoxyribonucleosides, ribonucleosides, α-anomeric forms thereof, peptide nucleic acids (PNAs), and the like, capable of specifically binding to a target (e.g. complementary or partially complementary) polynucleotide by way of a regular pattern of monomer-to-monomer interactions, such as Watson-Crick type of base pairing, base stacking, Hoogsteen or reverse Hoogsteen types of base pairing, or the like. Usually, monomers are linked by phosphodiester bonds or analogs thereof to form oligonucleotides ranging in size from a few monomeric units, e.g. 3-4, to several tens of monomeric units. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. Analogs of phosphodiester linkages include phosphorothioate, phosphorodithioate, phosphoranilidate, phosphoramidate, and the like. It is clear to those skilled in the art when oligonucleotides having natural or non-natural nucleotides may be employed, e.g. where processing by enzymes is called for, usually oligonucleotides consisting of natural nucleotides are required.

The term “vector” is used to refer to a carrier nucleic acid molecule into which a heterologous nucleic acid sequence can be inserted for introduction into a cell where it can be replicated and expressed and/or integrated into the host cell's genome. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used in a host cell to produce an antibody.

The term “expression vector” refers to a vector containing a nucleic acid sequence coding for at least part of a gene product capable of being transcribed or stably integrate into a host cell's genome and subsequently be transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

The vectors disclosed herein can be any nucleic acid vector known in the art. Exemplary vectors include plasmids, cosmids, bacterial artificial chromosomes (BACs) and viral vectors as well as CRISPR/Cas based systems.

Any expression vector for animal cell can be used. Examples of suitable vectors include pAGE107 (Miyaji et al., 1990), pAGE103 (Mizukami and Itoh, 1987), pHSG274 (Brady et al., 1984), pKCR (O'Hare et al., 1981), pSG1 beta d2-4 (Miyaji et al., 1990) and the like.

Other examples of plasmids include replicating plasmids comprising an origin of replication, or integrative plasmids, such as for instance pUC, pcDNA, pBR, and the like.

Other examples of viral vectors include adenoviral, lentiviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Examples of promoters and enhancers used in the expression vector for animal cell include early promoter and enhancer of SV40 (Mizukami and Itoh, 1987), LTR promoter and enhancer of Moloney mouse leukemia virus (Kuwana et al., 1987), murine myeloproliferative sarcoma virus promoter (MPSV, Baum et al. 1995), eukaryotic translation elongation factor 1 alpha promoter (EF-1 alpha), promoter (Mason et al., 1985) and enhancer (Gillies et al., 1983) of immunoglobulin H chain and the like.

A specific initiation signal also may be required for efficient translation of coding sequences. These signals include the ATG initiation codon or adjacent sequences such as the Kozak sequence. Exogenous translational control signals, including the ATG initiation codon, may need to be provided. One of ordinary skill in the art would readily be capable of determining this and providing the necessary signals.

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.)

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.) In aspects of the disclosure, condon-optimized vectors and nucleic acids are contemplated.

The vectors or constructs will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is preferred that that terminator comprises a signal for the cleavage of the RNA, and it is more preferred that the terminator signal promotes polyadenylation of the message.

In expression, particularly eukaryotic expression, one will typically include a polyadenylation signal to effect proper polyadenylation of the transcript.

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

A further aspect of the disclosure relates to a cell or cells. In some embodiments, a prokaryotic or eukaryotic cell is genetically transformed or transfected with at least one nucleic acid molecule or vector according to the disclosure. In some embodiments, the cells are infected with a viral particle of the current disclosure. In some embodiments, the cells are transfected with plasmids/vectors by electroporation.

The term “transformation” or “transfection” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed” or “transfected.” The construction of expression vectors in accordance with the current disclosure, and the transformation or transfection of the host cells can be carried out using conventional molecular biology techniques.

Suitable methods for nucleic acid delivery for transformation/transfection of a cell, a tissue or an organism for use with the current invention are believed to include virtually any method by which a nucleic acid (e.g., DNA) can be introduced into a cell, a tissue or an organism, as described herein or as would be known to one of ordinary skill in the art (e.g., Stadtfeld and Hochedlinger, Nature Methods 6(5):329-330 (2009); Yusa et al., Nat. Methods 6:363-369 (2009); Woltjen et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7(8):2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference

The nucleic acids of the disclosure may comprise or further comprise a barcode region that can identify the subject, gene, or biological sample. The barcode region can be a polynucleotide of at least, at most, or exactly 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200 or more (or any range derivable therein) nucleotides in length. The barcode may comprise or further comprise one or more universal PCR regions, adaptors, linkers, or a combination thereof. The barcode may represent a unique molecular identifier that may be used to determine whether a subject has a certain genetic mutation and/or the variant allele frequency of the genetic mutations.

Methods of the disclosure may include determining the identity of the barcode by determining the nucleotide sequence of the index region in order to identify which receptor(s) has been activated in a population of cells.

Nucleic acid constructs are generated by any means known in the art, including through the use of polymerases and solid state nucleic acid synthesis (e.g., on a column, multiwall plate, or microarray). The barcodes may correspond to a gene, subject, or biological sample.

The unique portions of the barcodes may be continuous along the length of the barcode sequence or the barcode may include stretches of nucleic acid sequence that is not unique to any one barcode. In one application, the unique portions of the barcodes may be separated by a stretch of nucleic acids that is removed by the cellular machinery during transcription into mRNA (e.g., an intron).

The barcodes and/or index regions are quantified or determined by methods known in the art, including quantitative sequencing (e.g., using an Illumina® sequencer) or quantitative hybridization techniques (e.g., microarray hybridization technology or using a Luminex® bead system). Sequencing methods are further described herein.

### II. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple cancer samples may be obtained for diagnosis by the methods described herein. In other cases, multiple samples, such as one or more samples from one tissue type (for example saliva) and one or more samples from another specimen (for example serum) may be obtained for diagnosis by the methods. In some cases, multiple samples such as one or more samples from one tissue type (e.g. saliva) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### III. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. Oral Cavity Squamous Cell Carcinoma

The OCSCC may be further classified as a verrucous carcinoma, minor salivary gland carcinoma, or lymphoma. OCSCC may also exclude a verrucous carcinoma, minor salivary gland carcinoma, or lymphoma. The OCSCC may include or comprise cancer of the lip, tongue, palate, cheek, jaw, gum, soft palate, hard palate, uvula, or floor of the mouth. The OCSCC may exclude cancer of the lip, palate, cheek, jaw, gum, soft palate, hard palate, uvula, or floor of the mouth.

The OCSCC may be one that is caused by human papillomavirus (HPV) or may be independent of HPV, meaning that the subject has tested negative for a current or past HPV infection in the oral cavity.

In some aspects, the methods of treatment and detection may be for premalignant lesions of the oral cavity. A premalignant (or precancerous) lesion may be defined as “a morphologically altered tissue that has a greater than normal risk of malignant transformation.” There are several different types of premalignant lesion that occur in the mouth. Some oral cancers may begin as white patches (leukoplakia), red patches (erythroplakia) or mixed red and white patches (erythroleukoplakia or “speckled leukoplakia”). Other common premalignant lesions include oral submucous fibrosis and actinic cheilitis.

The methods of the disclosure may be performed in combination with one or more additional diagnostic procedures. Diagnostic procedures can include a CT scan, MRI, PET scan, endoscopy of the nasal cavity/pharynx, larynx, bronchus, and esophagus, biopsy, fine needle aspiration, CVTE, adjunctive screening, light-based screening (autofluoresence/tissue reflectance), and/or cytology screening.

The OCSCC may be one that is limited to a specific cancer stage, according to TNM classification. TNM classification for oral cancer is exemplified in the tables below:

T: Primary tumor

| TX | Primary tumor cannot be assessed | |
|---|---|---|
| Tis | Carcinoma in situ | |
| T1 | Tumor ≤2 cm with depth of invasion (DOI*) ≤5 mm | |
| T2 | Tumor ≤2 cm with DOI* >5 mm or tumor >2 cm and ≤4 cm with | |
| | DOI* ≥10 mm | |
| T3 | Tumor >2 cm and ≤4 cm with DOI* >10 mm or tumor >4 cm with DOI* ≤10 mm | |
| T4 | Moderately advanced or very advanced local disease | |
| | T4a | Moderately advanced or very advanced local disease, tumor >4 cm DOI* >10 mm or |
| | | tumor invades adjacent structures only (cortical bone of the mandible or |
| | | maxilla (excluding superficial erosion of tooth socket alone in gingival |
| | | tumors) or involves the maxillary sinus or skin of the face) |
| | T4b | Very advanced local disease. Tumor invades masticator space, pterygoid |
| | | plates, or skull base and/or encases the internal carotid artery |

*DOI is depth of invasion and not tumor thickness.
N: Clinical Lymph nodes (separate classification for pathologic classification)

| NX | Regional lymph nodes cannot be assessed | |
|---|---|---|
| N0 | No regional lymph node metastasis | |
| N1 | Metastasis in a single ipsilateral lymph node, <3 cm and ENE(−) | |
| N2 | Metastasis in a single ipsilateral lymph node, ≤3 cm or smaller and ENE(+) or >3 | |
| | cm and ≤6 cm and ENE(−); or metastases in multiple ispsilateral lymph nodes, none >6 | |
| | cm and ENE(−); or in bilateral or contralateral lymph nodes(s), non >6 cm | |
| | ENE(−) | |
| | N2a | Metastasis in a single ipsilateral node <3 cm and ENE(+); or a single |
| | | ipsilatereral node ≥3 cm and <6 cm and ENE(−) |
| | N2b | Metastases in multiple ipsilateral nodes, <6 cm and ENE(−) |
| | N2c | Metastases in bilateral or contralateral lymph nodes(s); <6 cm and ENE(−) |
| N3 | Metastasis in a lymph node ≥6 cm and ENE(−); or metastasis in any nbodes(s) and | |
| | clinically overt ENE(+) | |
| | N3a | Metastasis in a lymph node ≥6 cm and ENE(−) |
| | N3b | Metastasis in any nodes(s) and clinically overt ENE(+) |

Note: A designation of “U” or “L” may be used for any N category to indicate metastasis
above (U) or below (L) the lower border of the cricoid. ENE(+/−) indicates presence or
absence of extranodal disease
M: Metastasis

| | |
|---|---|
| cM0 | No distant metastasis |
| cM1 | Distant metastasis |
| pM1 | Distant metastasis, microscopically confirmed |

TMN evaluation allows the person to be classified into a prognostic staging group:

AJCC Prognostic Stage Groups

| | | | Then the stage group |
|---|---|---|---|
| When T is . . . | And N is . . . | And M is . . . | is . . . |
| Tis | N0 | M0 | 0 |
| T1 | N0 | M0 | I |
| T2 | N0 | M0 | II |
| T3 | N0 | M0 | III |
| T1, T2, T3 | N1 | M0 | III |
| T4a | N0, N1 | M0 | IVA |
| T1, T2, T3, T4a | N2 | M0 | IVA |
| Any T | N3 | M0 | IVB |
| T4b | Any N | M0 | IVB |
| Any T | Any N | M1 | IVC |

The OCSCC in the methods of the disclosure may comprise Tis, T1, T2, T3, T4a, T4b, N0, N1, N2, N3, M0, M1, stage 0, I, II, III, IVA, IVB, or IVC, or combinations thereof. In some aspects, the OCSCC excludes Tis, T1, T2, T3, T4a, T4b, N0, N1, N2, N3, M0, M1, stage 0, I, II, III, IVA, IVB, or IVC.

In some aspects, the methods of the disclosure may be combined with a treatment for OCSCC. Treatments may include, for example, radiotherapy and chemotherapy, or surgery. The treatment may also include monoclonal antibody therapy, such as cetuximab

Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

In some aspects, the may include radiotherapy, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### V. Immunotherapy

In some aspects, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

A. Checkpoint Inhibitors and Combination Treatment

Aspects of the disclosure may include administration of immune checkpoint inhibitors, which are further described below.

1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDLL on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDLL and PDL2.

In some aspects, the PD-1 inhibitor is a molecule that inhibits the binding of PD-1 to its ligand binding partners. In a specific aspect, the PD-1 ligand binding partners are PDLL and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDLL or PDL2 fused to a constant region (e.g., an Fc region of an immunoglobulin sequence). In some aspects, the PDLL inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDLL inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B 7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

2. CTLA-4, B7-1, and B7-2 Inhibitors

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

B. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

C. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

D. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

E. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### VI. Detecting a Genetic Signature

Particular embodiments concern the methods of detecting a genetic signature in an individual. In some embodiments, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

A. Single Nucleotide Polymorphism (SNP) Detection

Particular embodiments of the disclosure concern methods of detecting a SNP in an individual. One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof.

In some embodiments of the disclosure, the method used to detect the SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some embodiments, the nucleotide at the particular SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a C or a T nucleotide, but not both, at the SNP. Thus, any method to determine binding of one selective oligonucleotide probe over another selective oligonucleotide probe could be used to determine the nucleotide present at the SNP.

One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one embodiment of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some embodiments, the probe is generally designed so the 3′ end of the probe pairs with the SNP. Thus, if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR.

In some embodiments, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G, A, or T nucleotide or G, A, or C nucleotide respectively. In particular embodiments, the PCR primer is at least or no more than 10, 11, 12, 13, 14, 15, 16, 17, 18, 19, 20, 21, 22, 23, 24, 25, 26, 27, 28, 29, 30, 31, 32, 33, 34, 3 5, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

B. DNA Sequencing

In some embodiments, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar.

C. Detection Kits and Systems

One can recognize that based on the methods described herein, detection reagents, kits, and/or systems can be utilized to detect the genetic mutation related to the genetic signature for diagnosing an individual (the detection either individually or in combination). The reagents can be combined into at least one of the established formats for kits and/or systems as known in the art. As used herein, the terms “kits” and “systems” refer to embodiments such as combinations of at least one detection reagent, for example at least one selective oligonucleotide probe or at least one PCR primer. The kits could also contain other reagents, chemicals, buffers, enzymes, packages, containers, electronic hardware components, etc. The kits/systems could also contain packaged sets of PCR primers, oligonucleotides, arrays, beads, or other detection reagents. Any number of probes could be implemented for a detection array. In some embodiments, the detection reagents and/or the kits/systems are paired with chemiluminescent or fluorescent detection reagents. Particular embodiments of kits/systems include the use of electronic hardware components, such as DNA chips or arrays, or microfluidic systems, for example. In specific embodiments, the kit also comprises one or more therapeutic or prophylactic interventions in the event the individual is determined to be in need of.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Ultra-sensitive detection of tumor-specific mutations in saliva of patients with oral cavity squamous cell carcinoma

Oral cavity squamous cell carcinoma (OCSCC) is the most common head and neck malignancy. While survival of patients with advanced stage disease remains ˜20-60%, when detected at early stage, survival approaches 80%, posing a pressing need for a well-validated profiling method for patients with high risk of developing OCSCC. Tumor DNA detection in saliva may provide a robust biomarker platform that overcomes limitations of current diagnostic tests. However, there is no routine saliva-based screening method for patients with OCSCC. The inventors have designed a custom next generation sequencing panel with unique molecular identifiers that covers coding regions of 7 frequently mutated genes in OCSCC, and applied it on DNA extracted from 121 treatment-naive OCSCCs and matched preoperative saliva specimens. Using stringent variants calling criteria, mutations were detected in 106 tumors, consistent with a predicted detection of at least 88%. Moreover, mutations identified in primary malignancies, were also detected in 93% of saliva samples. To ensure that variants are not errors resulting in false positive calls, the inventors performed a multistep analytical validation of this approach: (i) re-sequencing of 46 saliva samples confirmed 88% of somatic variants; (ii) no functionally relevant mutations were detected in saliva samples from 11 healthy subjects without history of tobacco and alcohol; (iii) using a panel of 7 synthetic loci across 8 sequencing runs, the inventors confirmed that this platform is reproducible and provides sensitivity on par with droplet digital PCR. These data highlight the feasibility of somatic mutation identification in driver genes in saliva collected upon OCSCC diagnosis.

### I. Materials and Methods

A. Ethics and Patient Recruitment

The study was approved by the Medical Ethics Committees of the three participating cancer centers, namely (a) HCG Cancer Centre, Bangalore, (b) HCG Panda Cancer Hospital, Cuttack, and (c) Tata Memorial Hospital, Mumbai. 121 treatment-naïve patients clinically diagnosed with OCSCC were enrolled into the study after obtaining their informed written consent. Staging was performed using the American Joint Committee on Cancer guidelines; clinical staging was used wherever histopathological evaluation was unavailable (9 subjects in the cohort). 44% of the subjects (n=53) had early stage disease (Stages I and II) while the cancer was advanced (Stages III and IV) in the remaining 68 subjects (56%). Three-quarters of the cohort were male and 52% of the subjects were above the age of 50 (n=64). Eleven age-matched normal subjects with no history of tobacco usage or alcohol consumption, and with no prior oral cancer or pre-cancer lesions were recruited. Detailed demographic and clinicopathological data for all individuals used in this study is presented in Supplementary Table S1 and summarized in Table 1.

B. Control Samples for Analytical Validation

To determine sensitivity of the panel, Seraseq® ctDNA Mutation Mix v2 variant allele frequencies (VAF) 0.25% (SeraCare Life Sciences Inc., Milford, MA, USA) was used. The specificity of the panel was evaluated using genomic DNA from the NA12878 cell line (Coriell Institute for Medical Research, Camden, NJ, USA).

C. Sample Collection

Matched primary tumor and oral rinse samples were collected from each subject. For formalin fixed and paraffin embedded (FFPE) samples with ≥20% tumor content the entire histological section was processed. For tumors with neoplastic content of <20%, the tumor areas were marked by the pathologist and scraped from the FFPE block for downstream processing. Oral rinse samples were collected prior to surgery or biopsy. Subjects were requested to swish 15 ml of 0.9% saline solution in their mouths for 15-30 seconds before spitting it into a collection tube. Immediately after collection, the oral rinse was centrifuged at 3000 g for 10 minutes at 4° C. The resulting pellet was resuspended in 10 ml of ThinPrep® PreservCyt® Solution (Hologic, Inc., Marlborough, MA, USA), which allows the long-term preservation of saliva samples at room temperature. Primary tumor samples from surgery or biopsy were formalin fixed and paraffin embedded (FFPE) as per standard protocols. Both sample types were transported at room temperature to the central NGS testing laboratory.

D. Selection of Genes and Panel Design

Since one purpose of the study was to design a low-cost test for OCSCC, the inventors developed a panel to maximize the number of unique patients who could be profiled with a minimal panel footprint. Three datasets were used for identifying the genes, (a) OSCC tumors from TCGA Head and Neck Squamous Cell Carcinoma dataset (n=329) (43), (b) ICGC Gingivo-buccal cohort (n=50) (44), and (c) MD Anderson Oral Squamous Cell Carcinoma cohort (n=40) (45). Seven genes were identified which would cover at least 85% of the cohort across the datasets, namely CASP8, PIK3CA, FAT I, CDKN2A, NOTCH I, HRAS, and TP53. Hybridization probes were designed to capture all coding exons in the selected genes and were manufactured by IDT (Integrated DNA Technologies, Coralville, IA, USA) with 2× tiling for the target regions. The total number of target bases for this panel was 29.8 Kbp.

E. Tumor DNA Extraction and Profiling

DNA from FFPE tissue was extracted using the QIAamp DNA FFPE Tissue Kit (Qiagen, Hilden, Germany) as per manufacturer's recommendations. DNA quality was assessed by estimating percent amplifiable DNA using Alu-based qPCR quantification. Libraries were then prepared from 200 ng of FFPE DNA using the KAPA Hyper plus Kit (Roche, Basel, Switzerland) with IDT's xGen Dual Index unique molecular identifiers (UMI) Adapters (Integrated DNA Technologies, Coralville, IA, USA) for molecular and sample based barcoding. Targeted enrichment was performed using IDT's custom synthesized xGen Lockdown Probes (Integrated DNA Technologies, Coralville, IA, USA) with modifications to hybridization temperature and time. Library quality was assessed using Agilent TapeStation 2200 (Agilent Technologies, Santa Clara, CA, USA) and were quantified using Qubit 2.0 Fluorometer (Invitrogen, Carlsbad, CA, USA). The final libraries were sequenced in on MiSeq (Illumina, Inc., San Diego, CA, USA), with loading optimized to achieve 1-2 million reads per sample.

F. Oral Rinse DNA Extraction and Profiling

DNA from oral rinse was isolated using QIAamp DNA Mini Kit (Qiagen, Hilden, Germany) as per manufacturer's instructions. An input of 100 ng was used to prepare libraries; the process is the same as described above. Final libraries were sequenced in on NextSeq500 (Illumina, Inc., San Diego, CA, USA). The library loading was optimized such that 35-45 million reads were sequenced per sample.

G. Bioinformatics and Interpretation Pipeline

All bioinformatics analyses were carried out on Strand NGS (ver. 3.3).

H. Alignment and Read Filters

Paired 150 bp reads were aligned against the GRCh37 (hg19) human genome assembly with minimum alignment identity set to 90%. Read pairs with identical UMI tags mapping to the exact same genomic position were clustered into UMI families. A consensus read was created from each UMI family. At each position in the read, the consensus base was that which occurred in more than 60% of reads in the UMI family. N was used to indicate bases where no consensus could be obtained. Quality of the consensus base was set to the maximum base quality at that position within the family. Consensus reads with alignment identity less than 95% and reads with indeterminate bases (Ns) were filtered out. Additionally, partially aligned and translocated reads were filtered out. Reads were removed if either paired read failed any of the above filters. An additional filter of UMI family size ≥3 (i.e. a consensus read must have been derived from at least three raw reads) was applied in case of the oral rinse samples. Quality control (QC) parameters such as total reads, average coverage, and percentage of reads on-target (% on-target), were determined for both raw reads and consensus reads. An average of 1,066 unique consensus reads (2,050× raw reads) per sample per base with an average of 2.26% LC and 86% on-target reads was achieved in FFPE samples. In saliva samples, an average coverage of 8,879× unique consensus reads (56,117× raw reads) per sample per base was achieved after applying read filters to eliminate background noise, with an average of 86% on-target reads. The data is presented in Supplementary FIG. 51 and summarized in Table 2.

I. Variant Calling

Single nucleotide variants (SNVs), and insertions/deletions (InDels) were detected from the final read list using a binomial SNV caller (46). Only variants supported by at least 5 different consensus reads were called in both FFPE and saliva samples. FFPE is prone to noise due to artifacts of formalin fixation, which in the assay can be suppressed with the use of UMIs. A subset of data from FFPE was evaluated for variant calling (with UMIs) reproducibility with thresholds between 2% and 5% VAF. The data was found to be >99% reproducible at 4% VAF. Therefore, the threshold for variant detection in FFPE was set at 4% VAF. In case of tDNA from saliva, the threshold was set at 0.1% VAF in oral rinse, at par with emerging literature on somatic variant calling in liquid biopsies (47-49). In addition to the 5 consensus reads requirement for variant calling in oral rinse, the inventors only considered high quality consensus reads that had been created from at least 3 raw reads. For both matrices, the pile-up at each base position of the panel was analyzed after discarding bases with quality <20, and paired-reads with different base calls at the position. Variants in homopolymer stretches and those with high strand and tail distance bias were also filtered out.

J. Interpretation

StrandOms, a clinical genomics interpretation and reporting platform from Strand Life Sciences, was used to prioritize and interpret variants identified in the samples. The primary tumor and oral rinse samples were interpreted independently. StrandOms contains an annotation engine with algorithms to identify the impact of the variant from public databases (dbSNP, 1000 Genomes, COSMIC etc) and bioinformatics prediction tools, along with proprietary content (data from over 15,000 samples) on genes, diseases, and therapeutic impact of somatic variants. A list of variants is categorized and annotated with likely functional effects as described in Sen et al (46). The final list of clinically relevant somatic variants was shortlisted manually using the following rules. Germline variants were excluded if a variant had a recorded population allele frequency above 0.01 in any public database. The list was further pruned by checking against an internal database of germline mutations from 15,000 patients of the same ethnicity to avoid any cohort-specific germline variants. Additionally, if a variant had variant allele frequency (% VAF)>30% in both the matched FFPE and saliva samples, it was eliminated as a likely germline variant and not included in the concordance analysis. Final list of clinically relevant variants includes functionally damaging, and likely functionally damaging events including variants of unknown significance. Therefore, the final list of shortlisted variants per sample are most likely somatic.

K. Reproducibility and Concordance Analysis

For reproducibility analysis, the inventors processed four primary tumor samples and 57 oral rinse samples (46 from OCSCC subjects and 11 from healthy subjects) in duplicates and independently sequenced. In primary tumor samples, the inventors assessed reproducibility of all variants ≥4% VAF. Overall reproducibility in FFPE was calculated as follows:

$$\frac{\begin{matrix}100 \times Number of variants reproduced in the \\ replicates at \geq 4 \% VAF\end{matrix}}{Total number of variants at 4 \% VAP}$$

In case of oral rinse samples, all variants in the range of 0.2 to 30% VAF from one replicate were assessed for their presence in the other replicate at variant calling threshold of 0.1% VAF. Reproducibility was calculated as the fraction of variants from a sample present in its replicate. Note that reproducibility was calculated in both directions—percentage of variants called in replicate 1 present in replicate 2 and vice versa. Overall reproducibility in oral rinse was calculated as follows:

$$\frac{\begin{matrix}100 \times Number of variant calls reproduced in \\ the replicates at \geq 0.1 \% VAF\end{matrix}}{Total number of variant calls between 0.2 to 30 \% VAF}$$

For concordance analysis, the variants obtained from interpreting the primary tumor sample were assessed for their presence in the matched saliva sample. Sample-pairs were called concordant if at least one tumor-specific mutation was found in the matched oral rinse specimen. Overall concordance was calculated as follows:

$$\frac{100 \times Number of concordant sample pairs}{\begin{matrix}Total number of primary tumor samples with \\ clinically relevant variants\end{matrix}}$$

L. ddPCR

The 20 μl ddPCR reaction containing Supermix (Bio-Rad), primers, mutant and wild-type probe and template DNA were loaded into a droplet generator. The emulsion was transferred into a 96 well plate, sealed, and cycled using a C-1000 thermal cycler (Bio-Rad) under the following conditions: 10 min hold at 95° C., 45 cycles of 95° C. for 15 s then 60° C. for 60 s. After amplification, the plate was transferred to a droplet reader from which raw fluorescence amplitude data is extracted to the Quantasoft software for downstream analysis.

### II. Results

A. Design of custom oral cancer panel

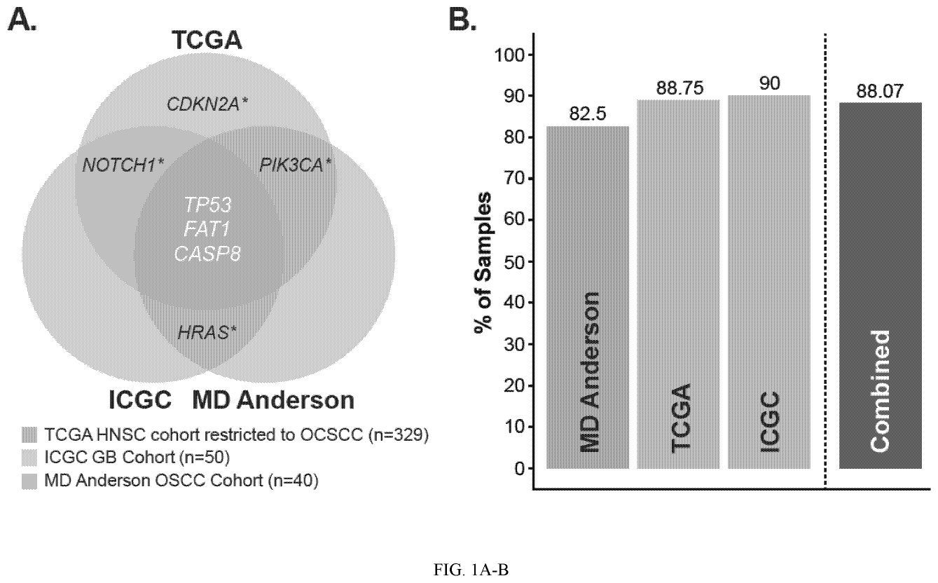

Tumor-specific mutations in body fluids are generally present at low frequencies. NGS-based tests for detecting variants present below 0.5% VAF require the usage of unique molecular identifiers (UMIs) for noise suppression in conjunction with high depth of sequencing (typically >50,000× per locus). Consequently, costs of such tests would be prohibitively high for large gene panels. The inventors aimed to select an optimal number of genes to build a panel that would cover >85% OCSCC patients in any cohort without redundancies. To this end, the inventors have obtained WES data from 3 independent publicly available OCSCC databases (n=419): TCGA-HNSC dataset, MD Anderson oral squamous cell carcinoma dataset, and ICGC gingivo-buccal cohort. For TCGA-HNSC dataset, only tumors of oral cavity ( ) were included to the analysis, while other anatomical sites (larynx, hypopharynx, oropharynx and tonsil) were excluded. For each cohort, the inventors have selected a minimum number of frequently mutated genes with at least 80% of the patients harboring at least one genomic alteration in any gene in the panel. TP53, FAT1 and CASP8 were the top three mutated genes in all three databases. From the remaining genes specific to databases, the inventors prioritized CDKN2A, NOTCH1, PIK3CA and HRAS for their clinical and biological significance in OCSCC (FIG. 1A). Subsequently, a panel was designed with probes covering the coding exons of these seven genes. With this panel design, the inventors observed that 82%, 89%, and 90% of the MD Anderson, TCGA, and ICGC cohorts respectively presented at least one mutation (FIG. 1B). Upon combining cohorts from the three datasets, approximately 88% of the subjects were represented minimally by one mutation in this panel. The analysis of the public data shows that the remaining 12% of the cohort is represented by a long tail of genes whose inclusion would significantly add to the sequencing costs per sample. This gives confidence that the panel should be able to profile >85% of OCSCC patients on a population-based level.

B. Ultra-Deep Targeted Sequencing of Primary OCSCC Tumors

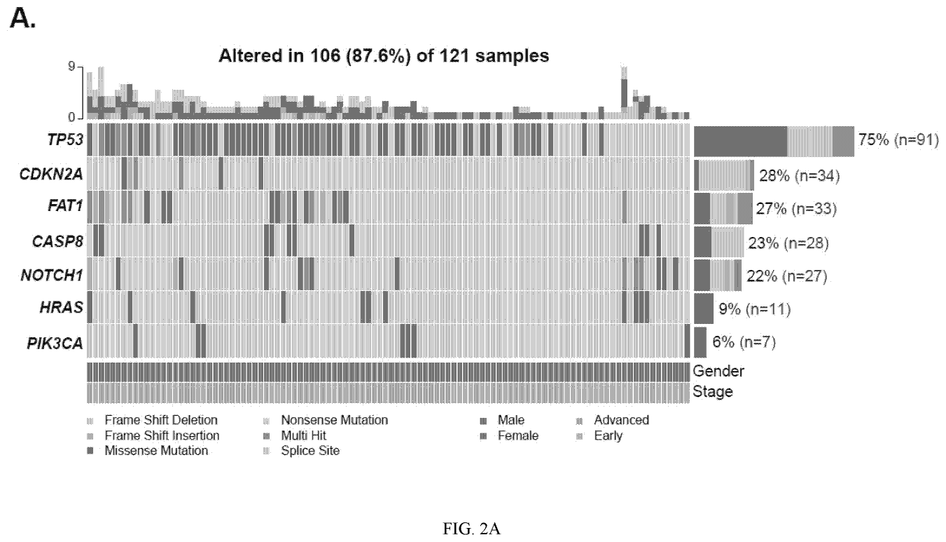

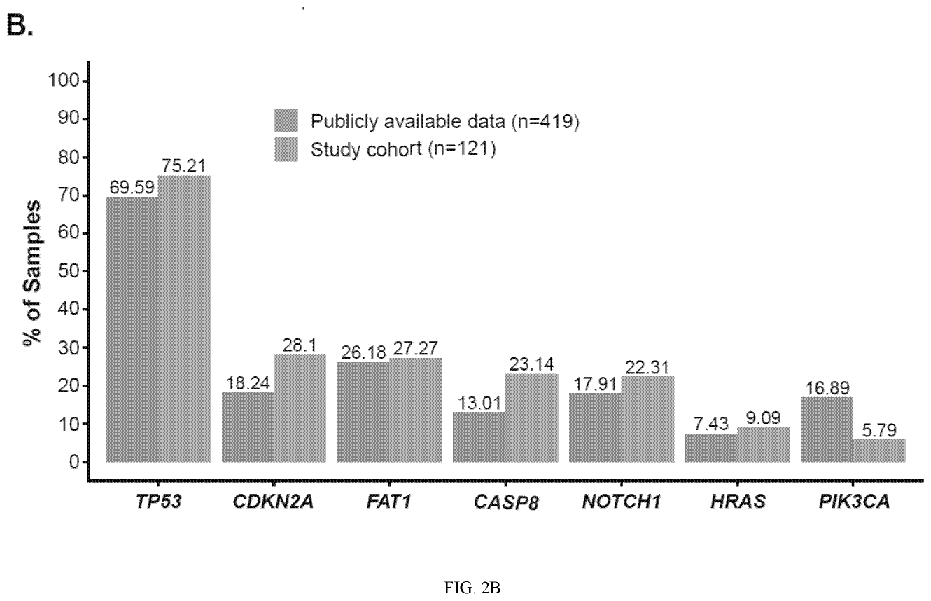

The inventors first applied this targeted sequencing approach on DNA extracted from 121 treatment naïve FFPE-derived primary OCSCC surgical specimens (Table 1). These patients had not been treated with chemotherapy or radiation before their tumor biopsy, so the spectrum of changes will largely reflect lesions in their naturally occurring malignant state. The inventors obtained 86% average on-target coverage with a median average consensus depth of 1,066× (2,050× raw read depth) across all sequenced tumor samples (Table 2, upper row). Using the stringent variant calling criteria of at least 5 mutant reads at 4% VAF, followed by filtering for functionally relevant somatic variants (see Methods section for details), 106 (87.6%) of the 121 sequenced specimens had at least one mutation detected in the seven genes included in the panel. Missense mutations were the most common type of variants identified in the cohort constituting 48.6% of the 278 variants identified, followed by nonsense mutations (28.8%). Mutations were detected in 75.5% of stage I/II and 97% of stage III/IV tumors (FIG. 2A). In the samples where variants were found, 71 of 106 samples carried more than one reported mutation (21 in early and 50 in late stage disease). TP53 (n=91) was the most frequently mutated gene, followed by CDKN2A (n=34), FAT1 (n=33), and CASP8 (n=28). While mutation frequencies in the cohort of 121 OCSCC tumors largely resembled mutation pattern in the combined patient cohort from TCGA, ICGC, and MD Anderson WES datasets (FIG. 2B), 6 of 7 genes in the sequencing panel showed higher mutation frequency in the study cohort compared to the frequencies for these genes seen in publicly available data (FIG. 2B). Detection of additional variants is likely due to a much higher coverage achieved with the targeted sequencing approach compared to the coverage usually obtained with WES in FFPE samples, which typically range between 70× and 100×(43-45). While there was a trend toward higher proportion of variant detected in patients with advanced disease, the inventors did not find an enrichment of a specific mutation by stage (Supplementary Tables S3A and S3B).

To validate the reproducibility of the sequencing and analytical workflow, new libraries were prepared from DNA extracted from 4 FFPE samples (subject OC-02-021, OC-020-035, OC-03-008, and OC-03-015). These libraries were sequenced and analyzed independently using variant calling threshold of 4% VAF. All variants (somatic and germline) detected in these 4 samples were considered for analysis. The reproducibility analysis has confirmed over 99% of the variants, and the prevalence of the mutant reads was very consistent between the two independent sequencing runs (Supplementary Table S4). Taken together, these observations support the credibility of the targeted biomarkers sequencing as a promising screening approach.

C. Analytical Validation of the Assay Performance for Low Frequency Variants Detection in Saliva

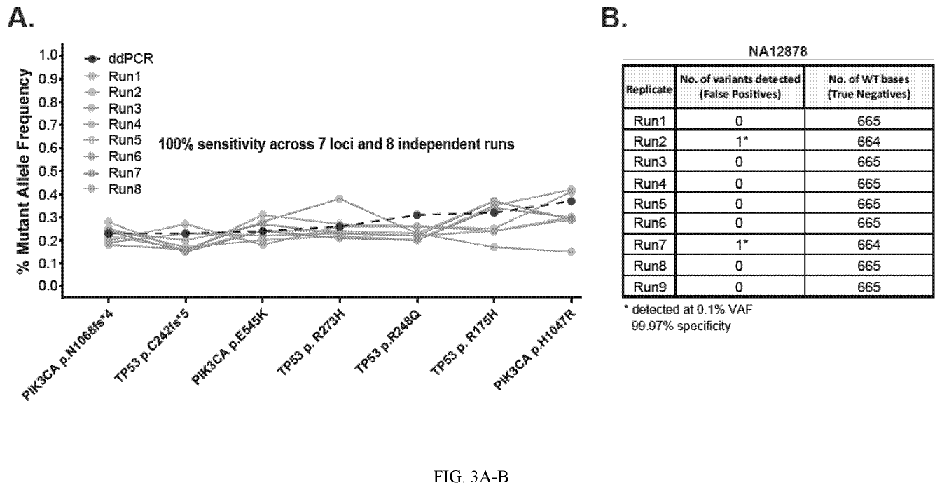

Unlike the FFPE tumor specimens, which contain higher degree of neoplastic cellularity (Supplementary Table S1), the presence of tDNA in body fluids is small, and high-DNase activity in saliva specimens further enhances tDNA degradation and abates its quality. As such, the inventors have performed a vigorous multistep analytical validation of the sequencing method to ensure its ability to reliably detect mutant variants at low tDNA concentrations. To this end, a reference synthetic positive control containing seven well-characterized mutations in TP53 and PIK3CA genes with 0.25% VAF was ordered from SeraCare (Supplementary Table S5). This sample was sequenced across 8 independent runs. Orthogonal validation of the variants in the positive control by droplet digital PCR (ddPCR) assays were provided by the manufacturer. Notably, all expected variants were reproducibly detected across all independent sequencing runs (FIG. 3A), thereby establishing the analytical sensitivity of the test at 100%. Furthermore, sensitivity for mutation detection was on par with that of the ddPCR assay, a gold-standard method for detecting low prevalence tumor-associated mutations (FIG. 3A). To assess the analytical specificity of the targeted sequencing panel for detecting single nucleotide variations (SNVs) and InDels, the inventors used a well-characterized reference material derived from the NA12878 normal cell line. From the Sanger-sequenced regions of NA12878 which were confirmed to be devoid of variants, five regions, comprising of 665 bp in the HRAS gene, overlapped with probes in the gene panel. This negative control sample was sequenced across nine independent runs with only 2 false positive SNV calls detected at 0.1% VAF, resulting in a specificity of ˜99.97% (FIG. 3B).

D. Targeted Sequencing of the Matched Pre-Treatment Oral Rinse Specimens

As the next step, the inventors have applied the targeted sequencing panel that was used to sequence 121 OCSCC tumors described in FIG. 2, on DNA extracted from the matched pre-treatment salivary oral rinses collected from these patients. Sequencing on Illumina NextSeq sequencer resulted in an average consensus read depth of 8,879× per sample, after applying read filters to eliminate background noise (with an average of 86% on-target reads) (Table 2, bottom row). Based on previous experience with detecting tumor associated mutations in bodily fluids (31), the cut-off for variant calling in oral rinses was set at 0.1%. Additionally, a variant was required to be supported by 5 distinct consensus reads of a minimum family size of 3 to ensure that it was not a sequencing artifact. Of the 121 oral rinse samples, 95.87% (n=116) had at least one somatic variant identified. The oral rinse samples, on an average, had 3 somatic mutations with 75% of samples having ≥2 somatic mutations, similar to the primary tumors. Missense mutations accounted for 45.35% of the 377 variants identified in the cohort, while nonsense mutations constituted 28.9%. The top four mutated genes in the oral rinse specimens were the same as those observed in the primary tumors, with TP53 remaining the most mutated gene (n=91), followed by FAT1 (n=50), CDKN2A (n=49), and NOTCH1 (n=43) (Supplementary Table S6).

In liquid biopsies including saliva, background noise introduced during library preparation and known errors of sequencing-by-synthesis chemistry (50,51) may contribute to false positives at low frequencies. Thus, the inventors have re-sequenced 46 of the 121 oral rinse specimens to ensure that detected variants are not an error resulting in a false positive call. For reproducibility analysis, the inventors confirmed presence of all somatic variants called at ≥0.2% VAF, a more stringent approach compared to evaluating reproducibility from a selected set of variants (52,53). It is important to note that with a variant calling threshold of 0.1% VAF, even variants genuinely present at 0.1% in the biological sample will often manifest in the data at frequencies slightly above or below the threshold, due to experimental variation. Hence, for reproducibility analysis, the inventors increased the variant query set to 0.2-30% VAF in one replicate, and found that 87.6% of the variants were detected in the other replicate at 0.1% VAF and above (Supplementary Table S7). The inventors further assessed the oral rinse specimens collected from 15 patients for whom no variants were reported in the primary FFPE tumors. Only two of these subjects showed clinically actionable variants at >=0.2% VAF. To validate that these variants are true somatic mutations and not sequencing artifacts, the specimens were re-sequenced in an independent run. Both variants were present at >0.1% allele frequency in the replicate, thereby indicating that high sensitivity of mutation detection in the saliva of OCSCC subjects. Notably, of the 11 oral rinse specimens collected from confirmed normal subjects without a visible oral cavity lesion and without history of tobacco usage, only one sample showed a single variant at >0.2% VAF, and one patient carried mutation between 0.1 and 0.2% VAF. However, these variants were not reproducible in the independent resequencing analysis (Supplementary Table S7). Furthermore, 9 oral rinse samples in which no mutations were detected remained free of genetic aberrations during the re-sequencing analysis, further supporting the specificity of the oral rinse sequencing assay.

E. Concordance Between Primary Tumor and Oral Rinse Specimens

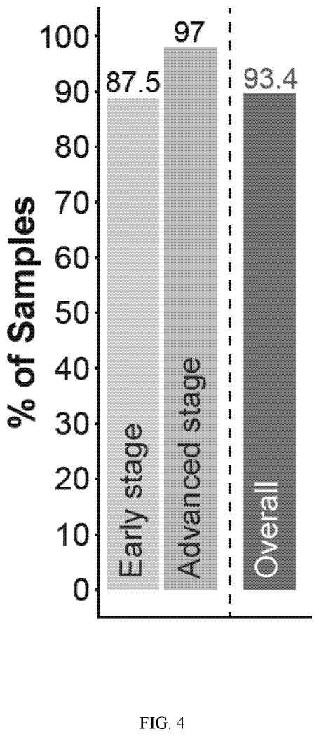

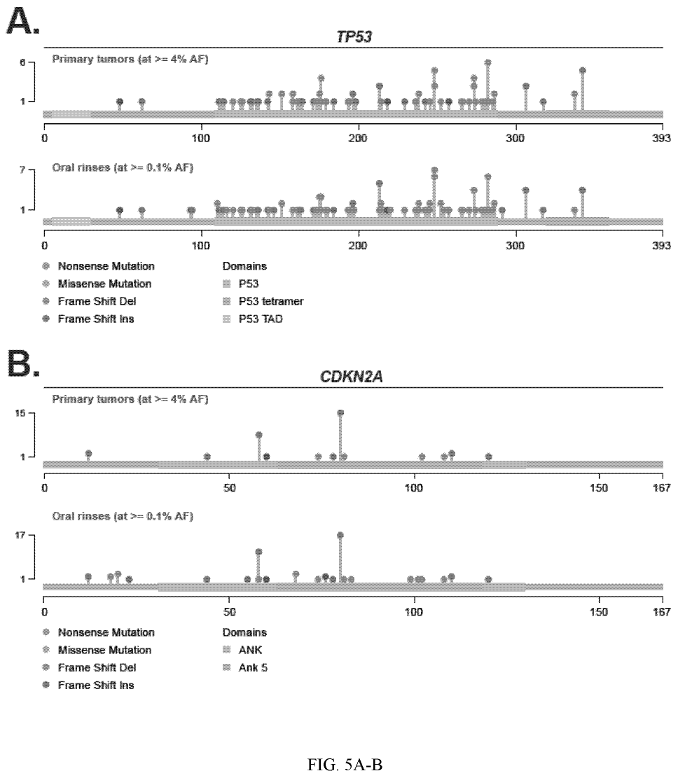

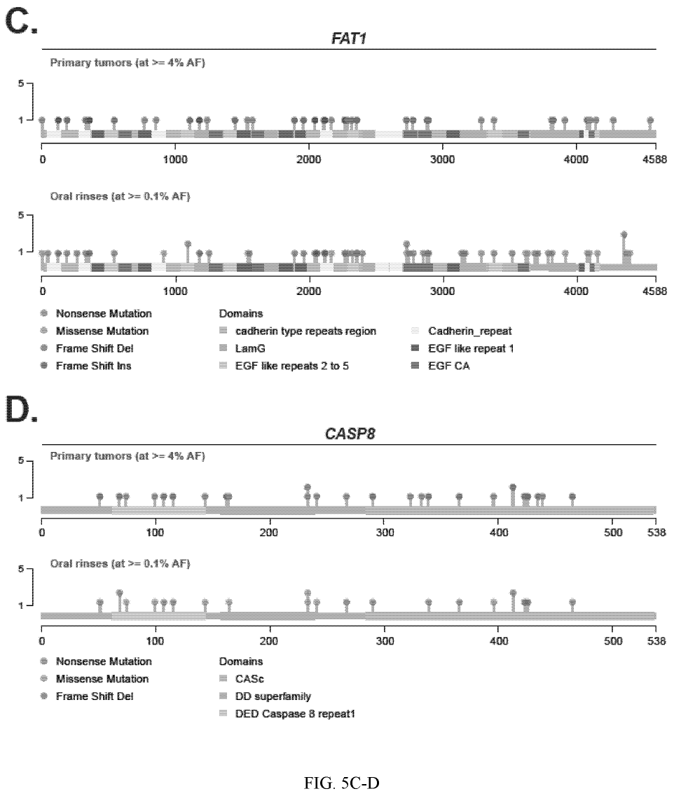

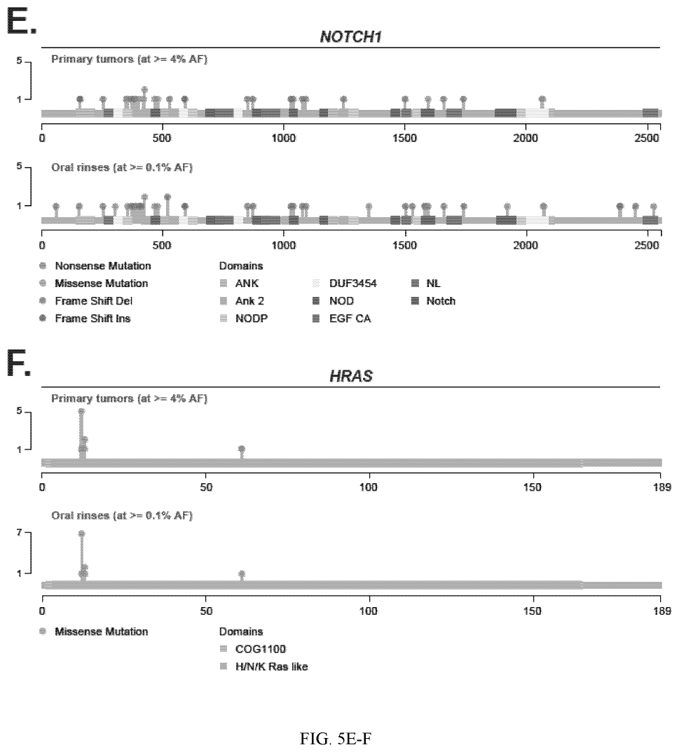

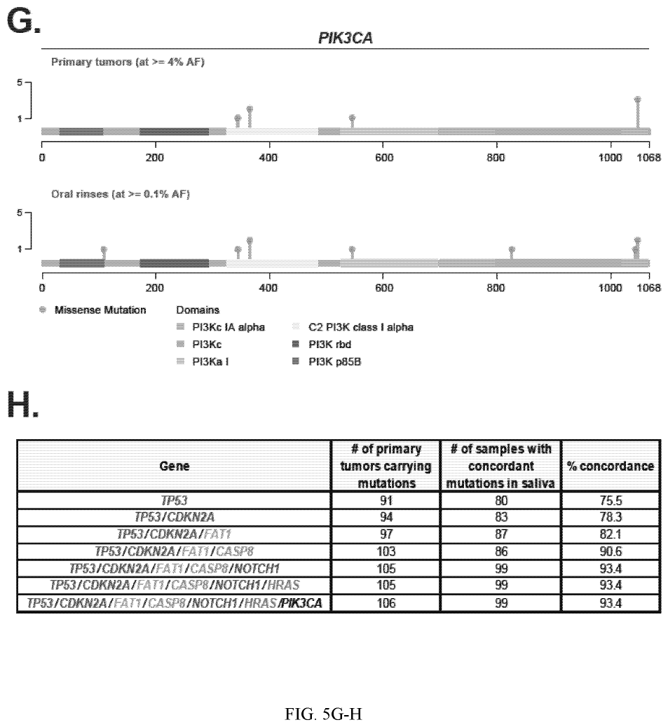

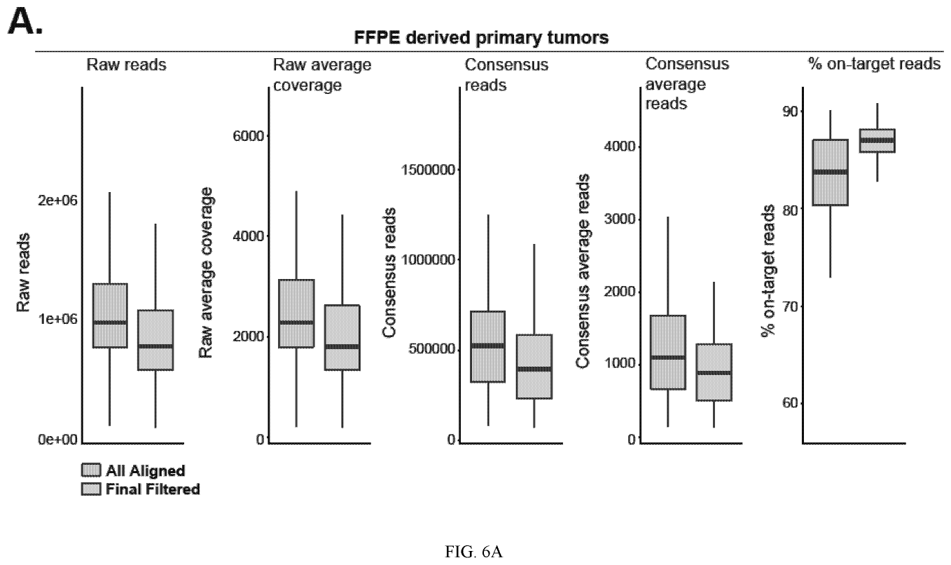

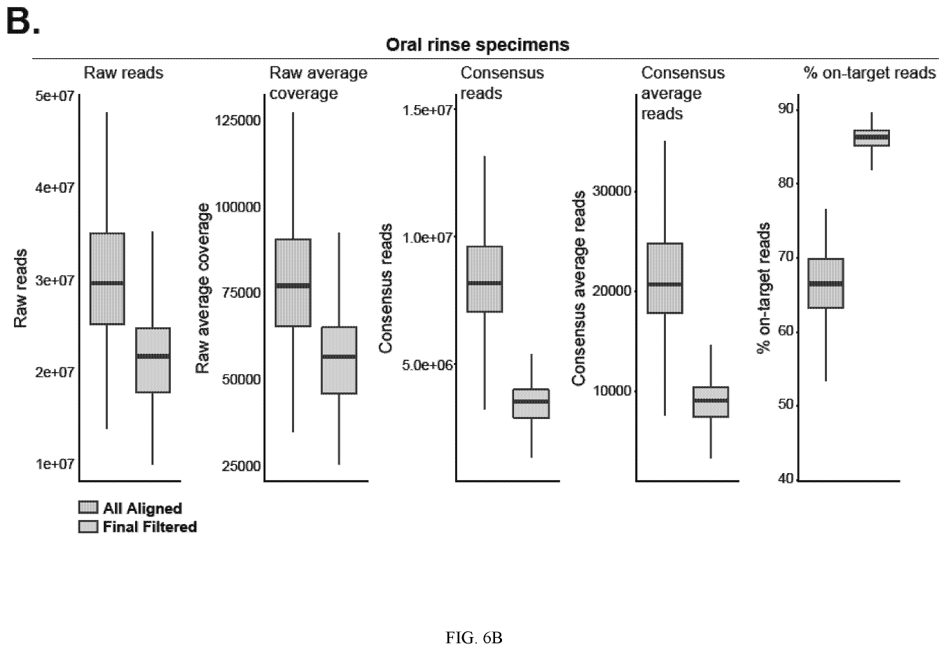

To assess if saliva-derived DNA is a good matrix for non-invasive detection of cancer in OCSCC patients, the inventors evaluated whether somatic mutations present in the solid tumor were represented in the saliva. Given that the oral rinse specimens contain a significant number of non-tumor cells from the oral cavity, allele frequencies of tumor associated mutations are expected to be <1% VAF (29,31,54-56). With the stringent filters applied by the variant calling algorithm, which only calls a sample concordant if the variant is present at ≥0.1% VAF, the overall concordance was 93.4% (99 of 106 sample pairs) (FIG. 4 and Supplementary Table S8). The high concordance in mutation distribution across the tested genes between primary tumors and saliva specimens is shown in FIG. 5. Although the inventors notice that 93% of the OCSCC tumors in the cohort could have been detected in paired oral rinse specimens by an even smaller 5 gene panel (TP53, CDKN2A, FAT1, CASP8 and NOTCH1) (FIG. 5H), all 7 genes in the assay are mutated in at least 5% of the patients with OCSCC (FIG. 2B). Therefore, inclusion of PIK3CA and HRAS into the panel may increase the detection rate in a larger cohort or population based screening. While the average allele frequency across primary tumor variants was 22.84% (S.D. ±16.05), the concordant variants in the oral rinses were detected at a mean of 0.68% VAF (S.D. ±0.665). Interestingly, in late stage cancers (Stages III and IV), the concordance between mutations detected in primary tumors and matched oral rinse specimens was as high as 97%, with concordance frequency decreased to 88% in patients with early stage disease (Stages I and II) (FIG. 4). Taken together, the data supports the feasibility of reliable somatic mutation identification in driver genes in saliva samples for OCSCC diagnosis, even at early stages of the disease.

### III. Discussion

Despite improved locoregional control and reduced treatment-related morbidity, 5-year survival for patients with OCSCC remains low, in part due to failure in early diagnosis. While early detection of OCSCC substantially increases overall survival (5-10), histopathologic examination of incisional tissue biopsy (a gold standard approach for cancer diagnosis) is invasive, costly, and depends on examiner experience (15-17). Novel strategies based on detection of genetic biomarkers offer new hope for improved diagnosis of cancer. However, a single tumor biopsy may fall short of accurately capturing clinically relevant genetic variants in a heterogeneous malignancy (57,58), resulting in improper molecular classification of the lesion and subsequently, inadvertent down-staging of the disease. Therefore, there is a pressing need for a non-invasive, rapid, accurate, and cost-effective screening approach that would overcome these challenges.

Over the last decade, there has been increasing interest in liquid biopsies—detection of cancer specific biomarkers in patients' body fluids (59,60). While a majority of liquid biopsy based diagnostic tests for solid malignancies rely on serum or plasma specimens (59,60), saliva is a better medium for detection of OCSCC. Saliva is in direct contact with oral cavity lesions, its collection is non-invasive, painless, and requires minimal training, making saliva an ideal biofluid to screen individuals with a high risk of developing OCSCC and early diagnosis of the disease (33-35,60). Using PCR-based assays, several retrospective studies, including those by the members of the inventors' group, have reported that tumor specific mutations are detectable in saliva of patients with OCSCC (31,36-38). Furthermore, saliva-based detection of tumor DNA performed better than plasma-based detection, especially in patients with early stage disease (29). However, the clinical adoption of PCR or ddPCR assays as a routine screening practice for OCSCC detection is hindered by their low scalability (they can only interrogate a limited set of variants) and limited multiplex capability. Targeted NGS technology overcomes these complications, and offers an advantageous approach for high-throughput and highly sensitive detection of tumor specific variants in small biopsies, FFPE-derived material, and saliva specimens (61,62). However, this method is not widely used for OCSCC diagnosis, and its accuracy is yet to be confirmed.

This motivated us to develop an ultra-deep NGS-based assay for rapid sequencing of the entire coding regions of 7 frequently mutated driver genes in OCSCC. The inventors have focused on targeted sequencing rather than a strategy based on WES, whole genome sequencing, copy number analysis, epigenetic changes, expression analysis, and/or proteomics (52,53,63). While each of these classes of alterations play a critical role in carcinogenesis, the goal was to develop highly specific and easily reproducible diagnostic and screening platforms that could be widely used in the clinical setting. The inventors focused on minimizing the panel size with the goal of achieving at least 85% overall clinical utility for the entire panel. Selected genes were rank-ordered and mutated in at least 5% of the patients in each of the three publicly available databases. These genes, for the most part, were mutually exclusive, and have well characterized clinical and biological significance in OCSCC.

A targeted UMI tagged NGS panel with a small footprint was designed to accurately call low-level somatic variants at 0.1% VAF. Targeted panels that do not use UMI, rely on modeling of background sequencing errors, which can distinguish true positives from background noise at a minimum of 0.3 to 0.5% VAF (64,65). Molecular tagging substantially lowers the limit of detection, which is essential for reliable detection of rare alleles in body fluids. Previous target enrichment attempts have steered away from hybridization-based approaches for rare allele detection, primarily due to high percentage of off-target capture. In the assay, the inventors circumvent this problem by applying two rounds of hybridization. Targeted hybridization approaches have previously been explored in the context of pan-cancer panels with footprints of 16 or more genes (52,53). While large panel size reduces off-target capture, it requires a higher number of reads per sample to achieve sufficient depth for variant allele detection at 0.1%, which increases the cost and offsets the use of such assays for early detection screening. The targeted saliva-based seven-gene panel used in this study costs a fraction of other plasma-based NGS panels currently available in the market (such as FoundationOne® Liquid CDx and Guardant360° C.Dx). Therefore, targeted dual-capture hybridization enrichment coupled with UMI-tagged minimal panel footprint provides a sensitive and cost-effective alternative for accurate detection of low frequency alleles in a complex genomic background of saliva specimens.

To test this sequencing workflow, the inventors first applied it on DNA extracted from 121 FFPE-derived primary OCSCC tumors. Overall, 86% of reads mapped to the reference sequence and average depth was over 1,000× across all tested specimens (14-fold higher compared to −70× that could be achieved with WES of FFPE-derived samples (43,45)). Furthermore, nearly 99% of mutations were concurrently detected in two parallel sequencing runs, supporting the high reproducibility of this targeted sequencing approach. Notably, somatic mutations were detected in 88% of the specimens, confirming the clinical utility of this gene panel predicted from the public datasets. While the inventors acknowledge that the inventors won't be able to identify patients with low prevalent mutations of unknown biologic and clinical relevance, inclusion of rarely mutated genes has limited prognostic utility in a heterogeneous population of patients with OCSCC and also would substantially increase the screening cost.

Cell-free (cf) DNA is often the source of material for most liquid biopsy assays. However, in saliva, DNA is extracted primarily from the shedding mucosal cells. Compared to cfDNA, which is subject to degradation by high DNase activity, DNA extracted from the cellular fraction of saliva is far less fragmented, thereby increasing detection accuracy of rare alleles. As such, salivary oral rinse specimens used in this study were collected by asking the patients to swish and gargle with saline in order to increase the cellular fraction. As high sequencing depth is required for accurate low-level variants calling, these oral rinse specimens were sequenced on Illumina NextSeq sequencer, resulting in an average depth of 8,879× consensus reads. Such depth overcomes the shortcomings of sequencing even highly degraded material. At ≥0.1% VAF, independent re-sequencing of 46 oral rinse specimens confirmed presence of mutant alleles with 87.7% concordance, and 93.4% of mutations detected in primary tumors were also identified in matched oral rinse specimens. Furthermore, although early stage malignancies have lower levels of neoplastic cells and therefore more likely to yield false negative results, an 88% concordance in early stage disease confirms the success of the sequencing and analytical approach. Somatic mutations that were not seen in the primary tumors were detected in five of the sequenced oral rinse specimens. While these results are consistent with previous reports on higher mutational prevalence in body fluids (29, 31) and the nature of these mutations remains to be investigated, these variants most likely have been missed due to the undersampling of the heterogeneous primary tumor, suggesting that saliva is highly representative of the intratumor mutational heterogeneity.

Taken together, these results demonstrate that this quick, sensitive, cost-efficient, and non-invasive method can be used for detection of low frequency tumor-associated mutations in salivary oral rinse specimens collected from patients with OCSCC. These findings provide the foundation for using this sequencing platform for risk assessment by screening high-risk individuals, early detection, monitoring during treatment, and tumor surveillance after completion of treatment. With an annual incidence of over 350,000 new cases of OCSCC and approximately two-thirds of these cases occurring in developing nations, the value of this tool in addressing the continuing challenges in screening the high risk population will likely increase over time.

### IV. Supplemental Tables

TABLE 1
Subject Demographics
OCSCC

| | |
|---|---|
| Total Enrolled | 121 |

Age

| | | |
|---|---|---|
| Mean (±SD) | 49.2 | (±12.8) |
| ≤40 | 29 | (23.97%) |
| 41-50 | 34 | (28.10%) |
| 51-60 | 30 | (24.79%) |
| 61-70 | 21 | (17.36%) |
| ≥71 | 7 | (5.79%) |

Gender

| | | |
|---|---|---|
| Female | 30 | (24.8%) |
| Male | 91 | (75.2%) |

Stage-wise

| | | |
|---|---|---|
| Stage I | 17 | (14%) |
| Stage II | 36 | (29.8%) |
| Stage III | 26 | (21.5%) |
| Stage IV | 42 | (34.7%) |
| Early (I and II) | 53 | (43.8%) |
| Late (III and IV) | 68 | (56.2%) |

Risk Factors

| | | |
|---|---|---|
| Tobacco Use | 56 | (46.28%) |
| (including belel quid and areca nut) | | |
| Alcohol Consumption | 4 | (3.31%) |
| Both | 38 | (31.40%) |
| Unknown | 23 | (19.01%) |

Healthy Controls

| | |
|---|---|
| Total Enrolled | 11 |

Age

| | | |
|---|---|---|
| Mean (+−SD) | 47 | (+−4.45) |

Gender

| | | |
|---|---|---|
| Female | 5 | (45.45%) |
| Male | 6 | (54.55%) |

TABLE 2

| | | | Total consensus | % Reads mapped | Average coverage | Average coverage |
|---|---|---|---|---|---|---|
| Sample type | Platform | Total raw reads | reads | to targets | (raw reads) | (consensus reads) |
| FFPE | MSeq | 1,098,707 | 580,917 | 86 | 2,050 | 1,066 |
| Oral rinse | NextSeq500 | 30,024,859 | 8,247,771 | 86 | 55,117 | 8,879 |

SUPPLEMENTAL TABLE S1
PATIENT DEMOGRAPHIC AND CLINICOPATHOLOGICAL DETAILS

| | | | | | TNM | | TNM | | | | | Tumor | |
|---|---|---|---|---|---|---|---|---|---|---|---|---|---|
| Subject | Subject | | | Cancer Type | Clinical | Clinical | Pathological | Histopathological | Final | | Tumor | block | |
| ID | Category | Age | Gender | Diagnosis | Stage | Stage | Stage | Stage | Stage | Category | Content | obtained | Comment |
| OC-01- | OCSCC | 56 | Male | Carcinoma | cT1N0 | Stage I | pT1N0 | Stage I | Stage I | Early | <20% | Surgery | Overall, tumor |
| 001-AR | positive | | | tongue | | | | | | | | | content of |
| | | | | | | | | | | | | | block is <20%. |
| | | | | | | | | | | | | | Areas |
| | | | | | | | | | | | | | marked as |
| | | | | | | | | | | | | | tumor were |
| | | | | | | | | | | | | | specifically |
| | | | | | | | | | | | | | sampled for |
| | | | | | | | | | | | | | DNA |
| | | | | | | | | | | | | | extraction |
| OC-01- | OCSCC | 45 | Female | Carcinoma | cT4aN1M0 | Stage IV | pT4N2b | Stage IV | Stage IV | Advanced | 80% | Surgery | |
| 002-ABR | positive | | | left buccal | | | | | | | | | |
| | | | | mucosa | | | | | | | | | |
| OC-01- | OCSCC | 54 | Male | Carcinoma | cT4aN2aM0 | Stage IV | pT3N3a | Stage IV | Stage IV | Advanced | 20% | Surgery | |
| 003-BM | positive | | | right buccal | | | | | | | | | |
| | | | | mucosa | | | | | | | | | |
| OC-01- | OCSCC | 57 | Female | Carcinoma | cT4aN2cM0 | Stage IV | pT1N2c | Stage IV | Stage IV | Advanced | 95% | Surgery | |
| 006-SA | positive | | | right buccal | | | | | | | | | |
| | | | | mucosa | | | | | | | | | |
| OC-01- | OCSCC | 53 | Male | Carcinoma | cT1N0M0 | Stage I | pT1N0 | Stage I | Stage I | Early | 20% | Surgery | |
| 007-ST | positive | | | tongue | | | | | | | | | |
| OC-01- | OCSCC | 50 | Female | Carcinoma | cT4aN0M0 | Stage IV | pT2Nx | Stage II | Stage II | Early | 80% | Surgery | |
| 008-KA | positive | | | right buccal | | | | | | | | | |
| | | | | mucosa | | | | | | | | | |
| OC-01- | OCSCC | 36 | Male | Carcinoma of | cT3N2 | Stage IV | pT2N2b | Stage IV | Stage IV | Advanced | 90% | Surgery | |
| 013-SR | positive | | | buccal | | | | | | | | | |
| | | | | mucosa | | | | | | | | | |
| OC-01- | OCSCC | 84 | Female | Carcinoma | cT4aN2cM0 | Stage IV | NA | NA | Stage IV | Advanced | 70 to 80% | Clinical | |
| 014-SU | positive | | | tongue | | | | | | | | evaluation | |
| | | | | | | | | | | | | only | |
| OC-01- | OCSCC | 48 | Male | Carcinoma | cT3N3b | Stage IV | pT3N3b | Stage IV | Stage IV | Advanced | 70% | Surgery | |
| 018-SSR | positive | | | alveolus | | | | | | | | | |
| OC-01- | OCSCC | 64 | Male | Carcinoma | cT4N2M0 | Stage IV | NA | NA | Stage IV | Advanced | 75% | Biopsy | |
| 019-MA | positive | | | gingivo- | | | | | | | | | |
| | | | | buccal sulcus | | | | | | | | | |
| OC-01- | OCSCC | 38 | Female | Carcinoma | cT2N2b | Stage IV | pT2N2b | Stage IV | Stage IV | Advanced | 50% | Surgery | |
| 020-TSA | positive | | | tongue | | | | | | | | | |
| OC-01- | OCSCC | 66 | Female | Carcinoma | cT4aN2cM0 | Stage IV | pT3N3b | Stage IV | Stage IV | Advanced | 90% | Surgery | |
| 023-CS | positive | | | hard palate | | | | | | | | | |
| OC-01- | OCSCC | 60 | Female | Carcinoma of | cT1N0M0 | Stage I | pT2N0 | Stage II | Stage II | Early | <20% | Surgery | Overall, tumor |
| 024-KB | positive | | | right lower | | | | | | | | | content of |
| | | | | lip | | | | | | | | | block is <20%. |
| | | | | | | | | | | | | | Areas |
| | | | | | | | | | | | | | marked as |
| | | | | | | | | | | | | | tumor were |
| | | | | | | | | | | | | | specifically |
| | | | | | | | | | | | | | sampled for |
| | | | | | | | | | | | | | DNA |
| | | | | | | | | | | | | | extraction |
| OC-01- | OCSCC | 50 | Male | Carcinoma | cT3N0M0 | Stage III | pT1N0 | Stage I | Stage I | Early | <20% | Biopsy | Overall, tumor |
| 025-PC | positive | | | tongue | | | | | | | | | content of |
| | | | | | | | | | | | | | block is <20%. |
| | | | | | | | | | | | | | Areas |
| | | | | | | | | | | | | | marked as |
| | | | | | | | | | | | | | tumor were |
| | | | | | | | | | | | | | specifically |
| | | | | | | | | | | | | | sampled for |
| | | | | | | | | | | | | | DNA |
| | | | | | | | | | | | | | extraction |
| OC-01- | OCSCC | 69 | Male | Carcinoma | cT2N0M0 | Stage II | pT2N1 | Stage III | Stage III | Advanced | 50% | Surgery | |
| 026-GKD | positive | | | retromolar | | | | | | | | | |
| | | | | trigone | | | | | | | | | |
| OC-01- | OCSCC | 46 | Female | Carcinoma | cT4aN0M0 | Stage IV | pT4aN0 | Stage IV | Stage IV | Advanced | 50% | Surgery | |
| 028-MD | positive | | | left buccal | | | | | | | | | |
| | | | | mucosa | | | | | | | | | |
| OC-01- | OCSCC | 26 | Male | Carcinoma | cT4aN1M0 | Stage IV | NA | NA | Stage IV | Advanced | 80% | Clinical | |
| 029-JR | positive | | | right buccal | | | | | | | | evaluation | |
| | | | | mucosa | | | | | | | | only | |
| OC-01- | OCSCC | 68 | Male | Carcinoma | cT4aN0M0 | Stage IV | pT4aN2b | Stage IV | Stage IV | Advanced | 90% | Surgery | |
| 030-RP | positive | | | left buccal | | | | | | | | | |
| | | | | mucosa | | | | | | | | | |
| OC-01- | OCSCC | 59 | Male | Carcinoma | cT3N3b | Stage IV | pT3N3b | Stage IV | Stage IV | Advanced | 60% | Biopsy | |
| 032-SUS | positive | | | tongue | | | | | | | | | |
| OC-01- | OCSCC | 83 | Male | Carcinoma of | cT2N0 | Stage II | pT2N0 | Stage II | Stage II | Early | 20 to 30% | Surgery | |
| 033-PS | positive | | | buccal | | | | | | | | | |
| | | | | mucosa | | | | | | | | | |
| OC-01- | OCSCC | 50 | Female | Carcinoma | cT4aN2b | Stage IV | NA | NA | Stage IV | Advanced | 60 to 70% | Clinical | |
| 034-KS | positive | | | left alveolus | | | | | | | | evaluation | |
| | | | | | | | | | | | | only | |
| OC-01- | OCSCC | 56 | Male | Carcinoma | cT3N0 | Stage III | pT4bN0 | Stage IV | Stage IV | Advanced | 70% | Surgery | |
| 035-JR | positive | | | left lower | | | | | | | | | |
| | | | | alveolus | | | | | | | | | |
| OC-01- | OCSCC | 44 | Male | Carcinoma | cT1N0M0 | Stage I | pT1N0 | Stage I | Stage I | Early | <20% | Surgery | Overall, tumor |
| 037-JK | positive | | | tongue | | | | | | | | | content of |
| | | | | | | | | | | | | | block is <20%. |
| | | | | | | | | | | | | | Areas |
| | | | | | | | | | | | | | marked as |
| | | | | | | | | | | | | | tumor were |
| | | | | | | | | | | | | | specifically |
| | | | | | | | | | | | | | sampled for |
| | | | | | | | | | | | | | DNA |
| | | | | | | | | | | | | | extraction |
| OC-01- | OCSCC | 65 | Female | Carcinoma of | cT3N0 | Stage III | pT3N0 | Stage III | Stage III | Advanced | 60 to 70% | Surgery | |
| 038-GA | positive | | | buccal | | | | | | | | | |
| | | | | mucosa | | | | | | | | | |
| OC-01- | OCSCC | 72 | Male | Carcinoma | cT2N0 | Stage II | pT3N0 | Stage III | Stage III | Advanced | 60 to 70% | Surgery | |
| 040-AJ | positive | | | tongue | | | | | | | | | |
| OC-01- | OCSCC | 50 | Male | Carcinoma | cT1N0M0 | Stage I | pT2N0 | Stage II | Stage II | Early | <20% | Surgery | Overall, tumor |
| 045-PH | positive | | | tongue | | | | | | | | | content of |
| | | | | | | | | | | | | | block is <20%. |
| | | | | | | | | | | | | | Areas |
| | | | | | | | | | | | | | marked as |
| | | | | | | | | | | | | | tumor were |
| | | | | | | | | | | | | | specifically |
| | | | | | | | | | | | | | sampled for |
| | | | | | | | | | | | | | DNA |
| | | | | | | | | | | | | | extraction |
| OC-01- | OCSCC | 41 | Female | Carcinoma | cT2N2b | Stage IV | pT2N2b | Stage IV | Stage IV | Advanced | 25% | Surgery | |
| 046-NB | positive | | | left buccal | | | | | | | | | |
| | | | | mucosa | | | | | | | | | |
| OC-01- | OCSCC | 59 | Male | Carcinoma | cT2N0M0 | Stage II | pT3N0 | Stage III | Stage III | Advanced | 60% | Surgery | |
| 047-GM | positive | | | tongue | | | | | | | | | |
| OC-01- | OCSCC | 57 | Male | Carcinoma | cT1N0M0 | Stage I | pT2N0 | Stage II | Stage II | Early | 30% | Surgery | |
| 048-MC | positive | | | tongue | | | | | | | | | |
| OC-01- | OCSCC | 48 | Male | Carcinoma | cT4aN0M0 | Stage IV | pT4aN0 | Stage IV | Stage IV | Advanced | 60% | Surgery | |
| 049-NA | positive | | | alveolus | | | | | | | | | |
| OC-01- | OCSCC | 45 | Male | Carcinoma | cT3N3b | Stage IV | pT3N3b | Stage IV | Stage IV | Advanced | 50% | Surgery | |
| 050-MP | positive | | | right buccal | | | | | | | | | |
| | | | | mucosa | | | | | | | | | |
| OC-01- | OCSCC | 48 | Male | Carcinoma | cT4aN1M0 | Stage IV | pT2N0 | Stage II | Stage II | Early | 70% | Surgery | |
| 054-GPC | positive | | | left buccal | | | | | | | | | |
| | | | | mucosa | | | | | | | | | |
| OC-01- | OCSCC | 65 | Female | Carcinoma | cT4bN0M0 | Stage IV | pT3N2a | Stage IV | Stage IV | Advanced | 50% | Biopsy | |
| 055-CA | positive | | | left buccal | | | | | | | | | |
| | | | | mucosa | | | | | | | | | |
| OC-01- | OCSCC | 62 | Male | Carcinoma | cT4aN2M0 | Stage IV | NA | NA | Stage IV | Advanced | 60% | Biopsy | |
| 056-SKG | positive | | | hard palate | | | | | | | | | |
| OC-01- | OCSCC | 60 | Female | Carcinoma | cT2N0M0 | Stage II | NA | NA | Stage II | Early | 50% | Surgery | |
| 057-NM | positive | | | tongue | | | | | | | | | |
| OC-01- | OCSCC | 56 | Female | Carcinoma | cT1N0M0 | Stage I | pT2N0 | Stage II | Stage II | Early | 50% | Surgery | |
| 059-LB | positive | | | tongue | | | | | | | | | |
| OC-01- | OCSCC | 57 | Female | Carcinoma of | cT3N0M0 | Stage III | pT3N2b | Stage IV | Stage IV | Advanced | 80% | Surgery | |
| 060-SJ | positive | | | left upper | | | | | | | | | |
| | | | | alveolus | | | | | | | | | |
| OC-01- | OCSCC | 38 | Male | Carcinoma | cT4aN1M0 | Stage IV | pT2N0 | Stage II | Stage II | Early | 60% | Surgery | |
| 061-MaS | positive | | | right buccal | | | | | | | | | |
| | | | | mucosa | | | | | | | | | |
| OC-01- | OCSCC | 62 | Male | Carcinoma | cT4bN1M0 | Stage IV | pT3N0 | Stage III | Stage III | Advanced | 80% | Surgery | |
| 064-GSH | positive | | | left retro | | | | | | | | | |
| | | | | molar trigone | | | | | | | | | |
| OC-01- | OCSCC | 85 | Female | Carcinoma | cT2N0M0 | Stage II | pT2N0 | Stage II | Stage II | Early | 30% | Surgery | |
| 065-RAM | positive | | | tongue | | | | | | | | | |
| OC-01- | OCSCC | 66 | Male | Carcinoma | cT3N0 | Stage III | pT3N0 | Stage III | Stage III | Advanced | 90% | Surgery | |
| 066-TS | positive | | | maxilla | | | | | | | | | |
| OC-01- | OCSCC | 78 | Male | Carcinoma | cT4aN1M0 | Stage IV | pT3N3b | Stage IV | Stage IV | Advanced | <20% | Surgery | Overall, tumor |
| 068-GG | positive | | | right lower | | | | | | | | | content of |
| | | | | alveolus | | | | | | | | | block is <20%. |
| | | | | | | | | | | | | | Areas |
| | | | | | | | | | | | | | marked as |
| | | | | | | | | | | | | | tumor were |
| | | | | | | | | | | | | | specifically |
| | | | | | | | | | | | | | sampled for |
| | | | | | | | | | | | | | DNA |
| | | | | | | | | | | | | | extraction |
| OC-01- | OCSCC | 57 | Female | Carcinoma | cT4aN2aM0 | Stage IV | pT2N0 | Stage II | Stage II | Early | 60% | Surgery | |
| 070-RA | positive | | | right RMT | | | | | | | | | |
| OC-01- | OCSCC | 40 | Male | Carcinoma | cT2N0M0 | Stage II | pT2N0 | Stage II | Stage II | Early | 60% | Surgery | |
| 071-DT | positive | | | left buccal | | | | | | | | | |
| | | | | mucosa | | | | | | | | | |
| OC-01- | OCSCC | 64 | Female | Carcinoma | cT2N1M0 | Stage III | pT3N2b | Stage IV | Stage IV | Advanced | 60% | Surgery | |
| 072-CH | positive | | | right buccal | | | | | | | | | |
| | | | | mucosa | | | | | | | | | |
| OC-01- | OCSCC | 32 | Male | Carcinoma | cT2N1M0 | Stage III | pT3N0 | Stage III | Stage III | Advanced | 50% | Surgery | |
| 074-AK | positive | | | left lateral | | | | | | | | | |
| | | | | border tongue | | | | | | | | | |
| OC-01- | OCSCC | 52 | Male | Carcinoma | cT4aN0M0 | Stage IV | NA | NA | Stage IV | Advanced | 100% | Biopsy | |
| 075-GST | positive | | | retromolar | | | | | | | | | |
| | | | | trigone | | | | | | | | | |
| OC-01- | OCSCC | 44 | Female | Carcinoma | cT1N3b | Stage IV | NA | NA | Stage IV | Advanced | 50% | Biopsy | |
| 081-RAT | positive | | | tongue | | | | | | | | | |
| OC-02- | OCSCC | 55 | Male | Carcinoma | cT4aN1M0 | Stage IV | pT2N1a | Stage III | Stage III | Advanced | 75% | Surgery | |
| 001-DK | positive | | | left lower GB | | | | | | | | | |
| | | | | sulcus | | | | | | | | | |
| OC-02- | OCSCC | 61 | Male | Carcinoma | cT3N2bM0 | Stage IV | pT2N2b | Stage IV | Stage IV | Advanced | 80% | Surgery | |
| 002-AP | positive | | | right buccal | | | | | | | | | |
| | | | | mucosa | | | | | | | | | |
| OC-02- | OCSCC | 39 | Male | Carcinoma | cT3N2bM0 | Stage IV | pT2N3 | Stage IV | Stage IV | Advanced | 55% | Surgery | |
| 003-BK | positive | | | left buccal | | | | | | | | | |
| | | | | mucosa | | | | | | | | | |
| OC-02- | OCSCC | 47 | Male | Carcinoma | cT2N1M0 | Stage III | pT1N0 | Stage I | Stage I | Early | 70% | Surgery | |
| 004-SuD | positive | | | right buccal | | | | | | | | | |
| | | | | mucosa | | | | | | | | | |
| OC-02- | OCSCC | 51 | Male | Carcinoma | cT4aN2bM0 | Stage IV | pT4aN0 | Stage IV | Stage IV | Advanced | 90% | Surgery | |
| 005-SaD | positive | | | left buccal | | | | | | | | | |
| | | | | mucosa | | | | | | | | | |
| OC-02- | OCSCC | 34 | Male | Carcinoma | cT3N2bM0 | Stage IV | pT3N3b | Stage IV | Stage IV | Advanced | 50% | Surgery | |
| 006-BS | positive | | | tongue right | | | | | | | | | |
| | | | | lateral border | | | | | | | | | |
| OC-02- | OCSCC | 42 | Male | Carcinoma | cT2N0M0 | Stage II | pT3N0 | Stage III | Stage III | Advanced | 50% | Surgery | |
| 007-DP | positive | | | right buccal | | | | | | | | | |
| | | | | mucosa | | | | | | | | | |
| OC-02- | OCSCC | 35 | Male | Carcinoma | cT3N1M0 | Stage III | pT3N2b | Stage IV | Stage IV | Advanced | 70% | Surgery | |
| 009-RS | positive | | | tongue left | | | | | | | | | |
| | | | | lateral border | | | | | | | | | |
| OC-02- | OCSCC | 43 | Male | Carcinoma | cT2N1M0 | Stage III | pT3N0 | Stage III | Stage III | Advanced | 50% | Surgery | |
| 010-BKS | positive | | | tongue | | | | | | | | | |
| OC-02- | OCSCC | 41 | Male | Carcinoma | cT2N1M0 | Stage III | pT1N0 | Stage I | Stage I | Early | 20% | Surgery | |
| 012-MK | positive | | | left buccal | | | | | | | | | |
| | | | | mucosa | | | | | | | | | |
| OC-02- | OCSCC | 32 | Male | Carcinoma | cT4aN2bM0 | Stage IV | pT3N2b | Stage IV | Stage IV | Advanced | 50% | Surgery | |
| 014-RP | positive | | | tongue left | | | | | | | | | |
| | | | | lateral border | | | | | | | | | |
| OC-02- | OCSCC | 36 | Male | Carcinoma | cT3N1M0 | Stage III | pT3N0 | Stage III | Stage III | Advanced | 75% | Surgery | |
| 015-KM | positive | | | left buccal | | | | | | | | | |
| | | | | mucosa | | | | | | | | | |
| OC-02- | OCSCC | 76 | Male | Carcinoma | cT4aN1M0 | Stage IV | pT3N0 | Stage III | Stage III | Advanced | 40% | Surgery | |
| 016-DC | positive | | | tongue | | | | | | | | | |
| OC-02- | OCSCC | 39 | Male | Carcinoma | cT4bN1M0 | Stage IV | pT4aN0 | Stage IV | Stage IV | Advanced | 50% | Surgery | |
| 018-PK | positive | | | right buccal | | | | | | | | | |
| | | | | mucosa | | | | | | | | | |
| OC-02- | OCSCC | 45 | Male | Carcinoma | CT2N1M0 | Stage III | pT4aN0 | Stage IV | Stage IV | Advanced | 70% | Surgery | |
| 019-PJ | positive | | | right buccal | | | | | | | | | |
| | | | | mucosa | | | | | | | | | |
| OC-02- | OCSCC | 60 | Male | Carcinoma | cT4aN1M0 | Stage IV | pT2N0 | Stage II | Stage II | Early | 80% | Surgery | |
| 020-NK | positive | | | lower gum | | | | | | | | | |
| OC-02- | OCSCC | 55 | Female | Carcinoma | cT4aN2cM0 | Stage IV | pT4aN0 | Stage IV | Stage IV | Advanced | 80% | Surgery | |
| 021-BUD | positive | | | anterior lower | | | | | | | | | |
| | | | | gum | | | | | | | | | |
| OC-02- | OCSCC | 49 | Male | Carcinoma | cT3N1M0 | Stage III | pT3N0 | Stage III | Stage III | Advanced | 70% | Surgery | |
| 022-SAB | positive | | | right side | | | | | | | | | |
| | | | | tongue | | | | | | | | | |
| OC-02- | OCSCC | 41 | Male | Carcinoma | cT4bN1M0 | Stage IV | pT3N2a | Stage IV | Stage IV | Advanced | 70% | Surgery | |
| 023-SR | positive | | | left buccal | | | | | | | | | |
| | | | | mucosa | | | | | | | | | |
| OC-02- | OCSCC | 44 | Male | Carcinoma | cT2N0M0 | Stage II | pT2N0 | Stage II | Stage II | Early | 40% | Surgery | |
| 025-RC | positive | | | left side | | | | | | | | | |
| | | | | tongue | | | | | | | | | |
| OC-02- | OCSCC | 36 | Male | Carcinoma | cT4aN1M0 | Stage IV | pT3N1 | Stage III | Stage III | Advanced | 80% | Surgery | |
| 027-SS | positive | | | tongue | | | | | | | | | |
| OC-02- | OCSCC | 68 | Male | Carcinoma | cT4aN1M0 | Stage IV | pT2N0 | Stage II | Stage II | Early | 50% | Surgery | |
| 029-KAM | positive | | | left lower | | | | | | | | | |
| | | | | gum | | | | | | | | | |
| OC-02- | OCSCC | 33 | Male | Carcinoma | cT3N2bM0 | Stage IV | pT3N0 | Stage III | Stage III | Advanced | 80% | Surgery | |
| 030-RB | positive | | | right Buccal | | | | | | | | | |
| | | | | Mucosa | | | | | | | | | |
| OC-02- | OCSCC | 58 | Male | Carcinoma | cT4aN1M0 | Stage IV | pT2N1 | Stage III | Stage III | Advanced | 40% | Surgery | |
| 033-TRM | positive | | | left lower | | | | | | | | | |
| | | | | GBS | | | | | | | | | |
| OC-02- | OCSCC | 68 | Male | Carcinoma | cT4aN1M0 | Stage IV | pT3N0 | Stage III | Stage III | Advanced | 90% | Surgery | |
| 034-US | positive | | | left lower | | | | | | | | | |
| | | | | gum | | | | | | | | | |
| OC-02- | OCSCC | 32 | Male | Carcinoma | cT3N1M0 | Stage III | pT3N0 | Stage III | Stage III | Advanced | 70% | Surgery | |
| 035-PKM | positive | | | right oral | | | | | | | | | |
| | | | | cavity | | | | | | | | | |
| OC-02- | OCSCC | 51 | Male | Carcinoma | cT3N1M0 | Stage III | pT3N2a | Stage IV | Stage IV | Advanced | 70% | Surgery | |
| 036-DAK | positive | | | right buccal | | | | | | | | | |
| | | | | mucosa | | | | | | | | | |
| OC-02- | OCSCC | 64 | Male | Carcinoma | cT4aN1M0 | Stage IV | pT3N0 | Stage III | Stage III | Advanced | 80% | Surgery | |
| 037-BM | positive | | | left hard | | | | | | | | | |
| | | | | palate | | | | | | | | | |
| OC-02- | OCSCC | 36 | Male | Carcinoma | cT4aN1M0 | Stage IV | pT3N0 | Stage III | Stage III | Advanced | 90% | Surgery | |
| 038-AM | positive | | | right lower | | | | | | | | | |
| | | | | gum | | | | | | | | | |
| OC-02- | OCSCC | 55 | Male | Carcinoma | cT3N1M0 | Stage III | pT3N0 | Stage III | Stage III | Advanced | 90% | Surgery | |
| 039-KS | positive | | | right side | | | | | | | | | |
| | | | | tongue | | | | | | | | | |
| OC-02- | OCSCC | 50 | Female | Carcinoma | cT4bN2bM0 | Stage IV | pT3N3b | Stage IV | Stage IV | Advanced | 60% | Surgery | |
| 040-KG | positive | | | left buccal | | | | | | | | | |
| | | | | mucosa | | | | | | | | | |
| OC-02- | OCSCC | 49 | Male | Carcinoma | cT4aN2cM0 | Stage IV | pT2N0 | Stage II | Stage II | Early | 70% | Surgery | |
| 041-GG | positive | | | left lower | | | | | | | | | |
| | | | | gum | | | | | | | | | |
| OC-02- | OCSCC | 43 | Male | Carcinoma | cT3N1M0 | Stage III | pT3N0 | Stage III | Stage III | Advanced | 75% | Surgery | |
| 042-BJ | positive | | | right lateral | | | | | | | | | |
| | | | | border tongue | | | | | | | | | |
| OC-02- | OCSCC | 63 | Male | Carcinoma | cT4aN2bM0 | Stage IV | pT3N3a | Stage IV | Stage IV | Advanced | 60% | Surgery | |
| 043-DCS | positive | | | right | | | | | | | | | |
| | | | | retromolar | | | | | | | | | |
| | | | | trigone | | | | | | | | | |
| OC-02- | OCSCC | 67 | Male | Carcinoma | cT3N0M0 | Stage III | pT3N0 | Stage III | Stage III | Advanced | 50% | Surgery | |
| 044-HB | positive | | | tongue | | | | | | | | | |
| OC-02- | OCSCC | 40 | Male | Carcinoma | cT2N0M0 | Stage II | pT2N2a | Stage IV | Stage IV | Advanced | 40% | Surgery | |
| 045-KCD | positive | | | left buccal | | | | | | | | | |
| | | | | mucosa | | | | | | | | | |
| OC-02- | OCSCC | 69 | Female | Carcinoma | cT4aN2cM0 | Stage IV | pT2N0 | Stage II | Stage II | Early | 60% | Surgery | |
| 050-SN | positive | | | lower gum | | | | | | | | | |
| | | | | (central) | | | | | | | | | |
| OC-02- | OCSCC | 40 | Female | Carcinoma | cT3N1M0 | Stage III | pT3N0 | Stage III | Stage III | Advanced | 70% | Surgery | |
| 055-RJ | positive | | | left | | | | | | | | | |
| | | | | gingvobuccal | | | | | | | | | |
| | | | | sulcus | | | | | | | | | |
| OC-02- | OCSCC | 51 | Male | Carcinoma | cT4bN2bM0 | Stage IV | pT2N1 | Stage III | Stage III | Advanced | 60% | Surgery | |
| 059-GOS | positive | | | right buccal | | | | | | | | | |
| | | | | mucosa | | | | | | | | | |
| OC-02- | OCSCC | 33 | Female | Carcinoma | cT4bN2bM0 | Stage IV | pT3N3 | Stage IV | Stage IV | Advanced | 60% | Surgery | |
| 061-SRO | positive | | | right | | | | | | | | | |
| | | | | retromolar | | | | | | | | | |
| | | | | trigone | | | | | | | | | |
| OC-02- | OCSCC | 35 | Male | Carcinoma | cT3N1M0 | Stage III | pT2N2b | Stage IV | Stage IV | Advanced | 60% | Surgery | |
| 066-AB | positive | | | left buccal | | | | | | | | | |
| | | | | mucosa | | | | | | | | | |
| OC-02- | OCSCC | 62 | Male | Carcinoma | cT2N2bM0 | Stage IV | pT2N1a | Stage III | Stage III | Advanced | 50% | Surgery | |
| 067-KN | positive | | | left | | | | | | | | | |
| | | | | retromolar | | | | | | | | | |
| | | | | trigone | | | | | | | | | |
| OC-02- | OCSCC | 47 | Female | Carcinoma | cT3N2bM0 | Stage IV | pT2N2b | Stage IV | Stage IV | Advanced | 40% | Surgery | |
| 068-NIK | positive | | | left | | | | | | | | | |
| | | | | alveobuccal | | | | | | | | | |
| | | | | sulcus | | | | | | | | | |
| OC-01- | OCSCC | 62 | Male | Carcinoma | cT2N1M0 | Stage III | pT2N0 | Stage II | Stage II | Early | 70% | Surgery | |
| 082-BB | positive | | | tongue | | | | | | | | | |
| OC-01- | OCSCC | 56 | Male | Carcinoma | cT2N0M0 | Stage II | pT2N0 | Stage II | Stage II | Early | 20% | Surgery | |
| 086-DKH | positive | | | left buccal | | | | | | | | | |
| | | | | mucosa | | | | | | | | | |
| OC-01- | OCSCC | 27 | Male | Carcinoma | cT1N0M0 | Stage I | pT1N0 | Stage I | Stage I | Early | <20% | Surgery | Overall, tumor |
| 088-SAA | positive | | | tongue | | | | | | | | | content of |
| | | | | | | | | | | | | | block is <20%. |
| | | | | | | | | | | | | | Areas |
| | | | | | | | | | | | | | marked as |
| | | | | | | | | | | | | | tumor were |
| | | | | | | | | | | | | | specifically |
| | | | | | | | | | | | | | sampled for |
| | | | | | | | | | | | | | DNA |
| | | | | | | | | | | | | | extraction |
| OC-01- | OCSCC | 70 | Female | Carcinoma | cT1N0M0 | Stage I | pT2N0 | Stage II | Stage II | Early | 40% | Surgery | |
| 092-LA | positive | | | lower | | | | | | | | | |
| | | | | gingivobuccal | | | | | | | | | |
| | | | | sulcus | | | | | | | | | |
| OC-01- | OCSCC | 53 | Male | Cariconma | cT1N0M0 | Stage I | pT1N0 | Stage I | Stage I | Early | 30% | Biopsy | |
| 095-LY | positive | | | lower lip | | | | | | | | | |
| OC-01- | OCSCC | 55 | Male | Carcinoma | cT1N0M0 | Stage I | pT1Nx | Stage I | Stage I | Early | <20% | Surgery | Overall, tumor |
| 096-VE | positive | | | Tongue | | | | | | | | | content of |
| | | | | | | | | | | | | | block is <20%. |
| | | | | | | | | | | | | | Areas |
| | | | | | | | | | | | | | marked as |
| | | | | | | | | | | | | | tumor were |
| | | | | | | | | | | | | | specifically |
| | | | | | | | | | | | | | sampled for |
| | | | | | | | | | | | | | DNA |
| | | | | | | | | | | | | | extraction |
| OC-01- | OCSCC | 55 | Female | Carcinoma | cT1N0M0 | Stage I | pT1N0 | Stage I | Stage I | Early | 30% | Surgery | |
| 098- | positive | | | left Buccal | | | | | | | | | |
| MHM | | | | mucosa | | | | | | | | | |
| OC-01- | OCSCC | 38 | Male | Carcinoma | cT2N0M0 | Stage II | pT2N0 | Stage II | Stage II | Early | 30% | Surgery | |
| 100-GNH | positive | | | right side of | | | | | | | | | |
| | | | | tongue | | | | | | | | | |
| OC-01- | OCSCC | 47 | Male | Carcinoma | cT2N0M0 | Stage II | pT2N0 | Stage II | Stage II | Early | 30% | Surgery | |
| 101-JMA | positive | | | left lower | | | | | | | | | |
| | | | | alveolus | | | | | | | | | |
| OC-01- | OCSCC | 72 | Male | Carcinoma | cT2N0M0 | Stage II | pT2N0 | Stage II | Stage II | Early | 30% | Surgery | |
| 102-NV | positive | | | left lower | | | | | | | | | |
| | | | | alveolus | | | | | | | | | |
| OC-01- | OCSCC | 49 | Male | Carcinoma of | cT2N0M0 | Stage II | pT2N0 | Stage II | Stage II | Early | less than 30% | Surgery | |
| 103-PGM | positive | | | left cheek | | | | | | | | | |
| OC-02- | OCSCC | 48 | Male | Carcinoma | cT2N0M0 | Stage II | pT2N0 | Stage II | Stage II | Early | 45% | Surgery | |
| 070-UM | positive | | | right lateral | | | | | | | | | |
| | | | | tongue | | | | | | | | | |
| OC-02- | OCSCC | 35 | Male | Carcinoma | cT2N0M0 | Stage II | pT2N0 | Stage II | Stage II | Early | 40% | Surgery | |
| 071-DIB | positive | | | right buccal | | | | | | | | | |
| | | | | mucosa | | | | | | | | | |
| OC-02- | OCSCC | 50 | Male | Carcinoma | cT1N0M0 | Stage I | pT1N0 | Stage I | Stage I | Early | 30% | Surgery | |
| 073-BAB | positive | | | left buccal | | | | | | | | | |
| | | | | mucosa | | | | | | | | | |
| OC-02- | OCSCC | 56 | Male | carcinoma | cT2N0M0 | Stage II | pT2N0 | Stage II | Stage II | Early | 30% | Surgery | |
| 077-SHU | positive | | | left side | | | | | | | | | |
| | | | | tongue | | | | | | | | | |
| OC-02- | OCSCC | 43 | Male | carcinoma | cT2N0M0 | Stage II | pT1N0 | Stage I | Stage I | Early | 30% | Surgery | |
| 079-PKN | positive | | | left side | | | | | | | | | |
| | | | | tongue | | | | | | | | | |
| OC-03- | OCSCC | 35 | Male | Carcinoma | cT2N0M0 | Stage II | pT2N0M0 | Stage II | Stage II | Early | >50% | Biopsy | |
| 002 | positive | | | Left Tongue | | | | | | | | | |
| OC-03- | OCSCC | 32 | Male | Carcinoma | cT1N0M0 | Stage I | pT1N0M0 | Stage I | Stage I | Early | >50% | Biopsy | |
| 003 | positive | | | Right Tongue | | | | | | | | | |
| OC-03- | OCSCC | 52 | Male | Carcinoma | cT2N0M0 | Stage II | pT2N0M0 | Stage II | Stage II | Early | >50% | Biopsy | |
| 004 | positive | | | Left Tongue | | | | | | | | | |
| OC-03- | OCSCC | 56 | Male | Carcinoma | cT1N0M0 | Stage I | pT2N0M0 | Stage II | Stage II | Early | >50% | Biopsy | |
| 005 | positive | | | Left buccal | | | | | | | | | |
| | | | | mucosa | | | | | | | | | |
| OC-03- | OCSCC | 46 | Female | Carcinoma | cT2N0M0 | Stage II | pT2N0M0 | Stage II | Stage II | Early | >50% | Biopsy | |
| 006 | positive | | | Right Tongue | | | | | | | | | |
| OC-03- | OCSCC | 32 | Male | Carcinoma | cT2N0M0 | Stage II | pT2N0M0 | Stage II | Stage II | Early | >50% | Biopsy | |
| 008 | positive | | | Right Tongue | | | | | | | | | |
| OC-03- | OCSCC | 52 | Male | Carcinoma | cT1N0M0 | Stage I | pT1N0M0 | Stage I | Stage I | Early | >50% | Biopsy | |
| 009 | positive | | | Right buccal | | | | | | | | | |
| | | | | mucosa | | | | | | | | | |
| OC-03- | OCSCC | 25 | Male | Carcinoma | cT1N0M0 | Stage I | pT1N0M0 | Stage I | Stage I | Early | >50% | Biopsy | |
| 010 | positive | | | Left Tongue | | | | | | | | | |
| OC-03- | OCSCC | 40 | Male | Carcinoma | cT1N0M0 | Stage I | pT1N0M0 | Stage I | Stage I | Early | >50% | Biopsy | |
| 011 | positive | | | Right buccal | | | | | | | | | |
| | | | | mucosa | | | | | | | | | |
| OC-03- | OCSCC | 49 | Male | Carcinoma | cT2N0M0 | Stage II | pT2N0M0 | Stage II | Stage II | Early | >50% | Biopsy | |
| 012 | positive | | | Right lower | | | | | | | | | |
| | | | | Lip | | | | | | | | | |
| OC-03- | OCSCC | 47 | Female | Carcinoma | cT2N0M0 | Stage II | pT2N0M0 | Stage II | Stage II | Early | >50% | Biopsy | |
| 013 | positive | | | Left Tongue | | | | | | | | | |
| OC-03- | OCSCC | 41 | Male | Carcinoma | cT2N0M0 | Stage II | pT2N0M0 | Stage II | Stage II | Early | >50% | Biopsy | |
| 014 | positive | | | Right Tongue | | | | | | | | | |
| OC-03- | OCSCC | 38 | Male | Carcinoma | cT1N0M0 | Stage I | pT1N0M0 | Stage I | Stage I | Early | >50% | Biopsy | |
| 015 | positive | | | Left buccal | | | | | | | | | |
| | | | | mucosa | | | | | | | | | |
| OC-03- | OCSCC | 61 | Female | Carcinoma | cT2N0M0 | Stage II | pT2N0M0 | Stage II | Stage II | Early | >50% | Biopsy | |
| 017 | positive | | | Right buccal | | | | | | | | | |
| | | | | mucosa | | | | | | | | | |
| NOC-01- | Healthy | 49 | Female | NA | NA | NA | NA | NA | NA | NA | NA | NA | |
| 004 | controls | | | | | | | | | | | | |
| NOC-01- | Healthy | 44 | Female | NA | NA | NA | NA | NA | NA | NA | NA | NA | |
| 005 | controls | | | | | | | | | | | | |
| NOC-01- | Healthy | 46 | Female | NA | NA | NA | NA | NA | NA | NA | NA | NA | |
| 007 | controls | | | | | | | | | | | | |
| NOC-01- | Healthy | 47 | Male | NA | NA | NA | NA | NA | NA | NA | NA | NA | |
| 010 | controls | | | | | | | | | | | | |
| NOC-01- | Healthy | 40 | Male | NA | NA | NA | NA | NA | NA | NA | NA | NA | |
| 011 | controls | | | | | | | | | | | | |
| NOC-01- | Healthy | 53 | Male | NA | NA | NA | NA | NA | NA | NA | NA | NA | |
| 012 | controls | | | | | | | | | | | | |
| NOC-01- | Healthy | 50 | Male | NA | NA | NA | NA | NA | NA | NA | NA | NA | |
| 013 | controls | | | | | | | | | | | | |
| NOC-01- | Healthy | 43 | Female | NA | NA | NA | NA | NA | NA | NA | NA | NA | |
| 014 | controls | | | | | | | | | | | | |
| NOC-01- | Healthy | 46 | Male | NA | NA | NA | NA | NA | NA | NA | NA | NA | |
| 015 | controls | | | | | | | | | | | | |
| NOC-01- | Healthy | 55 | Male | NA | NA | NA | NA | NA | NA | NA | NA | NA | |
| 020 | controls | | | | | | | | | | | | |
| NOC-01- | Healthy | 44 | Female | NA | NA | NA | NA | NA | NA | NA | NA | NA | |
| 024 | controls | | | | | | | | | | | | |

Supplemental Table S3 Variants in FFPE

TABLE 3A
Details of variants identified in samples

| | NCBI | Hugo | | Start | End | Reference | Tumor | | | |
|---|---|---|---|---|---|---|---|---|---|---|
| Subject ID | Build | Symbol | Chromosome | position | position | Allele | Seq_Allele2 | Variant Classification | Variant Type | Variant |
| OC-02-055-RJ | hg19 | TP53 | chr17 | 7577118 | 7577118 | C | A | Missense_Mutation | SNP | p.Val274Phe |
| OC-02-055-RJ | hg19 | TP53 | chr17 | 7579328 | 7579328 | T | A | Missense_Mutation | SNP | p.Lys120Met |
| OC-02-055-RJ | hg19 | CASP8 | chr2 | 202131411 | 202131411 | C | T | Nonsense_Mutation | SNP | p.Arg68Ter |
| OC-02-055-RJ | hg19 | NOTCH1 | chr9 | 139414006 | 139414006 | G | A | Nonsense_Mutation | SNP | p.Gln252Ter |
| OC-03-005 | hg19 | TP53 | chr17 | 7577120 | 7577120 | C | T | Missense_Mutation | SNP | p.Arg273His |
| OC-03-005 | hg19 | FAT1 | chr4 | 187540784 | 187540784 | G | C | Nonsense_Mutation | SNP | p.Ser2319Ter |
| OC-03-005 | hg19 | FAT1 | chr4 | 187541246 | 187541246 | G | A | Missense_Mutation | SNP | p.Ser2165Leu |
| OC-03-005 | hg19 | NOTCH1 | chr9 | 139405649 | 139405649 | C | A | Nonsense_Mutation | SNP | p.Glu848Ter |
| OC-03-005 | hg19 | CDKN2A | chr9 | 21971116 | 21971116 | G | C | Missense_Mutation | SNP | p.Pro81Arg |
| OC-01-102-NV | hg19 | CASP8 | chr2 | 202137440 | 202137440 | G | A | Missense_Mutation | SNP | p.Cys164Tyr |
| OC-01-102-NV | hg19 | NOTCH1 | chr9 | 139412212 | 139412212 | C | A | Missense_Mutation | SNP | p.Cys478Phe |
| OC-02-038-AM | hg19 | TP53 | chr17 | 7577141 | 7577141 | C | T | Missense_Mutation | SNP | p.Gly266Glu |
| OC-02-038-AM | hg19 | FAT1 | chr4 | 187540677 | 187540678 | C | — | Frame_Shift_Del | DEL | p.Gln2355fs |
| OC-02-038-AM | hg19 | CDKN2A | chr9 | 21971120 | 21971120 | G | A | Nonsense_Mutation | SNP | p.Arg80Ter |
| OC-02-045-KCD | hg19 | HRAS | chr11 | 533874 | 533874 | T | A | Missense_Mutation | SNP | p.Gln61Leu |
| OC-02-045-KCD | hg19 | TP53 | chr17 | 7577094 | 7577094 | G | A | Missense_Mutation | SNP | p.Arg282Trp |
| OC-02-045-KCD | hg19 | TP53 | chr17 | 7578503 | 7578503 | C | T | Missense_Mutation | SNP | p.Val143Met |
| OC-02-045-KCD | hg19 | FAT1 | chr4 | 187518926 | 187518926 | C | T | Missense_Mutation | SNP | p.Cys4093Tyr |
| OC-02-045-KCD | hg19 | FAT1 | chr4 | 187540950 | 187540950 | — | A | Frame_Shift_Ins | INS | p.His2264fs |
| OC-02-045-KCD | hg19 | CDKN2A | chr9 | 21971179 | 21971179 | — | CCACT | Frame_Shift_Ins | INS | p.Ala60fs |
| OC-02-045-KCD | hg19 | CDKN2A | chr9 | 21971179 | 21971179 | — | CCACTA | Frame_Shift_Ins | INS | p.Ala60fs |
| OC-02-068-NIK | hg19 | TP53 | chr17 | 7577538 | 7577538 | C | T | Missense_Mutation | SNP | p.Arg248Gln |
| OC-02-068-NIK | hg19 | CDKN2A | chr9 | 21971028 | 21971028 | C | T | Nonsense_Mutation | SNP | p.Trp110Ter |
| OC-02-036-DAK | hg19 | TP53 | chr17 | 7574018 | 7574018 | G | A | Missense_Mutation | SNP | p.Arg337Cys |
| OC-02-036-DAK | hg19 | TP53 | chr17 | 7577105 | 7577105 | G | C | Missense_Mutation | SNP | p.Pro278Arg |
| OC-02-036-DAK | hg19 | CDKN2A | chr9 | 21971120 | 21971120 | G | A | Nonsense_Mutation | SNP | p.Arg80Ter |
| OC-01-013-SR | hg19 | TP53 | chr17 | 7578413 | 7578413 | C | T | Missense_Mutation | SNP | p.Val173Met |
| OC-01-013-SR | hg19 | CDKN2A | chr9 | 21971186 | 21971186 | G | A | Nonsense_Mutation | SNP | p.Arg58Ter |
| OC-01-014-SU | hg19 | TP53 | chr17 | 7577094 | 7577094 | G | A | Missense_Mutation | SNP | p.Arg282Trp |
| OC-01-014-SU | hg19 | CDKN2A | chr9 | 21974792 | 21974792 | G | T | Nonsense_Mutation | SNP | p.Ser12Ter |
| OC-02-022-SAB | hg19 | HRAS | chr11 | 534289 | 534289 | C | T | Missense_Mutation | SNP | p.Gly12Ser |
| OC-02-022-SAB | hg19 | TP53 | chr17 | 7577121 | 7577121 | G | A | Missense_Mutation | SNP | p.Arg273Cys |
| OC-02-022-SAB | hg19 | TP53 | chr17 | 7578449 | 7578449 | C | T | Missense_Mutation | SNP | p.Ala161Thr |
| OC-02-022-SAB | hg19 | CDKN2A | chr9 | 21971186 | 21971186 | G | A | Nonsense_Mutation | SNP | p.Arg58Ter |
| OC-01-032-SUS | hg19 | TP53 | chr17 | 7578197 | 7578197 | — | CA | Frame_Shift_Ins | INS | p.Val218fs |
| OC-01-032-SUS | hg19 | PIK3CA | chr3 | 178922324 | 178922324 | G | A | Missense_Mutation | SNP | p.Glu365Lys |
| OC-01-032-SUS | hg19 | FAT1 | chr4 | 187630417 | 187630418 | A | — | Frame_Shift_Del | DEL | p.Phe188fs |
| OC-01-032-SUS | hg19 | CDKN2A | chr9 | 21971186 | 21971186 | G | A | Nonsense_Mutation | SNP | p.Arg58Ter |
| OC-01-032-SUS | hg19 | CDKN2A | chr9 | 21971208 | 21971208 | C | T | Splice_Site | SNP | c.151 − 1G > A |
| OC-02-003-BK | hg19 | TP53 | chr17 | 7577094 | 7577094 | G | A | Missense_Mutation | SNP | p.Arg282Trp |
| OC-01-050-MP | hg19 | TP53 | chr17 | 7577539 | 7577539 | G | A | Missense_Mutation | SNP | p.Arg248Trp |
| OC-01-050-MP | hg19 | NOTCH1 | chr9 | 139412645 | 139412645 | C | A | Missense_Mutation | SNP | p.Cys400Phe |
| OC-01-050-MP | hg19 | CDKN2A | chr9 | 21971053 | 21971053 | G | A | Missense_Mutation | SNP | p.Ala102Val |
| OC-01-050-MP | hg19 | CDKN2A | chr9 | 21971120 | 21971120 | G | A | Nonsense_Mutation | SNP | p.Arg80Ter |
| OC-02-007-DP | hg19 | TP53 | chr17 | 7578212 | 7578212 | G | A | Nonsense_Mutation | SNP | p.Arg213Ter |
| OC-02-007-DP | hg19 | FAT1 | chr4 | 187549306 | 187549306 | A | G | Splice_Site | SNP | c.4810 + 2T > C |
| OC-02-007-DP | hg19 | CDKN2A | chr9 | 21971120 | 21971120 | G | A | Nonsense_Mutation | SNP | p.Arg80Ter |
| OC-02-007-DP | hg19 | CDKN2A | chr9 | 21971186 | 21971186 | G | A | Nonsense_Mutation | SNP | p.Arg58Ter |
| OC-01-018-SSR | hg19 | TP53 | chr17 | 7579503 | 7579503 | C | A | Nonsense_Mutation | SNP | p.Glu62Ter |
| OC-01-018-SSR | hg19 | CDKN2A | chr9 | 21971036 | 21971036 | C | G | Missense_Mutation | SNP | p.Asp108His |
| OC-02-016-DC | hg19 | TP53 | chr17 | 7578406 | 7578406 | C | T | Missense_Mutation | SNP | p.Arg175His |
| OC-02-016-DC | hg19 | CDKN2A | chr9 | 21971186 | 21971186 | G | A | Nonsense_Mutation | SNP | p.Arg58Ter |
| OC-01-002-ABR | hg19 | CASP8 | chr2 | 202149832 | 202149832 | C | T | Nonsense_Mutation | SNP | p.Gln366Ter |
| OC-01-002-ABR | hg19 | CDKN2A | chr9 | 21971120 | 21971120 | G | A | Nonsense_Mutation | SNP | p.Arg80Ter |
| OC-01-006-SA | hg19 | TP53 | chr17 | 7578457 | 7578457 | C | A | Missense_Mutation | SNP | p.Arg158Leu |
| OC-01-006-SA | hg19 | CDKN2A | chr9 | 21971120 | 21971120 | G | A | Nonsense_Mutation | SNP | p.Arg80Ter |
| OC-01-029-JR | hg19 | TP53 | chr17 | 7577120 | 7577120 | C | T | Missense_Mutation | SNP | p.Arg273His |
| OC-02-009-RS | hg19 | HRAS | chr1l | 534289 | 534289 | C | T | Missense_Mutation | SNP | p.Gly12Ser |
| OC-02-009-RS | hg19 | TP53 | chr17 | 7576897 | 7576897 | G | A | Nonsense_Mutation | SNP | p.Gln317Ter |
| OC-02-009-RS | hg19 | TP53 | chr17 | 7577130 | 7577130 | A | T | Missense_Mutation | SNP | p.Phe270Ile |
| OC-02-009-RS | hg19 | CASP8 | chr2 | 202149973 | 202149973 | C | T | Nonsense_Mutation | SNP | p.Arg413Ter |
| OC-01-075-GST | hg19 | TP53 | chr17 | 7577144 | 7577144 | A | G | Missense_Mutation | SNP | p.Leu265Pro |
| OC-01-075-GST | hg19 | CASP8 | chr2 | 202141611 | 202141611 | A | G | Missense_Mutation | SNP | p.Asn241Ser |
| OC-01-075-GST | hg19 | NOTCH1 | chr9 | 139399171 | 139399171 | C | A | Nonsense_Mutation | SNP | p.Glu1658Ter |
| OC-01-075-GST | hg19 | NOTCH1 | chr9 | 139402742 | 139402742 | C | T | Nonsense_Mutation | SNP | p.Trp1089Ter |
| OC-02-030-RB | hg19 | TP53 | chr17 | 7579307 | 7579307 | C | A | Splice_Site | SNP | c.375 + 5G > T |
| OC-02-030-RB | hg19 | CDKN2A | chr9 | 21971186 | 21971186 | G | A | Nonsense_Mutation | SNP | p.Arg58Ter |
| OC-03-017 | hg19 | TP53 | chr17 | 7577121 | 7577121 | G | A | Missense_Mutation | SNP | p.Arg273Cys |
| OC-03-017 | hg19 | TP53 | chr17 | 7579313 | 7579313 | G | A | Missense_Mutation | SNP | p.Thr125Met |
| OC-03-002 | hg19 | TP53 | chr17 | 7577539 | 7577539 | G | A | Missense_Mutation | SNP | p.Arg248Trp |
| OC-03-002 | hg19 | CDKN2A | chr9 | 21974695 | 21974695 | G | C | Nonsense_Mutation | SNP | p.Tyr44Ter |
| OC-03-004 | hg19 | TP53 | chr17 | 7579346 | 7579346 | A | T | Nonsense_Mutation | SNP | p.Leu114Ter |
| OC-03-004 | hg19 | FAT1 | chr4 | 187630979 | 187630979 | C | A | Missense_Mutation | SNP | p.Met1Ile |
| OC-03-004 | hg19 | CDKN2A | chr9 | 21971120 | 21971120 | G | A | Nonsense_Mutation | SNP | p.Arg80Ter |
| OC-01-101-JMA | hg19 | CDKN2A | chr9 | 21974792 | 21974792 | G | T | Nonsense_Mutation | SNP | p.Ser12Ter |
| OC-02-037-BM | hg19 | HRAS | chr11 | 534289 | 534289 | C | T | Missense_Mutation | SNP | p.Gly12Ser |
| OC-02-037-BM | hg19 | CASP8 | chr2 | 202137378 | 202137378 | C | A | Missense_Mutation | SNP | p.Phe143Leu |
| OC-02-037-BM | hg19 | CASP8 | chr2 | 202149922 | 202149922 | G | A | Missense_Mutation | SNP | p.Glu396Lys |
| OC-02-037-BM | hg19 | NOTCH1 | chr9 | 139399847 | 139399848 | G | — | Frame_Shift_Del | DEL | p.Phe1500fs |
| OC-01-035-JR | hg19 | TP53 | chr17 | 7577538 | 7577538 | C | T | Missense_Mutation | SNP | p.Arg248Gln |
| OC-01-035-JR | hg19 | FAT1 | chr4 | 187540686 | 187540686 | G | A | Nonsense_Mutation | SNP | p.Gln2352Ter |
| OC-01-035-JR | hg19 | NOTCH1 | chr9 | 139391988 | 139391988 | G | T | Missense_Mutation | SNP | p.Ala2068Asp |
| OC-01-038-GA | hg19 | TP53 | chr17 | 7577085 | 7577085 | C | T | Missense_Mutation | SNP | p.Glu285Lys |
| OC-01-038-GA | hg19 | TP53 | chr17 | 7578290 | 7578290 | C | T | Splice_Site | SNP | c.560 − 1G > A |
| OC-01-038-GA | hg19 | FAT1 | chr4 | 187541617 | 187541617 | — | C | Frame_Shift_Ins | INS | p.Glu2042fs |
| OC-01-019-MA | hg19 | TP53 | chr17 | 7578413 | 7578413 | C | A | Missense_Mutation | SNP | p.Val173Leu |
| OC-02-014-RP | hg19 | HRAS | chr11 | 534285 | 534285 | C | A | Missense_Mutation | SNP | p.Gly13Val |
| OC-02-014-RP | hg19 | TP53 | chr17 | 7578526 | 7578526 | C | A | Missense_Mutation | SNP | p.Cys135Phe |
| OC-02-014-RP | hg19 | FAT1 | chr4 | 187630618 | 187630618 | — | TT | Frame_Shift_Ins | INS | p.Ala122fs |
| OC-01-066-TS | hg19 | TP53 | chr17 | 7578212 | 7578212 | G | A | Nonsense_Mutation | SNP | p.Arg213Ter |
| OC-01-046-NB | hg19 | TP53 | chr17 | 7578504 | 7578505 | G | — | Frame_Shift_Del | DEL | p.Pro142fs |
| OC-01-046-NB | hg19 | CASP8 | chr2 | 202150009 | 202150011 | TT | — | Frame_Shift_Del | DEL | p.Cys426fs |
| OC-02-005-SaD | hg19 | NOTCHI | chr9 | 139400334 | 139400334 | C | G | Splice_Site | SNP | c.4015 − 1G > C |
| OC-01-068-GG | hg19 | TP53 | chr17 | 7577094 | 7577094 | G | C | Missense_Mutation | SNP | p.Arg282Gly |
| OC-01-064-GSH | hg19 | TP53 | chr17 | 7577557 | 7577558 | G | — | Frame_Shift_Del | DEL | p.Cys242fs |
| OC-01-064-GSH | hg19 | PIK3CA | chr3 | 178921549 | 178921549 | T | G | Missense_Mutation | SNP | p.Val344Gly |
| OC-01-072-CH | hg19 | TP53 | chr17 | 7578269 | 7578269 | G | A | Missense_Mutation | SNP | p.Leu194Phe |
| OC-01-072-CH | hg19 | CASP8 | chr2 | 202141689 | 202141689 | C | T | Missense_Mutation | SNP | p.Ala267Val |
| OC-01-072-CH | hg19 | FAT1 | chr4 | 187539071 | 187539071 | G | C | Nonsense_Mutation | SNP | p.Ser2890Ter |
| OC-01-072-CH | hg19 | NOTCH1 | chr9 | 139412252 | 139412252 | C | A | Missense_Mutation | SNP | p.Ala465Ser |
| OC-02-034-US | hg19 | TP53 | chr17 | 7577094 | 7577094 | G | A | Missense_Mutation | SNP | p.Arg282Trp |
| OC-02-034-US | hg19 | TP53 | chr17 | 7579312 | 7579312 | C | T | Missense_Mutation | SNP | p.Thr125Thr |
| OC-02-034-US | hg19 | FAT1 | chr4 | 187539564 | 187539564 | G | A | Nonsense_Mutation | SNP | p.Arg2726Ter |
| OC-02-034-US | hg19 | FAT1 | chr4 | 187629998 | 187630002 | TGAC | — | Frame_Shift_Del | DEL | p.Ser327fs |
| OC-02-044-HB | hg19 | TP53 | chr17 | 7578403 | 7578403 | C | A | Missense_Mutation | SNP | p.Cys176Phe |
| OC-02-042-BJ | hg19 | TP53 | chr17 | 7577568 | 7577568 | C | T | Missense_Mutation | SNP | p.Cys238Tyr |
| OC-01-071-DT | hg19 | TP53 | chr17 | 7578212 | 7578212 | G | A | Nonsense_Mutation | SNP | p.Arg213Ter |
| OC-02-071-DIB | hg19 | TP53 | chr17 | 7577022 | 7577022 | G | A | Nonsense_Mutation | SNP | p.Arg306Ter |
| OC-01-086-DKH | hg19 | TP53 | chr17 | 7577568 | 7577568 | C | T | Missense_Mutation | SNP | p.Cys238Tyr |
| OC-01-086-DKH | hg19 | FAT1 | chr4 | 187539417 | 187539417 | G | A | Nonsense_Mutation | SNP | p.Gln2775Ter |
| OC-03-014 | hg19 | TP53 | chr17 | 7578263 | 7578263 | G | A | Nonsense_Mutation | SNP | p.Arg196Ter |
| OC-02-015-KM | hg19 | TP53 | chr17 | 7577082 | 7577082 | C | T | Missense_Mutation | SNP | p.Glu286Lys |
| OC-02-070-UM | hg19 | HRAS | chr11 | 534285 | 534285 | C | T | Missense_Mutation | SNP | p.Gly13Asp |
| OC-02-070-UM | hg19 | TP53 | chr17 | 7577094 | 7577094 | G | A | Missense_Mutation | SNP | p.Arg282Trp |
| OC-02-040-KG | hg19 | NOTCH1 | chr9 | 139412375 | 139412375 | C | T | Missense_Mutation | SNP | p.Glu424Lys |
| OC-02-002-AP | hg19 | TP53 | chr17 | 7578524 | 7578524 | G | A | Nonsense_Mutation | SNP | p.Gln136Ter |
| OC-02-002-AP | hg19 | NOTCH1 | chr9 | 139412390 | 139412390 | C | G | Splice_Site | SNP | c.1256 − 1G > C |
| OC-01-056-SKG | hg19 | TP53 | chr17 | 7578553 | 7578553 | T | C | Missense_Mutation | SNP | p.Tyr126Cys |
| OC-01-056-SKG | hg19 | FAT1 | chr4 | 187531169 | 187531169 | C | A | Missense_Mutation | SNP | p.Gly3285Val |
| OC-01-056-SKG | hg19 | NOTCH1 | chr9 | 139396889 | 139396890 | C | — | Frame_Shift_Del | DEL | p.Ala1740fs |
| OC-01-056-SKG | hg19 | NOTCH1 | chr9 | 139412375 | 139412375 | C | T | Missense_Mutation | SNP | p.Glu424Lys |
| OC-01-060-SJ | hg19 | TP53 | chr17 | 7577022 | 7577022 | G | A | Nonsense_Mutation | SNP | p.Arg306Ter |
| OC-02-006-BS | hg19 | TP53 | chr17 | 7577120 | 7577120 | C | T | Missense_Mutation | SNP | p.Arg273His |
| OC-01-023-CS | hg19 | CASP8 | chr2 | 202136277 | 202136277 | C | G | Nonsense_Mutation | SNP | p.Ser115Ter |
| OC-01-023-CS | hg19 | NOTCH1 | chr9 | 139405234 | 139405235 | G | — | Frame_Shift_Del | DEL | p.Asn871fs |
| OC-01-020-TSA | hg19 | TP53 | chr17 | 7578263 | 7578263 | G | A | Nonsense_Mutation | SNP | p.Arg196Ter |
| OC-03-012 | hg19 | TP53 | chr17 | 7578532 | 7578534 | TC | — | Frame_Shift_Del | DEL | p.Lys132fs |
| OC-01-028-MD | hg19 | TP53 | chr17 | 7578403 | 7578403 | C | A | Missense_Mutation | SNP | p.Cys176Phe |
| OC-02-059-GOS | hg19 | TP53 | chr17 | 7578272 | 7578272 | G | T | Missense_Mutation | SNP | p.His193Asn |
| OC-02-059-GOS | hg19 | NOTCH1 | chr9 | 139412684 | 139412684 | C | A | Missense_Mutation | SNP | p.Cys387Phe |
| OC-01-034-KS | hg19 | CASP8 | chr2 | 202149973 | 202149973 | C | T | Nonsense_Mutation | SNP | p.Arg413Ter |
| OC-02-018-PK | hg19 | TP53 | chr17 | 7577547 | 7577547 | C | T | Missense_Mutation | SNP | p.Gly245Asp |
| OC-02-061-SRO | hg19 | TP53 | chr17 | 7578441 | 7578441 | G | T | Nonsense_Mutation | SNP | p.Tyr163Ter |
| OC-02-077-SHU | hg19 | TP53 | chr17 | 7578419 | 7578419 | C | A | Nonsense_Mutation | SNP | p.Glu171Ter |
| OC-03-013 | hg19 | TP53 | chr17 | 7579353 | 7579355 | CA | — | Frame_Shift_Del | DEL | p.Leu111fs |
| OC-03-010 | hg19 | TP53 | chr17 | 7578479 | 7578479 | G | A | Missense_Mutation | SNP | p.Pro151Ser |
| OC-02-035-PKM | hg19 | TP53 | chr17 | 7578448 | 7578448 | G | T | Missense_Mutation | SNP | p.Ala161Asp |
| OC-02-035-PKM | hg19 | CASP8 | chr2 | 202131360 | 202131360 | A | T | Nonsense_Mutation | SNP | p.Lys51Ter |
| OC-02-035-PKM | hg19 | FAT1 | chr4 | 187584500 | 187584500 | — | TT | Frame_Shift_Ins | INS | p.Ile1178fs |
| OC-02-035-PKM | hg19 | CDKN2A | chr9 | 21968242 | 21968242 | C | T | Splice_Site | SNP | c.458 − 1G > A |
| OC-03-008 | hg19 | TP53 | chr17 | 7578479 | 7578479 | G | A | Missense_Mutation | SNP | p.Pro151Ser |
| OC-03-008 | hg19 | CDKN2A | chr9 | 21971138 | 21971138 | C | T | Missense_Mutation | SNP | p.Asp74Asn |
| OC-02-021-BUD | hg19 | TP53 | chr17 | 7578375 | 7578379 | CTAT | — | Frame_Shift_Del | DEL | p.Asp184fs |
| OC-01-001-AR | hg19 | FAT1 | chr4 | 187541872 | 187541873 | T | — | Frame_Shift_Del | DEL | p.Asp1956fs |
| OC-01-007-ST | hg19 | CASP8 | chr2 | 202150007 | 202150007 | C | G | Nonsense_Mutation | SNP | p.Ser424Ter |
| OC-01-007-ST | hg19 | NOTCH1 | chr9 | 139412697 | 139412697 | — | TT | Frame_Shift_Ins | INS | p.Glu383fs |
| OC-01-007-ST | hg19 | CDKN2A | chr9 | 21971000 | 21971000 | C | A | Nonsense_Mutation | SNP | p.Glu120Ter |
| OC-01-007-ST | hg19 | TP53 | chr17 | 7577538 | 7577538 | C | T | Missense_Mutation | SNP | p.Arg248Gln |
| OC-01-008-KA | hg19 | HRAS | chr11 | 534289 | 534289 | C | T | Missense_Mutation | SNP | p.Gly12Ser |
| OC-01-008-KA | hg19 | FAT1 | chr4 | 187539097 | 187539099 | TC | — | Frame_Shift_Del | DEL | p.Asp288lfs |
| OC-01-008-KA | hg19 | FAT1 | chr4 | 187541413 | 187541415 | TC | — | Frame_Shift_Del | DEL | p.Asp2109fs |
| OC-01-008-KA | hg19 | NOTCHI | chr9 | 139418164 | 139418164 | C | G | Splice_Site | SNP | c.403 + 5G > C |
| OC-01-008-KA | hg19 | NOTCH1 | chr9 | 139403409 | 139403411 | TG | — | Frame_Shift_Del | DEL | p.Gln1028fs |
| OC-01-025-PC | hg19 | CASP8 | chr2 | 202151181 | 202151181 | G | C | Splice_Site | SNP | c.1305 − 1G > C |
| OC-01-030-RP | hg19 | TP53 | chr17 | 7577526 | 7577526 | A | G | Missense_Mutation | SNP | p.Leu252Pro |
| OC-01-030-RP | hg19 | FAT1 | chr4 | 187521420 | 187521420 | T | A | Missense_Mutation | SNP | p.His3912Leu |
| OC-01-030-RP | hg19 | CDKN2A | chr9 | 21971186 | 21971186 | G | A | Nonsense_Mutation | SNP | p.Arg58Ter |
| OC-01-033-PS | hg19 | TP53 | chr17 | 7578403 | 7578403 | C | A | Missense_Mutation | SNP | p.Cys176Phe |
| OC-01-037-JK | hg19 | CDKN2A | chr9 | 21971120 | 21971120 | G | A | Nonsense_Mutation | SNP | p.Arg80Ter |
| OC-01-040-AJ | hg19 | TP53 | chr17 | 7578537 | 7578538 | T | — | Frame_Shift_Del | DEL | p.Asn131fs |
| OC-01-040-AJ | hg19 | FAT1 | chr4 | 187629360 | 187629360 | G | C | Nonsense_Mutation | SNP | p.Ser541Ter |
| OC-01-040-AJ | hg19 | CDKN2A | chr9 | 21971186 | 21971186 | G | A | Nonsense_Mutation | SNP | p.Arg58Ter |
| OC-01-047-GM | hg19 | TP53 | chr17 | 7577575 | 7577575 | A | C | Missense_Mutation | SNP | p.Tyr236Asp |
| OC-01-047-GM | hg19 | PIK3CA | chr3 | 178922324 | 178922324 | G | A | Missense_Mutation | SNP | p.Glu365Lys |
| OC-01-049-NA | hg19 | HRAS | chr11 | 534289 | 534289 | C | T | Missense_Mutation | SNP | p.Gly12Ser |
| OC-01-049-NA | hg19 | CASP8 | chr2 | 202141587 | 202141587 | G | A | Missense_Mutation | SNP | p.Arg233Gln |
| OC-01-049-NA | hg19 | NOTCH1 | chr9 | 139405104 | 139405104 | C | A | Splice_Site | SNP | c.2740 + 1G > T |
| OC-01-054-GPC | hg19 | CASP8 | chr2 | 202131505 | 202131505 | C | G | Missense_Mutation | SNP | p.Ser99Cys |
| OC-01-054-GPC | hg19 | FAT1 | chr4 | 187541395 | 187541395 | — | GTTTC | Frame_Shift_Ins | INS | p.Val2116fs |
| OC-01-054-GPC | hg19 | NOTCH1 | chr9 | 139403384 | 139403384 | G | A | Nonsense_Mutation | SNP | p.Gln1037Ter |
| OC-01-054-GPC | hg19 | CDKN2A | chr9 | 21971120 | 21971120 | G | A | Nonsense_Mutation | SNP | p.Arg80Ter |
| OC-01-055-CA | hg19 | CDKN2A | chr9 | 21971120 | 21971120 | G | A | Nonsense_Mutation | SNP | p.Arg80Ter |
| OC-01-055-CA | hg19 | FAT1 | chr4 | 187540884 | 187540884 | G | A | Nonsense_Mutation | SNP | p.Gln2286Ter |
| OC-01-059-LB | hg19 | TP53 | chr17 | 7577548 | 7577548 | C | T | Missense_Mutation | SNP | p.Gly245Ser |
| OC-01-061-MaS | hg19 | TP53 | chr17 | 7577121 | 7577121 | G | A | Missense_Mutation | SNP | p.Arg273Cys |
| OC-01-065-RAM | hg19 | TP53 | chr17 | 7577538 | 7577538 | C | T | Missense_Mutation | SNP | p.Arg248Gln |
| OC-01-074-AK | hg19 | TP53 | chr17 | 7578403 | 7578403 | C | T | Missense_Mutation | SNP | p.Cys176Tyr |
| OC-01-074-AK | hg19 | CDKN2A | chr9 | 21971120 | 21971120 | G | A | Nonsense_Mutation | SNP | p.Arg80Ter |
| OC-01-096-VE | hg19 | CDKN2A | chr9 | 21971120 | 21971120 | G | A | Nonsense_Mutation | SNP | p.Arg80Ter |
| OC-02-004-SuD | hg19 | TP53 | chr17 | 7574003 | 7574003 | G | A | Nonsense_Mutation | SNP | p.Arg342Ter |
| OC-02-004-SuD | hg19 | CASP8 | chr2 | 202141586 | 202141586 | C | T | Missense_Mutation | SNP | p.Arg233Trp |
| OC-02-004-SuD | hg19 | FAT1 | chr4 | 187549509 | 187549509 | G | C | Missense_Mutation | SNP | p.Gln1537Glu |
| OC-02-004-SuD | hg19 | NOTCH1 | chr9 | 139402787 | 139402787 | G | T | Nonsense_Mutation | SNP | p.Cys1074Ter |
| OC-02-010-BKS | hg19 | TP53 | chr17 | 7574003 | 7574003 | G | A | Nonsense_Mutation | SNP | p.Arg342Ter |
| OC-02-010-BKS | hg19 | TP53 | chr17 | 7579350 | 7579350 | A | T | Missense_Mutation | SNP | p.Phe113Ile |
| OC-02-012-MK | hg19 | TP53 | chr17 | 7578260 | 7578260 | C | T | Missense_Mutation | SNP | p.Val197Met |
| OC-02-012-MK | hg19 | CASP8 | chr2 | 202141586 | 202141586 | C | T | Missense_Mutation | SNP | p.Arg233Trp |
| OC-02-012-MK | hg19 | FAT1 | chr4 | 187524105 | 187524105 | C | A | Nonsense_Mutation | SNP | p.Glu3812Ter |
| OC-02-012-MK | hg19 | FAT1 | chr4 | 187542073 | 187542075 | AA | — | Frame_Shift_Del | DEL | p.Leu1889fs |
| OC-02-019-PJ | hg19 | TP53 | chr17 | 7577097 | 7577097 | C | G | Missense_Mutation | SNP | p.Asp281His |
| OC-02-019-PJ | hg19 | CASP8 | chr2 | 202136252 | 202136252 | C | T | Nonsense_Mutation | SNP | p.Gln107Ter |
| OC-02-020-NK | hg19 | NOTCH1 | chr9 | 139412735 | 139412739 | ACAG | — | Frame_Shift_Del | DEL | p.Leu369fs |
| OC-02-023-SR | hg19 | HRAS | chr11 | 534285 | 534285 | C | A | Missense_Mutation | SNP | p.Gly13Val |
| OC-02-023-SR | hg19 | TP53 | chr17 | 7577094 | 7577094 | G | A | Missense_Mutation | SNP | p.Arg282Trp |
| OC-02-023-SR | hg19 | TP53 | chr17 | 7577538 | 7577538 | C | T | Missense_Mutation | SNP | p.Arg248Gln |
| OC-02-023-SR | hg19 | CASP8 | chr2 | 202149604 | 202149604 | G | T | Nonsense_Mutation | SNP | p.Glu290Ter |
| OC-02-023-SR | hg19 | FAT1 | chr4 | 187519154 | 187519154 | G | A | Nonsense_Mutation | SNP | p.Gln4077Ter |
| OC-02-023-SR | hg19 | FAT1 | chr4 | 187525133 | 187525133 | T | A | Splice_Site | SNP | c.10549 − 2A > T |
| OC-02-023-SR | hg19 | NOTCH1 | chr9 | 139410069 | 139410069 | — | TCTG | Frame_Shift_Ins | INS | p.Leu590fs |
| OC-02-023-SR | hg19 | NOTCHI | chr9 | 139410069 | 139410069 | — | ATCTG | Frame_Shift_Ins | INS | p.Leu590fs |
| OC-02-023-SR | hg19 | CDKN2A | chr9 | 21971120 | 21971120 | G | A | Nonsense_Mutation | SNP | p.Arg80Ter |
| OC-02-025-RC | hg19 | TP53 | chr17 | 7574003 | 7574003 | G | A | Nonsense_Mutation | SNP | p.Arg342Ter |
| OC-02-025-RC | hg19 | PIK3CA | chr3 | 178952085 | 178952085 | A | G | Missense_Mutation | SNP | p.His1047Arg |
| OC-02-025-RC | hg19 | CDKN2A | chr9 | 21971120 | 21971120 | G | A | Nonsense_Mutation | SNP | p.Arg80Ter |
| OC-02-027-SS | hg19 | TP53 | chr17 | 7577157 | 7577157 | T | C | Splice_Site | SNP | c.783 − 2A > G |
| OC-02-027-SS | hg19 | TP53 | chr17 | 7579546 | 7579546 | — | G | Frame_Shift_Ins | INS | p.Asp48fs |
| OC-02-027-SS | hg19 | CASP8 | chr2 | 202131431 | 202131431 | G | T | Missense_Mutation | SNP | p.Leu74Phe |
| OC-02-029-KAM | hg19 | CASP8 | chr2 | 202150003 | 202150003 | C | T | Nonsense_Mutation | SNP | p.Gln423Ter |
| OC-02-033-TRM | hg19 | FAT1 | chr4 | 187629920 | 187629920 | — | TGAA | Frame_Shift_Ins | INS | p.Val355fs |
| OC-02-033-TRM | hg19 | NOTCH1 | chr9 | 139399362 | 139399362 | C | T | Missense_Mutation | SNP | p.Arg1594Gln |
| OC-02-033-TRM | hg19 | TP53 | chr17 | 7577539 | 7577539 | G | A | Missense_Mutation | SNP | p.Arg248Trp |
| OC-02-043-DCS | hg19 | TP53 | chr17 | 7577513 | 7577513 | — | G | Frame_Shift_Ins | INS | p.Leu257fs |
| OC-02-043-DCS | hg19 | TP53 | chr17 | 7578209 | 7578209 | G | A | Missense_Mutation | SNP | p.His214Tyr |
| OC-02-043-DCS | hg19 | FAT1 | chr4 | 187531170 | 187531170 | C | A | Splice_Site | SNP | c.9854 − 1G > T |
| OC-02-043-DCS | hg19 | NOTCH1 | chr9 | 139413085 | 139413085 | G | A | Missense_Mutation | SNP | p.Arg353Cys |
| OC-02-050-SN | hg19 | CASP8 | chr2 | 202149751 | 202149751 | C | T | Nonsense_Mutation | SNP | p.Gln339Ter |
| OC-02-066-AB | hg19 | TP53 | chr17 | 7577022 | 7577022 | G | A | Nonsense_Mutation | SNP | p.Arg306Ter |
| OC-02-067-KN | hg19 | TP53 | chr17 | 7577098 | 7577098 | T | G | Missense_Mutation | SNP | p.Arg280Ser |
| OC-02-067-KN | hg19 | CDKN2A | chr9 | 21971120 | 21971120 | G | A | Nonsense_Mutation | SNP | p.Arg80Ter |
| OC-03-009 | hg19 | TP53 | chr17 | 7574003 | 7574003 | G | A | Nonsense_Mutation | SNP | p.Arg342Ter |
| OC-03-015 | hg19 | TP53 | chr17 | 7577082 | 7577082 | C | T | Missense_Mutation | SNP | p.Glu286Lys |
| OC-03-015 | hg19 | CDKN2A | chr9 | 21971028 | 21971028 | C | T | Nonsense_Mutation | SNP | p.Trp110Ter |
| OC-03-015 | hg19 | PIK3CA | chr3 | 178952085 | 178952085 | A | G | Missense_Mutation | SNP | p.His1047Arg |
| OC-01-026-GKD | hg19 | TP53 | chr17 | 7578393 | 7578393 | A | T | Missense_Mutation | SNP | p.His179Gln |
| OC-03-005 | hg19 | TP53 | chr17 | 7573009 | 7573009 | C | T | Splice_Site | SNP | c.1101 − 1G > A |
| OC-02-038-AM | hg19 | CASP8 | chr2 | 202151270 | 202151270 | C | T | Nonsense_Mutation | SNP | p.Gln465Ter |
| OC-02-003-BK | hg19 | TP53 | chr17 | 7578503 | 7578503 | C | T | Missense_Mutation | SNP | p.Val143Met |
| OC-01-050-MP | hg19 | CASP8 | chr2 | 202149537 | 202149537 | A | G | Splice_Site | SNP | c.803 − 2A > G |
| OC-02-007-DP | hg19 | TP53 | chr17 | 7574018 | 7574018 | G | A | Missense_Mutation | SNP | p.Arg337Cys |
| OC-01-002-ABR | hg19 | TP53 | chr17 | 7577120 | 7577120 | C | T | Missense_Mutation | SNP | p.Arg273His |
| OC-01-002-ABR | hg19 | TP53 | chr17 | 7578257 | 7578257 | C | A | Nonsense_Mutation | SNP | p.Glu198Ter |
| OC-01-006-SA | hg19 | FAT1 | chr4 | 187549907 | 187549914 | TGATGA | — | Frame_Shift_Del | DEL | p.Phe1443fs |
| OC-02-030-RB | hg19 | FAT1 | chr4 | 187509861 | 187509861 | G | C | Missense_Mutation | SNP | p.Ala4551Gly |
| OC-01-092-LA | hg19 | TP53 | chr17 | 7578394 | 7578394 | T | C | Missense_Mutation | SNP | p.His179Arg |
| OC-01-092-LA | hg19 | FAT1 | chr4 | 187558005 | 187558005 | G | A | Missense_Mutation | SNP | p.Leu1236Phe |
| OC-01-019-MA | hg19 | TP53 | chr17 | 7578440 | 7578440 | T | A | Nonsense_Mutation | SNP | p.Lys164Ter |
| OC-02-014-RP | hg19 | CASP8 | chr2 | 202137429 | 202137431 | AA | — | Frame_Shift_Del | DEL | p.Arg162fs |
| OC-02-042-BJ | hg19 | TP53 | chr17 | 7578457 | 7578457 | C | T | Missense_Mutation | SNP | p.Arg158His |
| OC-02-042-BJ | hg19 | PIK3CA | chr3 | 178952085 | 178952085 | A | G | Missense_Mutation | SNP | p.His1047Arg |
| OC-03-014 | hg19 | FAT1 | chr4 | 187518260 | 187518260 | G | A | Missense_Mutation | SNP | p.Thr4145Met |
| OC-02-070-UM | hg19 | NOTCH1 | chr9 | 139401331 | 139401332 | T | — | Frame_Shift_Del | DEL | p.Asp1246fs |
| OC-02-040-KG | hg19 | CASP8 | chr2 | 202150039 | 202150039 | C | T | Nonsense_Mutation | SNP | p.Arg435Ter |
| OC-02-006-BS | hg19 | TP53 | chr17 | 7574003 | 7574003 | G | A | Nonsense_Mutation | SNP | p.Arg342Ter |
| OC-01-034-KS | hg19 | HRAS | chr11 | 533873 | 533873 | C | A | Missense_Mutation | SNP | p.Gln61His |
| OC-01-034-KS | hg19 | TP53 | chr17 | 7578366 | 7578366 | C | T | Splice_Site | SNP | c.559 + 5G > A |
| OC-01-001-AR | hg19 | TP53 | chr17 | 7578403 | 7578403 | C | A | Missense_Mutation | SNP | p.Cys176Phe |
| OC-01-003-BM | hg19 | TP53 | chr17 | 7577574 | 7577574 | T | C | Missense_Mutation | SNP | p.Tyr236Cys |
| OC-01-007-ST | hg19 | TP53 | chr17 | 7578457 | 7578457 | C | T | Missense_Mutation | SNP | p.Arg158His |
| OC-01-025-PC | hg19 | FAT1 | chr4 | 187584714 | 187584715 | C | — | Frame_Shift_Del | DEL | p.Trp1106fs |
| OC-01-045-PH | hg19 | TP53 | chr17 | 7577593 | 7577595 | AC | — | Frame_Shift_Del | DEL | p.Cys229fs |
| OC-01-048-MC | hg19 | TP53 | chr17 | 7578442 | 7578442 | T | C | Missense_Mutation | SNP | p.Tyr163Cys |
| OC-01-049-NA | hg19 | NOTCH1 | chr9 | 139412389 | 139412389 | C | T | Missense_Mutation | SNP | p.Gly419Asp |
| OC-01-054-GPO | hg19 | TP53 | chr17 | 7574034 | 7574034 | C | T | Splice_Site | SNP | c.994 − 1G > A |
| OC-01-055-CA | hg19 | TP53 | chr17 | 7577093 | 7577093 | C | T | Missense_Mutation | SNP | p.Arg282Gln |
| OC-01-055-CA | hg19 | TP53 | chr17 | 7578366 | 7578366 | C | T | Splice_Site | SNP | c.559 + 5G > A |
| OC-01-055-CA | hg19 | CASP8 | chr2 | 202149705 | 202149705 | C | G | Missense_Mutation | SNP | p.Ile323Met |
| OC-01-055-CA | hg19 | CASP8 | chr2 | 202149735 | 202149735 | C | G | Missense_Mutation | SNP | p.Ile333Met |
| OC-01-055-CA | hg19 | CASP8 | chr2 | 202151193 | 202151193 | T | G | Missense_Mutation | SNP | p.Ile439Ser |
| OC-01-055-CA | hg19 | FAT1 | chr4 | 187527368 | 187527368 | C | G | Splice_Site | SNP | c.10207 − 1G > C |
| OC-01-055-CA | hg19 | NOTCHI | chr9 | 139410521 | 139410521 | G | C | Nonsense_Mutation | SNP | p.Tyr527Ter |
| OC-01-070-RA | hg19 | NOTCH1 | chr9 | 139392000 | 139392000 | G | C | Missense_Mutation | SNP | p.Pro2064Arg |
| OC-01-081-RAT | hg19 | TP53 | chr17 | 7578555 | 7578555 | C | T | Splice_Site | SNP | c.376 − 1G > A |
| OC-02-020-NK | hg19 | HRAS | chr11 | 534288 | 534288 | C | G | Missense_Mutation | SNP | p.Gly12Ala |
| OC-02-020-NK | hg19 | CASP8 | chr2 | 202131515 | 202131515 | G | A | Splice_Site | SNP | c.305 + 1G > A |
| OC-02-020-NK | hg19 | FAT1 | chr4 | 187518900 | 187518900 | C | T | Missense_Mutation | SNP | p.Gly4102Arg |
| OC-02-020-NK | hg19 | FAT1 | chr4 | 187524066 | 187524066 | G | A | Nonsense_Mutation | SNP | p.Gln3825Ter |
| OC-02-020-NK | hg19 | FAT1 | chr4 | 187628418 | 187628418 | C | T | Missense_Mutation | SNP | p.Gly855Glu |
| OC-02-020-NK | hg19 | FAT1 | chr4 | 187628680 | 187628680 | C | T | Missense_Mutation | SNP | p.Asp768Asn |
| OC-02-020-NK | hg19 | NOTCH1 | chr9 | 139413100 | 139413100 | C | T | Missense_Mutation | SNP | p.Ala348Thr |
| OC-02-020-NK | hg19 | NOTCH1 | chr9 | 139417572 | 139417572 | — | G | Frame_Shift_Ins | INS | p.Phe158fs |
| OC-02-025-RC | hg19 | TP53 | chr17 | 7579307 | 7579307 | C | A | Splice_Site | SNP | c.375 + 5G > T |
| OC-02-027-SS | hg19 | FAT1 | chr4 | 187549395 | 187549395 | C | T | Missense_Mutation | SNP | p.Ala1575Thr |
| OC-02-029-KAM | hg19 | TP53 | chr17 | 7578406 | 7578406 | C | T | Missense_Mutation | SNP | p.Arg175His |
| OC-02-029-KAM | hg19 | FAT1 | chr4 | 187530400 | 187530400 | G | C | Missense_Mutation | SNP | p.Ser3381Arg |
| OC-02-029-KAM | hg19 | NOTCH1 | chr9 | 139411722 | 139411722 | A | T | Splice_Site | SNP | c.1555 + 2T > A |
| OC-02-033-TRM | hg19 | FAT1 | chr4 | 187517872 | 187517872 | T | G | Missense_Mutation | SNP | p.Glu4274Asp |
| OC-02-043-DCS | hg19 | CDKN2A | chr9 | 21971123 | 21971125 | GA | — | Frame_Shift_Del | DEL | p.Leu78fs |
| OC-03-011 | hg19 | PIK3CA | chr3 | 178936091 | 178936091 | G | A | Missense_Mutation | SNP | p.Glu545Lys |
| OC-01-024-KB | hg19 | NA | NA | NA | NA | NA | NA | NA | NA | NA |
| OC-01-057-NM | hg19 | NA | NA | NA | NA | NA | NA | NA | NA | NA |
| OC-02-001-DK | hg19 | NA | NA | NA | NA | NA | NA | NA | NA | NA |
| OC-02-039-KS | hg19 | NA | NA | NA | NA | NA | NA | NA | NA | NA |
| OC-02-041-GG | hg19 | NA | NA | NA | NA | NA | NA | NA | NA | NA |
| OC-01-082-BB | hg19 | NA | NA | NA | NA | NA | NA | NA | NA | NA |
| OC-01-088-SAA | hg19 | NA | NA | NA | NA | NA | NA | NA | NA | NA |
| OC-01-095-LY | hg19 | NA | NA | NA | NA | NA | NA | NA | NA | NA |
| OC-01-098-MHM | hg19 | NA | NA | NA | NA | NA | NA | NA | NA | NA |
| OC-01-100-GNH | hg19 | NA | NA | NA | NA | NA | NA | NA | NA | NA |
| OC-01-103-PGM | hg19 | NA | NA | NA | NA | NA | NA | NA | NA | NA |
| OC-02-073-BAB | hg19 | NA | NA | NA | NA | NA | NA | NA | NA | NA |
| OC-02-079-PKN | hg19 | NA | NA | NA | NA | NA | NA | NA | NA | NA |
| OC-03-003 | hg19 | NA | NA | NA | NA | NA | NA | NA | NA | NA |
| OC-03-006 | hg19 | NA | NA | NA | NA | NA | NA | NA | NA | NA |

TABLE 3B
Variant summary by stage and gene

| Gene | Stage I | Stage II | Stage III | Stage IV | Total |
|---|---|---|---|---|---|
| CASP8 | 4 | 5 | 6 | 16 | 31 |
| CDKN2A | 4 | 7 | 10 | 17 | 38 |
| FAT1 | 5 | 14 | 11 | 14 | 44 |
| HRAS | 0 | 3 | 2 | 6 | 11 |
| NOTCH1 | 2 | 11 | 4 | 17 | 34 |
| PIK3CA | 2 | 1 | 3 | 1 | 7 |
| TP53 | 8 | 26 | 31 | 48 | 113 |
| Total | 25 | 67 | 67 | 119 | 278 |
| Samples with variants | 11 | 29 | 24 | 42 | 106 |

Supplemental Table S4. Reproducibility in FFPE

| | | | No. of | | |
|---|---|---|---|---|---|
| | | | Variants | Re- | |
| | Repli- | No. of | Detected in | produc- | |
| Subject ID | cate | Variants | Replicate | ibility | |
| OC-02-021-BUD | R1 | 36 | 36 | 100 | Replicate |
| OC-02-035-PKM | R1 | 21 | 20 | 95.24 | I |
| OC-03-008 | R1 | 24 | 24 | 100 | |
| OC-03-015 | R1 | 29 | 29 | 100 | |
| OC-02-021-BUD | R2 | 36 | 36 | 100 | Replicate |
| OC-02-035-PKM | R2 | 20 | 20 | 100 | II |
| OC-03-008 | R2 | 24 | 24 | 100 | |
| OC-03-015 | R2 | 29 | 29 | 100 | |
| TOTAL | | 219 | 218 | 99.54 | |

Supplemental Table S5. SERASEQ Positive Control % MAF Run-Wise

| | dPCR_AF % | | | | | | | | |
|---|---|---|---|---|---|---|---|---|---|
| | (values from | R01 | R02 | R03 | R04 | R05 | R06 | R07 | R08 |
| Variant | Seracare) | % AF | % AF | % AF | % AF | % AF | % AF | % AF | % AF |
| TP53 p.C242fs*5 | 0.23 | 0.27 | 0.15 | 0.23 | 0.17 | 0.16 | 0.2 | 0.15 | 0.16 |
| PIK3CA | 0.23 | 0.2 | 0.28 | 0.19 | 0.25 | 0.22 | 0.24 | 0.24 | 0.18 |
| p.N1068fs*4 | | | | | | | | | |
| PIK3CA p.E545K | 0.24 | 0.18 | 0.31 | 0.22 | 0.2 | 0.24 | 0.27 | 0.24 | 0.28 |
| PIK3CA p.H1047R | 0.37 | 0.3 | 0.41 | 0.29 | 0.42 | 0.29 | 0.29 | 0.3 | 0.15 |
| TP53 p.R273H | 0.26 | 0.26 | 0.27 | 0.23 | 0.22 | 0.24 | 0.23 | 0.21 | 0.38 |
| TP53 p.R248Q | 0.31 | 0.26 | 0.26 | 0.22 | 0.2 | 0.23 | 0.22 | 0.2 | 0.23 |

| | R01 | R02 | R03 | R04 | R05 | R06 | R07 | R08 |
|---|---|---|---|---|---|---|---|---|
| Variant | TotalReads | TotalReads | TotalReads | TotalReads | TotalReads | TotalReads | TotalReads | TotalReads |
| TP53 p.C242fs*5 | 5890 | 10340 | 5971 | 10547 | 14058 | 12913 | 15487 | 6086 |
| PIK3CA | 7902 | 12754 | 7908 | 13625 | 17893 | 17029 | 19724 | 3336 |
| p.N1068fs*4 | | | | | | | | |
| PIK3CA p.E545K | 7201 | 11762 | 6955 | 12823 | 15984 | 14886 | 18113 | 7278 |
| PIK3CA p.H1047R | 7746 | 10543 | 7140 | 12222 | 17521 | 17357 | 20066 | 7099 |
| TP53 p.R273H | 10617 | 14970 | 10025 | 17996 | 25070 | 24307 | 29222 | 8947 |
| TP53 p.R248Q | 9795 | 14274 | 9449 | 16822 | 23874 | 23100 | 27388 | 8641 |
| TP53 p.R175H | 10617 | 15731 | 9871 | 17257 | 24386 | 24247 | 27798 | 9306 |

Supplemental Table S6. Variants in Saliva

| Sample ID | NCBI_Build | Hugo_Symb | Chromosome | Start_position | End_position | Reference_Allele | Tumor_Seq_Allele2 | Variant_Classification | Variant_Type | Variant |
|---|---|---|---|---|---|---|---|---|---|---|
| OC-01-026-GKD | hg19 | TP53 | chr17 | 7578393 | 7578393 | A | T | Missense_Mutation | SNP | p.His179Gln |
| OC-02-055-RJ | hg19 | TP53 | chr17 | 7577118 | 7577118 | C | A | Missense_Mutation | SNP | p.Val274Phe |
| OC-02-055-RJ | hg19 | TP53 | chr17 | 7579328 | 7579328 | T | A | Missense_Mutation | SNP | p.Lys120Met |
| OC-02-055-RJ | hg19 | CASP8 | chr2 | 202131411 | 202131411 | C | T | Nonsense_Mutation | SNP | p.Arg68Ter |
| OC-02-055-RJ | hg19 | NOTCH1 | chr9 | 139414006 | 139414006 | G | A | Nonsense_Mutation | SNP | p.Gln252Ter |
| OC-03-005 | hg19 | TP53 | chr17 | 7577120 | 7577120 | C | T | Missense_Mutation | SNP | p.Arg273His |
| OC-03-005 | hg19 | FAT1 | chr4 | 187540784 | 187540784 | G | C | Nonsense_Mutation | SNP | p.Ser2319Ter |
| OC-03-005 | hg19 | FAT | chr4 | 187541246 | 187541246 | G | A | Missense_Mutation | SNP | p.Ser2165Leu |
| OC-03-005 | hg19 | NOTCH1 | chr9 | 139405649 | 139405649 | C | A | Nonsense_Mutation | SNP | p.Glu848Ter |
| OC-03-005 | hg19 | CDKN2A | chr9 | 21971116 | 21971116 | G | C | Missense_Mutation | SNP | p.Pro81Arg |
| OC-01-102-NV | hg19 | CASP8 | chr2 | 202137440 | 202137440 | G | A | Missense_Mutation | SNP | p.Cys164Tyr |
| OC-01-102-NV | hg19 | NOTCH1 | chr9 | 139412212 | 139412212 | C | A | Missense_Mutation | SNP | p.Cys478Phe |
| OC-02-038-AM | hg19 | TP53 | chr17 | 7577141 | 7577141 | C | T | Missense_Mutation | SNP | p.Gly266Glu |
| OC-02-038-AM | hg19 | FAT1 | chr4 | 187540677 | 187540678 | C | — | Frame_Shift_Del | DEL | p.Gln2355fs |
| OC-02-038-AM | hg19 | CDKN2A | chr9 | 21971120 | 21971120 | G | A | Nonsense_Mutation | SNP | p.Arg80Ter |
| OC-02-045-KCD | hg19 | HRAS | chr11 | 533874 | 533874 | T | A | Missense_Mutation | SNP | p.Gln61Leu |
| OC-02-045-KCD | hg19 | TP53 | chr17 | 7577094 | 7577094 | G | A | Missense_Mutation | SNP | p.Arg282Trp |
| OC-02-045-KCD | hg19 | TP53 | chr17 | 7578503 | 7578503 | C | T | Missense_Mutation | SNP | p.Val143Met |
| OC-02-045-KCD | hg19 | FAT1 | chr4 | 187518926 | 187518926 | C | T | Missense_Mutation | SNP | p.Cys4093Tyr |
| OC-02-045-KCD | hg19 | FAT | chr4 | 187540950 | 187540950 | — | A | Frame_Shift_Ins | INS | p.His2264fs |
| OC-02-045-KCD | hg19 | CDKN2A | chr9 | 21971179 | 21971179 | — | CCACT | Frame_Shift_Ins | INS | p.Ala60fs |
| OC-02-045-KCD | hg19 | CDKN2A | chr9 | 21971179 | 21971179 | — | CCACTA | Frame_Shift_Ins | INS | p.Ala60fs |
| OC-02-068-NIK | hg19 | TP53 | chr17 | 7577538 | 7577538 | C | T | Missense_Mutation | SNP | p.Arg248Gln |
| OC-02-068-NIK | hg19 | CDKN2A | chr9 | 21971028 | 21971028 | C | T | Nonsense_Mutation | SNP | p.Trp110Ter |
| OC-02-036-DAK | hg19 | TP53 | chr17 | 7574018 | 7574018 | G | A | Missense_Mutation | SNP | p.Arg337Cys |
| OC-02-036-DAK | hg19 | TP53 | chr17 | 7577105 | 7577105 | G | C | Missense_Mutation | SNP | p.Pro278Arg |
| OC-02-036-DAK | hg19 | CDKN2A | chr9 | 21971120 | 21971120 | G | A | Nonsense_Mutation | SNP | p.Arg80Ter |
| OC-01-013-SR | hg19 | TP53 | chr17 | 7578413 | 7578413 | C | T | Missense_Mutation | SNP | p.Val173Met |
| OC-01-013-SR | hg19 | CDKN2A | chr9 | 21971186 | 21971186 | G | A | Nonsense_Mutation | SNP | p.Arg58Ter |
| OC-01-014-SU | hg19 | TP53 | chr17 | 7577094 | 7577094 | G | A | Missense_Mutation | SNP | p.Arg282Trp |
| OC-01-014-SU | hg19 | CDKN2A | chr9 | 21974792 | 21974792 | G | T | Nonsense_Mutation | SNP | p.Ser12Ter |
| OC-02-022-SAB | hg19 | HRAS | chr11 | 534289 | 534289 | C | T | Missense_Mutation | SNP | p.Gly12Ser |
| OC-02-022-SAB | hg19 | TP53 | chr17 | 7577121 | 7577121 | G | A | Missense_Mutation | SNP | p.Arg273Cys |
| OC-02-022-SAB | hg19 | TP53 | chr17 | 7578449 | 7578449 | C | T | Missense_Mutation | SNP | p.Ala161Thr |
| OC-02-022-SAB | hg19 | CDKN2A | chr9 | 21971186 | 21971186 | G | A | Nonsense_Mutation | SNP | p.Arg58Ter |
| OC-01-032-SUS | hg19 | TP53 | chr17 | 7578197 | 7578197 | — | CA | Frame_Shift_Ins | INS | p.Val218fs |
| OC-01-032-SUS | hg19 | PIK3CA | chr3 | 178922324 | 178922324 | G | A | Missense_Mutation | SNP | p.Glu365Lys |
| OC-01-032-SUS | hg19 | FAT1 | chr4 | 187630417 | 187630418 | A | — | Frame_Shift_Del | DEL | p.Phe188fs |
| OC-01-032-SUS | hg19 | CDKN2A | chr9 | 21971186 | 21971186 | G | A | Nonsense_Mutation | SNP | p.Arg58Ter |
| OC-01-032-SUS | hg19 | CDKN2A | chr9 | 21971208 | 21971208 | C | T | Splice_Site | SNP | c.151 − 1G > A |
| OC-02-003-BK | hg19 | TP53 | chr17 | 7577094 | 7577094 | G | A | Missense_Mutation | SNP | p.Arg282Trp |
| OC-02-003-BK | hg19 | CDKN2A | chr9 | 21971111 | 21971111 | G | A | Missense_Mutation | SNP | p.His83Tyr |
| OC-01-050-MP | hg19 | TP53 | chr17 | 7577539 | 7577539 | G | A | Missense_Mutation | SNP | p.Arg248Trp |
| OC-01-050-MP | hg19 | NOTCH1 | chr9 | 139412645 | 139412645 | C | A | Missense_Mutation | SNP | p.Cys400Phe |
| OC-01-050-MP | hg19 | CDKN2A | chr9 | 21971053 | 21971053 | G | A | Missense_Mutation | SNP | p.Ala102Val |
| OC-01-050-MP | hg19 | CDKN2A | chr9 | 21971120 | 21971120 | G | A | Nonsense_Mutation | SNP | p.Arg80Ter |
| OC-02-007-DP | hg19 | TP53 | chr17 | 7578212 | 7578212 | G | A | Nonsense_Mutation | SNP | p.Arg213Ter |
| OC-02-007-DP | hg19 | FAT1 | chr4 | 187549306 | 187549306 | A | G | Splice_Site | SNP | c.4810 + 2T > C |
| OC-02-007-DP | hg19 | CDKN2A | chr9 | 21971120 | 21971120 | G | A | Nonsense_Mutation | SNP | p.Arg80Ter |
| OC-02-007-DP | hg19 | CDKN2A | chr9 | 21971186 | 21971186 | G | A | Nonsense_Mutation | SNP | p.Arg58Ter |
| OC-01-018-SSR | hg19 | TP53 | chr17 | 7579503 | 7579503 | C | A | Nonsense_Mutation | SNP | p.Glu62Ter |
| OC-01-018-SSR | hg19 | CDKN2A | chr9 | 21971036 | 21971036 | C | G | Missense_Mutation | SNP | p.Asp108His |
| OC-02-016-DC | hg19 | TP53 | chr17 | 7578406 | 7578406 | C | T | Missense_Mutation | SNP | p.Arg175His |
| OC-02-016-DC | hg19 | CDKN2A | chr9 | 21971186 | 21971186 | G | A | Nonsense_Mutation | SNP | p.Arg58Ter |
| OC-01-002-ABR | hg19 | CASP8 | chr2 | 202149832 | 202149832 | C | T | Nonsense_Mutation | SNP | p.Gln366Ter |
| OC-01-002-ABR | hg19 | CDKN2A | chr9 | 21971120 | 21971120 | G | A | Nonsense_Mutation | SNP | p.Arg80Ter |
| OC-01-006-SA | hg19 | TP53 | chr17 | 7578457 | 7578457 | C | A | Missense_Mutation | SNP | p.Arg158Leu |
| OC-01-006-SA | hg19 | CDKN2A | chr9 | 21971120 | 21971120 | G | A | Nonsense_Mutation | SNP | p.Arg80Ter |
| OC-01-029-JR | hg19 | TP53 | chr17 | 7577120 | 7577120 | C | T | Missense_Mutation | SNP | p.Arg273His |
| OC-02-009-RS | hg19 | HRAS | chr11 | 534289 | 534289 | C | T | Missense_Mutation | SNP | p.Gly12Ser |
| OC-02-009-RS | hg19 | TP53 | chr17 | 7576897 | 7576897 | G | A | Nonsense_Mutation | SNP | p.Gln317Ter |
| OC-02-009-RS | hg19 | TP53 | chr17 | 7577130 | 7577130 | A | T | Missense_Mutation | SNP | p.Phe270Ile |
| OC-02-009-RS | hg19 | CASP8 | chr2 | 202149973 | 202149973 | C | T | Nonsense_Mutation | SNP | p.Arg413Ter |
| OC-01-075-GST | hg19 | TP53 | chr17 | 7577144 | 7577144 | A | G | Missense_Mutation | SNP | p.Leu265Pro |
| OC-01-075-GST | hg19 | CASP8 | chr2 | 202141611 | 202141611 | A | G | Missense_Mutation | SNP | p.Asn241Ser |
| OC-01-075-GST | hg19 | NOTCH1 | chr9 | 139399171 | 139399171 | C | A | Nonsense_Mutation | SNP | p.Glu1658Ter |
| OC-01-075-GST | hg19 | NOTCH1 | chr9 | 139402742 | 139402742 | C | T | Nonsense_Mutation | SNP | p.Trp1089Ter |
| OC-02-030-RB | hg19 | TP53 | chr17 | 7579307 | 7579307 | C | A | Splice_Site | SNP | c.375 + 5G > T |
| OC-02-030-RB | hg19 | CDKN2A | chr9 | 21971186 | 21971186 | G | A | Nonsense_Mutation | SNP | p.Arg58Ter |
| OC-03-017 | hg19 | TP53 | chr17 | 7577121 | 7577121 | G | A | Missense_Mutation | SNP | p.Arg273Cys |
| OC-03-017 | hg19 | TP53 | chr17 | 7579313 | 7579313 | G | A | Missense_Mutation | SNP | p.Thr125Met |
| OC-03-002 | hg19 | TP53 | chr17 | 7577539 | 7577539 | G | A | Missense_Mutation | SNP | p.Arg248Trp |
| OC-03-002 | hg19 | CDKN2A | chr9 | 21974695 | 21974695 | G | C | Nonsense_Mutation | SNP | p.Tyr44Ter |
| OC-03-004 | hg19 | TP53 | chr17 | 7579346 | 7579346 | A | T | Nonsense_Mutation | SNP | p.Leu114Ter |
| OC-03-004 | hg19 | FAT1 | chr4 | 187630979 | 187630979 | C | A | Missense_Mutation | SNP | p.Met1Ile |
| OC-03-004 | hg19 | CDKN2A | chr9 | 21971120 | 21971120 | G | A | Nonsense_Mutation | SNP | p.Arg80Ter |
| OC-01-101-JMA | hg19 | CDKN2A | chr9 | 21974792 | 21974792 | G | T | Nonsense_Mutation | SNP | p.Ser12Ter |
| OC-02-037-BM | hg19 | HRAS | chr11 | 534289 | 534289 | C | T | Missense_Mutation | SNP | p.Gly12Ser |
| OC-02-037-BM | hg19 | CASP8 | chr2 | 202137378 | 202137378 | C | A | Missense_Mutation | SNP | p.Phe143Leu |
| OC-02-037-BM | hg19 | CASP8 | chr2 | 202149922 | 202149922 | G | A | Missense_Mutation | SNP | p.Glu396Lys |
| OC-02-037-BM | hg19 | NOTCH1 | chr9 | 139399847 | 139399848 | G | — | Frame_Shift_Del | DEL | p.Phe1500fs |
| OC-01-035-JR | hg19 | TP53 | chr17 | 7577538 | 7577538 | C | T | Missense_Mutation | SNP | p.Arg248Gln |
| OC-01-035-JR | hg19 | FAT1 | chr4 | 187540686 | 187540686 | G | A | Nonsense_Mutation | SNP | p.Gln2352Ter |
| OC-01-035-JR | hg19 | NOTCH1 | chr9 | 139391988 | 139391988 | G | T | Missense_Mutation | SNP | p.Ala2068As |
| OC-01-038-GA | hg19 | TP53 | chr17 | 7577085 | 7577085 | C | T | Missense_Mutation | SNP | p.Glu285Lys |
| OC-01-038-GA | hg19 | TP53 | chr17 | 7578290 | 7578290 | C | T | Splice_Site | SNP | c.560 − 1G > A |
| OC-01-038-GA | hg19 | FAT1 | chr4 | 187541617 | 187541617 | — | C | Frame_Shift_Ins | INS | p.Glu2042fs |
| OC-01-019-MA | hg19 | TP53 | chr17 | 7578413 | 7578413 | C | A | Missense_Mutation | SNP | p.Val173Leu |
| OC-02-014-RP | hg19 | HRAS | chr11 | 534285 | 534285 | C | A | Missense_Mutation | SNP | p.Gly13Val |
| OC-02-014-RP | hg19 | TP53 | chr17 | 7578526 | 7578526 | C | A | Missense_Mutation | SNP | p.Cys135Phe |
| OC-02-014-RP | hg19 | FAT1 | chr4 | 187630618 | 187630618 | — | TT | Frame_Shift_Ins | INS | p.Ala122fs |
| OC-01-066-TS | hg19 | TP53 | chr17 | 7578212 | 7578212 | G | A | Nonsense_Mutation | SNP | p.Arg213Ter |
| OC-01-046-NB | hg19 | TP53 | chr17 | 7578504 | 7578505 | G | — | Frame_Shift_Del | DEL | p.Pro142fs |
| OC-01-046-NB | hg19 | CASP8 | chr2 | 202150009 | 202150011 | TT | — | Frame_Shift_Del | DEL | p.Cys426fs |
| OC-02-005-SaD | hg19 | NOTCH1 | chr9 | 139400334 | 139400334 | C | G | Splice_Site | SNP | c.4015 − 1G > C |
| OC-01-068-GG | hg19 | TP53 | chr17 | 7577094 | 7577094 | G | C | Missense_Mutation | SNP | p.Arg282Gly |
| OC-01-064-GSH | hg19 | TP53 | chr17 | 7577557 | 7577558 | G | — | Frame_Shift_Del | DEL | p.Cys242fs |
| OC-01-064-GSH | hg19 | PIK3CA | chr3 | 178921549 | 178921549 | T | G | Missense_Mutation | SNP | p.Val344Gly |
| OC-01-072-CH | hg19 | TP53 | chr17 | 7578269 | 7578269 | G | A | Missense_Mutation | SNP | p.Leu194Phe |
| OC-01-072-CH | hg19 | CASP8 | chr2 | 202141689 | 202141689 | C | T | Missense_Mutation | SNP | p.Ala267Val |
| OC-01-072-CH | hg19 | FAT1 | chr4 | 187539071 | 187539071 | G | C | Nonsense_Mutation | SNP | p.Ser2890Ter |
| OC-01-072-CH | hg19 | NOTCH1 | chr9 | 139412252 | 139412252 | C | A | Missense_Mutation | SNP | p.Ala465Ser |
| OC-02-034-US | hg19 | TP53 | chr17 | 7577094 | 7577094 | G | A | Missense_Mutation | SNP | p.Arg282Trp |
| OC-02-034-US | hg19 | TP53 | chr17 | 7579312 | 7579312 | C | T | Missense_Mutation | SNP | p.Thr125Thr |
| OC-02-034-US | hg19 | FAT1 | chr4 | 187539564 | 187539564 | G | A | Nonsense_Mutation | SNP | p.Arg2726Ter |
| OC-02-034-US | hg19 | FAT1 | chr4 | 187629998 | 187630002 | TGAC | — | Frame_Shift_Del | DEL | p.Ser327fs |
| OC-02-044-HB | hg19 | TP53 | chr17 | 7578403 | 7578403 | C | A | Missense_Mutation | SNP | p.Cys176Phe |
| OC-02-042-BJ | hg19 | TP53 | chr17 | 7577568 | 7577568 | C | T | Missense_Mutation | SNP | p.Cys238Tyr |
| OC-01-071-DT | hg19 | TP53 | chr17 | 7578212 | 7578212 | G | A | Nonsense_Mutation | SNP | p.Arg213Ter |
| OC-02-071-DIB | hg19 | TP53 | chr17 | 7577022 | 7577022 | G | A | Nonsense_Mutation | SNP | p.Arg306Ter |
| OC-01-086-DKH | hg19 | TP53 | chr17 | 7577568 | 7577568 | C | T | Missense_Mutation | SNP | p.Cys238Tyr |
| OC-01-086-DKH | hg19 | FAT1 | chr4 | 187539417 | 187539417 | G | A | Nonsense_Mutation | SNP | p.Gln2775Ter |
| OC-03-014 | hg19 | TP53 | chr17 | 7578263 | 7578263 | G | A | Nonsense_Mutation | SNP | p.Arg196Ter |
| OC-02-015-KM | hg19 | TP53 | chr17 | 7577082 | 7577082 | C | T | Missense_Mutation | SNP | p.Glu286Lys |
| OC-02-070-UM | hg19 | HRAS | chr11 | 534285 | 534285 | C | T | Missense_Mutation | SNP | p.Gly13Asp |
| OC-02-070-UM | hg19 | TP53 | chr17 | 7577094 | 7577094 | G | A | Missense_Mutation | SNP | p.Arg282Trp |
| OC-02-040-KG | hg19 | NOTCH1 | chr9 | 139412375 | 139412375 | C | T | Missense_Mutation | SNP | p.Glu424Lys |
| OC-02-002-AP | hg19 | TP53 | chr17 | 7578524 | 7578524 | G | A | Nonsense_Mutation | SNP | p.Gln136Ter |
| OC-02-002-AP | hg19 | NOTCH1 | chr9 | 139412390 | 139412390 | C | G | Splice_Site | SNP | c.1256 − 1G > C |
| OC-01-056-SKG | hg19 | TP53 | chr17 | 7578553 | 7578553 | T | C | Missense_Mutation | SNP | p.Tyr126Cys |
| OC-01-056-SKG | hg19 | FAT1 | chr4 | 187531169 | 187531169 | C | A | Missense_Mutation | SNP | p.Gly3285Val |
| OC-01-056-SKG | hg19 | NOTCH1 | chr9 | 139396889 | 139396890 | C | — | Frame_Shift_Del | DEL | p.Ala1740fs |
| OC-01-056-SKG | hg19 | NOTCH1 | chr9 | 139412375 | 139412375 | C | T | Missense_Mutation | SNP | p.Glu424Lys |
| OC-01-060-SJ | hg19 | TP53 | chr17 | 7577022 | 7577022 | G | A | Nonsense_Mutation | SNP | p.Arg306Ter |
| OC-02-006-BS | hg19 | TP53 | chr17 | 7577120 | 7577120 | C | T | Missense_Mutation | SNP | p.Arg273His |
| OC-01-023-CS | hg19 | CASP8 | chr2 | 202136277 | 202136277 | C | G | Nonsense_Mutation | SNP | p.Ser115Ter |
| OC-01-023-CS | hg19 | NOTCH1 | chr9 | 139405234 | 139405235 | G | — | Frame_Shift_Del | DEL | p.Asn871fs |
| OC-01-020-TSA | hg19 | TP53 | chr17 | 7578263 | 7578263 | G | A | Nonsense_Mutation | SNP | p.Arg196Ter |
| OC-03-012 | hg19 | TP53 | chr17 | 7578532 | 7578534 | TC | — | Frame_Shift_Del | DEL | p.Lys132fs |
| OC-01-028-MD | hg19 | TP53 | chr17 | 7578403 | 7578403 | C | A | Missense_Mutation | SNP | p.Cys176Phe |
| OC-02-059-GOS | hg19 | TP53 | chr17 | 7578272 | 7578272 | G | T | Missense_Mutation | SNP | p.His193Asn |
| OC-02-059-GOS | hg19 | NOTCH1 | chr9 | 139412684 | 139412684 | C | A | Missense_Mutation | SNP | p.Cys387Phe |
| OC-01-034-KS | hg19 | CASP8 | chr2 | 202149973 | 202149973 | C | T | Nonsense_Mutation | SNP | p.Arg413Ter |
| OC-02-018-PK | hg19 | TP53 | chr17 | 7577547 | 7577547 | C | T | Missense_Mutation | SNP | p.Gly245Asp |
| OC-02-061-SRO | hg19 | TP53 | chr17 | 7578441 | 7578441 | G | T | Nonsense_Mutation | SNP | p.Tyr163Ter |
| OC-02-077-SHU | hg19 | TP53 | chr17 | 7578419 | 7578419 | C | A | Nonsense_Mutation | SNP | p.Glu171Ter |
| OC-03-013 | hg19 | TP53 | chr17 | 7579353 | 7579355 | CA | — | Frame_Shift_Del | DEL | p.Leu111fs |
| OC-03-010 | hg19 | TP53 | chr17 | 7578479 | 7578479 | G | A | Missense_Mutation | SNP | p.Pro151Ser |
| OC-02-035-PKM | hg19 | TP53 | chr17 | 7578448 | 7578448 | G | T | Missense_Mutation | SNP | p.Ala161Asp |
| OC-02-035-PKM | hg19 | CASP8 | chr2 | 202131360 | 202131360 | A | T | Nonsense_Mutation | SNP | p.Lys51Ter |
| OC-02-035-PKM | hg19 | FAT1 | chr4 | 187584500 | 187584500 | — | TT | Frame_Shift_Ins | INS | p.Ile1178fs |
| OC-02-035-PKM | hg19 | CDKN2A | chr9 | 21968242 | 21968242 | C | T | Splice_Site | SNP | c.458 − 1G > A |
| OC-03-008 | hg19 | TP53 | chr17 | 7578479 | 7578479 | G | A | Missense_Mutation | SNP | p.Pro151Ser |
| OC-03-008 | hg19 | CDKN2A | chr9 | 21971138 | 21971138 | C | T | Missense_Mutation | SNP | p.Asp74Asn |
| OC-02-021-BUD | hg19 | TP53 | chr17 | 7578375 | 7578379 | CTAT | — | Frame_Shift_Del | DEL | p.Asp184fs |
| OC-01-001-AR | hg19 | FAT1 | chr4 | 187541872 | 187541873 | T | — | Frame_Shift_Del | DEL | p.Asp1956fs |
| OC-01-007-ST | hg19 | CASP8 | chr2 | 202150007 | 202150007 | C | G | Nonsense_Mutation | SNP | p.Ser424Ter |
| OC-01-007-ST | hg19 | NOTCH1 | chr9 | 139412697 | 139412697 | — | TT | Frame_Shift_Ins | INS | p.Glu383fs |
| OC-01-007-ST | hg19 | CDKN2A | chr9 | 21971000 | 21971000 | C | A | Nonsense_Mutation | SNP | p.Glu120Ter |
| OC-01-007-ST | hg19 | TP53 | chr17 | 7577538 | 7577538 | C | T | Missense_Mutation | SNP | p.Arg248Gln |
| OC-01-008-KA | hg19 | HRAS | chr11 | 534289 | 534289 | C | T | Missense_Mutation | SNP | p.Gly12Ser |
| OC-01-008-KA | hg19 | FAT1 | chr4 | 187539097 | 187539099 | TC | — | Frame_Shift_Del | DEL | p.Asp2881fs |
| OC-01-008-KA | hg19 | FAT1 | chr4 | 187541413 | 187541415 | TC | — | Frame_Shift_Del | DEL | p.Asp2109fs |
| OC-01-008-KA | hg19 | NOTCH1 | chr9 | 139418164 | 139418164 | C | G | Splice_Site | SNP | c.403 + 5G > C |
| OC-01-008-KA | hg19 | NOTCH1 | chr9 | 139403409 | 139403411 | TG | — | Frame_Shift_Del | DEL | p.Gln1028fs |
| OC-01-025-PC | hg19 | CASP8 | chr2 | 202151181 | 202151181 | G | C | Splice_Site | SNP | c.1305 − 1G > C |
| OC-01-030-RP | hg19 | TP53 | chr17 | 7577526 | 7577526 | A | G | Missense_Mutation | SNP | p.Leu252Pro |
| OC-01-030-RP | hg19 | FAT1 | chr4 | 187521420 | 187521420 | T | A | Missense_Mutation | SNP | p.His3912Leu |
| OC-01-030-RP | hg19 | CDKN2A | chr9 | 21971186 | 21971186 | G | A | Nonsense_Mutation | SNP | p.Arg58Ter |
| OC-01-033-PS | hg19 | TP53 | chr17 | 7578403 | 7578403 | C | A | Missense_Mutation | SNP | p.Cys176Phe |
| OC-01-037-JK | hg19 | CDKN2A | chr9 | 21971120 | 21971120 | G | A | Nonsense_Mutation | SNP | p.Arg80Ter |
| OC-01-040-AJ | hg19 | TP53 | chr17 | 7578537 | 7578538 | T | — | Frame_Shift_Del | DEL | p.Asn131fs |
| OC-01-040-AJ | hg19 | FAT1 | chr4 | 187629360 | 187629360 | G | C | Nonsense_Mutation | SNP | p.Ser541Ter |
| OC-01-040-AJ | hg19 | CDKN2A | chr9 | 21971186 | 21971186 | G | A | Nonsense_Mutation | SNP | p.Arg58Ter |
| OC-01-047-GM | hg19 | TP53 | chr17 | 7577575 | 7577575 | A | C | Missense_Mutation | SNP | p.Tyr236Asp |
| OC-01-047-GM | hg19 | PIK3CA | chr3 | 178922324 | 178922324 | G | A | Missense_Mutation | SNP | p.Glu365Lys |
| OC-01-049-NA | hg19 | HRAS | chr11 | 534289 | 534289 | C | T | Missense_Mutation | SNP | p.Gly12Ser |
| OC-01-049-NA | hg19 | CASP8 | chr2 | 202141587 | 202141587 | G | A | Missense_Mutation | SNP | p.Arg233Gln |
| OC-01-049-NA | hg19 | NOTCH1 | chr9 | 139405104 | 139405104 | C | A | Splice_Site | SNP | c.2740 + 1G > T |
| OC-01-054-GPC | hg19 | CASP8 | chr2 | 202131505 | 202131505 | C | G | Missense_Mutation | SNP | p.Ser99Cys |
| OC-01-054-GPC | hg19 | FAT1 | chr4 | 187541395 | 187541395 | | GTTTC | Frame_Shift_Ins | INS | p.Val2116fs |
| OC-01-054-GPC | hg19 | NOTCH1 | chr9 | 139403384 | 139403384 | G | A | Nonsense_Mutation | SNP | p.Gln1037Ter |
| OC-01-054-GPC | hg19 | CDKN2A | chr9 | 21971120 | 21971120 | G | A | Nonsense_Mutation | SNP | p.Arg80Ter |
| OC-01-055-CA | hg19 | CDKN2A | chr9 | 21971120 | 21971120 | G | A | Nonsense_Mutation | SNP | p.Arg80Ter |
| OC-01-055-CA | hg19 | FAT1 | chr4 | 187540884 | 187540884 | G | A | Nonsense_Mutation | SNP | p.Gln2286Ter |
| OC-01-059-LB | hg19 | TP53 | chr17 | 7577548 | 7577548 | C | T | Missense_Mutation | SNP | p.Gly245Ser |
| OC-01-061-MaS | hg19 | TP53 | chr17 | 7577121 | 7577121 | G | A | Missense_Mutation | SNP | p.Arg273Cys |
| OC-01-065-RAM | hg19 | TP53 | chr17 | 7577538 | 7577538 | C | T | Missense_Mutation | SNP | p.Arg248Gln |
| OC-01-074-AK | hg19 | TP53 | chr17 | 7578403 | 7578403 | C | T | Missense_Mutation | SNP | p.Cys176Tyr |
| OC-01-074-AK | hg19 | CDKN2A | chr9 | 21971120 | 21971120 | G | A | Nonsense_Mutation | SNP | p.Arg80Ter |
| OC-01-096-VE | hg19 | CDKN2A | chr9 | 21971120 | 21971120 | G | A | Nonsense_Mutation | SNP | p.Arg80Ter |
| OC-01-098-MHM | hg19 | NOTCH1 | chr9 | 139413277 | 139413277 | C | G | Splice_Site | SNP | c.866 − 1G > C |
| OC-02-004-SuD | hg19 | TP53 | chr17 | 7574003 | 7574003 | G | A | Nonsense_Mutation | SNP | p.Arg342Ter |
| OC-02-004-SuD | hg19 | CASP8 | chr2 | 202141586 | 202141586 | C | T | Missense_Mutation | SNP | p.Arg233Trp |
| OC-02-004-SuD | hg19 | FAT1 | chr4 | 187549509 | 187549509 | G | C | Missense_Mutation | SNP | p.Gln1537Glu |
| OC-02-004-SuD | hg19 | NOTCH1 | chr9 | 139402787 | 139402787 | G | T | Nonsense_Mutation | SNP | p.Cys1074Ter |
| OC-02-010-BKS | hg19 | TP53 | chr17 | 7574003 | 7574003 | G | A | Nonsense_Mutation | SNP | p.Arg342Ter |
| OC-02-010-BKS | hg19 | TP53 | chr17 | 7579350 | 7579350 | A | T | Missense_Mutation | SNP | p.Phe113Ile |
| OC-02-012-MK | hg19 | TP53 | chr17 | 7578260 | 7578260 | C | T | Missense_Mutation | SNP | p.Val197Met |
| OC-02-012-MK | hg19 | CASP8 | chr2 | 202141586 | 202141586 | C | T | Missense_Mutation | SNP | p.Arg233Trp |
| OC-02-012-MK | hg19 | FAT1 | chr4 | 187524105 | 187524105 | C | A | Nonsense_Mutation | SNP | p.Glu3812Ter |
| OC-02-012-MK | hg19 | FAT1 | chr4 | 187542073 | 187542075 | AA | — | Frame_Shift_Del | DEL | p.Leu1889fs |
| OC-02-019-PJ | hg19 | TP53 | chr17 | 7577097 | 7577097 | C | G | Missense_Mutation | SNP | p.Asp281His |
| OC-02-019-PJ | hg19 | CASP8 | chr2 | 202136252 | 202136252 | C | T | Nonsense_Mutation | SNP | p.Gln107Ter |
| OC-02-020-NK | hg19 | NOTCH1 | chr9 | 139412735 | 139412739 | ACAG | — | Frame_Shift_Del | DEL | p.Leu369fs |
| OC-02-023-SR | hg19 | HRAS | chr11 | 534285 | 534285 | C | A | Missense_Mutation | SNP | p.Gly13Val |
| OC-02-023-SR | hg19 | TP53 | chr17 | 7577094 | 7577094 | G | A | Missense_Mutation | SNP | p.Arg282Trp |
| OC-02-023-SR | hg19 | TP53 | chr17 | 7577538 | 7577538 | C | T | Missense_Mutation | SNP | p.Arg248Gln |
| OC-02-023-SR | hg19 | CASP8 | chr2 | 202149604 | 202149604 | G | T | Nonsense_Mutation | SNP | p.Glu290Ter |
| OC-02-023-SR | hg19 | FAT1 | chr4 | 187519154 | 187519154 | G | A | Nonsense_Mutation | SNP | p.Gln4077Ter |
| OC-02-023-SR | hg19 | FAT1 | chr4 | 187525133 | 187525133 | T | A | Splice_Site | SNP | c.10549 − 2A > T |
| OC-02-023-SR | hg19 | NOTCH1 | chr9 | 139410069 | 139410069 | — | TCTG | Frame_Shift_Ins | INS | p.Leu590fs |
| OC-02-023-SR | hg19 | NOTCH1 | chr9 | 139410069 | 139410069 | — | ATCTG | Frame_Shift_Ins | INS | p.Leu590fs |
| OC-02-023-SR | hg19 | CDKN2A | chr9 | 21971120 | 21971120 | G | A | Nonsense_Mutation | SNP | p.Arg80Ter |
| OC-02-025-RC | hg19 | TP53 | chr17 | 7574003 | 7574003 | G | A | Nonsense_Mutation | SNP | p.Arg342Ter |
| OC-02-025-RC | hg19 | PIK3CA | chr3 | 178952085 | 178952085 | A | G | Missense_Mutation | SNP | p.His1047Arg |
| OC-02-025-RC | hg19 | CDKN2A | chr9 | 21971120 | 21971120 | G | A | Nonsense_Mutation | SNP | p.Arg80Ter |
| OC-02-027-SS | hg19 | TP53 | chr17 | 7577157 | 7577157 | T | C | Splice_Site | SNP | c.783 − 2A > G |
| OC-02-027-SS | hg19 | TP53 | chr17 | 7579546 | 7579546 | — | G | Frame_Shift_Ins | INS | p.Asp48fs |
| OC-02-027-SS | hg19 | CASP8 | chr2 | 202131431 | 202131431 | G | T | Missense_Mutation | SNP | p.Leu74Phe |
| OC-02-029-KAM | hg19 | CASP8 | chr2 | 202150003 | 202150003 | C | T | Nonsense_Mutation | SNP | p.Gln423Ter |
| OC-02-033-TRM | hg19 | FAT1 | chr4 | 187518243 | 187518243 | A | C | Missense_Mutation | SNP | p.Cys4151Gly |
| OC-02-033-TRM | hg19 | FAT1 | chr4 | 187629920 | 187629920 | — | TGAA | Frame_Shift_Ins | INS | p.Val355fs |
| OC-02-033-TRM | hg19 | NOTCH1 | chr9 | 139399362 | 139399362 | C | T | Missense_Mutation | SNP | p.Arg1594Gln |
| OC-02-033-TRM | hg19 | TP53 | chr17 | 7577539 | 7577539 | G | A | Missense_Mutation | SNP | p.Arg248Trp |
| OC-02-043-DCS | hg19 | TP53 | chr17 | 7577513 | 7577513 | — | G | Frame_Shift_Ins | INS | p.Leu257fs |
| OC-02-043-DCS | hg19 | TP53 | chr17 | 7578209 | 7578209 | G | A | Missense_Mutation | SNP | p.His214Tyr |
| OC-02-043-DCS | hg19 | FAT1 | chr4 | 187531170 | 187531170 | C | A | Splice_Site | SNP | c.9854 − 1G > T |
| OC-02-043-DCS | hg19 | NOTCH1 | chr9 | 139413085 | 139413085 | G | A | Missense_Mutation | SNP | p.Arg353Cys |
| OC-02-050-SN | hg19 | CASP8 | chr2 | 202149751 | 202149751 | C | T | Nonsense_Mutation | SNP | p.Gln339Ter |
| OC-02-066-AB | hg19 | TP53 | chr17 | 7577022 | 7577022 | G | A | Nonsense_Mutation | SNP | p.Arg306Ter |
| OC-02-067-KN | hg19 | TP53 | chr17 | 7577098 | 7577098 | T | G | Missense_Mutation | SNP | p.Arg280Ser |
| OC-02-067-KN | hg19 | CDKN2A | chr9 | 21971120 | 21971120 | G | A | Nonsense_Mutation | SNP | p.Arg80Ter |
| OC-03-009 | hg19 | TP53 | chr17 | 7574003 | 7574003 | G | A | Nonsense_Mutation | SNP | p.Arg342Ter |
| OC-03-011 | hg19 | TP53 | chr17 | 7577067 | 7577067 | T | A | Nonsense_Mutation | SNP | p.Lys291Ter |
| OC-03-015 | hg19 | TP53 | chr17 | 7577082 | 7577082 | C | T | Missense_Mutation | SNP | p.Glu286Lys |
| OC-03-015 | hg19 | CDKN2A | chr9 | 21971028 | 21971028 | C | T | Nonsense_Mutation | SNP | p.Trp110Ter |
| OC-03-015 | hg19 | PIK3CA | chr3 | 178952085 | 178952085 | A | G | Missense_Mutation | SNP | p.His1047Arg |
| OC-02-055-RJ | hg19 | CDKN2A | chr9 | 21971132 | 21971132 | — | G | Frame_Shift_Ins | INS | p.Ala76fs |
| OC-03-005 | hg19 | TP53 | chr17 | 7577539 | 7577539 | G | A | Missense_Mutation | SNP | p.Arg248Trp |
| OC-03-005 | hg19 | TP53 | chr17 | 7578211 | 7578211 | C | T | Missense_Mutation | SNP | p.Arg213Gln |
| OC-03-005 | hg19 | FAT1 | chr4 | 187524791 | 187524791 | T | C | Missense_Mutation | SNP | p.His3630Arg |
| OC-03-005 | hg19 | CDKN2A | chr9 | 21971186 | 21971186 | G | A | Nonsense_Mutation | SNP | p.Arg58Ter |
| OC-01-102-NV | hg19 | PIK3CA | chr3 | 178936091 | 178936091 | G | A | Missense_Mutation | SNP | p.Glu545Lys |
| OC-02-036-DAK | hg19 | TP53 | chr17 | 7577099 | 7577099 | C | T | Missense_Mutation | SNP | p.Arg280Lys |
| OC-01-013-SR | hg19 | FAT1 | chr4 | 187630852 | 187630852 | G | A | Nonsense_Mutation | SNP | p.Gln44Ter |
| OC-01-013-SR | hg19 | NOTCH1 | chr9 | 139391035 | 139391035 | G | A | Nonsense_Mutation | SNP | p.Gln2386Ter |
| OC-01-014-SU | hg19 | TP53 | chr17 | 7578406 | 7578406 | C | T | Missense_Mutation | SNP | p.Arg175His |
| OC-01-014-SU | hg19 | FAT1 | chr4 | 187584451 | 187584451 | A | G | Splice_Site | SNP | c.3580 + 2T > C |
| OC-01-014-SU | hg19 | NOTCH1 | chr9 | 139438474 | 139438474 | A | G | Splice_Site | SNP | c.140 + 2T > C |
| OC-01-014-SU | hg19 | CDKN2A | chr9 | 21971193 | 21971194 | C | — | Frame_Shift_Del | DEL | p.Gly55fs |
| OC-02-022-SAB | hg19 | FAT1 | chr4 | 187558070 | 187558070 | T | C | Splice_Site | SNP | c.3643 − 2A > G |
| OC-01-032-SUS | hg19 | NOTCH1 | chr9 | 139399408 | 139399411 | CAC | — | Frame_Shift_Del | DEL | p.Val1578fs |
| OC-01-032-SUS | hg19 | CDKN2A | chr9 | 21971120 | 21971120 | G | A | Nonsense_Mutation | SNP | p.Arg80Ter |
| OC-02-007-DP | hg19 | CDKN2A | chr9 | 21971208 | 21971208 | C | T | Splice_Site | SNP | c.151 − 1G > A |
| OC-02-001-DK | hg19 | FAT1 | chr4 | 187517689 | 187517689 | C | T | Splice_Site | SNP | c.13000 + 5G > |
| OC-02-015-KM | hg19 | HRAS | chr11 | 534289 | 534289 | C | T | Missense_Mutation | SNP | p.Gly12Ser |
| OC-02-015-KM | hg19 | TP53 | chr17 | 7577090 | 7577090 | C | G | Missense_Mutation | SNP | p.Arg283Pro |
| OC-01-056-SKG | hg19 | TP53 | chr17 | 7577539 | 7577539 | G | A | Missense_Mutation | SNP | p.Arg248Trp |
| OC-01-056-SKG | hg19 | TP53 | chr17 | 7579340 | 7579340 | G | C | Missense_Mutation | SNP | p.Ser116Cys |
| OC-02-006-BS | hg19 | NOTCH1 | chr9 | 139411722 | 139411722 | A | C | Splice_Site | SNP | c.1555 + 2T > G |
| OC-01-024-KB | hg19 | NOTCH1 | chr9 | 139417584 | 139417584 | G | A | Nonsense_Mutation | SNP | p.Gln154Ter |
| OC-01-020-TSA | hg19 | NOTCH1 | chr9 | 139391041 | 139391041 | G | A | Nonsense_Mutation | SNP | p.Gln2384Ter |
| OC-03-012 | hg19 | TP53 | chr17 | 7577596 | 7577596 | A | G | Missense_Mutation | SNP | p.Cys229Arg |
| OC-03-012 | hg19 | NOTCHI | chr9 | 139399764 | 139399764 | G | T | Nonsense_Mutation | SNP | p.Cys1528Ter |
| OC-03-008 | hg19 | TP53 | chr17 | 7579406 | 7579406 | G | T | Nonsense_Mutation | SNP | p.Ser94Ter |
| OC-03-008 | hg19 | PIK3CA | chr3 | 178916935 | 178916935 | C | A | Missense_Mutation | SNP | p.Arg108Ser |
| OC-03-008 | hg19 | FAT | chr4 | 187524630 | 187524630 | G | A | Nonsense_Mutation | SNP | p.Gln3684Ter |
| OC-03-008 | hg19 | FAT1 | chr4 | 187557961 | 187557961 | G | T | Nonsense_Mutation | SNP | p.Tyr1250Ter |
| OC-03-008 | hg19 | FAT | chr4 | 187584759 | 187584759 | C | A | Nonsense_Mutation | SNP | p.Glu1092Ter |
| OC-03-008 | hg19 | NOTCH1 | chr9 | 139391978 | 139391978 | C | A | Missense_Mutation | SNP | p.Glu2071Asp |
| OC-03-008 | hg19 | NOTCH1 | chr9 | 139411727 | 139411728 | G | — | Frame_Shift_Del | DEL | p.Thr518fs |
| OC-03-008 | hg19 | CDKN2A | chr9 | 21971185 | 21971185 | C | T | Missense_Mutation | SNP | p.Arg58Gln |
| OC-01-001-AR | hg19 | TP53 | chr17 | 7578190 | 7578190 | T | G | Missense_Mutation | SNP | p.Tyr220Ser |
| OC-01-003-BM | hg19 | TP53 | chr17 | 7578556 | 7578556 | T | C | Splice_Site | SNP | c.376 − 2A > G |
| OC-01-026-GKD | hg19 | TP53 | chr17 | 7578392 | 7578392 | C | T | Missense_Mutation | SNP | p.Glu180Lys |
| OC-01-026-GKD | hg19 | NOTCH1 | chr9 | 139404184 | 139404184 | C | G | Splice_Site | SNP | c.2969 + 1G > C |
| OC-01-026-GKD | hg19 | TP53 | chr17 | 7577538 | 7577538 | C | T | Missense_Mutation | SNP | p.Arg248Gln |
| OC-01-026-GKD | hg19 | TP53 | chr17 | 7578208 | 7578208 | T | C | Missense_Mutation | SNP | p.His214Arg |
| OC-01-026-GKD | hg19 | NOTCH1 | chr9 | 139412607 | 139412608 | G | | Frame_Shift_Del | DEL | p.Asp412fs |
| OC-01-030-RP | hg19 | TP53 | chr17 | 7577017 | 7577017 | A | T | Splice_Site | SNP | c.919 + 2T > A |
| OC-01-030-RP | hg19 | TP53 | chr17 | 7577569 | 7577569 | A | C | Missense_Mutation | SNP | p.Cys238Gly |
| OC-01-030-RP | hg19 | TP53 | chr17 | 7578406 | 7578406 | C | T | Missense_Mutation | SNP | p.Arg175His |
| OC-01-030-RP | hg19 | CASP8 | chr2 | 202141690 | 202141690 | A | G | Missense_Mutation | SNP | p.Ala267Ala |
| OC-01-030-RP | hg19 | TP53 | chr17 | 7577539 | 7577539 | G | A | Missense_Mutation | SNP | p.Arg248Trp |
| OC-01-030-RP | hg19 | NOTCH1 | chr9 | 139411722 | 139411722 | A | G | Splice_Site | SNP | c.1555 + 2T > C |
| OC-01-033-PS | hg19 | TP53 | chr17 | 7573009 | 7573009 | C | T | Splice_Site | SNP | c.1101 − 1G > A |
| OC-01-033-PS | hg19 | TP53 | chr17 | 7577106 | 7577106 | G | A | Missense_Mutation | SNP | p.Pro278Ser |
| OC-01-033-PS | hg19 | NOTCH1 | chr9 | 139395179 | 139395179 | A | G | Missense_Mutation | SNP | p.Leu1920Pro |
| OC-01-033-PS | hg19 | NOTCH1 | chr9 | 139399380 | 139399383 | TGT | — | InFrame_Del | DEL | p.Asn1587del |
| OC-01-033-PS | hg19 | CDKN2A | chr9 | 21974775 | 21974775 | T | C | Missense_Mutation | SNP | p.Thr18Ala |
| OC-01-033-PS | hg19 | TP53 | chr17 | 7577511 | 7577511 | A | G | Missense_Mutation | SNP | p.Leu257Pro |
| OC-01-033-PS | hg19 | FAT1 | chr4 | 187524560 | 187524560 | G | T | Nonsense_Mutation | SNP | p.Ser3707Ter |
| OC-01-037-JK | hg19 | TP53 | chr17 | 7578202 | 7578202 | A | C | Missense_Mutation | SNP | p.Val216Gly |
| OC-01-037-JK | hg19 | NOTCH1 | chr9 | 139405259 | 139405259 | T | C | Splice_Site | SNP | c.2588 − 2A > G |
| OC-01-037-JK | hg19 | CDKN2A | chr9 | 21974768 | 21974768 | G | A | Missense_Mutation | SNP | p.Ala20Val |
| OC-01-040-AJ | hg19 | TP53 | chr17 | 7577547 | 7577547 | C | T | Missense_Mutation | SNP | p.Gly245Asp |
| OC-01-040-AJ | hg19 | TP53 | chr17 | 7578534 | 7578534 | C | A | Missense_Mutation | SNP | p.Lys132Asn |
| OC-01-040-AJ | hg19 | TP53 | chr17 | 7579409 | 7579410 | G | — | Frame_Shift_Del | DEL | p.Leu93fs |
| OC-01-040-AJ | hg19 | TP53 | chr17 | 7579358 | 7579358 | C | G | Missense_Mutation | SNP | p.Arg110Pro |
| OC-01-045-PH | hg19 | TP53 | chr17 | 7577556 | 7577556 | C | T | Missense_Mutation | SNP | p.Cys242Tyr |
| OC-01-047-GM | hg19 | TP53 | chr17 | 7577539 | 7577539 | G | A | Missense_Mutation | SNP | p.Arg248Trp |
| OC-01-048-MC | hg19 | FAT1 | chr4 | 187518327 | 187518327 | T | C | Splice_Site | SNP | c.12369 − 2A > G |
| OC-01-048-MC | hg19 | FAT1 | chr4 | 187516928 | 187516929 | T | — | Frame_Shift_Del | DEL | p.Lys4351fs |
| OC-01-049-NA | hg19 | FAT1 | chr4 | 187516885 | 187516885 | G | A | Nonsense_Mutation | SNP | p.Gln4366Ter |
| OC-01-054-GPC | hg19 | TP53 | chr17 | 7578208 | 7578208 | T | C | Missense_Mutation | SNP | p.His214Arg |
| OC-01-055-CA | hg19 | FAT1 | chr4 | 187521047 | 187521047 | C | T | Splice_Site | SNP | c.12103 + 5G > A |
| OC-01-055-CA | hg19 | FAT1 | chr4 | 187534320 | 187534320 | C | A | Nonsense_Mutation | SNP | p.Glu3136Ter |
| OC-01-057-NM | hg19 | FAT1 | chr4 | 187516928 | 187516929 | T | — | Frame_Shift_Del | DEL | p.Lys4351fs |
| OC-01-059-LB | hg19 | TP53 | chr17 | 7577520 | 7577520 | A | G | Missense_Mutation | SNP | p.Ile254Thr |
| OC-01-059-LB | hg19 | FAT1 | chr4 | 187516928 | 187516929 | T | — | Frame_Shift_Del | DEL | p.Lys4351fs |
| OC-01-070-RA | hg19 | TP53 | chr17 | 7577106 | 7577106 | G | C | Missense_Mutation | SNP | p.Pro278Ala |
| OC-01-070-RA | hg19 | TP53 | chr17 | 7578446 | 7578446 | T | A | Missense_Mutation | SNP | p.Ile162Phe |
| OC-01-070-RA | hg19 | NOTCH1 | chr9 | 139390846 | 139390847 | G | — | Frame_Shift_Del | DEL | p.Ser2449fs |
| OC-01-070-RA | hg19 | NOTCH1 | chr9 | 139405104 | 139405104 | C | T | Splice_Site | SNP | c.2740 + 1G > A |
| OC-01-070-RA | hg19 | NOTCH1 | chr9 | 139409740 | 139409740 | A | G | Splice_Site | SNP | c.2014 + 2T > C |
| OC-01-070-RA | hg19 | NOTCH1 | chr9 | 139390621 | 139390621 | A | C | Missense_Mutation | SNP | p.Ser2524Ala |
| OC-01-070-RA | hg19 | NOTCHI | chr9 | 139405259 | 139405259 | T | C | Splice_Site | SNP | c.2588 − 2A > G |
| OC-01-074-AK | hg19 | FAT1 | chr4 | 187525133 | 187525133 | T | C | Splice_Site | SNP | c.10549 − 2A > G |
| OC-01-074-AK | hg19 | FAT1 | chr4 | 187538158 | 187538158 | C | T | Splice_Site | SNP | c.9075 + 1G > A |
| OC-01-088-SAA | hg19 | PIK3CA | chr3 | 178943810 | 178943810 | A | G | Missense_Mutation | SNP | p.Asn826Ser |
| OC-01-095-LY | hg19 | NOTCH1 | chr9 | 139405104 | 139405104 | C | T | Splice_Site | SNP | c.2740 + 1G > A |
| OC-01-095-LY | hg19 | CDKN2A | chr9 | 21974768 | 21974768 | G | A | Missense_Mutation | SNP | p.Ala20Val |
| OC-01-095-LY | hg19 | TP53 | chr17 | 7578535 | 7578535 | T | C | Missense_Mutation | SNP | p.Lys132Arg |
| OC-01-096-VE | hg19 | FAT1 | chr4 | 187510329 | 187510329 | G | A | Missense_Mutation | SNP | p.Pro4395Leu |
| OC-01-096-VE | hg19 | FAT1 | chr4 | 187539564 | 187539564 | G | A | Nonsense_Mutation | SNP | p.Arg2726Ter |
| OC-02-004-SuD | hg19 | CASP8 | chr2 | 202131411 | 202131411 | C | T | Nonsense_Mutation | SNP | p.Arg68Ter |
| OC-02-019-PJ | hg19 | CDKN2A | chr9 | 21968243 | 21968243 | T | C | Splice_Site | SNP | c.458 − 2A > G |
| OC-02-025-RC | hg19 | CDKN2A | chr9 | 21971186 | 21971186 | G | A | Nonsense_Mutation | SNP | p.Arg58Ter |
| OC-02-025-RC | hg19 | CDKN2A | chr9 | 21974775 | 21974775 | T | C | Missense_Mutation | SNP | p.Thr18Ala |
| OC-02-027-SS | hg19 | TP53 | chr17 | 7577550 | 7577550 | C | T | Missense_Mutation | SNP | p.Gly244Asp |
| OC-02-027-SS | hg19 | NOTCH1 | chr9 | 139400335 | 139400335 | T | A | Splice_Site | SNP | c.4015 − 2A > T |
| OC-02-027-SS | hg19 | CDKN2A | chr9 | 21968242 | 21968242 | C | T | Splice_Site | SNP | c.458 − 1G > A |
| OC-02-027-SS | hg19 | TP53 | chr17 | 7578212 | 7578212 | G | A | Nonsense_Mutation | SNP | p.Arg213Ter |
| OC-02-033-TRM | hg19 | CDKN2A | chr9 | 21971120 | 21971120 | G | A | Nonsense_Mutation | SNP | p.Arg80Ter |
| OC-02-043-DCS | hg19 | HRAS | chr11 | 534288 | 534288 | C | T | Missense_Mutation | SNP | p.Gly12Asp |
| OC-02-043-DCS | hg19 | TP53 | chr17 | 7578492 | 7578492 | C | T | Nonsense_Mutation | SNP | p.Trp146Ter |
| OC-02-043-DCS | hg19 | FAT1 | chr4 | 187527369 | 187527369 | T | A | Splice_Site | SNP | c.10207 − 2A > T |
| OC-02-043-DCS | hg19 | FAT1 | chr4 | 187541574 | 187541574 | — | CT | Frame_Shift_Ins | INS | p.Glu2056fs |
| OC-02-043-DCS | hg19 | CDKN2A | chr9 | 21971123 | 21971125 | GA | — | Frame_Shift_Del | DEL | p.Leu78fs |
| OC-02-050-SN | hg19 | TP53 | chr17 | 7577526 | 7577526 | A | G | Missense_Mutation | SNP | p.Leu252Pro |
| OC-02-066-AB | hg19 | FAT1 | chr4 | 187557735 | 187557735 | T | C | Splice_Site | SNP | c.3972 + 4A > G |
| OC-02-067-KN | hg19 | FAT1 | chr4 | 187557735 | 187557735 | T | C | Splice_Site | SNP | c.3972 + 4A > G |
| OC-02-073-BAB | hg19 | HRAS | chr11 | 534289 | 534289 | C | T | Missense_Mutation | SNP | p.Gly12Ser |
| OC-02-073-BAB | hg19 | CASP8 | chr2 | 202151270 | 202151270 | C | T | Nonsense_Mutation | SNP | p.Gln465Ter |
| OC-02-073-BAB | hg19 | PIK3CA | chr3 | 178952074 | 178952074 | G | T | Missense_Mutation | SNP | p.Met1043Ile |
| OC-03-009 | hg19 | FAT1 | chr4 | 187539563 | 187539563 | C | T | Missense_Mutation | SNP | p.Arg2726Gln |
| OC-03-009 | hg19 | NOTCH1 | chr9 | 139411727 | 139411728 | G | — | Frame_Shift_Del | DEL | p.Thr518fs |
| OC-03-009 | hg19 | NOTCH1 | chr9 | 139413234 | 139413234 | G | T | Missense_Mutation | SNP | p.Pro303Gln |
| OC-03-009 | hg19 | CDKN2A | chr9 | 21971057 | 21971057 | C | A | Missense_Mutation | SNP | p.Gly101Trp |
| OC-03-009 | hg19 | CDKN2A | chr9 | 21971063 | 21971063 | G | A | Missense_Mutation | SNP | p.Arg99Trp |
| OC-03-009 | hg19 | FAT1 | chr4 | 187540554 | 187540554 | C | A | Nonsense_Mutation | SNP | p.Glu2396Ter |
| OC-03-009 | hg19 | FAT1 | chr4 | 187584759 | 187584759 | C | A | Nonsense_Mutation | SNP | p.Glu1092Ter |
| OC-03-011 | hg19 | FAT1 | chr4 | 187527368 | 187527368 | C | G | Splice_Site | SNP | c.10207 − 1G > C |
| OC-03-015 | hg19 | CDKN2A | chr9 | 21970899 | 21970899 | A | G | Splice_Site | SNP | c.457 + 2T > C |
| OC-01-081-RAT | hg19 | NA | NA | NA | NA | NA | NA | NA | NA | NA |
| OC-02-039-KS | hg19 | NA | NA | NA | NA | NA | NA | NA | NA | NA |
| OC-01-082-BB | hg19 | NA | NA | NA | NA | NA | NA | NA | NA | NA |
| OC-01-100-GNH | hg19 | NA | NA | NA | NA | NA | NA | NA | NA | NA |
| OC-01-103-PGM | hg19 | NA | NA | NA | NA | NA | NA | NA | NA | NA |

Supplemental Table S7. Reproducibility in Saliva

| OCSCC positive cases | | | | | |
|---|---|---|---|---|---|
| | | | No. of | | |
| | | | Variants | | |
| | | No. of | Detected in | | |
| | | Variants | Replicate | | |
| | | (% VAF | (% VAF | Re- | |
| | Repli- | in range | in range | produc- | |
| Sample ID | cate | 0.2 to 30) | 0.1 to 30) | ibility | |
| OC-01-003-BM | R1 | 3 | 3 | 100.00 | Repli- |
| OC-01-025-PC | R1 | 1 | 1 | 100.00 | cate I |
| OC-01-026-GKD | R1 | 3 | 2 | 66.67 | |
| OC-01-030-RP | R1 | 21 | 21 | 100.00 | |
| OC-01-045-PH | R1 | 2 | 1 | 50.00 | |
| OC-01-048-MC | R1 | 2 | 2 | 100.00 | |
| OC-01-057-NM | R1 | 2 | 2 | 100.00 | |
| OC-01-059-LB | R1 | 3 | 2 | 66.67 | |
| OC-01-061-MaS | R1 | 3 | 2 | 66.67 | |
| OC-01-070-RA | R1 | 2 | 2 | 100.00 | |
| OC-01-081-RAT | R1 | 1 | 1 | 100.00 | |
| OC-02-010-BKS | R1 | 10 | 4 | 40.00 | |
| OC-02-012-MK | R1 | 6 | 6 | 100.00 | |
| OC-02-023-SR | R1 | 11 | 11 | 100.00 | |
| OC-02-029-KAM | R1 | 2 | 1 | 50.00 | |
| OC-02-039-KS | R1 | 1 | 1 | 100.00 | |
| OC-02-043-DCS | R1 | 10 | 10 | 100.00 | |
| OC-02-066-AB | R1 | 3 | 3 | 100.00 | |
| OC-02-067-KN | R1 | 4 | 4 | 100.00 | |
| OC-01-001-AR | R1 | 6 | 2 | 33.33 | |
| OC-01-007-ST | R1 | 4 | 4 | 100.00 | |
| OC-01-008-KA | R1 | 4 | 4 | 100.00 | |
| OC-01-033-PS | R1 | 5 | 3 | 60.00 | |
| OC-01-037-JK | R1 | 4 | 3 | 75.00 | |
| OC-01-040-AJ | R1 | 6 | 6 | 100.00 | |
| OC-01-055-CA | R1 | 2 | 2 | 100.00 | |
| OC-01-074-AK | R1 | 4 | 3 | 75.00 | |
| OC-01-088-SAA | R1 | 2 | 2 | 100.00 | |
| OC-01-096-VE | R1 | 5 | 5 | 100.00 | |
| OC-02-004-SuD | R1 | 10 | 6 | 60.00 | |
| OC-02-025-RC | R1 | 3 | 2 | 66.67 | |
| OC-02-027-SS | R1 | 5 | 5 | 100.00 | |
| OC-02-033-TRM | R1 | 3 | 3 | 100.00 | |
| OC-02-073-BAB | R1 | 6 | 6 | 100.00 | |
| OC-03-009 | R1 | 4 | 4 | 100.00 | |
| OC-03-011 | R1 | 1 | 1 | 100.00 | |
| OC-01-095-LY | R1 | 1 | 1 | 100.00 | |
| OC-03-015 | R1 | 5 | 5 | 100.00 | |
| OC-01-065-RAM | R1 | 3 | 2 | 66.67 | |
| OC-02-020-NK | R1 | 1 | 1 | 100.00 | |
| OC-02-019-PJ | R1 | 3 | 3 | 100.00 | |
| OC-01-054-GPC | R1 | 6 | 6 | 100.00 | |
| OC-01-098-MHM | R1 | 1 | 1 | 100.00 | |
| OC-01-047-GM | R1 | 2 | 2 | 100.00 | |
| OC-02-050-SN | R1 | 2 | 2 | 100.00 | |
| OC-01-049-NA | R1 | 1 | 1 | 100.00 | |
| OC-01-003-BM | R2 | 3 | 3 | 100.00 | Repli- |
| OC-01-025-PC | R2 | 2 | 2 | 100.00 | cate II |
| OC-01-026-GKD | R2 | 7 | 5 | 71.43 | |
| OC-01-030-RP | R2 | 19 | 19 | 100.00 | |
| OC-01-045-PH | R2 | 2 | 2 | 100.00 | |
| OC-01-048-MC | R2 | 2 | 2 | 100.00 | |
| OC-01-057-NM | R2 | 1 | 1 | 100.00 | |
| OC-01-059-LB | R2 | 2 | 2 | 100.00 | |
| OC-01-061-MaS | R2 | 2 | 1 | 50.00 | |
| OC-01-070-RA | R2 | 2 | 1 | 50.00 | |
| OC-01-081-RAT | R2 | 2 | 2 | 100.00 | |
| OC-02-010-BKS | R2 | 3 | 3 | 100.00 | |
| OC-02-012-MK | R2 | 6 | 6 | 100.00 | |
| OC-02-023-SR | R2 | 12 | 11 | 91.67 | |
| OC-02-029-KAM | R2 | 1 | 1 | 100.00 | |
| OC-02-039-KS | R2 | 1 | 1 | 100.00 | |
| OC-02-043-DCS | R2 | 11 | 11 | 100.00 | |
| OC-02-066-AB | R2 | 3 | 2 | 66.67 | |
| OC-02-067-KN | R2 | 4 | 4 | 100.00 | |
| OC-01-001-AR | R2 | 3 | 2 | 66.67 | |
| OC-01-007-ST | R2 | 3 | 3 | 100.00 | |
| OC-01-008-KA | R2 | 3 | 3 | 100.00 | |
| OC-01-033-PS | R2 | 5 | 3 | 60.00 | |
| OC-01-037-JK | R2 | 3 | 3 | 100.00 | |
| OC-01-040-AJ | R2 | 4 | 4 | 100.00 | |
| OC-01-055-CA | R2 | 2 | 2 | 100.00 | |
| OC-01-074-AK | R2 | 4 | 3 | 75.00 | |
| OC-01-088-SAA | R2 | 2 | 2 | 100.00 | |
| OC-01-096-VE | R2 | 6 | 5 | 83.33 | |
| OC-02-004-SuD | R2 | 5 | 5 | 100.00 | |
| OC-02-025-RC | R2 | 2 | 2 | 100.00 | |
| OC-02-027-SS | R2 | 5 | 5 | 100.00 | |
| OC-02-033-TRM | R2 | 4 | 3 | 75.00 | |
| OC-02-073-BAB | R2 | 5 | 5 | 100.00 | |
| OC-03-009 | R2 | 5 | 4 | 80.00 | |
| OC-03-011 | R2 | 1 | 1 | 100.00 | |
| OC-01-095-LY | R2 | 3 | 1 | 33.33 | |
| OC-03-015 | R2 | 5 | 4 | 80.00 | |
| OC-01-065-RAM | R2 | 2 | 2 | 100.00 | |
| OC-02-020-NK | R2 | 1 | 1 | 100.00 | |
| OC-02-019-PJ | R2 | 4 | 3 | 75.00 | |
| OC-01-054-GPC | R2 | 5 | 4 | 80.00 | |
| OC-01-098-MHM | R2 | 2 | 2 | 100.00 | |
| OC-01-047-GM | R2 | 1 | 1 | 100.00 | |
| OC-02-050-SN | R2 | 3 | 2 | 66.67 | |
| OC-01-049-NA | R2 | 2 | 1 | 50.00 | |
| TOTAL | | 364 | 319 | 87.64 | |

Healthy controls

| | | | No. of | |
|---|---|---|---|---|
| | | | Variants | |
| | | | Detected in | |
| | | No. of Variants | Replicate | |
| | | (% VAF | (% VAF | |
| | | in range | in range | |
| Sample ID | Replicate | 0.2 to 30) | 0.1 to 30) | Reproducibility |
| NOC-01-011 | R1 | 1 | 0 | 0.00 |
| NOC-01-020 | R1 | 1 | 0 | 0.00 |

Supplemental Table S8. Concordance of FFPE Variants in Saliva

| Sample ID | Chromosome | Start | End | Reference | Variant | Gene | Variant | gHGVS |
|---|---|---|---|---|---|---|---|---|
| OC-02-055-RJ | chr17 | 7577118 | 7577118 | C | A | TP53 | p.Val274Phe | g.7577118C > A |
| OC-02-055-RJ | chr17 | 7579328 | 7579328 | T | A | TP53 | p.Lys120Met | g.7579328T > A |
| OC-02-055-RJ | chr2 | 202131411 | 202131411 | C | T | CASP8 | p.Arg68Ter | g.202131411C > T |
| OC-02-055-RJ | chr9 | 139414006 | 139414006 | G | A | NOTCH1 | p.Gln252Ter | g.139414006G > A |
| OC-03-005 | chr17 | 7573009 | 7573009 | C | T | TP53 | c.1101 − | g.7573009C > T |
| | | | | | | | 1G > A | |
| OC-03-005 | chr17 | 7577120 | 7577120 | C | T | TP53 | p.Arg273His | g.7577120C > T |
| OC-03-005 | chr4 | 187540784 | 187540784 | G | C | FAT1 | p.Ser2319Ter | g.187540784G > C |
| OC-03-005 | chr4 | 187541246 | 187541246 | G | A | FAT1 | p.Ser2165Leu | g.187541246G > A |
| OC-03-005 | chr9 | 139405649 | 139405649 | C | A | NOTCHI | p.Glu848Ter | g.139405649C > A |
| OC-03-005 | chr9 | 21971116 | 21971116 | G | C | CDKN2A | p.Pro81Arg | g.21971116G > C |
| OC-01-102-NV | chr2 | 202137440 | 202137440 | G | A | CASP8 | p.Cys164Tyr | g.202137440G > A |
| OC-01-102-NV | chr9 | 139412212 | 139412212 | C | A | NOTCH1 | p.Cys478Phe | g.139412212C > A |
| OC-02-038-AM | chr17 | 7577141 | 7577141 | C | T | TP53 | p.Gly266Glu | g.7577141C > T |
| OC-02-038-AM | chr2 | 202151270 | 202151270 | C | T | CASP8 | p.Gln465Ter | g.202151270C > T |
| OC-02-038-AM | chr4 | 187540677 | 187540678 | C | — | FAT1 | p.Gln2355fs | g.187540678delC |
| OC-02-038-AM | chr9 | 21971120 | 21971120 | G | A | CDKN2A | p.Arg80Ter | g.21971120G > A |
| OC-02-045-KCD | chr11 | 533874 | 533874 | T | A | HRAS | p.Gln61Leu | g.533874T > A |
| OC-02-045-KCD | chr17 | 7577094 | 7577094 | G | A | TP53 | p.Arg282Trp | g.7577094G > A |
| OC-02-045-KCD | chr17 | 7578503 | 7578503 | C | T | TP53 | p.Val143Met | g.7578503C > T |
| OC-02-045-KCD | chr4 | 187518926 | 187518926 | C | T | FAT1 | p.Cys4093Tyr | g.187518926C > T |
| OC-02-045-KCD | chr4 | 187540950 | 187540950 | — | A | FAT1 | p.His2264fs | g.187540950_{—} |
| | | | | | | | | 187540951insA |
| OC-02-045-KCD | chr9 | 21971179 | 21971179 | — | CCACTA | CDKN2A | p.Ala60fS | g.21971179_{—} |
| | | | | | | | | 21971180insCCACTA |
| OC-02-068-NIK | chr17 | 7577538 | 7577538 | C | T | TP53 | p.Arg248Gln | g.7577538C > T |
| OC-02-068-NIK | chr9 | 21971028 | 21971028 | C | T | CDKN2A | p.Trp110Ter | g.21971028C > T |
| OC-02-036-DAK | chr17 | 7574018 | 7574018 | G | A | TP53 | p.Arg337Cys | g.7574018G > A |
| OC-02-036-DAK | chr17 | 7577105 | 7577105 | G | C | TP53 | p.Pro278Arg | g.7577105G > C |
| OC-02-036-DAK | chr9 | 21971120 | 21971120 | G | A | CDKN2A | p.Arg80Ter | g.21971120G > A |
| OC-01-013-SR | chr17 | 7578413 | 7578413 | C | T | TP53 | p.Val173Met | g.7578413C > T |
| OC-01-013-SR | chr9 | 21971186 | 21971186 | G | A | CDKN2A | p.Arg58Ter | g.21971186G > A |
| OC-01-014-SU | chr17 | 7577094 | 7577094 | G | A | TP53 | p.Arg282Trp | g.7577094G > A |
| OC-01-014-SU | chr9 | 21974792 | 21974792 | G | T | CDKN2A | p.Ser12Ter | g.21974792G > T |
| OC-02-022-SAB | chr11 | 534289 | 534289 | C | T | HRAS | p.Gly12Ser | g.534289C > T |
| OC-02-022-SAB | chr17 | 7577121 | 7577121 | G | A | TP53 | p.Arg273Cys | g.7577121G > A |
| OC-02-022-SAB | chr17 | 7578449 | 7578449 | C | T | TP53 | p.Ala161Thr | g.7578449C > T |
| OC-02-022-SAB | chr9 | 21971186 | 21971186 | G | A | CDKN2A | p.Arg58Ter | g.21971186G > A |
| OC-01-032-SUS | chr17 | 7578197 | 7578197 | — | CA | TP53 | p.Val218fs | g.7578197insCA |
| OC-01-032-SUS | chr3 | 178922324 | 178922324 | G | A | PIK3CA | p.Glu365Lys | g.178922324G > A |
| OC-01-032-SUS | chr4 | 187630417 | 187630418 | A | — | FAT1 | p.Phe188fs | g.187630418delA |
| OC-01-032-SUS | chr9 | 21971186 | 21971186 | G | A | CDKN2A | p.Arg58Ter | g.21971186G > A |
| OC-01-032-SUS | chr9 | 21971208 | 21971208 | C | T | CDKN2A | c.151 − | g.21971208C > T |
| | | | | | | | 1G > A | |
| OC-02-003-BK | chr17 | 7577094 | 7577094 | G | A | TP53 | p.Arg282Trp | g.7577094G > A |
| OC-02-003-BK | chr17 | 7578503 | 7578503 | C | T | TP53 | p.Val143Met | g.7578503C > T |
| OC-01-050-MP | chr17 | 7577539 | 7577539 | G | A | TP53 | p.Arg248Trp | g.7577539G > A |
| OC-01-050-MP | chr2 | 202149537 | 202149537 | A | G | CASP8 | c.803 − | g.202149537A > G |
| | | | | | | | 2A > G | |
| OC-01-050-MP | chr9 | 139412645 | 139412645 | C | A | NOTCH1 | p.Cys400Phe | g.139412645C > A |
| OC-01-050-MP | chr9 | 21971053 | 21971053 | G | A | CDKN2A | p.Ala102Val | g.21971053G > A |
| OC-01-050-MP | chr9 | 21971120 | 21971120 | G | A | CDKN2A | p.Arg80Ter | g.21971120G > A |
| OC-02-007-DP | chr17 | 7574018 | 7574018 | G | A | TP53 | p.Arg337Cys | g.7574018G > A |
| OC-02-007-DP | chr17 | 7578212 | 7578212 | G | A | TP53 | p.Arg213Ter | g.7578212G > A |
| OC-02-007-DP | chr4 | 187549306 | 187549306 | A | G | FAT1 | c.4810 + | g.187549306A > G |
| | | | | | | | 2T > C | |
| OC-02-007-DP | chr9 | 21971120 | 21971120 | G | A | CDKN2A | p.Arg80Ter | g.21971120G > A |
| OC-02-007-DP | chr9 | 21971186 | 21971186 | G | A | CDKN2A | p.Arg58Ter | g.21971186G > A |
| OC-01-018-SSR | chr17 | 7579503 | 7579503 | C | A | TP53 | p.Glu62Ter | g.7579503C > A |
| OC-01-018-SSR | chr9 | 21971036 | 21971036 | C | G | CDKN2A | p.Asp108His | g.21971036C > G |
| OC-02-016-DC | chr17 | 7578406 | 7578406 | C | T | TP53 | p.Arg175His | g.7578406C > T |
| OC-02-016-DC | chr9 | 21971186 | 21971186 | G | A | CDKN2A | p.Arg58Ter | g.21971186G > A |
| OC-01-002-ABR | chr17 | 7577120 | 7577120 | C | T | TP53 | p.Arg273His | g.7577120C > T |
| OC-01-002-ABR | chr17 | 7578257 | 7578257 | C | A | TP53 | p.Glu198Ter | g.7578257C > A |
| OC-01-002-ABR | chr2 | 202149832 | 202149832 | C | T | CASP8 | p.Gln366Ter | g.202149832C > T |
| OC-01-002-ABR | chr9 | 21971120 | 21971120 | G | A | CDKN2A | p.Arg80Ter | g.21971120G > A |
| OC-01-006-SA | chr17 | 7578457 | 7578457 | C | A | TP53 | p.Arg158Leu | g.7578457C > A |
| OC-01-006-SA | chr4 | 187549907 | 187549914 | TGATGAA | — | FAT1 | p.Phe1443fs | g.187549908_{—} |
| | | | | | | | | 187549914delTGATGAA |
| OC-01-006-SA | chr9 | 21971120 | 21971120 | G | A | CDKN2A | p.Arg80Ter | g.21971120G > A |
| OC-01-029-JR | chr17 | 7577120 | 7577120 | C | T | TP53 | p.Arg273His | g.7577120C > T |
| OC-02-009-RS | chr11 | 534289 | 534289 | C | T | HRAS | p.Gly12Ser | g.534289C > T |
| OC-02-009-RS | chr17 | 7576897 | 7576897 | G | A | TP53 | p.Gln317Ter | g.7576897G > A |
| OC-02-009-RS | chr17 | 7577130 | 7577130 | A | T | TP53 | p.Phe270Ile | g.7577130A > T |
| OC-02-009-RS | chr2 | 202149973 | 202149973 | C | T | CASP8 | p.Arg413Ter | g.202149973C > T |
| OC-01-075-GST | chr17 | 7577144 | 7577144 | A | G | TP53 | p.Leu265Pro | g.7577144A > G |
| OC-01-075-GST | chr2 | 202141611 | 202141611 | A | G | CASP8 | p.Asn241Ser | g.202141611A > G |
| OC-01-075-GST | chr9 | 139399171 | 139399171 | C | A | NOTCH1 | p.Glu1658Ter | g.139399171C > A |
| OC-01-075-GST | chr9 | 139402742 | 139402742 | C | T | NOTCH1 | p.Trp1089Ter | g.139402742C > T |
| OC-02-030-RB | chr17 | 7579307 | 7579307 | C | A | TP53 | c.375 + | g.7579307C > A |
| | | | | | | | 5G > T | |
| OC-02-030-RB | chr4 | 187509861 | 187509861 | G | C | FAT1 | p.Ala4551Gly | g.187509861G > C |
| OC-02-030-RB | chr9 | 21971186 | 21971186 | G | A | CDKN2A | p.Arg58Ter | g.21971186G > A |
| OC-03-017 | chr17 | 7577121 | 7577121 | G | A | TP53 | p.Arg273Cys | g.7577121G > A |
| OC-03-017 | chr17 | 7579313 | 7579313 | G | A | TP53 | p.Thr125Met | g.7579313G > A |
| OC-03-002 | chr17 | 7577539 | 7577539 | G | A | TP53 | p.Arg248Trp | g.7577539G > A |
| OC-03-002 | chr9 | 21974695 | 21974695 | G | C | CDKN2A | p.Tyr44Ter | g.21974695G > C |
| OC-01-092-LA | chr17 | 7578394 | 7578394 | T | C | TP53 | p.His179Arg | g.7578394T > C |
| OC-01-092-LA | chr4 | 187558005 | 187558005 | G | A | FAT1 | p.Leu1236Phe | g.187558005G > A |
| OC-03-004 | chr17 | 7579346 | 7579346 | A | T | TP53 | p.Leu114Ter | g.7579346A > T |
| OC-03-004 | chr4 | 187630979 | 187630979 | C | A | FAT1 | p.Met1Ile | g.187630979C > A |
| OC-03-004 | chr9 | 21971120 | 21971120 | G | A | CDKN2A | p.Arg80Ter | g.21971120G > A |
| OC-01-101-JMA | chr9 | 21974792 | 21974792 | G | T | CDKN2A | p.Ser12Ter | g.21974792G > T |
| OC-02-037-BM | chr11 | 534289 | 534289 | C | T | HRAS | p.Gly12Ser | g.534289C > T |
| OC-02-037-BM | chr2 | 202137378 | 202137378 | C | A | CASP8 | p.Phe143Leu | g.202137378C > A |
| OC-02-037-BM | chr2 | 202149922 | 202149922 | G | A | CASP8 | p.Glu396Lys | g.202149922G > A |
| OC-02-037-BM | chr9 | 139399847 | 139399848 | G | — | NOTCH1 | p.Phe1500fs | g.139399848delG |
| OC-01-035-JR | chr17 | 7577538 | 7577538 | C | T | TP53 | p.Arg248Gln | g.7577538C > T |
| OC-01-035-JR | chr4 | 187540686 | 187540686 | G | A | FAT1 | p.Gln2352Ter | g.187540686G > A |
| OC-01-035-JR | chr9 | 139391988 | 139391988 | G | T | NOTCH1 | p.Ala2068Asp | g.139391988G > T |
| OC-01-038-GA | chr17 | 7577085 | 7577085 | C | T | TP53 | p.Glu285Lys | g.7577085C > T |
| OC-01-038-GA | chr17 | 7578290 | 7578290 | C | T | TP53 | c.560 − | g.7578290C > T |
| | | | | | | | 1G > A | |
| OC-01-038-GA | chr4 | 187541617 | 187541617 | — | C | FAT1 | p.Glu2042fs | g.187541617insC |
| OC-01-038-GA | chr4 | 187541617 | 187541617 | — | C | FAT1 | p.Glu2042fs | g.187541617_{—} |
| | | | | | | | | 187541618insC |
| OC-01-019-MA | chr17 | 7578413 | 7578413 | C | A | TP53 | p.Val173Leu | g.7578413C > A |
| OC-01-019-MA | chr17 | 7578440 | 7578440 | T | A | TP53 | p.Lys164Ter | g.7578440T > A |
| OC-02-014-RP | chr11 | 534285 | 534285 | C | A | HRAS | p.Gly13Val | g.534285C > A |
| OC-02-014-RP | chr17 | 7578526 | 7578526 | C | A | TP53 | p.Cys135Phe | g.7578526C > A |
| OC-02-014-RP | chr2 | 202137429 | 202137431 | AA | — | CASP8 | p.Arg162fs | g.202137430_{—} |
| | | | | | | | | 202137431delAA |
| OC-02-014-RP | chr4 | 187630618 | 187630618 | — | TT | FAT1 | p.Ala122fs | g.187630618insTT |
| OC-02-014-RP | chr4 | 187630618 | 187630618 | — | TT | FAT1 | p.Ala122fs | g.187630618_{—} |
| | | | | | | | | 187630619insTT |
| OC-01-066-TS | chr17 | 7578212 | 7578212 | G | A | TP53 | p.Arg213Ter | g.7578212G > A |
| OC-01-046-NB | chr17 | 7578504 | 7578505 | G | — | TP53 | p.Pro142fs | g.7578505delG |
| OC-01-046-NB | chr2 | 202150009 | 202150011 | TT | — | CASP8 | p.Cys426fs | g.202150010_{—} |
| | | | | | | | | 202150011delTT |
| OC-02-005-SaD | chr9 | 139400334 | 139400334 | C | G | NOTCH1 | c.4015 − | g.139400334C > G |
| | | | | | | | 1G > C | |
| OC-01-068-GG | chr17 | 7577094 | 7577094 | G | C | TP53 | p.Arg282Gly | g.7577094G > C |
| OC-01-064-GSH | chr17 | 7577557 | 7577558 | G | — | TP53 | p.Cys242fs | g.7577558delG |
| OC-01-064-GSH | chr3 | 178921549 | 178921549 | T | G | PIK3CA | p.Val344Gly | g.178921549T > G |
| OC-01-072-CH | chr17 | 7578269 | 7578269 | G | A | TP53 | p.Leu194Phe | g.7578269G > A |
| OC-01-072-CH | chr2 | 202141689 | 202141689 | C | T | CASE8 | p.Ala267Val | g.202141689C > T |
| OC-01-072-CH | chr4 | 187539071 | 187539071 | G | C | FAT1 | p.Ser2890Ter | g.187539071G > C |
| OC-01-072-CH | chr9 | 139412252 | 139412252 | C | A | NOTCH1 | p.Ala465Ser | g.139412252C > A |
| OC-02-034-US | chr17 | 7577094 | 7577094 | G | A | TP53 | p.Arg282Trp | g.7577094G > A |
| OC-02-034-US | chr17 | 7579312 | 7579312 | C | T | TP53 | p.Thr125Thr | g.7579312C > T |
| OC-02-034-US | chr4 | 187539564 | 187539564 | G | A | FAT1 | p.Arg2726Ter | g.187539564G > A |
| OC-02-034-US | chr4 | 187629998 | 187630002 | TGAC | — | FAT1 | p.Ser327fs | g.187629999_{—} |
| | | | | | | | | 187630002delTGAC |
| OC-02-044-HB | chr17 | 7578403 | 7578403 | C | A | TP53 | p.Cys176Phe | g.7578403C > A |
| OC-02-042-BJ | chr17 | 7577568 | 7577568 | C | T | TP53 | p.Cys238Tyr | g.7577568C > T |
| OC-02-042-BJ | chr17 | 7578457 | 7578457 | C | T | TP53 | p.Arg158His | g.7578457C > T |
| OC-02-042-BJ | chr3 | 178952085 | 178952085 | A | G | PIK3CA | p.His1047Arg | g.178952085A > G |
| OC-01-071-DT | chr17 | 7578212 | 7578212 | G | A | TP53 | p.Arg213Ter | g.7578212G > A |
| OC-02-071-DIB | chr17 | 7577022 | 7577022 | G | A | TP53 | p.Arg306Ter | g.7577022G > A |
| OC-01-086-DKH | chr17 | 7577568 | 7577568 | C | T | TP53 | p.Cys238Tyr | g.7577568C > T |
| OC-01-086-DKH | chr4 | 187539417 | 187539417 | G | A | FAT1 | p.Gln2775Ter | g.187539417G > A |
| OC-03-014 | chr17 | 7578263 | 7578263 | G | A | TP53 | p.Arg196Ter | g.7578263G > A |
| OC-03-014 | chr4 | 187518260 | 187518260 | G | A | FAT1 | p.Thr4145Met | g.187518260G > A |
| OC-02-015-KM | chr17 | 7577082 | 7577082 | C | T | TP53 | p.Glu286Lys | g.7577082C > T |
| OC-02-070-UM | chr11 | 534285 | 534285 | C | T | HRAS | p.Gly13Asp | g.534285C > T |
| OC-02-070-UM | chr17 | 7577094 | 7577094 | G | A | TP53 | p.Arg282Trp | g.7577094G > A |
| OC-02-070-UM | chr9 | 139401331 | 139401332 | T | — | NOTCH1 | p.Asp1246fs | g.139401332delT |
| OC-02-040-KG | chr2 | 202150039 | 202150039 | C | T | CASP8 | p.Arg435Ter | g.202150039C > T |
| OC-02-040-KG | chr9 | 139412375 | 139412375 | C | T | NOTCH1 | p.Glu424Lys | g.139412375C > T |
| OC-02-002-AP | chr17 | 7578524 | 7578524 | G | A | TP53 | p.Gln136Ter | g.7578524G > A |
| OC-02-002-AP | chr9 | 139412390 | 139412390 | C | G | NOTCH1 | c.1256 − | g.139412390C > G |
| | | | | | | | 1G > C | |
| OC-01-056-SKG | chr17 | 7578553 | 7578553 | T | C | TP53 | p.Tyr126Cys | g.7578553T > C |
| OC-01-056-SKG | chr4 | 187531169 | 187531169 | C | A | FAT1 | p.Gly3285Val | g.187531169C > A |
| OC-01-056-SKG | chr9 | 139396889 | 139396890 | C | — | NOTCH1 | p.Ala1740fs | g.139396890delC |
| OC-01-056-SKG | chr9 | 139412375 | 139412375 | C | T | NOTCH1 | p.Glu424Lys | g.139412375C > T |
| OC-01-060-SJ | chr17 | 7577022 | 7577022 | G | A | TP53 | p.Arg306Ter | g.7577022G > A |
| OC-02-006-BS | chr17 | 7574003 | 7574003 | G | A | TP53 | p.Arg342Ter | g.7574003G > A |
| OC-02-006-BS | chr17 | 7577120 | 7577120 | C | T | TP53 | p.Arg273His | g.7577120C > T |
| OC-01-023-CS | chr2 | 202136277 | 202136277 | C | G | CASP8 | p.Ser115Ter | g.202136277C > G |
| OC-01-023-CS | chr9 | 139405234 | 139405235 | G | — | NOTCH1 | p.Asn871fs | g.139405235delG |
| OC-01-020-TSA | chr17 | 7578263 | 7578263 | G | A | TP53 | p.Arg196Ter | g.7578263G > A |
| OC-03-012 | chr17 | 7578532 | 7578534 | TC | — | TP53 | p.Lys132fs | g.7578533_{—} |
| | | | | | | | | 7578534delTC |
| OC-01-028-MD | chr17 | 7578403 | 7578403 | C | A | TP53 | p.Cys176Phe | g.7578403C > A |
| OC-02-059-GOS | chr17 | 7578272 | 7578272 | G | T | TP53 | p.His193Asn | g.7578272G > T |
| OC-02-059-GOS | chr9 | 139412684 | 139412684 | C | A | NOTCH1 | p.Cys387Phe | g.139412684C > A |
| OC-01-034-KS | chr11 | 533873 | 533873 | C | A | HRAS | p.Gln61His | g.533873C > A |
| OC-01-034-KS | chr17 | 7578366 | 7578366 | C | T | TP53 | c.559 + | g.7578366C > T |
| | | | | | | | 5G > A | |
| OC-01-034-KS | chr2 | 202149973 | 202149973 | C | T | CASP8 | p.Arg413Ter | g.202149973C > T |
| OC-02-018-PK | chr17 | 7577547 | 7577547 | C | T | TP53 | p.Gly245Asp | g.7577547C > T |
| OC-02-061-SRO | chr17 | 7578441 | 7578441 | G | T | TP53 | p.Tyr163Ter | g.7578441G > T |
| OC-02-077-SHU | chr17 | 7578419 | 7578419 | C | A | TP53 | p.Glu171Ter | g.7578419C > A |
| OC-03-013 | chr17 | 7579353 | 7579355 | CA | — | TP53 | p.Leu111fs | g.7579354_{—} |
| | | | | | | | | 7579355delCA |
| OC-03-010 | chr17 | 7578479 | 7578479 | G | A | TP53 | p.Pro151Ser | g.7578479G > A |
| OC-02-035-PKM | chr17 | 7578448 | 7578448 | G | T | TP53 | p.Ala161Asp | g.7578448G > T |
| OC-02-035-PKM | chr2 | 202131360 | 202131360 | A | T | CASP8 | p.Lys51Ter | g.202131360A > T |
| OC-02-035-PKM | chr4 | 187584500 | 187584500 | — | TT | FAT1 | p.Ile1178fs | g.187584500_{—} |
| | | | | | | | | 187584501insTT |
| OC-02-035-PKM | chr9 | 21968242 | 21968242 | C | T | CDKN2A | c.458 − | g.21968242C > T |
| | | | | | | | 1G > A | |
| OC-03-008 | chr17 | 7578479 | 7578479 | G | A | TP53 | p.Pro151Ser | g.7578479G > A |
| OC-03-008 | chr9 | 21971138 | 21971138 | C | T | CDKN2A | p.Asp74Asn | g.21971138C > T |
| OC-02-021-BUD | chr17 | 7578375 | 7578379 | CTAT | — | TP53 | p.Asp184fs | g.7578376_{—} |
| | | | | | | | | 7578379delCTAT |
| OC-02-035-PKM | chr17 | 7578448 | 7578448 | G | T | TP53 | p.Ala161Asp | g.7578448G > T |
| OC-02-035-PKM | chr2 | 202131360 | 202131360 | A | T | CASP8 | p.Lys51Ter | g.202131360A > T |
| OC-02-035-PKM | chr4 | 187584500 | 187584500 | — | TT | FAT1 | p.Ile1178fs | g.187584500_{—} |
| | | | | | | | | 187584501insTT |
| OC-02-035-PKM | chr9 | 21968242 | 21968242 | C | T | CDKN2A | c.458 − | g.21968242C > T |
| | | | | | | | 1G > A | |
| OC-03-008 | chr17 | 7578479 | 7578479 | G | A | TP53 | p.Pro151Ser | g.7578479G > A |
| OC-03-008 | chr9 | 21971138 | 21971138 | C | T | CDKN2A | p.Asp74Asn | g.21971138C > T |
| OC-02-021-BUD | chr17 | 7578375 | 7578379 | CTAT | — | TP53 | p.Asp184fs | g.7578376_{—} |
| | | | | | | | | 7578379delCTAT |
| OC-01-001-AR | chr17 | 7578403 | 7578403 | C | A | TP53 | p.Cys176Phe | g.7578403C > A |
| OC-01-001-AR | chr4 | 187541872 | 187541873 | T | — | FAT1 | p.Asp1956fs | g.187541873delT |
| OC-01-001-AR | chr4 | 187541872 | 187541873 | T | — | FAT1 | p.Asp1956fs | g.187541873delT |
| OC-01-003-BM | chr17 | 7577574 | 7577574 | T | C | TP53 | p.Tyr236Cys | g.7577574T > C |
| OC-01-007-ST | chr17 | 7577538 | 7577538 | C | T | TP53 | p.Arg248Gln | g.7577538C > T |
| OC-01-007-ST | chr17 | 7578457 | 7578457 | C | T | TP53 | p.Arg158His | g.7578457C > T |
| OC-01-007-ST | chr2 | 202150007 | 202150007 | C | G | CASP8 | p.Ser424Ter | g.202150007C > G |
| OC-01-007-ST | chr9 | 139412697 | 139412697 | — | TT | NOTCHI | p.Glu383fs | g.139412697_{—} |
| | | | | | | | | 139412698insTT |
| OC-01-007-ST | chr9 | 21971000 | 21971000 | C | A | CDKN2A | p.Glu120Ter | g.21971000C > A |
| OC-01-007-ST | chr17 | 7577538 | 7577538 | C | T | TP53 | p.Arg248Gln | g.7577538C > T |
| OC-01-007-ST | chr2 | 202150007 | 202150007 | C | G | CASP8 | p.Ser424Ter | g.202150007C > G |
| OC-01-007-ST | chr9 | 139412697 | 139412697 | — | TT | NOTCH1 | p.Glu383fs | g.139412697_{—} |
| | | | | | | | | 139412698insTT |
| OC-01-007-ST | chr9 | 21971000 | 21971000 | C | A | CDKN2A | p.Glu120Ter | g.21971000C > A |
| OC-01-008-KA | chr11 | 534289 | 534289 | C | T | HRAS | p.Gly12Ser | g.534289C > T |
| OC-01-008-KA | chr4 | 187539097 | 187539099 | TC | — | FAT1 | p.Asp2881fs | g.187539098_{—} |
| | | | | | | | | 187539099delTC |
| OC-01-008-KA | chr4 | 187541413 | 187541415 | TC | — | FAT1 | p.Asp2109fs | g.187541414_{—} |
| | | | | | | | | 187541415delTC |
| OC-01-008-KA | chr9 | 139403409 | 139403411 | TG | — | NOTCH1 | p.Gln1028fs | g.139403410_{—} |
| | | | | | | | | 139403411delTG |
| OC-01-008-KA | chr9 | 139418164 | 139418164 | C | G | NOTCH1 | c.403 + | g.139418164C > G |
| | | | | | | | 5G > C | |
| OC-01-008-KA | chr11 | 534289 | 534289 | C | T | HRAS | p.Gly12Ser | g.534289C > T |
| OC-01-008-KA | chr4 | 187539097 | 187539099 | TC | — | FAT1 | p.Asp2881fs | g.187539098_{—} |
| | | | | | | | | 187539099delTC |
| OC-01-008-KA | chr4 | 187541413 | 187541415 | TC | — | FAT1 | p.Asp2109fs | g.187541414_{—} |
| | | | | | | | | 187541415delTC |
| OC-01-008-KA | chr9 | 139403409 | 139403411 | TG | — | NOTCH1 | p.Gln1028fs | g.139403410_{—} |
| | | | | | | | | 139403411delTG |
| OC-01-008-KA | chr9 | 139418164 | 139418164 | C | G | NOTCH1 | c.403 + | g.139418164C > G |
| | | | | | | | 5G > C | |
| OC-01-025-PC | chr2 | 202151181 | 202151181 | G | C | CASP8 | c.1305 − | g.202151181G > C |
| | | | | | | | 1G > C | |
| OC-01-025-PC | chr4 | 187584714 | 187584715 | C | — | FAT1 | p.Trp1106fs | g.187584715delC |
| OC-01-025-PC | chr2 | 202151181 | 202151181 | G | C | CASP8 | c.1305 − | g.202151181G > C |
| | | | | | | | 1G > C | |
| OC-01-026-GKD | chr17 | 7578393 | 7578393 | A | T | TP53 | p.His179Gln | g.7578393A > T |
| OC-01-030-RP | chr17 | 7577526 | 7577526 | A | G | TP53 | p.Leu252Pro | g.7577526A > G |
| OC-01-030-RP | chr4 | 187521420 | 187521420 | T | A | FAT1 | p.His3912Leu | g.187521420T > A |
| OC-01-030-RP | chr9 | 21971186 | 21971186 | G | A | CDKN2A | p.Arg58Ter | g.21971186G > A |
| OC-01-030-RP | chr17 | 7577526 | 7577526 | A | G | TP53 | p.Leu252Pro | g.7577526A > G |
| OC-01-030-RP | chr4 | 187521420 | 187521420 | T | A | FAT1 | p.His3912Leu | g.187521420T > A |
| OC-01-030-RP | chr9 | 21971186 | 21971186 | G | A | CDKN2A | p.Arg58Ter | g.21971186G > A |
| OC-01-033-PS | chr17 | 7578403 | 7578403 | C | A | TP53 | p.Cys176Phe | g.7578403C > A |
| OC-01-033-PS | chr17 | 7578403 | 7578403 | C | A | TP53 | p.Cys176Phe | g.7578403C > A |
| OC-01-037-JK | chr9 | 21971120 | 21971120 | G | A | CDKN2A | p.Arg80Ter | g.21971120G > A |
| OC-01-037-JK | chr9 | 21971120 | 21971120 | G | A | CDKN2A | p.Arg80Ter | g.21971120G > A |
| OC-01-040-AJ | chr17 | 7578537 | 7578538 | T | — | TP53 | p.Asn131fs | g.7578538delT |
| OC-01-040-AJ | chr4 | 187629360 | 187629360 | G | C | FAT1 | p.Ser541Ter | g.187629360G > C |
| OC-01-040-AJ | chr9 | 21971186 | 21971186 | G | A | CDKN2A | p.Arg58Ter | g.21971186G > A |
| OC-01-040-AJ | chr17 | 7578537 | 7578538 | T | — | TP53 | p.Asn131fs | g.7578538delT |
| OC-01-040-AJ | chr4 | 187629360 | 187629360 | G | C | FAT1 | p.Ser541Ter | g.187629360G > C |
| OC-01-040-AJ | chr9 | 21971186 | 21971186 | G | A | CDKN2A | p.Arg58Ter | g.21971186G > A |
| OC-01-045-PH | chr17 | 7577593 | 7577595 | AC | — | TP53 | p.Cys229fs | g.7577594_{—} |
| | | | | | | | | 7577595delAC |
| OC-01-047-GM | chr17 | 7577575 | 7577575 | A | C | TP53 | p.Tyr236Asp | g.7577575A > C |
| OC-01-047-GM | chr3 | 178922324 | 178922324 | G | A | PIK3CA | p.Glu365Lys | g.178922324G > A |
| OC-01-047-GM | chr17 | 7577575 | 7577575 | A | C | TP53 | p.Tyr236Asp | g.7577575A > C |
| OC-01-047-GM | chr3 | 178922324 | 178922324 | G | A | PIK3CA | p.Glu365Lys | g.178922324G > A |
| OC-01-048-MC | chr17 | 7578442 | 7578442 | T | C | TP53 | p.Tyr163Cys | g.7578442T > C |
| OC-01-049-NA | chr11 | 534289 | 534289 | C | T | HRAS | p.Gly12Ser | g.534289C > T |
| OC-01-049-NA | chr2 | 202141587 | 202141587 | G | A | CASP8 | p.Arg233Gln | g.202141587G > A |
| OC-01-049-NA | chr9 | 139405104 | 139405104 | C | A | NOTCH1 | c.2740 + | g.139405104C > A |
| | | | | | | | 1G > T | |
| OC-01-049-NA | chr9 | 139412389 | 139412389 | C | T | NOTCH1 | p.Gly419Asp | g.139412389C > T |
| OC-01-049-NA | chr11 | 534289 | 534289 | C | T | HRAS | p.Gly12Ser | g.534289C > T |
| OC-01-049-NA | chr2 | 202141587 | 202141587 | G | A | CASP8 | p.Arg233Gln | g.202141587G > A |
| OC-01-049-NA | chr9 | 139405104 | 139405104 | C | A | NOTCH1 | c.2740 + | g.139405104C > A |
| | | | | | | | 1G > T | |
| OC-01-054-GPC | chr17 | 7574034 | 7574034 | C | T | TP53 | c.994 − | g.7574034C > T |
| | | | | | | | 1G > A | |
| OC-01-054-GPC | chr2 | 202131505 | 202131505 | C | G | CASP8 | p.Ser99Cys | g.202131505C > G |
| OC-01-054-GPC | chr4 | 187541395 | 187541395 | — | GTTTC | FAT1 | p.Val2116fs | g.187541395_{—} |
| | | | | | | | | 1875413961nsGTTTC |
| OC-01-054-GPC | chr4 | 187541395 | 187541395 | — | GTTTC | FAT1 | p.Val2116fs | g.187541395insGTTTC |
| OC-01-054-GPC | chr9 | 139403384 | 139403384 | G | A | NOTCH1 | p.Gln1037Ter | g.139403384G > A |
| OC-01-054-GPC | chr9 | 21971120 | 21971120 | G | A | CDKN2A | p.Arg80Ter | g.21971120G > A |
| OC-01-054-GPC | chr2 | 202131505 | 202131505 | C | G | CASP8 | p.Ser99Cys | g.202131505C > G |
| OC-01-054-GPC | chr4 | 187541395 | 187541395 | — | GTTTC | FAT1 | p.Val2116fs | g.187541395_{—} |
| | | | | | | | | 1875413961nsGTTTC |
| OC-01-054-GPC | chr4 | 187541395 | 187541395 | — | GTTTC | FAT1 | p.Val2116fs | g.187541395insGTTTC |
| OC-01-054-GPC | chr9 | 139403384 | 139403384 | G | A | NOTCH1 | p.Gln1037Ter | g.139403384G > A |
| OC-01-054-GPC | chr9 | 21971120 | 21971120 | G | A | CDKN2A | p.Arg80Ter | g.21971120G > A |
| OC-01-055-CA | chr17 | 7577093 | 7577093 | C | T | TP53 | p.Arg282Gln | g.7577093C > T |
| OC-01-055-CA | chr17 | 7578366 | 7578366 | C | T | TP53 | c.559 + | g.7578366C > T |
| | | | | | | | 5G > A | |
| OC-01-055-CA | chr2 | 202149705 | 202149705 | C | G | CASP8 | p.Ile323Met | g.202149705C > G |
| OC-01-055-CA | chr2 | 202149735 | 202149735 | C | G | CASP8 | p.Ile333Met | g.202149735C > G |
| OC-01-055-CA | chr2 | 202151193 | 202151193 | T | G | CASP8 | p.Ile439Ser | g.202151193T > G |
| OC-01-055-CA | chr4 | 187527368 | 187527368 | C | G | FAT1 | c.10207 − | g.187527368C > G |
| | | | | | | | 1G > C | |
| OC-01-055-CA | chr4 | 187540884 | 187540884 | G | A | FAT1 | p.Gln2286Ter | g.187540884G > A |
| OC-01-055-CA | chr9 | 139410521 | 139410521 | G | C | NOTCH1 | p.Tyr527Ter | g.139410521G > C |
| OC-01-055-CA | chr9 | 21971120 | 21971120 | G | A | CDKN2A | p.Arg80Ter | g.21971120G > A |
| OC-01-055-CA | chr4 | 187540884 | 187540884 | G | A | FAT1 | p.Gln2286Ter | g.187540884G > A |
| OC-01-055-CA | chr9 | 21971120 | 21971120 | G | A | CDKN2A | p.Arg80Ter | g.21971120G > A |
| OC-01-059-LB | chr17 | 7577548 | 7577548 | C | T | TP53 | p.Gly245Ser | g.7577548C > T |
| OC-01-059-LB | chr17 | 7577548 | 7577548 | C | T | TP53 | p.Gly245Ser | g.7577548C > T |
| OC-01-061-MaS | chr17 | 7577121 | 7577121 | G | A | TP53 | p.Arg273Cys | g.7577121G > A |
| OC-01-061-MaS | chr17 | 7577121 | 7577121 | G | A | TP53 | p.Arg273Cys | g.7577121G > A |
| OC-01-065-RAM | chr17 | 7577538 | 7577538 | C | T | TP53 | p.Arg248Gln | g.7577538C > T |
| OC-01-065-RAM | chr17 | 7577538 | 7577538 | C | T | TP53 | p.Arg248Gln | g.7577538C > T |
| OC-01-070-RA | chr9 | 139392000 | 139392000 | G | C | NOTCH1 | p.Pro2064Arg | g.139392000G > C |
| OC-01-074-AK | chr17 | 7578403 | 7578403 | C | T | TP53 | p.Cys176Tyr | g.7578403C > T |
| OC-01-074-AK | chr9 | 21971120 | 21971120 | G | A | CDKN2A | p.Arg80Ter | g.21971120G > A |
| OC-01-074-AK | chr17 | 7578403 | 7578403 | C | T | TP53 | p.Cys176Tyr | g.7578403C > T |
| OC-01-074-AK | chr9 | 21971120 | 21971120 | G | A | CDKN2A | p.Arg80Ter | g.21971120G > A |
| OC-01-081-RAT | chr17 | 7578555 | 7578555 | C | T | TP53 | c.376 − | g.7578555C > T |
| | | | | | | | 1G > A | |
| OC-01-096-VE | chr9 | 21971120 | 21971120 | G | A | CDKN2A | p.Arg80Ter | g.21971120G > A |
| OC-01-096-VE | chr9 | 21971120 | 21971120 | G | A | CDKN2A | p.Arg80Ter | g.21971120G > A |
| OC-02-004-SuD | chr17 | 7574003 | 7574003 | G | A | TP53 | p.Arg342Ter | g.7574003G > A |
| OC-02-004-SuD | chr2 | 202141586 | 202141586 | C | T | CASP8 | p.Arg233Trp | g.202141586C > T |
| OC-02-004-SuD | chr4 | 187549509 | 187549509 | G | C | FAT1 | p.Gln1537Glu | g.187549509G > C |
| OC-02-004-SuD | chr9 | 139402787 | 139402787 | G | T | NOTCH1 | p.Cys1074Ter | g.139402787G > T |
| OC-02-004-SuD | chr17 | 7574003 | 7574003 | G | A | TP53 | p.Arg342Ter | g.7574003G > A |
| OC-02-004-SuD | chr2 | 202141586 | 202141586 | C | T | CASP8 | p.Arg233Trp | g.202141586C > T |
| OC-02-004-SuD | chr4 | 187549509 | 187549509 | G | C | FAT1 | p.Gln1537Glu | g.187549509G > C |
| OC-02-004-SuD | chr9 | 139402787 | 139402787 | G | T | NOTCH1 | p.Cys1074Ter | g.139402787G > T |
| OC-02-010-BKS | chr17 | 7574003 | 7574003 | G | A | TP53 | p.Arg342Ter | g.7574003G > A |
| OC-02-010-BKS | chr17 | 7579350 | 7579350 | A | T | TP53 | p.Phe113Ile | g.7579350A > T |
| OC-02-010-BKS | chr17 | 7574003 | 7574003 | G | A | TP53 | p.Arg342Ter | g.7574003G > A |
| OC-02-010-BKS | chr17 | 7579350 | 7579350 | A | T | TP53 | p.Phe113Ile | g.7579350A > T |
| OC-02-012-MK | chr17 | 7578260 | 7578260 | C | T | TP53 | p.Val197Met | g.7578260C > T |
| OC-02-012-MK | chr2 | 202141586 | 202141586 | C | T | CASP8 | p.Arg233Trp | g.202141586C > T |
| OC-02-012-MK | chr4 | 187524105 | 187524105 | C | A | FAT1 | p.Glu3812Ter | g.187524105C > A |
| OC-02-012-MK | chr4 | 187542073 | 187542075 | AA | — | FAT1 | p.Leu1889fs | g.187542074_{—} |
| | | | | | | | | 187542075delAA |
| OC-02-012-MK | chr17 | 7578260 | 7578260 | C | T | TP53 | p.Val197Met | g.7578260C > T |
| OC-02-012-MK | chr2 | 202141586 | 202141586 | C | T | CASP8 | p.Arg233Trp | g.202141586C > T |
| OC-02-012-MK | chr4 | 187524105 | 187524105 | C | A | FAT1 | p.Glu3812Ter | g.187524105C > A |
| OC-02-012-MK | chr4 | 187542073 | 187542075 | AA | — | FAT1 | p.Leu1889fs | g.187542074_{—} |
| | | | | | | | | 187542075delAA |
| OC-02-019-PJ | chr17 | 7577097 | 7577097 | C | G | TP53 | p.Asp281His | g.7577097C > G |
| OC-02-019-PJ | chr2 | 202136252 | 202136252 | C | T | CASP8 | p.Gln107Ter | g.202136252C > T |
| OC-02-019-PJ | chr2 | 202136252 | 202136252 | C | T | CASP8 | p.Gln107Ter | g.202136252C > T |
| OC-02-019-PJ | chr17 | 7577097 | 7577097 | C | G | TP53 | p.Asp281His | g.7577097C > G |
| OC-02-019-PJ | chr2 | 202136252 | 202136252 | C | T | CASP8 | p.Gln107Ter | g.202136252C > T |
| OC-02-019-PJ | chr2 | 202136252 | 202136252 | C | T | CASP8 | p.Gln107Ter | g.202136252C > T |
| OC-02-020-NK | chr11 | 534288 | 534288 | C | G | HRAS | p.Gly12Ala | g.534288C > G |
| OC-02-020-NK | chr2 | 202131515 | 202131515 | G | A | CASP8 | c.305 + | g.202131515G > A |
| | | | | | | | 1G > A | |
| OC-02-020-NK | chr4 | 187518900 | 187518900 | C | T | FAT1 | p.Gly4102Arg | g.187518900C > T |
| OC-02-020-NK | chr4 | 187524066 | 187524066 | G | A | FAT1 | p.Gln3825Ter | g.187524066G > A |
| OC-02-020-NK | chr4 | 187628418 | 187628418 | C | T | FAT1 | p.Gly855Glu | g.187628418C > T |
| OC-02-020-NK | chr4 | 187628680 | 187628680 | C | T | FAT1 | p.Asp768Asn | g.187628680C > T |
| OC-02-020-NK | chr9 | 139412735 | 139412739 | ACAG | — | NOTCH1 | p.Leu369fs | g.139412736_{—} |
| | | | | | | | | 139412739delACAG |
| OC-02-020-NK | chr9 | 139413100 | 139413100 | C | T | NOTCH1 | p.Ala348Thr | g.139413100C > T |
| OC-02-020-NK | chr9 | 139417572 | 139417572 | — | G | NOTCH1 | p.Phe158fs | g.139417572insG |
| OC-02-020-NK | chr9 | 139412735 | 139412739 | ACAG | — | NOTCH1 | p.Leu369fs | g.139412736_{—} |
| | | | | | | | | 139412739delACAG |
| OC-02-023-SR | chr11 | 534285 | 534285 | C | A | HRAS | p.Gly13Val | g.534285C > A |
| OC-02-023-SR | chr17 | 7577094 | 7577094 | G | A | TP53 | p.Arg282Trp | g.7577094G > A |
| OC-02-023-SR | chr17 | 7577538 | 7577538 | C | T | TP53 | p.Arg248Gln | g.7577538C > T |
| OC-02-023-SR | chr2 | 202149604 | 202149604 | G | T | CASP8 | p.Glu290Ter | g.202149604G > T |
| OC-02-023-SR | chr4 | 187519154 | 187519154 | G | A | FAT1 | p.Gln4077Ter | g.187519154G > A |
| OC-02-023-SR | chr4 | 187525133 | 187525133 | T | A | FAT1 | c.10549 − | g.187525133T > A |
| | | | | | | | 2A > T | |
| OC-02-023-SR | chr9 | 139410069 | 139410069 | — | TCTG | NOTCH1 | p.Leu590fs | g.139410069_{—} |
| | | | | | | | | 139410070insTCTG |
| OC-02-023-SR | chr9 | 139410069 | 139410069 | — | ATCTG | NOTCH1 | p.Leu590fs | g.139410069insATCTG |
| OC-02-023-SR | chr9 | 21971120 | 21971120 | G | A | CDKN2A | p.Arg80Ter | g.21971120G > A |
| OC-02-023-SR | chr11 | 534285 | 534285 | C | A | HRAS | p.Gly13Val | g.534285C > A |
| OC-02-023-SR | chr17 | 7577094 | 7577094 | G | A | TP53 | p.Arg282Trp | g.7577094G > A |
| OC-02-023-SR | chr17 | 7577538 | 7577538 | C | T | TP53 | p.Arg248Gln | g.7577538C > T |
| OC-02-023-SR | chr2 | 202149604 | 202149604 | G | T | CASP8 | p.Glu290Ter | g.202149604G > T |
| OC-02-023-SR | chr4 | 187519154 | 187519154 | G | A | FAT1 | p.Gln4077Ter | g.187519154G > A |
| OC-02-023-SR | chr4 | 187525133 | 187525133 | T | A | FAT1 | c.10549 − | g.187525133T > A |
| | | | | | | | 2A > T | |
| OC-02-023-SR | chr9 | 139410069 | 139410069 | — | TCTG | NOTCH1 | p.Leu590fs | g.139410069_{—} |
| | | | | | | | | 139410070insTCTG |
| OC-02-023-SR | chr9 | 139410069 | 139410069 | — | ATCTG | NOTCH1 | p.Leu590fs | g.139410069insATCTG |
| OC-02-023-SR | chr9 | 21971120 | 21971120 | G | A | CDKN2A | p.Arg80Ter | g.21971120G > A |
| OC-02-025-RC | chr17 | 7574003 | 7574003 | G | A | TP53 | p.Arg342Ter | g.7574003G > A |
| OC-02-025-RC | chr17 | 7579307 | 7579307 | C | A | TP53 | c.375 + 5G > T | g.7579307C > A |
| OC-02-025-RC | chr3 | 178952085 | 178952085 | A | G | PIK3CA | p.His1047Arg | g.178952085A > G |
| OC-02-025-RC | chr9 | 21971120 | 21971120 | G | A | CDKN2A | p.Arg80Ter | g.21971120G > A |
| OC-02-025-RC | chr17 | 7574003 | 7574003 | G | A | TP53 | p.Arg342Ter | g.7574003G > A |
| OC-02-025-RC | chr3 | 178952085 | 178952085 | A | G | PIK3CA | p.His1047Arg | g.178952085A > G |
| OC-02-025-RC | chr9 | 21971120 | 21971120 | G | A | CDKN2A | p.Arg80Ter | g.21971120G > A |
| OC-02-027-SS | chr17 | 7577157 | 7577157 | T | C | TP53 | c.783 − | g.7577157T > C |
| | | | | | | | 2A > G | |
| OC-02-027-SS | chr17 | 7579546 | 7579546 | — | G | TP53 | p.Asp48fS | g.7579546_{—} |
| | | | | | | | | 7579547insG |
| OC-02-027-SS | chr2 | 202131431 | 202131431 | G | T | CASP8 | p.Leu74Phe | g.202131431G > T |
| OC-02-027-SS | chr4 | 187549395 | 187549395 | C | T | FAT1 | p.Ala1575Thr | g.187549395C > T |
| OC-02-027-SS | chr17 | 7577157 | 7577157 | T | C | TP53 | c.783 − | g.7577157T > C |
| | | | | | | | 2A > G | |
| OC-02-027-SS | chr17 | 7577157 | 7577157 | T | C | TP53 | c.783 − | g.7577157T > C |
| | | | | | | | 2A > G | |
| OC-02-027-SS | chr17 | 7579546 | 7579546 | — | G | TP53 | p.Asp48fS | g.7579546_{—} |
| | | | | | | | | 7579547insG |
| OC-02-027-SS | chr2 | 202131431 | 202131431 | G | T | CASP8 | p.Leu74Phe | g.202131431G > T |
| OC-02-029-KAM | chr17 | 7578406 | 7578406 | C | T | TP53 | p.Arg175His | g.7578406C > T |
| OC-02-029-KAM | chr2 | 202150003 | 202150003 | C | T | CASP8 | p.Gln423Ter | g.202150003C > T |
| OC-02-029-KAM | chr4 | 187530400 | 187530400 | G | C | FAT1 | p.Ser3381Arg | g.187530400G > C |
| OC-02-029-KAM | chr9 | 139411722 | 139411722 | A | T | NOTCH1 | c. 1555 + | g.139411722A > T |
| | | | | | | | 2T > A | |
| OC-02-029-KAM | chr2 | 202150003 | 202150003 | C | T | CASI8 | p.Gln423Ter | g.202150003C > T |
| OC-02-033-TRM | chr17 | 7577539 | 7577539 | G | A | TP53 | p.Arg248Trp | g.7577539G > A |
| OC-02-033-TRM | chr4 | 187517872 | 187517872 | T | G | FAT1 | p.Glu4274Asp | g.187517872T > G |
| OC-02-033-TRM | chr4 | 187629920 | 187629920 | — | TGAA | FAT1 | p. Val355fs | g.187629920_{—} |
| | | | | | | | | 187629921insTGAA |
| OC-02-033-TRM | chr9 | 139399362 | 139399362 | C | T | NOTCH1 | p.Arg1594Gln | g.139399362C > T |
| OC-02-033-TRM | chr17 | 7577539 | 7577539 | G | A | TP53 | p.Arg248Trp | g.7577539G > A |
| OC-02-033-TRM | chr4 | 187629920 | 187629920 | — | TGAA | FAT1 | p. Val355fs | g.187629920_{—} |
| | | | | | | | | 187629921insTGAA |
| OC-02-033-TRM | chr9 | 139399362 | 139399362 | C | T | NOTCH1 | p.Arg1594Gln | g.139399362C > T |
| OC-02-043-DCS | chr17 | 7577513 | 7577513 | — | G | TP53 | p.Leu257fs | g.7577513_{—} |
| | | | | | | | | 7577514insG |
| OC-02-043-DCS | chr17 | 7578209 | 7578209 | G | A | TP53 | p.His214Tyr | g.7578209G > A |
| OC-02-043-DCS | chr4 | 187531170 | 187531170 | C | A | FAT1 | c.9854 − | g.187531170C > A |
| | | | | | | | 1G > T | |
| OC-02-043-DCS | chr9 | 139413085 | 139413085 | G | A | NOTCHI | p.Arg353Cys | g.139413085G > A |
| OC-02-043-DCS | chr9 | 21971123 | 21971125 | GA | — | CDKN2A | p.Leu78fS | g.21971124_{—} |
| | | | | | | | | 21971125delGA |
| OC-02-043-DCS | chr17 | 7577513 | 7577513 | — | G | TP53 | p.Leu257fs | g.7577513_{—} |
| | | | | | | | | 7577514insG |
| OC-02-043-DCS | chr17 | 7578209 | 7578209 | G | A | TP53 | p.His214Tyr | g.7578209G > A |
| OC-02-043-DCS | chr4 | 187531170 | 187531170 | C | A | FAT1 | c.9854-1G>T | g.187531170C > A |
| OC-02-043-DCS | chr9 | 139413085 | 139413085 | G | A | NOTCH1 | p.Arg353Cys | g.139413085G > A |
| OC-02-050-SN | chr2 | 202149751 | 202149751 | C | T | CASP8 | p.Gln339Ter | g.202149751C > T |
| OC-02-050-SN | chr2 | 202149751 | 202149751 | C | T | CASP8 | p.Gln339Ter | g.202149751C > T |
| OC-02-066-AB | chr17 | 7577022 | 7577022 | G | A | TP53 | p.Arg306Ter | g.7577022G > A |
| OC-02-066-AB | chr17 | 7577022 | 7577022 | G | A | TP53 | p.Arg306Ter | g.7577022G > A |
| OC-02-067-KN | chr17 | 7577098 | 7577098 | T | G | TP53 | p.Arg280Ser | g.7577098T > G |
| OC-02-067-KN | chr9 | 21971120 | 21971120 | G | A | CDKN2A | p.Arg80Ter | g.21971120G > A |
| OC-02-067-KN | chr17 | 7577098 | 7577098 | T | G | TP53 | p.Arg280Ser | g.7577098T > G |
| OC-02-067-KN | chr9 | 21971120 | 21971120 | G | A | CDKN2A | p.Arg80Ter | g.21971120G > A |
| OC-03-009 | chr17 | 7574003 | 7574003 | G | A | TP53 | p.Arg342Ter | g.7574003G > A |
| OC-03-009 | chr17 | 7574003 | 7574003 | G | A | TP53 | p.Arg342Ter | g.7574003G > A |
| OC-03-011 | chr3 | 178936091 | 178936091 | G | A | PIK3CA | p.Glu545Lys | g.178936091G > A |
| OC-03-015 | chr17 | 7577082 | 7577082 | C | T | TP53 | p.Glu286Lys | g.7577082C > T |
| OC-03-015 | chr3 | 178952085 | 178952085 | A | G | PIK3CA | p.His1047Arg | g.178952085A > G |
| OC-03-015 | chr9 | 21971028 | 21971028 | C | T | CDKN2A | p.Trp110Ter | g.21971028C > T |
| OC-03-015 | chr17 | 7577082 | 7577082 | C | T | TP53 | p.Glu286Lys | g.7577082C > T |
| OC-03-015 | chr3 | 178952085 | 178952085 | A | G | PIK3CA | p.His1047Arg | g.178952085A > G |
| OC-03-015 | chr9 | 21971028 | 21971028 | C | T | CDKN2A | p.Trp110Ter | g.21971028C > T |
| OC-03-015 | chr17 | 7577082 | 7577082 | C | T | TP53 | p.Glu286Lys | g.7577082C > T |
| OC-03-015 | chr3 | 178952085 | 178952085 | A | G | PIK3CA | p.His1047Arg | g.178952085A > G |
| OC-03-015 | chr9 | 21971028 | 21971028 | C | T | CDKN2A | p.Trp110Ter | g.21971028C > T |
| OC-03-015 | chr17 | 7577082 | 7577082 | C | T | TP53 | p.Glu286Lys | g.7577082C > T |
| OC-03-015 | chr3 | 178952085 | 178952085 | A | G | PIK3CA | p.His1047Arg | g.178952085A > G |
| OC-03-015 | chr9 | 21971028 | 21971028 | C | T | CDKN2A | p.Trp110Ter | g.21971028C > T |

| | | FFPE | | | SALIVA | | |
|---|---|---|---|---|---|---|---|
| | | | Supporting | | | Supporting | Percent_VAF |
| Sample ID | Status | TotalReads | Reads | Percent_VAF | TotalReads | Reads | VAF |
| OC-02-055-RJ | Shortlisted | 342 | 98 | 28.65 | 8357 | 69 | 0.83 |
| OC-02-055-RJ | Shortlisted | 280 | 61 | 21.79 | 7173 | 58 | 0.81 |
| OC-02-055-RJ | Shortlisted | 281 | 78 | 27.76 | 11502 | 87 | 0.76 |
| OC-02-055-RJ | Shortlisted | 238 | 65 | 27.31 | 12692 | 66 | 0.52 |
| OC-03-005 | Shortlisted | 977 | 68 | 6.96 | NA | NA | NA |
| OC-03-005 | Shortlisted | 2737 | 263 | 9.61 | 9872 | 33 | 0.33 |
| OC-03-005 | Shortlisted | 1692 | 233 | 13.77 | 10220 | 16 | 0.16 |
| OC-03-005 | VUS | 1409 | 181 | 12.85 | 10163 | 15 | 0.15 |
| OC-03-005 | Shortlisted | 3067 | 581 | 18.94 | 11132 | 18 | 0.16 |
| OC-03-005 | Shortlisted | 1993 | 446 | 22.38 | 10911 | 57 | 0.52 |
| OC-01-102-NV | Shortlisted | 812 | 106 | 13.05 | 13513 | 26 | 0.19 |
| OC-01-102-NV | Shortlisted | 3094 | 892 | 28.83 | 22503 | 113 | 0.5 |
| OC-02-038-AM | Shortlisted | 284 | 17 | 5.99 | 12849 | 23 | 0.18 |
| OC-02-038-AM | VUS | 748 | 55 | 7.35 | NA | NA | NA |
| OC-02-038-AM | Shortlisted | 352 | 25 | 7.1 | 6410 | 20 | 0.31 |
| OC-02-038-AM | Shortlisted | 27 | 8 | 29.63 | 6649 | 21 | 0.32 |
| OC-02-045-KCD | Shortlisted | 653 | 171 | 26.19 | 22603 | 181 | 0.8 |
| OC-02-045-KCD | Shortlisted | 777 | 195 | 25.1 | 15292 | 141 | 0.92 |
| OC-02-045-KCD | Shortlisted | 713 | 175 | 24.54 | 15018 | 140 | 0.93 |
| OC-02-045-KCD | VUS | 712 | 129 | 18.12 | 8693 | 58 | 0.67 |
| OC-02-045-KCD | Shortlisted | 634 | 191 | 30.13 | 11165 | 184 | 1.65 |
| OC-02-045-KCD | Shortlisted | 106 | 30 | 28.3 | 8783 | 196 | 2.23 |
| OC-02-068-NIK | Shortlisted | 564 | 48 | 8.51 | 8251 | 15 | 0.18 |
| OC-02-068-NIK | Shortlisted | 156 | 45 | 28.85 | 6411 | 24 | 0.37 |
| OC-02-036-DAK | Shortlisted | 524 | 151 | 28.82 | 7771 | 232 | 2.99 |
| OC-02-036-DAK | Shortlisted | 465 | 79 | 16.99 | 9409 | 150 | 1.59 |
| OC-02-036-DAK | Shortlisted | 187 | 68 | 36.36 | 5297 | 235 | 4.44 |
| OC-01-013-SR | Shortlisted | 625 | 317 | 50.72 | 5644 | 28 | 0.5 |
| OC-01-013-SR | Shortlisted | 270 | 89 | 32.96 | 4704 | 25 | 0.53 |
| OC-01-014-SU | Shortlisted | 1204 | 409 | 33.97 | 5657 | 10 | 0.18 |
| OC-01-014-SU | Shortlisted | 983 | 337 | 34.28 | 4196 | 13 | 0.31 |
| OC-02-022-SAB | Shortlisted | 1084 | 343 | 31.64 | 8464 | 60 | 0.71 |
| OC-02-022-SAB | Shortlisted | 811 | 212 | 26.14 | 8465 | 40 | 0.47 |
| OC-02-022-SAB | Shortlisted | 877 | 215 | 24.52 | 9141 | 33 | 0.36 |
| OC-02-022-SAB | Shortlisted | 449 | 110 | 24.5 | 5847 | 12 | 0.21 |
| OC-01-032-SUS | Shortlisted | 808 | 131 | 16.21 | 10319 | 41 | 0.4 |
| OC-01-032-SUS | Shortlisted | 1101 | 65 | 5.9 | 9555 | 14 | 0.15 |
| OC-01-032-SUS | Shortlisted | 1145 | 239 | 20.87 | 9774 | 40 | 0.41 |
| OC-01-032-SUS | Shortlisted | 320 | 34 | 10.62 | 8494 | 33 | 0.39 |
| OC-01-032-SUS | Shortlisted | 402 | 65 | 16.17 | 7341 | 16 | 0.22 |
| OC-02-003-BK | Shortlisted | 1401 | 73 | 5.21 | 9010 | 10 | 0.11 |
| OC-02-003-BK | Shortlisted | 2141 | 86 | 4.02 | NA | NA | NA |
| OC-01-050-MP | Shortlisted | 724 | 125 | 17.27 | 7063 | 22 | 0.31 |
| OC-01-050-MP | Shortlisted | 793 | 74 | 9.33 | NA | NA | NA |
| OC-01-050-MP | VUS | 502 | 42 | 8.37 | 11554 | 23 | 0.2 |
| OC-01-050-MP | VUS | 1078 | 583 | 54.08 | 8276 | 42 | 0.51 |
| OC-01-050-MP | Shortlisted | 454 | 246 | 54.19 | 8457 | 31 | 0.37 |
| OC-02-007-DP | Shortlisted | 523 | 55 | 10.52 | NA | NA | NA |
| OC-02-007-DP | Shortlisted | 557 | 44 | 7.9 | 8812 | 22 | 0.25 |
| OC-02-007-DP | Shortlisted | 589 | 58 | 9.85 | 5081 | 16 | 0.31 |
| OC-02-007-DP | Shortlisted | 124 | 22 | 17.74 | 6308 | 15 | 0.24 |
| OC-02-007-DP | Shortlisted | 193 | 20 | 10.36 | 5533 | 13 | 0.23 |
| OC-01-018-SSR | Shortlisted | 773 | 394 | 50.97 | 3587 | 13 | 0.36 |
| OC-01-018-SSR | Shortlisted | 995 | 609 | 61.21 | 7193 | 21 | 0.29 |
| OC-02-016-DC | Shortlisted | 435 | 173 | 39.77 | 7914 | 99 | 1.25 |
| OC-02-016-DC | Shortlisted | 139 | 44 | 31.65 | 6124 | 34 | 0.56 |
| OC-01-002-ABR | Shortlisted | 580 | 111 | 19.14 | NA | NA | NA |
| OC-01-002-ABR | Shortlisted | 537 | 93 | 17.32 | NA | NA | NA |
| OC-01-002-ABR | VUS | 901 | 372 | 41.29 | 6287 | 18 | 0.29 |
| OC-01-002-ABR | Shortlisted | 456 | 234 | 51.32 | 5983 | 33 | 0.55 |
| OC-01-006-SA | Shortlisted | 946 | 118 | 12.47 | 7426 | 11 | 0.15 |
| OC-01-006-SA | Shortlisted | 758 | 53 | 6.99 | NA | NA | NA |
| OC-01-006-SA | Shortlisted | 206 | 35 | 16.99 | 7225 | 14 | 0.19 |
| OC-01-029-JR | Shortlisted | 1019 | 240 | 23.55 | 8336 | 42 | 0.5 |
| OC-02-009-RS | Shortlisted | 529 | 49 | 9.26 | 6512 | 12 | 0.18 |
| OC-02-009-RS | Shortlisted | 680 | 39 | 5.74 | 5728 | 12 | 0.21 |
| OC-02-009-RS | Shortlisted | 800 | 65 | 8.12 | 5925 | 16 | 0.27 |
| OC-02-009-RS | VUS | 692 | 58 | 8.38 | 6058 | 9 | 0.15 |
| OC-01-075-GST | Shortlisted | 803 | 286 | 35.62 | 6733 | 22 | 0.33 |
| OC-01-075-GST | VUS | 1018 | 197 | 19.35 | 6735 | 23 | 0.34 |
| OC-01-075-GST | Shortlisted | 496 | 103 | 20.77 | 9640 | 14 | 0.15 |
| OC-01-075-GST | Shortlisted | 784 | 123 | 15.69 | 9920 | 40 | 0.4 |
| OC-02-030-RB | Shortlisted | 217 | 24 | 11.06 | 8545 | 90 | 1.05 |
| OC-02-030-RB | VUS | 143 | 6 | 4.2 | NA | NA | NA |
| OC-02-030-RB | Shortlisted | 98 | 27 | 27.55 | 6379 | 104 | 1.63 |
| OC-03-017 | Shortlisted | 1978 | 457 | 23.1 | 8467 | 41 | 0.48 |
| OC-03-017 | Shortlisted | 1858 | 342 | 18.41 | 6884 | 11 | 0.16 |
| OC-03-002 | Shortlisted | 813 | 176 | 21.65 | 7211 | 30 | 0.42 |
| OC-03-002 | Shortlisted | 422 | 109 | 25.83 | 8271 | 30 | 0.36 |
| OC-01-092-LA | Shortlisted | 327 | 106 | 32.42 | NA | NA | NA |
| OC-01-092-LA | VUS | 404 | 45 | 11.14 | NA | NA | NA |
| OC-03-004 | Shortlisted | 2106 | 478 | 22.7 | 7997 | 66 | 0.83 |
| OC-03-004 | Shortlisted | 883 | 139 | 15.74 | 6799 | 28 | 0.41 |
| OC-03-004 | Shortlisted | 242 | 18 | 7.44 | 7684 | 40 | 0.52 |
| OC-01-101-JMA | Shortlisted | 1442 | 633 | 43.9 | 6036 | 167 | 2.77 |
| OC-02-037-BM | Shortlisted | 420 | 213 | 50.71 | 15078 | 149 | 0.99 |
| OC-02-037-BM | VUS | 445 | 105 | 23.6 | 8895 | 57 | 0.64 |
| OC-02-037-BM | VUS | 607 | 129 | 21.25 | 14909 | 78 | 0.52 |
| OC-02-037-BM | Shortlisted | 611 | 226 | 36.99 | 17310 | 152 | 0.88 |
| OC-01-035-JR | Shortlisted | 536 | 142 | 26.49 | 9584 | 48 | 0.5 |
| OC-01-035-JR | Shortlisted | 857 | 391 | 45.62 | 8204 | 134 | 1.63 |
| OC-01-035-JR | VUS | 292 | 68 | 23.29 | 4144 | 20 | 0.48 |
| OC-01-038-GA | Shortlisted | 1139 | 167 | 14.66 | 11514 | 35 | 0.3 |
| OC-01-038-GA | Shortlisted | 1374 | 225 | 16.38 | 11445 | 37 | 0.32 |
| OC-01-038-GA | Shortlisted | 1592 | 486 | 30.53 | 10635 | 99 | 0.93 |
| OC-01-038-GA | Shortlisted | 1592 | 486 | 30.53 | 10635 | 99 | 0.93 |
| OC-01-019-MA | Shortlisted | 3149 | 429 | 13.62 | 9303 | 12 | 0.13 |
| OC-01-019-MA | Shortlisted | 3158 | 483 | 15.29 | NA | NA | NA |
| OC-02-014-RP | Shortlisted | 2571 | 1008 | 39.21 | 6393 | 23 | 0.36 |
| OC-02-014-RP | Shortlisted | 1304 | 486 | 37.27 | 6873 | 20 | 0.29 |
| OC-02-014-RP | Shortlisted | 810 | 135 | 16.67 | NA | NA | NA |
| OC-02-014-RP | Shortlisted | 367 | 102 | 27.79 | 7734 | 8 | 0.1 |
| OC-02-014-RP | Shortlisted | 367 | 102 | 27.79 | 7734 | 8 | 0.1 |
| OC-01-066-TS | Shortlisted | 740 | 266 | 35.95 | 8264 | 137 | 1.66 |
| OC-01-046-NB | Shortlisted | 888 | 172 | 19.37 | 8033 | 32 | 0.4 |
| OC-01-046-NB | VUS | 1318 | 138 | 10.47 | 8190 | 11 | 0.13 |
| OC-02-005-SaD | Shortlisted | 249 | 10 | 4.02 | 3731 | 62 | 1.66 |
| OC-01-068-GG | Shortlisted | 488 | 355 | 72.75 | 6873 | 38 | 0.55 |
| OC-01-064-GSH | Shortlisted | 430 | 69 | 16.05 | 4633 | 9 | 0.19 |
| OC-01-064-GSH | VUS | 1064 | 85 | 7.99 | 5694 | 16 | 0.28 |
| OC-01-072-CH | Shortlisted | 456 | 54 | 11.84 | 7596 | 36 | 0.47 |
| OC-01-072-CH | VUS | 531 | 70 | 13.18 | 5572 | 31 | 0.56 |
| OC-01-072-CH | Shortlisted | 649 | 67 | 10.32 | 6676 | 31 | 0.46 |
| OC-01-072-CH | VUS | 504 | 57 | 11.31 | 11361 | 56 | 0.49 |
| OC-02-034-US | Shortlisted | 596 | 164 | 27.52 | 12281 | 69 | 0.56 |
| OC-02-034-US | Shortlisted | 446 | 101 | 22.65 | 10073 | 40 | 0.4 |
| OC-02-034-US | Shortlisted | 514 | 130 | 25.29 | 8123 | 47 | 0.58 |
| OC-02-034-US | Shortlisted | 496 | 96 | 19.35 | 9766 | 80 | 0.82 |
| OC-02-044-HB | Shortlisted | 82 | 10 | 12.2 | 9395 | 22 | 0.23 |
| OC-02-042-BJ | Shortlisted | 175 | 10 | 5.71 | 8736 | 13 | 0.15 |
| OC-02-042-BJ | Shortlisted | 142 | 12 | 8.45 | NA | NA | NA |
| OC-02-042-BJ | Shortlisted | 254 | 20 | 7.87 | NA | NA | NA |
| OC-01-071-DT | Shortlisted | 434 | 129 | 29.72 | 4257 | 14 | 0.33 |
| OC-02-071-DIB | Shortlisted | 587 | 69 | 11.75 | 10054 | 28 | 0.28 |
| OC-01-086-DKH | Shortlisted | 644 | 59 | 9.16 | 7284 | 21 | 0.29 |
| OC-01-086-DKH | Shortlisted | 414 | 19 | 4.59 | 8265 | 9 | 0.11 |
| OC-03-014 | Shortlisted | 3864 | 270 | 6.99 | 9134 | 18 | 0.2 |
| OC-03-014 | VUS | 3184 | 221 | 6.94 | NA | NA | NA |
| OC-02-015-KM | Shortlisted | 133 | 32 | 24.06 | 9477 | 85 | 0.9 |
| OC-02-070-UM | Shortlisted | 462 | 25 | 5.41 | 13899 | 22 | 0.16 |
| OC-02-070-UM | Shortlisted | 478 | 22 | 4.6 | 11267 | 23 | 0.2 |
| OC-02-070-UM | Shortlisted | 194 | 10 | 5.15 | NA | NA | NA |
| OC-02-040-KG | VUS | 558 | 33 | 5.91 | NA | NA | NA |
| OC-02-040-KG | VUS | 221 | 11 | 4.98 | 11591 | 17 | 0.15 |
| OC-02-002-AP | Shortlisted | 967 | 233 | 24.1 | 10965 | 172 | 1.57 |
| OC-02-002-AP | Shortlisted | 1230 | 487 | 39.59 | 10949 | 264 | 2.41 |
| OC-01-056-SKG | Shortlisted | 357 | 85 | 23.81 | 12764 | 94 | 0.74 |
| OC-01-056-SKG | VUS | 576 | 189 | 32.81 | 9909 | 78 | 0.79 |
| OC-01-056-SKG | Shortlisted | 685 | 59 | 8.61 | 16074 | 23 | 0.14 |
| OC-01-056-SKG | VUS | 405 | 105 | 25.93 | 14559 | 90 | 0.62 |
| OC-01-060-SJ | Shortlisted | 365 | 148 | 40.55 | 6702 | 48 | 0.72 |
| OC-02-006-BS | Shortlisted | 545 | 67 | 12.29 | NA | NA | NA |
| OC-02-006-BS | Shortlisted | 820 | 200 | 24.39 | 8181 | 12 | 0.15 |
| OC-01-023-CS | Shortlisted | 778 | 200 | 25.71 | 12825 | 196 | 1.53 |
| OC-01-023-CS | Shortlisted | 567 | 81 | 14.29 | 16671 | 88 | 0.53 |
| OC-01-020-TSA | Shortlisted | 301 | 83 | 27.57 | 6933 | 54 | 0.78 |
| OC-03-012 | Shortlisted | 2918 | 401 | 13.74 | 9866 | 10 | 0.1 |
| OC-01-028-MD | Shortlisted | 1147 | 266 | 23.19 | 9883 | 62 | 0.63 |
| OC-02-059-GOS | Shortlisted | 1290 | 74 | 5.74 | 16693 | 27 | 0.16 |
| OC-02-059-GOS | VUS | 667 | 34 | 5.1 | 18788 | 23 | 0.12 |
| OC-01-034-KS | Shortlisted | 347 | 42 | 12.1 | NA | NA | NA |
| OC-01-034-KS | Shortlisted | 204 | 22 | 10.78 | NA | NA | NA |
| OC-01-034-KS | VUS | 340 | 85 | 25 | 13934 | 25 | 0.18 |
| OC-02-018-PK | Shortlisted | 356 | 141 | 39.61 | 5268 | 41 | 0.78 |
| OC-02-061-SRO | Shortlisted | 194 | 22 | 11.34 | 17034 | 112 | 0.66 |
| OC-02-077-SHU | Shortlisted | 2556 | 185 | 7.24 | 10562 | 13 | 0.12 |
| OC-03-013 | Shortlisted | 4374 | 1103 | 25.22 | 7628 | 19 | 0.25 |
| OC-03-010 | Shortlisted | 2527 | 1152 | 45.59 | 14086 | 46 | 0.33 |
| OC-02-035-PKM | Shortlisted | 663 | 54 | 8.14 | 14049 | 81 | 0.58 |
| OC-02-035-PKM | Shortlisted | 817 | 70 | 8.57 | 16123 | 61 | 0.38 |
| OC-02-035-PKM | Shortlisted | 838 | 234 | 27.92 | 6702 | 137 | 2.0 |
| OC-02-035-PKM | VUS | 555 | 39 | 7.03 | 6054 | 40 | 0.66 |
| OC-03-008 | Shortlisted | 2332 | 1289 | 55.27 | 8454 | 30 | 0.35 |
| OC-03-008 | Shortlisted | 330 | 151 | 45.76 | 5572 | 21 | 0.38 |
| OC-02-021-BUD | Shortlisted | 115 | 48 | 41.74 | 8423 | 32 | 0.38 |
| OC-02-035-PKM | Shortlisted | 1268 | 147 | 11.59 | 14049 | 81 | 0.58 |
| OC-02-035-PKM | Shortlisted | 1039 | 70 | 6.74 | 16123 | 61 | 0.38 |
| OC-02-035-PKM | Shortlisted | 784 | 208 | 26.53 | 6702 | 137 | 2.04 |
| OC-02-035-PKM | VUS | 727 | 69 | 9.49 | 6054 | 40 | 0.66 |
| OC-03-008 | Shortlisted | 5401 | 2943 | 54.49 | 8454 | 30 | 0.35 |
| OC-03-008 | Shortlisted | 865 | 354 | 40.92 | 5572 | 21 | 0.38 |
| OC-02-021-BUD | Shortlisted | 165 | 67 | 40.61 | 8423 | 32 | 0.38 |
| OC-01-001-AR | Shortlisted | 792 | 118 | 14.9 | NA | NA | NA |
| OC-01-001-AR | Shortlisted | 796 | 74 | 9.3 | 3015 | 6 | 0.2 |
| OC-01-001-AR | Shortlisted | 796 | 74 | 9.3 | NA | NA | NA |
| OC-01-003-BM | Shortlisted | 159 | 38 | 23.9 | NA | NA | NA |
| OC-01-007-ST | Shortlisted | 343 | 22 | 6.41 | NA | NA | NA |
| OC-01-007-ST | Shortlisted | 250 | 13 | 5.2 | NA | NA | NA |
| OC-01-007-ST | VUS | 551 | 55 | 9.98 | 9806 | 14 | 0.14 |
| OC-01-007-ST | Shortlisted | 239 | 44 | 18.41 | 11495 | 95 | 0.83 |
| OC-01-007-ST | Shortlisted | 251 | 72 | 28.69 | 8808 | 69 | 0.78 |
| OC-01-007-ST | Shortlisted | 343 | 22 | 6.41 | 8470 | 9 | 0.11 |
| OC-01-007-ST | VUS | 551 | 55 | 9.98 | 9602 | 14 | 0.15 |
| OC-01-007-ST | Shortlisted | 239 | 44 | 18.41 | 10914 | 91 | 0.83 |
| OC-01-007-ST | Shortlisted | 251 | 72 | 28.69 | 9140 | 63 | 0.69 |
| OC-01-008-KA | Shortlisted | 1903 | 678 | 35.63 | 10031 | 13 | 0.13 |
| OC-01-008-KA | Shortlisted | 1656 | 366 | 22.1 | 8967 | 11 | 0.12 |
| OC-01-008-KA | Shortlisted | 1704 | 348 | 20.42 | 9854 | 25 | 0.25 |
| OC-01-008-KA | Shortlisted | 1518 | 210 | 13.83 | NA | NA | NA |
| OC-01-008-KA | Shortlisted | 1382 | 202 | 14.62 | 10090 | 11 | 0.11 |
| OC-01-008-KA | Shortlisted | 1903 | 678 | 35.63 | 6478 | 12 | 0.19 |
| OC-01-008-KA | Shortlisted | 1656 | 366 | 22.1 | 5399 | 19 | 0.35 |
| OC-01-008-KA | Shortlisted | 1704 | 348 | 20.42 | 5494 | 8 | 0.15 |
| OC-01-008-KA | Shortlisted | 1518 | 210 | 13.83 | 6839 | 7 | 0.1 |
| OC-01-008-KA | Shortlisted | 1382 | 202 | 14.62 | 6032 | 11 | 0.18 |
| OC-01-025-PC | VUS | 1832 | 306 | 16.7 | 10638 | 11 | 0.1 |
| OC-01-025-PC | Shortlisted | 1640 | 75 | 4.57 | NA | NA | NA |
| OC-01-025-PC | VUS | 1832 | 306 | 16.7 | NA | NA | NA |
| OC-01-026-GKD | Shortlisted | 1331 | 385 | 28.93 | NA | NA | NA |
| OC-01-030-RP | Shortlisted | 537 | 339 | 63.13 | 5922 | 42 | 0.71 |
| OC-01-030-RP | VUS | 613 | 407 | 66.39 | 8392 | 42 | 0.5 |
| OC-01-030-RP | Shortlisted | 252 | 186 | 73.81 | 5835 | 35 | 0.6 |
| OC-01-030-RP | Shortlisted | 537 | 339 | 63.13 | 6967 | 61 | 0.88 |
| OC-01-030-RP | VUS | 613 | 407 | 66.39 | 10110 | 66 | 0.65 |
| OC-01-030-RP | Shortlisted | 252 | 186 | 73.81 | 6807 | 47 | 0.69 |
| OC-01-033-PS | Shortlisted | 673 | 157 | 23.33 | 5675 | 104 | 1.83 |
| OC-01-033-PS | Shortlisted | 673 | 157 | 23.33 | 7073 | 86 | 1.22 |
| OC-01-037-JK | Shortlisted | 140 | 6 | 4.29 | 7828 | 73 | 0.93 |
| OC-01-037-JK | Shortlisted | 140 | 6 | 4.29 | 6684 | 60 | 0.9 |
| OC-01-040-AJ | Shortlisted | 2139 | 717 | 33.52 | 8068 | 117 | 1.45 |
| OC-01-040-AJ | Shortlisted | 1281 | 223 | 17.4 | 6954 | 44 | 0.63 |
| OC-01-040-AJ | Shortlisted | 869 | 308 | 35.44 | 6679 | 121 | 1.81 |
| OC-01-040-AJ | Shortlisted | 2139 | 717 | 33.52 | 11108 | 174 | 1.57 |
| OC-01-040-AJ | Shortlisted | 1281 | 223 | 17.41 | 9358 | 60 | 0.64 |
| OC-01-040-AJ | Shortlisted | 869 | 308 | 35.44 | 7780 | 137 | 1.76 |
| OC-01-045-PH | Shortlisted | 1048 | 49 | 4.68 | NA | NA | NA |
| OC-01-047-GM | Shortlisted | 464 | 84 | 18.1 | 7425 | 11 | 0.15 |
| OC-01-047-GM | Shortlisted | 610 | 47 | 7.7 | 8940 | 14 | 0.16 |
| OC-01-047-GM | Shortlisted | 464 | 84 | 18.1 | 7816 | 10 | 0.13 |
| OC-01-047-GM | Shortlisted | 610 | 47 | 7.7 | NA | NA | NA |
| OC-01-048-MC | Shortlisted | 550 | 30 | 5.45 | NA | NA | NA |
| OC-01-049-NA | Shortlisted | 169 | 92 | 54.44 | 15117 | 20 | 0.13 |
| OC-01-049-NA | VUS | 460 | 232 | 50.43 | 12900 | 21 | 0.16 |
| OC-01-049-NA | Shortlisted | 92 | 28 | 30.43 | 14310 | 20 | 0.14 |
| OC-01-049-NA | VUS | 86 | 27 | 31.4 | NA | NA | NA |
| OC-01-049-NA | Shortlisted | 169 | 92 | 54.44 | NA | NA | NA |
| OC-01-049-NA | VUS | 460 | 232 | 50.43 | 8364 | 15 | 0.18 |
| OC-01-049-NA | Shortlisted | 92 | 28 | 30.43 | 8965 | 17 | 0.19 |
| OC-01-054-GPC | Shortlisted | 577 | 78 | 13.52 | NA | NA | NA |
| OC-01-054-GPC | VUS | 848 | 88 | 10.38 | 8576 | 15 | 0.17 |
| OC-01-054-GPC | Shortlisted | 823 | 69 | 8.38 | 8517 | 18 | 0.21 |
| OC-01-054-GPC | Shortlisted | 823 | 69 | 8.38 | 8517 | 18 | 0.21 |
| OC-01-054-GPC | Shortlisted | 582 | 127 | 21.82 | 9870 | 47 | 0.48 |
| OC-01-054-GPC | Shortlisted | 146 | 30 | 20.55 | 8991 | 34 | 0.38 |
| OC-01-054-GPC | VUS | 848 | 88 | 10.38 | 9557 | 19 | 0.2 |
| OC-01-054-GPC | Shortlisted | 823 | 69 | 8.38 | 10603 | 11 | 0.1 |
| OC-01-054-GPC | Shortlisted | 823 | 69 | 8.38 | 10603 | 11 | 0.1 |
| OC-01-054-GPC | Shortlisted | 582 | 127 | 21.82 | 10992 | 49 | 0.45 |
| OC-01-054-GPC | Shortlisted | 146 | 30 | 20.55 | 10182 | 52 | 0.51 |
| OC-01-055-CA | Shortlisted | 1180 | 99 | 8.39 | NA | NA | NA |
| OC-01-055-CA | Shortlisted | 856 | 45 | 5.26 | NA | NA | NA |
| OC-01-055-CA | VUS | 1740 | 142 | 8.16 | NA | NA | NA |
| OC-01-055-CA | VUS | 1767 | 139 | 7.87 | NA | NA | NA |
| OC-01-055-CA | VUS | 1332 | 129 | 9.68 | NA | NA | NA |
| OC-01-055-CA | Shortlisted | 1122 | 170 | 15.15 | NA | NA | NA |
| OC-01-055-CA | Shortlisted | 1520 | 88 | 5.79 | NA | NA | NA |
| OC-01-055-CA | Shortlisted | 798 | 106 | 13.28 | NA | NA | NA |
| OC-01-055-CA | Shortlisted | 345 | 42 | 12.17 | 9383 | 22 | 0.23 |
| OC-01-055-CA | Shortlisted | 1520 | 88 | 5.79 | 9248 | 12 | 0.13 |
| OC-01-055-CA | Shortlisted | 345 | 42 | 12.17 | 8663 | 16 | 0.18 |
| OC-01-059-LB | Shortlisted | 832 | 37 | 4.45 | NA | NA | NA |
| OC-01-059-LB | Shortlisted | 832 | 37 | 4.45 | 7562 | 10 | 0.13 |
| OC-01-061-MaS | Shortlisted | 1248 | 75 | 6.01 | 10631 | 15 | 0.14 |
| OC-01-061-MaS | Shortlisted | 1248 | 75 | 6.01 | NA | NA | NA |
| OC-01-065-RAM | Shortlisted | 516 | 80 | 15.5 | 6650 | 11 | 0.17 |
| OC-01-065-RAM | Shortlisted | 516 | 80 | 15.5 | 10788 | 22 | 0.2 |
| OC-01-070-RA | VUS | 127 | 6 | 4.72 | NA | NA | NA |
| OC-01-074-AK | Shortlisted | 360 | 81 | 22.5 | 11452 | 145 | 1.27 |
| OC-01-074-AK | Shortlisted | 84 | 51 | 60.71 | 5176 | 154 | 2.98 |
| OC-01-074-AK | Shortlisted | 360 | 81 | 22.5 | 12011 | 189 | 1.57 |
| OC-01-074-AK | Shortlisted | 84 | 51 | 60.71 | 5735 | 244 | 4.25 |
| OC-01-081-RAT | Shortlisted | 801 | 287 | 35.83 | NA | NA | NA |
| OC-01-096-VE | Shortlisted | 715 | 33 | 4.62 | 11475 | 100 | 0.87 |
| OC-01-096-VE | Shortlisted | 715 | 33 | 4.62 | 9149 | 69 | 0.75 |
| OC-02-004-SuD | Shortlisted | 653 | 41 | 6.28 | 2713 | 20 | 0.74 |
| OC-02-004-SuD | VUS | 744 | 46 | 6.18 | 3155 | 18 | 0.57 |
| OC-02-004-SuD | VUS | 655 | 36 | 5.5 | 2903 | 17 | 0.59 |
| OC-02-004-SuD | Shortlisted | 1347 | 74 | 5.49 | 3063 | 25 | 0.82 |
| OC-02-004-SuD | Shortlisted | 653 | 41 | 6.28 | 7358 | 93 | 1.26 |
| OC-02-004-SuD | VUS | 744 | 46 | 6.18 | 7447 | 81 | 1.09 |
| OC-02-004-SuD | VUS | 655 | 36 | 5.5 | 8767 | 50 | 0.57 |
| OC-02-004-SuD | Shortlisted | 1347 | 74 | 5.49 | 10937 | 61 | 0.56 |
| OC-02-010-BKS | Shortlisted | 736 | 31 | 4.21 | 4179 | 25 | 0.6 |
| OC-02-010-BKS | Shortlisted | 753 | 34 | 4.52 | 3920 | 29 | 0.74 |
| OC-02-010-BKS | Shortlisted | 736 | 31 | 4.21 | 9032 | 46 | 0.51 |
| OC-02-010-BKS | Shortlisted | 753 | 34 | 4.52 | 6177 | 25 | 0.4 |
| OC-02-012-MK | Shortlisted | 999 | 91 | 9.11 | 3957 | 46 | 1.16 |
| OC-02-012-MK | Shortlisted | 675 | 30 | 4.44 | 5062 | 32 | 0.63 |
| OC-02-012-MK | Shortlisted | 627 | 30 | 4.78 | 3278 | 8 | 0.24 |
| OC-02-012-MK | Shortlisted | 429 | 29 | 6.76 | 4296 | 16 | 0.37 |
| OC-02-012-MK | Shortlisted | 999 | 91 | 9.11 | 3736 | 45 | 1.2 |
| OC-02-012-MK | Shortlisted | 675 | 30 | 4.44 | 4813 | 31 | 0.64 |
| OC-02-012-MK | Shortlisted | 627 | 30 | 4.78 | 3030 | 7 | 0.23 |
| OC-02-012-MK | Shortlisted | 429 | 29 | 6.76 | 3937 | 12 | 0.3 |
| OC-02-019-PJ | Shortlisted | 422 | 107 | 25.36 | 8519 | 106 | 1.24 |
| OC-02-019-PJ | Shortlisted | 350 | 25 | 7.14 | 6486 | 21 | 0.32 |
| OC-02-019-PJ | VUS | 350 | 25 | 7.14 | 6486 | 21 | 0.32 |
| OC-02-019-PJ | Shortlisted | 422 | 107 | 25.36 | 10807 | 67 | 0.62 |
| OC-02-019-PJ | Shortlisted | 350 | 25 | 7.14 | 9553 | 22 | 0.23 |
| OC-02-019-PJ | VUS | 350 | 25 | 7.14 | 9553 | 22 | 0.23 |
| OC-02-020-NK | Shortlisted | 1164 | 309 | 26.55 | NA | NA | NA |
| OC-02-020-NK | Shortlisted | 789 | 208 | 26.36 | NA | NA | NA |
| OC-02-020-NK | VUS | 590 | 144 | 24.41 | NA | NA | NA |
| OC-02-020-NK | Shortlisted | 622 | 130 | 20.9 | NA | NA | NA |
| OC-02-020-NK | VUS | 856 | 46 | 5.37 | NA | NA | NA |
| OC-02-020-NK | VUS | 557 | 23 | 4.13 | NA | NA | NA |
| OC-02-020-NK | Shortlisted | 1148 | 444 | 38.68 | 7423 | 12 | 0.16 |
| OC-02-020-NK | VUS | 903 | 68 | 7.53 | NA | NA | NA |
| OC-02-020-NK | Shortlisted | 1226 | 148 | 12.07 | NA | NA | NA |
| OC-02-020-NK | Shortlisted | 1148 | 444 | 38.68 | NA | NA | NA |
| OC-02-023-SR | Shortlisted | 691 | 226 | 32.71 | 14764 | 168 | 1.14 |
| OC-02-023-SR | Shortlisted | 695 | 171 | 24.6 | 11966 | 136 | 1.14 |
| OC-02-023-SR | Shortlisted | 577 | 126 | 21.84 | 10105 | 110 | 1.09 |
| OC-02-023-SR | Shortlisted | 779 | 349 | 44.8 | 11470 | 194 | 1.69 |
| OC-02-023-SR | Shortlisted | 853 | 177 | 20.75 | 6673 | 94 | 1.41 |
| OC-02-023-SR | Shortlisted | 813 | 173 | 21.28 | 5741 | 87 | 1.52 |
| OC-02-023-SR | Shortlisted | 338 | 91 | 26.92 | 17223 | 158 | 0.92 |
| OC-02-023-SR | Shortlisted | 338 | 91 | 26.92 | 17223 | 158 | 0.92 |
| OC-02-023-SR | Shortlisted | 90 | 51 | 56.67 | 8675 | 271 | 3.12 |
| OC-02-023-SR | Shortlisted | 691 | 226 | 32.71 | 11465 | 144 | 1.26 |
| OC-02-023-SR | Shortlisted | 695 | 171 | 24.6 | 11591 | 129 | 1.11 |
| OC-02-023-SR | Shortlisted | 577 | 126 | 21.84 | 8320 | 57 | 0.69 |
| OC-02-023-SR | Shortlisted | 779 | 349 | 44.8 | 11423 | 169 | 1.48 |
| OC-02-023-SR | Shortlisted | 853 | 177 | 20.75 | 6060 | 74 | 1.22 |
| OC-02-023-SR | Shortlisted | 813 | 173 | 21.28 | 5026 | 74 | 1.47 |
| OC-02-023-SR | Shortlisted | 338 | 91 | 26.92 | 14759 | 109 | 0.74 |
| OC-02-023-SR | Shortlisted | 338 | 91 | 26.92 | 14759 | 109 | 0.74 |
| OC-02-023-SR | Shortlisted | 90 | 51 | 56.67 | 6291 | 197 | 3.13 |
| OC-02-025-RC | Shortlisted | 420 | 52 | 12.38 | 8901 | 13 | 0.15 |
| OC-02-025-RC | Shortlisted | 511 | 45 | 8.81 | NA | NA | NA |
| OC-02-025-RC | Shortlisted | 437 | 91 | 20.82 | 9190 | 19 | 0.21 |
| OC-02-025-RC | Shortlisted | 81 | 18 | 22.22 | 7792 | 11 | 0.14 |
| OC-02-025-RC | Shortlisted | 420 | 52 | 12.38 | NA | NA | NA |
| OC-02-025-RC | Shortlisted | 437 | 91 | 20.82 | 8191 | 18 | 0.22 |
| OC-02-025-RC | Shortlisted | 81 | 18 | 22.22 | NA | NA | NA |
| OC-02-027-SS | Shortlisted | 264 | 42 | 15.91 | 7891 | 70 | 0.89 |
| OC-02-027-SS | Shortlisted | 236 | 43 | 18.22 | 6082 | 47 | 0.77 |
| OC-02-027-SS | VUS | 291 | 56 | 19.24 | 10356 | 82 | 0.79 |
| OC-02-027-SS | VUS | 136 | 6 | 4.41 | NA | NA | NA |
| OC-02-027-SS | Shortlisted | 264 | 42 | 15.91 | 9083 | 61 | 0.67 |
| OC-02-027-SS | VUS | 264 | 42 | 15.91 | 9083 | 61 | 0.67 |
| OC-02-027-SS | Shortlisted | 236 | 43 | 18.22 | 6170 | 66 | 1.07 |
| OC-02-027-SS | VUS | 291 | 56 | 19.24 | 11005 | 79 | 0.72 |
| OC-02-029-KAM | Shortlisted | 254 | 22 | 8.66 | NA | NA | NA |
| OC-02-029-KAM | VUS | 560 | 46 | 8.21 | 13226 | 28 | 0.21 |
| OC-02-029-KAM | VUS | 396 | 32 | 8.08 | NA | NA | NA |
| OC-02-029-KAM | Shortlisted | 98 | 8 | 8.16 | NA | NA | NA |
| OC-02-029-KAM | VUS | 560 | 46 | 8.21 | NA | NA | NA |
| OC-02-033-TRM | Shortlisted | 318 | 58 | 18.24 | NA | NA | NA |
| OC-02-033-TRM | VUS | 242 | 16 | 6.61 | NA | NA | NA |
| OC-02-033-TRM | Shortlisted | 444 | 31 | 6.98 | 8042 | 13 | 0.16 |
| OC-02-033-TRM | Shortlisted | 152 | 12 | 7.89 | 24284 | 29 | 0.12 |
| OC-02-033-TRM | Shortlisted | 318 | 58 | 18.24 | 13274 | 35 | 0.26 |
| OC-02-033-TRM | Shortlisted | 444 | 31 | 6.98 | 6886 | 28 | 0.41 |
| OC-02-033-TRM | Shortlisted | 152 | 12 | 7.89 | 21401 | 28 | 0.13 |
| OC-02-043-DCS | Shortlisted | 286 | 39 | 13.64 | 9283 | 27 | 0.29 |
| OC-02-043-DCS | Shortlisted | 318 | 52 | 16.35 | 12730 | 76 | 0.6 |
| OC-02-043-DCS | Shortlisted | 407 | 163 | 40.05 | 6561 | 75 | 1.14 |
| OC-02-043-DCS | VUS | 143 | 13 | 9.09 | 17943 | 122 | 0.68 |
| OC-02-043-DCS | Shortlisted | 33 | 17 | 51.52 | NA | NA | NA |
| OC-02-043-DCS | Shortlisted | 286 | 39 | 13.64 | 6920 | 28 | 0.4 |
| OC-02-043-DCS | Shortlisted | 318 | 52 | 16.35 | 8487 | 42 | 0.49 |
| OC-02-043-DCS | Shortlisted | 407 | 163 | 40.05 | 6078 | 84 | 1.38 |
| OC-02-043-DCS | VUS | 143 | 13 | 9.09 | 14406 | 111 | 0.77 |
| OC-02-050-SN | VUS | 283 | 26 | 9.19 | 14886 | 36 | 0.24 |
| OC-02-050-SN | VUS | 283 | 26 | 9.19 | 10569 | 23 | 0.22 |
| OC-02-066-AB | Shortlisted | 1043 | 119 | 11.41 | 12493 | 51 | 0.41 |
| OC-02-066-AB | Shortlisted | 1043 | 119 | 11.41 | 12057 | 34 | 0.28 |
| OC-02-067-KN | Shortlisted | 429 | 99 | 23.08 | 8476 | 25 | 0.29 |
| OC-02-067-KN | Shortlisted | 50 | 22 | 44 | 4699 | 26 | 0.55 |
| OC-02-067-KN | Shortlisted | 429 | 99 | 23.08 | 11543 | 50 | 0.43 |
| OC-02-067-KN | Shortlisted | 50 | 22 | 44 | 7139 | 48 | 0.67 |
| OC-03-009 | Shortlisted | 1302 | 865 | 66.44 | 6463 | 134 | 2.07 |
| OC-03-009 | Shortlisted | 1302 | 865 | 66.44 | 10691 | 245 | 2.29 |
| OC-03-011 | Shortlisted | 1397 | 63 | 4.51 | NA | NA | NA |
| OC-03-015 | Shortlisted | 1972 | 867 | 43.97 | 12460 | 78 | 0.63 |
| OC-03-015 | Shortlisted | 1794 | 399 | 22.24 | NA | NA | NA |
| OC-03-015 | Shortlisted | 2164 | 989 | 45.7 | 10860 | 41 | 0.38 |
| OC-03-015 | Shortlisted | 3077 | 1377 | 44.75 | 12460 | 78 | 0.63 |
| OC-03-015 | Shortlisted | 2625 | 582 | 22.17 | NA | NA | NA |
| OC-03-015 | Shortlisted | 3643 | 1675 | 45.98 | 10860 | 41 | 0.38 |
| OC-03-015 | Shortlisted | 1972 | 867 | 43.97 | 9198 | 39 | 0.42 |
| OC-03-015 | Shortlisted | 1794 | 399 | 22.24 | 8635 | 29 | 0.34 |
| OC-03-015 | Shortlisted | 2164 | 989 | 45.7 | 7602 | 46 | 0.61 |
| OC-03-015 | Shortlisted | 3077 | 1377 | 44.75 | 9198 | 39 | 0.42 |
| OC-03-015 | Shortlisted | 2625 | 582 | 22.17 | 8635 | 29 | 0.34 |
| OC-03-015 | Shortlisted | 3643 | 1675 | 45.98 | 7602 | 46 | 0.61 |

### V. References for Example 1

The following references, to the extent that they provide exemplary procedural or other details supplementary to those set forth herein, are specifically incorporated herein by reference.
1. Dumache, R. Early Diagnosis of Oral Squamous Cell Carcinoma by Salivary microRNAs. Clin Lab 63, 1771-1776 (2017).
2. Bray, F., et al. Global cancer statistics 2018: GLOBOCAN estimates of incidence and mortality worldwide for 36 cancers in 185 countries. CA Cancer J Clin 68, 394-424 (2018).
3. Hunter, K. D., Parkinson, E. K. & Harrison, P. R. Profiling early head and neck cancer. Nat Rev Cancer 5, 127-135 (2005).
4. Bettendorf, O., Piffko, J. & Bankfalvi, A. Prognostic and predictive factors in oral squamous cell cancer: important tools for planning individual therapy? Oral Oncol 40, 110-119 (2004).
5. Axell, T., Pindborg, J. J., Smith, C. J. & van der Waal, I. Oral white lesions with special reference to precancerous and tobacco-related lesions: conclusions of an international symposium held in Uppsala, Sweden, May 18-21 1994. International Collaborative Group on Oral White Lesions. J Oral Pathol Med 25, 49-54 (1996).
6. Forastiere, A., Koch, W., Trotti, A. & Sidransky, D. Head and neck cancer. N Engl J Med 345, 1890-1900 (2001).
7. Rhodus, N. L. Oral cancer: leukoplakia and squamous cell carcinoma. Dent Clin North Am 49, 143-165, ix (2005).
8. Shiboski, C. H., Shiboski, S. C. & Silverman, S., Jr. Trends in oral cancer rates in the United States, 1973-1996. Community Dent Oral Epidemiol 28, 249-256 (2000).
9. Siegel, R. L., Miller, K. D. & Jemal, A. Cancer Statistics, 2017. CA: a cancer journal for clinicians 67, 7-30 (2017).
10. Warnakulasuriya, S. Global epidemiology of oral and oropharyngeal cancer. Oral Oncol 45, 309-316 (2009).
11. Poling, J. S., et al. Human papillomavirus (HPV) status of non-tobacco related squamous cell carcinomas of the lateral tongue. Oral Oncol 50, 306-310 (2014).
12. Castellsague, X., et al. HPV Involvement in Head and Neck Cancers: Comprehensive Assessment of Biomarkers in 3680 Patients. J Natl Cancer Inst 108, djv403 (2016).
13. Lingen, M. W., et al. Low etiologic fraction for high-risk human papillomavirus in oral cavity squamous cell carcinomas. Oral Oncol 49, 1-8 (2013).
14. Zafereo, M. E., et al. Squamous cell carcinoma of the oral cavity often overexpresses p16 but is rarely driven by human papillomavirus. Oral Oncol 56, 47-53 (2016).
15. Allen, K. & Farah, C. S. Screening and referral of oral mucosal pathology: a check-up of Australian dentists. Aust Dent J 60, 52-58 (2015).
16. Khurshid, Z., et al. Role of Salivary Biomarkers in Oral Cancer Detection. Adv Clin Chem 86, 23-70 (2018).
17. Ford, P. J. & Farah, C. S. Early detection and diagnosis of oral cancer: strategies for improvement. Journal of Cancer Policy 1, e2-e7 (2013).
18. Lingen, M. W., Kalmar, J. R., Karrison, T. & Speight, P. M. Critical evaluation of diagnostic aids for the detection of oral cancer. Oral Oncol 44, 10-22 (2008).
19. Patton, L. L., Epstein, J. B. & Kerr, A. R. Adjunctive techniques for oral cancer examination and lesion diagnosis: a systematic review of the literature. J Am Dent Assoc 139, 896-905; quiz 993-894 (2008).
20. Rethman, M. P., et al. Evidence-based clinical recommendations regarding screening for oral squamous cell carcinomas. J Am Dent Assoc 141, 509-520 (2010).
21. Macey, R., et al. Diagnostic tests for oral cancer and potentially malignant disorders in patients presenting with clinically evident lesions. The Cochrane database of systematic reviews, Cd010276 (2015).
22. Walsh, T., et al. Clinical assessment to screen for the detection of oral cavity cancer and potentially malignant disorders in apparently healthy adults. The Cochrane database of systematic reviews, Cd010173 (2013).
23. Lingen, M. W., et al. Evidence-based clinical practice guideline for the evaluation of potentially malignant disorders in the oral cavity: A report of the American Dental

Association. J Am Dent Assoc 148, 712-727.e710 (2017).
24. Lingen, M. W., et al. Adjuncts for the evaluation of potentially malignant disorders in the oral cavity: Diagnostic test accuracy systematic review and meta-analysis-a report of the American Dental Association. J Am Dent Assoc 148, 797-813.e752 (2017).
25. Agrawal, N., et al. Exome sequencing of head and neck squamous cell carcinoma reveals inactivating mutations in NOTCH1. Science 333, 1154-1157 (2011).
26. Stransky, N., et al. The mutational landscape of head and neck squamous cell carcinoma. Science 333, 1157-1160 (2011).
27. Comprehensive genomic characterization of head and neck squamous cell carcinomas. Nature 517, 576-582 (2015).
28. Anglim, P. P., et al. Identification of a panel of sensitive and specific DNA methylation markers for squamous cell lung cancer. Mol Cancer 7, 62 (2008).
29. Bettegowda, C., et al. Detection of circulating tumor DNA in early- and late-stage human malignancies. Sci Transl Med 6, 224ra224 (2014).
30. Diehl, F., et al. Circulating mutant DNA to assess tumor dynamics. Nat Med 14, 985-990 (2008).
31. Wang, Y., et al. Detection of somatic mutations and HPV in the saliva and plasma of patients with head and neck squamous cell carcinomas. Sci Transl Med 7, 293ra104 (2015).
32. Schubert, A. D., et al. Somatic mitochondrial mutation discovery using ultra-deep sequencing of the mitochondrial genome reveals spatial tumor heterogeneity in head and neck squamous cell carcinoma. Cancer letters 471, 49-60 (2020).
33. Lousada-Fernandez, F., et al. Liquid Biopsy in Oral Cancer. in Int J Mol Sci, Vol. 19 (2018).
34. Wang, X., Kaczor-Urbanowicz, K. E. & Wong, D. T. Salivary biomarkers in cancer detection. Med Oncol 34, 7 (2017).
35. Cristaldi, M., et al. Salivary Biomarkers for Oral Squamous Cell Carcinoma Diagnosis and Follow-Up: Current Status and Perspectives. Frontiers in Physiology 10(2019).
36. Diehl, F., et al. Analysis of mutations in DNA isolated from plasma and stool of colorectal cancer patients. Gastroenterology 135, 489-498 (2008).
37. Kinde, I., et al. Evaluation of DNA from the Papanicolaou test to detect ovarian and endometrial cancers. Sci Transl Med 5, 167ra164 (2013).
38. Wang, Y., et al. Detection of tumor-derived DNA in cerebrospinal fluid of patients with primary tumors of the brain and spinal cord. Proc Natl Acad Sci USA 112, 9704-9709 (2015).
39. Kinde, I., Wu, J., Papadopoulos, N., Kinzler, K. W. & Vogelstein, B. Detection and quantification of rare mutations with massively parallel sequencing. Proc Natl Acad Sci USA 108, 9530-9535 (2011).
40. Newman, A. M., et al. An ultrasensitive method for quantitating circulating tumor
DNA with broad patient coverage. Nat Med 20, 548-554 (2014).
41. Newman, A. M., et al. Integrated digital error suppression for improved detection of circulating tumor DNA. Nat Biotechnol 34, 547-555 (2016).
42. Kennedy, S. R., et al. Detecting ultralow-frequency mutations by Duplex Sequencing. Nat Protoc 9, 2586-2606 (2014).
43. Hoadley, K. A., et al. Cell-of-Origin Patterns Dominate the Molecular Classification of 10,000 Tumors from 33 Types of Cancer. Cell 173, 291-304.e296 (2018).
44. Mutational landscape of gingivo-buccal oral squamous cell carcinoma reveals new recurrently-mutated genes and molecular subgroups. Nat Commun 4, 2873 (2013).
45. Pickering, C. R., et al. Integrative genomic characterization of oral squamous cell carcinoma identifies frequent somatic drivers. Cancer Discov 3, 770-781 (2013).
46. Sen, M., et al. StrandAdvantage test for early-line and advanced-stage treatment decisions in solid tumors. Cancer Med 6, 883-901 (2017).
47. Hodara, E., et al. Multiparametric liquid biopsy analysis in metastatic prostate cancer. JCI insight 4(2019).
48. Onidani, K., et al. Monitoring of cancer patients via next-generation sequencing of patient-derived circulating tumor cells and tumor DNA. Cancer science 110, 2590-2599 (2019).
49. Nakagaki, T., et al. Targeted next-generation sequencing of 50 cancer-related genes in Japanese patients with oral squamous cell carcinoma. Tumour biology: the journal of the International Society for Oncodevelopmental Biology and Medicine 40, 1010428318800180 (2018).
50. Schirmer, M., D'Amore, R., Ijaz, U. Z., Hall, N. & Quince, C. Illumina error profiles: resolving fine-scale variation in metagenomic sequencing data. BMC bioinformatics 17, 125 (2016).
51. Nakamura, K., et al. Sequence-specific error profile of Illumina sequencers. Nucleic acids research 39, e90 (2011).
52. Lanman, R. B., et al. Analytical and Clinical Validation of a Digital Sequencing Panel for Quantitative, Highly Accurate Evaluation of Cell-Free Circulating Tumor DNA. PLoS One 10, e0140712 (2015).
53. Clark, T. A., et al. Analytical Validation of a Hybrid Capture-Based Next-Generation Sequencing Clinical Assay for Genomic Profiling of Cell-Free Circulating Tumor DNA. J Mol Diagn 20, 686-702 (2018).
54. Mattox, A. K., et al. Applications of liquid biopsies for cancer. Sci Transl Med 11(2019).
55. Mazurek, A. M., Rutkowski, T., Fiszer-Kierzkowska, A., Malusecka, E. & Skladowski, K. Assessment of the total cfDNA and HPV16/18 detection in plasma samples of head and neck squamous cell carcinoma patients. Oral oncology 54, 36-41 (2016).
56. Hamana, K., et al. Monitoring of circulating tumour-associated DNA as a prognostic tool for oral squamous cell carcinoma. British journal of cancer 92, 2181-2184 (2005).
57. Cao, W., et al. Multiple region whole-exome sequencing reveals dramatically evolving intratumor genomic heterogeneity in esophageal squamous cell carcinoma. Oncogenesis 4, e175-e175 (2015).
58. Gabusi, A., et al. Prognostic impact of intra-field heterogeneity in oral squamous cell carcinoma. Virchows Archiv 476, 585-595 (2020).
59. Siravegna, G., Marsoni, S., Siena, S. & Bardelli, A. Integrating liquid biopsies into the management of cancer. Nature reviews. Clinical oncology 14, 531-548 (2017).
60. Arantes, L. M. R. B., Carvalho, A. C.d., Melendez, M. E. & Carvalho, A. L. Serum, plasma and saliva biomarkers for head and neck cancer. Expert Review of Molecular

Diagnostics 18, 112-185 (2018).
61. Zakrzewski, F., et al. Targeted capture-based NGS is superior to multiplex PCR-based NGS for hereditary BRCA1 and BRCA2 gene analysis in FFPE tumor samples. BMC Cancer 19, 396 (2019).
62. Hirotsu, Y., et al. Dual-molecular barcode sequencing detects rare variants in tumor and cell free DNA in plasma. Sci Rep 10, 3391 (2020).
63. Cohen, J. D., et al. Detection and localization of surgically resectable cancers with a multi-analyte blood test. Science 359, 926-930 (2018).
64. Balaji, S. A., et al. Analysis of solid tumor mutation profiles in liquid biopsy. Cancer Med 7, 5439-5447 (2018).
65. Bennett, C. W., Berchem, G., Kim, Y. J. & El-Khoury, V. Cell-free DNA and next-generation sequencing in the service of personalized medicine for lung cancer. Oncotarget 7, 71013-71035 (2016).

### Example 2: Prognostic and Diagnostic Screen Tests to Identify Patients at High Risk for Oral Cavity Squamous Cell Carcinoma and Premalignant Oral Cavity Squamous Cell Carcinoma

Oral cavity squamous cell carcinoma (OCSCC) can be a lethal disease that is often preceded by premalignant lesions, making it is an ideal disease for screening initiatives. However, current screening protocols/tests cannot reliably differentiate between inflammatory and premalignant dysplastic lesions. Further, the histologic diagnosis of dysplasia is an imperfect predictor of malignant transformation as only −15% of premalignant oral lesions progress to cancer. The inventors sought to establish molecular-based diagnostic tests for prognostication and screening that are capable of identifying high-risk patients most likely to progress to oral cancer but would greatly benefit from closer surveillance and less morbid curative intent procedures. It is hypothesized that premalignant lesions contain identifiable genetic mutations that can be used for reliable biopsy prognostication (tissue biopsies) and screening (saliva). The inventors will identify dysplasia-specific mutations underlying the pathogenesis of OCSCC, and they will validate the mutations identified in a retrospective case-cohort study of dysplastic oral tissues with known clinical outcomes to investigate their potential as tissue-based prognostic biomarkers. The inventors will conduct a case-cohort study using saliva samples from five existing longitudinal population-based United States cohorts to determine whether driver somatic mutations can be identified in saliva prior to the diagnosis of oral cancer. These studies are conceptually innovative and likely to result in state-of-the-art risk stratification and screening. They would be the first to define the functional driver mutations of oral premalignancy. They would also be the first to determine if mutations in driver genes can be detected in saliva prior to oral cancer diagnosis, to define the time-course of mutation detection, and to test the predictive ability of identifying high-risk individuals with somatic mutations. They are technically innovative, as they evaluate the diagnostic accuracy of a novel non-invasive molecular salivary screening platform. This research will benefit human health by improving the ability of one to identify high-risk premalignant oral lesions likely to progress to cancer, thereby allowing for earlier and potentially more curative interventions with limited morbidity and mortality.

With an annual worldwide incidence of ˜600,000 cases including 50,000 cases in the United States, head and neck squamous cell carcinoma (HNSCC) is the world's 6th most common malignancy, with oral cavity SCC (OCSCC) representing approximately one third of United States cases and one half of worldwide cases. Despite therapeutic advances, OCSCC are frequently lethal, with a five-year survival of ˜55%. Because OCSCC is often preceded by premalignant lesions, it is an ideal disease for screening initiatives. The conventional visual and tactile exam (CVTE) coupled with tissue biopsy is the current gold standard. However, CVTE and commercially available adjunctive screening devices/tests have significant limitations as they cannot reliably differentiate between reactive/inflammatory and premalignant dysplastic lesions. Further, the histologic diagnosis of dysplasia is an imperfect predictor of malignant transformation as only ˜15% of dysplasias progress to OCSCC.

Using next generation sequencing, the inventors and others have characterized the mutational landscape of HNSCC. However, the oral premalignancy mutational landscape is unknown. In addition, genetic alterations in oral premalignancy have not been interrogated for their ability to prognostically stratify premalignant lesions into low- and high-risk categories. The inventors have also demonstrated that somatic mutations can be identified in the saliva/oral rinses of 100% of OCSCC patients, suggesting that the detection of driver somatic mutations in a less-invasive method may provide a promising modality for oral premalignancy screening. However, it is unknown if or when during the progression to cancer, these mutations can be detected in the saliva. A long-term goal is to establish a molecular-based diagnostic test for prognostication and screening that will identify high-risk patients most likely to progress to OCSCC. It is hypothesized that premalignant lesions contain identifiable genetic mutations that can be used for reliable tissue biopsy prognostication and saliva screening.

Aim 1: To define the presence of somatic mutations in key driver genes in dysplastic and control oral tissues. Building on previous work, the inventors will perform targeted sequencing to determine the mutation rate of the most commonly altered OCSCC genes in an existing collection of mild (n=200), moderate (n=200), and severe dysplasias (n=200), and reactive hyperplasias (n=100 as control group). These analyses are expected to identify dysplasia-specific changes underlying the pathogenesis of OCSCC.

Aim 2: To validate driver mutations in a retrospective cohort of dysplastic oral tissues with known clinical outcomes. The inventors will perform targeted sequencing on an independent and existing retrospective case-cohort study of biopsies from patients with oral dysplasia with (n=230) and without progression (n=460) to OCSCC to investigate the driver genes' potential as tissue-based prognostic biomarkers.

Aim 3: To investigate the presence of dysplasia-specific somatic mutations in key driver genes in saliva collected prior to the diagnosis of OCSCC. The inventors will conduct a case-cohort study of OCSCC (n=177) and controls (n=354) within five existing longitudinal population-based United States cohorts to determine whether driver somatic mutations can be identified in saliva prior to the diagnosis of OCSCC.

These studies are conceptually innovative and likely to result in state-of-the-art risk stratification and screening modalities for OCSCC. They would be the first to define the functional driver mutations of oral premalignancy. They would also be the first to determine if mutations in driver genes can be detected in saliva prior to OCSCC diagnosis, to define the time-course of mutation detection, and to test the predictive ability of identifying high-risk individuals with somatic mutations. They are technically innovative, as they evaluate the diagnostic accuracy of a novel non-invasive molecular salivary screening platform. This research will benefit human health by improving one's ability to identify high-risk premalignant oral lesions likely to progress to cancer, thereby allowing for earlier and potentially more curative interventions with limited morbidity and mortality.

Head and neck squamous cell carcinoma (HNSCC) is the sixth most common malignancy in the world and is associated with significant morbidity and mortality. Oral cavity SCC (OCSCC) represents about a third of the 50,000 cases of HNSCC in the United States and almost half of the 600,000 worldwide cases. Tobacco use and excessive alcohol consumption are the major etiologic factors for OCSCC. While prominent in oropharyngeal SCC, the human papilloma virus (HPV) is not a major etiologic factor for OCSCC (1-4). Despite numerous therapeutic advances, the long-term survival for patients with HPV-negative OCSCC has remained ˜55%, and earlier detection is critical (5-10). Because OCSCC is often preceded by premalignant lesions, it is an ideal disease for screening and early detection, thereby significantly increasing the 5-year survival rates (5,10). Therefore, methods that aid in improved prognostication, diagnosis, screening and intervention are paramount for improving outcomes. In fact, the World Health Organization prioritized and encouraged collaboration and research into cancers amenable for early detection, such as OCSCC (11).

The conventional visual and tactile exam (CVTE), coupled with a tissue biopsy, is the current gold standard. However, the CVTE has significant limitations. First, the CVTE cannot reliably discriminate between reactive/inflammatory and premalignant/early malignant lesions that require considerably different treatments. This presents a significant clinical challenge given that ˜10% of patients have some type of oral mucosal abnormality (12). While the vast majority of these lesions are benign, it is clinically challenging to differentiate between reactive/inflammatory and premalignant lesions. One explanation for this discrepancy is that premalignant lesions frequently do not demonstrate the clinical characteristics observed in OCSCC: ulceration, induration, pain, or cervical lymphadenopathy. Rather, the clinical presentation of premalignant oral lesions is highly heterogeneous and often mimics common reactive/inflammatory lesions. Second, some pre-cancerous lesions cannot be readily identified during a CVTE, as they are lurking undetected within the oral mucosa (13-16). It has been postulated that adjunctive screening devices/tests may aid in the identification and prognostication of premalignant lesions. The current adjuncts can be categorized as hand-held light-based devices (autofluorescence/tissue reflectance), cytology, and other adjuncts (17-21). In 2017, the Council on Scientific Affairs of the American Dental Association (ADA) convened an expert panel to perform a comprehensive systematic review of the published literature and providing primary care clinicians with practical, real world recommendations regarding the clinical utility of the commercially available adjuncts/tests in the context of screening for oral potentially malignant disorders (22,23). Dr. Lingen served as the Chair of the panel and Drs. Agrawal and Chaturvedi were critical contributing members. The conclusion of the meta-analysis and clinical guideline recommendations was that there was insufficient evidence to support the contention that any of the current devices/tests demonstrated sufficient diagnostic accuracy to be used in conjunction with the CVTE.

Since premalignant oral lesions cannot be accurately identified solely on the basis of their clinical characteristics, biopsy and histologic evaluation is recommended for all suspicious lesions. However, from a diagnostic perspective, a definition of oral premalignancy is problematic. Lesions are currently considered premalignant and at risk for progressing to OCSCC when a histologic diagnosis of dysplasia is rendered. Moreover, the criteria for diagnosing dysplasia are subjective and open to a wide range of interpretation, even among highly qualified pathologists (24-32). In addition, no validated histologic criteria currently exist for predicting the risk of malignant transformation of a dysplastic lesion. Therefore, the histological findings can only be used to indicate that a lesion has malignant potential. Several studies underscore this concept. Mincer et al. evaluated patients with oral dysplasias and followed them for up to 8 years. Only 11% of lesions underwent malignant transformation during the observation period (33). Likewise, Arduino et al. demonstrated that the 1-year outcomes of oral dysplasias were highly variable with ˜40% disappearing, ˜20% remaining stable and ˜7% progressing to OCSCC34. Finally, in a meta-analysis, the pooled overall malignant transformation rate for nearly 1,000 patients with oral dysplasias was 12.1% (CI: 8.1%, 17.9%) with great heterogeneity between studies with a range of 0%-36.4%35. The malignant transformation rate was 10.3% (CI: 6.1%, 16.8%) for mild/moderate dysplasia and 24.1% (CI: 13.3%, 39.5%) for severe dysplasia/carcinoma in situ. The mean time interval to malignant transformation for all grades of dysplasia was 4.3 years (range 0.5-16 years). These findings emphasize that the inventors are unable to accurately prognosticate on the basis of histologic alterations and underscore the need to develop molecular-based protocols to help refine one's diagnostic skills and address the diagnostic/prognostic dilemma outlined in FIG. 7.

The inability of conventional histopathology to prognostically stratify lesions accurately further underscores the need for molecular-based biomarkers. Somatic mutations are a hallmark of carcinogenic progression that allows reliable differentiation between cancer and normal tissues. The exclusive nature of tumor-defining driver genetic alterations makes them attractive biomarkers with a theoretical specificity approaching 100%. In this application, the inventors hypothesize that premalignant oral cavity lesions harbor somatic mutations which can be detected in both biopsy samples and saliva. Furthermore, the inventors hypothesize that tissue and saliva-based assays based on a defined panel of somatic mutations will dramatically improve one's ability to identify high-risk patients as well as prognosticate/quantify their risk for progressing to OCSCC.

These studies are conceptually innovative because they propose a systematic approach of identifying somatic driver mutations in histologically premalignant oral lesions. Mutations in candidate cancer genes have been identified in OCSCC. However, the timing and sequence of molecular alterations observed during progression are unknown. These studies will employ state-of-the-art technology to perform high depth targeted sequencing on dysplastic oral lesions to define the driver mutational landscape of the premalignant phase of this disease. The proposed research is also novel because it would be the first to comprehensively determine if mutations in key driver genes can be used as predictive biomarkers, in both a prognostic and screening setting, for oral premalignancy. Many groups have attempted to establish different biomarker platforms for oral dysplasia including aneuploidy, loss of heterozygosity (LOH), epigenetic markers, mRNA/miRNA profiling, and protein biomarker expression (14,36-64). While each of these lines of investigation have promise, some platforms (mRNA/miRNA profiling and protein expression) have been confronted with challenges with specificity. Conversely, other platforms (aneuploidy, LOH, epigenetic markers) are not easily incorporated into the workflow of molecular diagnostic pathology laboratories. Furthermore, there is limited evidence in the literature demonstrating replication of these biomarker studies across multiple studies or cohorts. The inventors are proposing an alternative approach involving interrogation of somatic mutations which, as a hallmark of cancer, should allow for the reliable and specific differentiation between normal and diseased (premalignant) tissue. The specificity of the driver mutations makes them reliable biomarkers when detectable as there is essentially no physiologic background. The proposed work is also technically innovative, as it will determine the diagnostic efficacy of a non-invasive molecular salivary screening platform for oral premalignancy. The substantial global burden of OCSCC and one's inability to identify high-risk individuals underscores the public health significance of the proposed work. The presence of DNA, including tumor DNA, in bodily fluids and blood is well-documented (65,66). In patients with cancer, a fraction of the DNA is tumor-derived and is termed tumor DNA (tDNA). For OCSCC, cells and fragments of DNA are shed into saliva from dividing cells during cell proliferation and/or cell death (67,68). Often, less than 1% of all DNA in saliva is derived from the primary tumor and albeit small, specific genomic regions of these DNA fragments can be amplified using PCR. Several studies have shown that mutations in released tDNA exactly correspond to mutations in the primary tumor (68-71). With the advent of digital-PCR technology and next generation sequencing, the inventors' team has shown that tDNA can be detected in saliva of patients with OCSCC (68), indicating that mutation detection in saliva may provide a robust molecular biomarker platform to overcome the limitations of current diagnostic/screening tests of oral premalignancy progression and address a major unmet clinical need in the field.

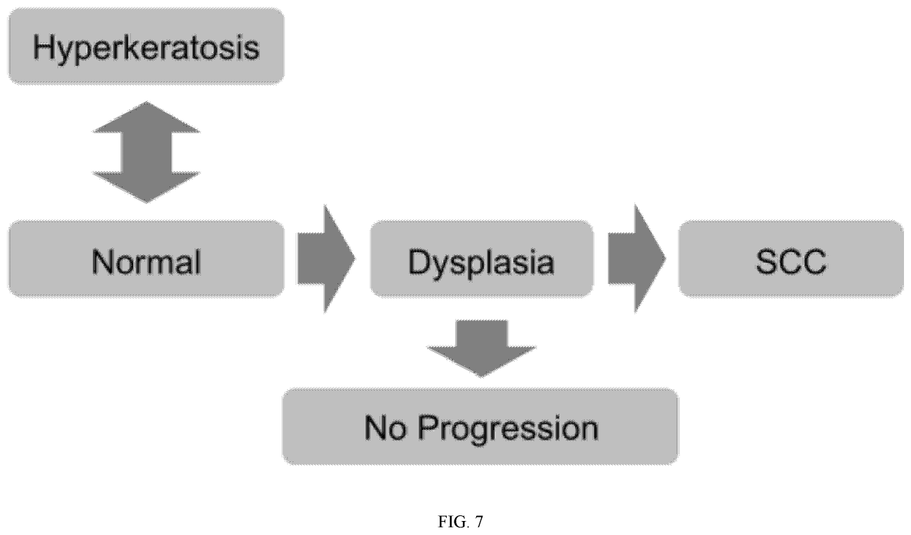

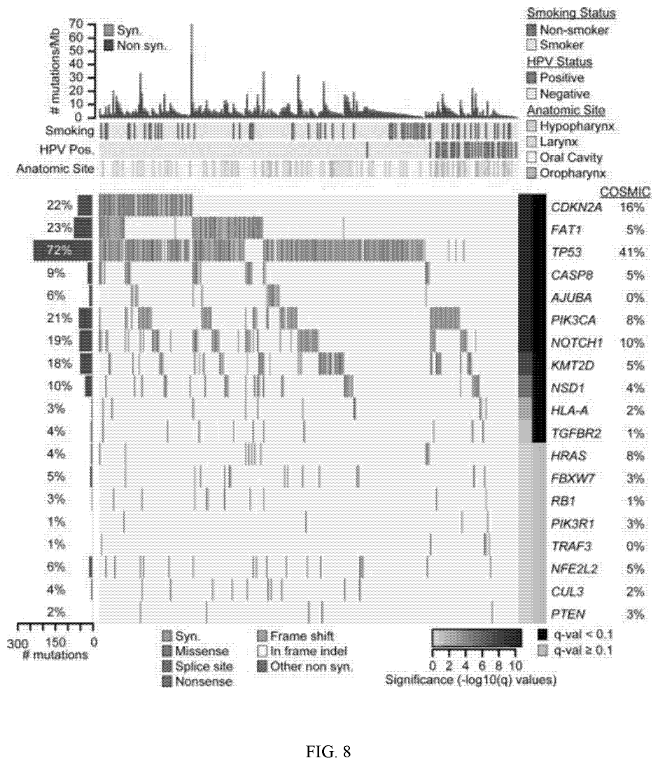

HNSCC mutational landscape. Previously, the inventors' group with MD Anderson Cancer Center and a collaborative group from the Broad Institute and University of Pittsburgh sequenced the protein-encoding genes in HNSCC and published companion papers (72,73). This early work was followed by the definitive study by the Cancer Genome Atlas (TCGA) (74). Although potentially targetable alterations were identified in most tumors, HNSCC were found to be largely driven by tumor suppressor mutations, with TP53 mutated in 86% of HPV negative samples followed by FAT1, CDKN2A, and NOTCH1 mutations in approximately 20% of samples (FIG. 8). In addition, mutations in the oncogene PIK3CA were also identified in approximately 20% of samples. Importantly, while TP53 was the most frequently mutated gene, the mutational frequency for the next 18 most commonly altered genes ranged from 1-23%, suggesting that there is considerable inter-tumoral heterogeneity with respect to the specific mutations harbored in a given tumor. The broad mutation spectrum detected in HNSCC by these early whole exome sequencing (WES) studies not only sheds light on its molecular pathogenesis, but also serves as a molecular signature that is exquisitely specific to the individual tumor. While recent advances in WES approaches allow generation of high-quality genetic data, their use as a modality for large-scale population-based screening has recognized limitations, such as long processing times, cost and most notably, difficulties in confidently calling driver and actionable genetic variants in part due to the low sequencing depth of approximately 100×.

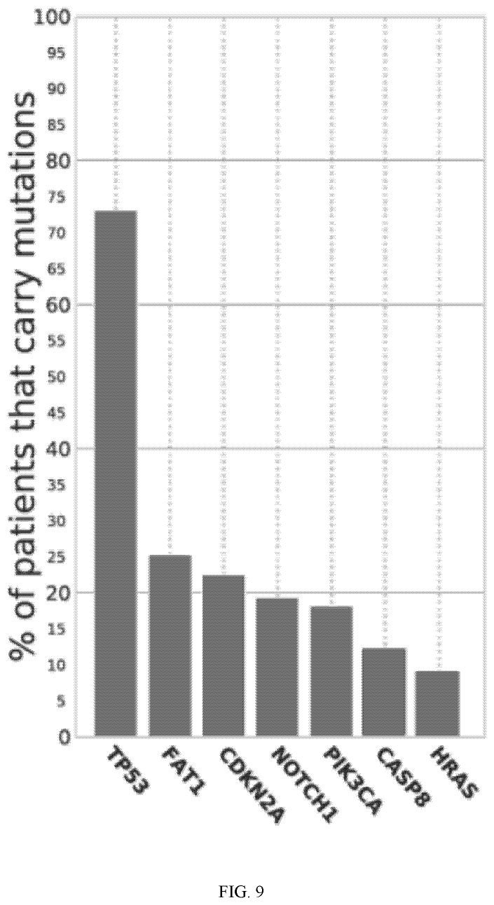

Ultra-deep targeted sequencing of primary OCSCC tumors. To overcome these limitations, the inventors have designed a custom targeted next-generation sequencing (NGS) panel that covers the entire exome regions of 7 most mutated genes in OCSCC (TP53, FAT1, CDKN2A, NOTCH1, PIK3CA, CASP8, and HRAS) (FIG. 9). Based on the analysis of the OCSCC sequencing from TCGA-HNSCC dataset [n=329], the incidence of at least one somatic aberration in any of these genes was 94%. Primers for target capture of the TRPM3 gene were included into the panel as a negative control, since this gene is rarely mutated in HNSCC. By applying this targeted sequencing approach on DNA extracted from 92 treatment naïve FFPE-derived OCSCC tissue specimens, the inventors obtained over 90% average on-target coverage with a median average depth of 2,550× across all sequenced samples. Using the stringent variant allele frequency (VAF) calling criteria of ≥2%, mutations in the gene panel were detected in 88 (96%) of the 92 sequenced specimens (21 stage I/II and 67 stage III/IV tumors) (FIG. 10A), with over 70% (62 of 88) of the samples carrying more than one reported variant (52.4% in early and 76.1% in late stage disease) (FIG. 10B). Notably, mutation frequencies for the 7 tumor associated genes sequenced in the cohort (FIG. 10A, left) highly resembled mutation frequencies reported for these genes in the TCGA dataset (FIG. 9), with no mutations detected in TRPM3 (negative control). These results support the credibility of the targeted sequencing of biomarkers as a promising screening approach. With a paucity of driver mutations in oncogenes and subsequent unavailability of targeted therapy, utilizing genetic biomarkers for prognosis, diagnosis, and screening are the optimal approaches for reducing morbidity and mortality from HNSCC.

Mutations in oral dysplasia in a discovery cohort. Building on this previous work, the inventors performed a proof-of-principle pilot study to determine whether the genetic alterations found in HNSCC could also be detected in oral premalignant lesions. For this, the inventors performed targeted sequencing on 6 of the 19 genes found to be most commonly mutated in the HNSCC TCGA (TP53, CDKN2A, PIK3CA, FBXW7, PIK3R1, and PTEN) in premalignant oral lesions. The inventors slightly adjusted the panel to include the 3 most commonly mutated genes in the above cohort of OCSCC as well as 3 genes potentially involved in premalignant progression. The inventors selected 36 moderate dysplasia and 35 severe dysplasia with 22 reactive hyperplasia as controls. It was found that TP53 and CDKN2A were often mutated in both moderate and severe dysplasia (Table 1). A PIK3CA mutation was identified in a moderate dysplasia specimen. Conversely mutations in FBXW7, PIK3R1, and PTEN were not identified, suggesting that these mutations seen in invasive SCC occur either at the point of malignant transformation or subsequently after that. In addition, 36.1% of moderate dysplasia harbored a mutation in either TP53 or CDKN2A but only 11.1% had more than one variant in TP53 (likely representing biallelic loss) or mutations in both TP53 and CDKN2A (Table 2). In severe dysplasia, 57.1% had a mutation in either TP53 or CDKN2A, and 25.7% had two mutations in TP53 or mutations in both TP53 and CDKN2A. Importantly, this frequency of 2 mutations in TP53, CDKN2A, and/or inactivation of both alleles in these genes of 11.1% in moderate dysplasia and 25.7% in severe dysplasia is provoking, as it approximates the malignant transformation rate of 10.3% for mild/moderate dysplasia and 24.1% for severe dysplasia (35). Although speculative and supportive of Knudsen's elegant two hit hypothesis, the inventors propose to test this in Specific Aim 2.

TABLE 1

| | Total | | | |
|---|---|---|---|---|
| Pathology | samples | TP53 | CDKNA2A | PIK3CA |
| reactive hyperplasia | 22 | 0.00% | 0.00% | 0.00% |
| (control) | | | | |
| moderate dysplasia | 36 | 30.60% | 8.30% | 2.80% |
| severe dysplasia | 35 | 51.40% | 14.30% | 0.00% |

TABLE 2

| | | % with 1 | % with 2 |
|---|---|---|---|
| | | mutation in | mutations in |
| | Total | CDKN2A or | CDKN2A and/ |
| Pathology | samples | TP53 | or TP53 |
| reactive hyperplasia | 22 | 0 | 0 |
| (control) | | | |
| moderate dysplasia | 36 | 36.1 | 11.1 |
| severe dysplasia | 35 | 57.1 | 25.7 |

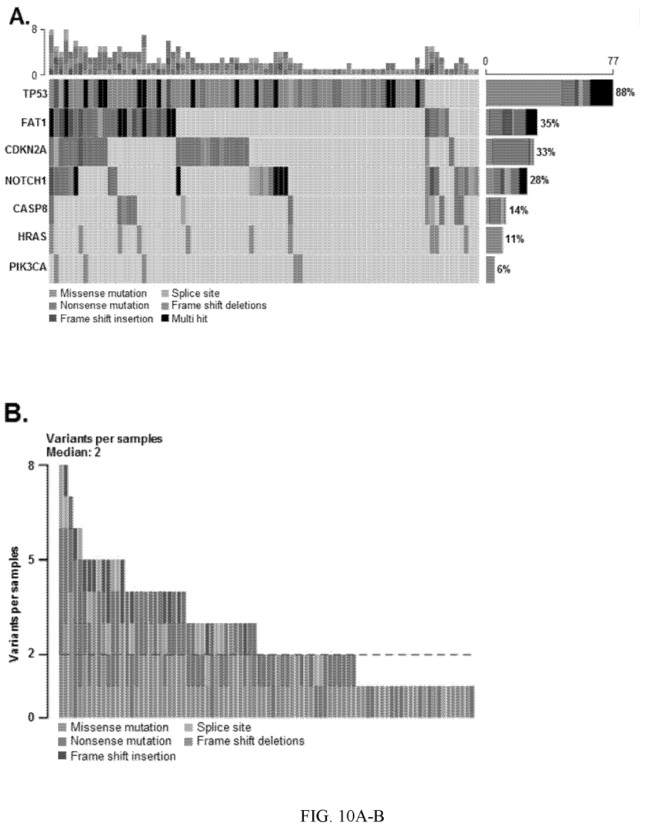

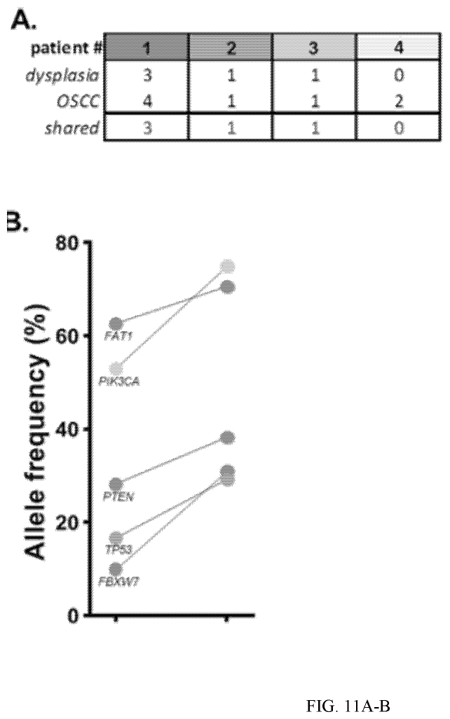

Mutations in matched longitudinal dysplasia/OCSCC samples. In a separate experiment, targeted sequencing of 7 genes that are frequently mutated in OCSCC (TP53, CDKN2A, PIK3CA, FAT1, FBXW7, PIK3R1, and PTEN) was performed on oral dysplastic lesions and OCSCC malignancies longitudinally collected from 4 patients. Matched lymphocyte DNA was used as a control. At 2% allelic frequency, in 3 patients the inventors identified somatic mutations that were present in both premalignant and invasive neoplasms (FIG. 11A). Interestingly, all shared mutations showed an increase in fractional abundance in tumor, compared to the precursor lesion collected from the same patient (FIG. 11B), providing the proof-of-concept that “drivers” that empower and cause territorial expansion of subclones en-route to malignancy typically occur early in tumor evolution and characterize relevant lesions that actually progress to cancer, unlike their “passenger” counterparts which are likely a passive by-product of tumorigenesis.

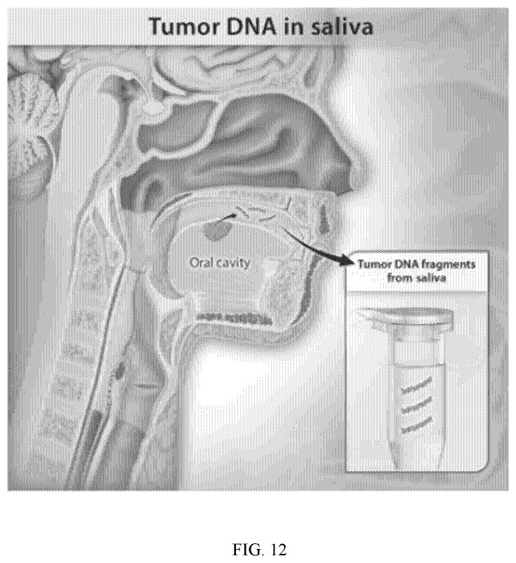

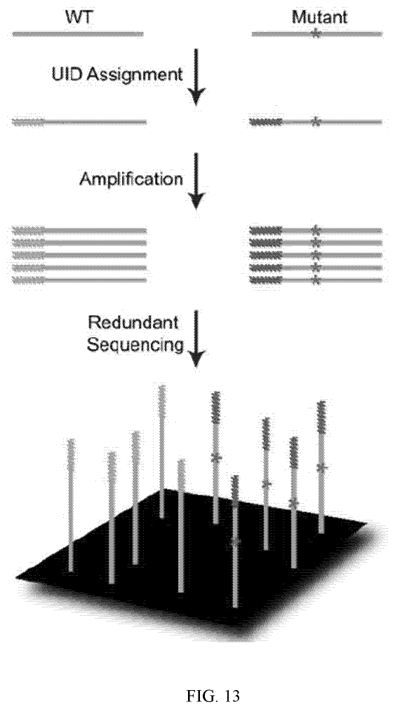

Tumor DNA in saliva as a biomarker for OCSCC. Various groups have used non-mutation based methods to detect tumors. While these approaches are promising, none are specific. Mutation based DNA biomarkers have several distinct advantages—unlike RNA and protein, they have no physiologic background and are not influenced by signaling changes induced during disease progression or therapy. Unlike RNA or protein-based assays, DNA based alterations should theoretically be found in appreciable levels only within premalignant and cancer cells and not normal cells. Moreover, DNA may better stoichiometrically correlate with disease burden. Further, nucleic acids are stable and amplifiable. Collectively, cancer specific genetic mutations allow for tremendous specificity. It is known that as cells divide and turnover, they shed DNA into various body fluids, including saliva (FIG. 12) (66-68,70,71,75-78). One of the major challenges in detecting mutant tDNA is the overwhelming abundance of normal DNA. In order to overcome this obstacle, the inventors' group has been developing sensitive PCR based assays for more than 10 years. Recent technological and computational advances have allowed the development of digital sequencing based rare mutation detection methods (78-80). Studies have shown that tDNA is commonly present at frequencies less than 1%, including in patients with HNSCC and even in patients with significant disease burden (67,68). The inventors' group has developed a sensitive digital approach error reduction technology based on massively parallel sequencing for the detection of rare variants, coined Safe-SeqS (Safe-Sequencing System) (FIG. 13) (79). The inventors have been able to employ this approach in different tumors and bodily fluids including saliva. One study attempted to detect tDNA from DNA extracted from conventional Pap smear as a screening method for gynecologic malignancies. While Pap smears are currently used to detect cervical cancers, using Safe-SeqS, Kinde et al. were able to develop a screening strategy that could detect 100% of endometrial cancers and 40% of ovarian cancers tested (70). The assay was based on directly querying the DNA from Pap fluid for mutations in the 12 most commonly altered genes in endometrial and uterine cancer without a priori knowledge of the tumor's genotype. A second study examined the presence of circulating tDNA in the plasma from over 600 advanced or localized malignancies in over 16 different tumor types, including HNSCC. The inventors found that the majority of cancers shed readily detectable levels of circulating tDNA into the bloodstream (67). The inventors performed a third proof-of-concept study to investigate the ability to identify somatic mutations from saliva of HNSCC patients (FIG. 12) (68). This study included a total 93 HNSCC patients of different sub-anatomic sites (46 oral cavity, 34 oropharynx, 3 hypopharynx, and 10 larynx). The inventors attempted to identify at least one genetic alteration in each tumor type, first searching for the presence of either HPV type 16 (HPV16) or HPV18 sequences in tumor DNA. HPV is a well-established etiologic agent for a growing subset of HNSCCs, specifically oropharyngeal SCC, but not OCSCC81,82. In the HPV-negative SCC, including OCSCC, the inventors searched for somatic mutations in genes or gene regions commonly altered in HNSCC, including TP53, PIK3CA, CDKN2A, FBXW7, HRAS, and NRAS67,70,79,80,83. When segregated by site, tDNA was detected in the saliva of 100% of patients with OCSCC (Table 3). Importantly, this study showed that a high-proportion of early stage OCSCC tumors (Stage I and II) had detectable mutations in 100% of the saliva samples including in TP53 and PIK3CA in 13 of 15 samples, which underscored the potential for risk stratification and early detection through somatic mutation testing of key driver genes in saliva.

TABLE 3
Prevalence of somatic mutations in a gene in cases for 95% CI estimates
and in controls for minimum detectable odds ratio estimates.

| | Total | % (95% CI) of oral rinses with detectable | |
|---|---|---|---|
| Anatomic site | samples | somatic mutations and/or HPV DNA^{a} | |
| Oral cavity | 46 | 100 | (92-100) |
| Oropharynx | 34 | 47 | (30-65) |
| Hypopharynx | 3 | 67 | (9.4-99) |
| Larynx | 10 | 70 | (35-93) |

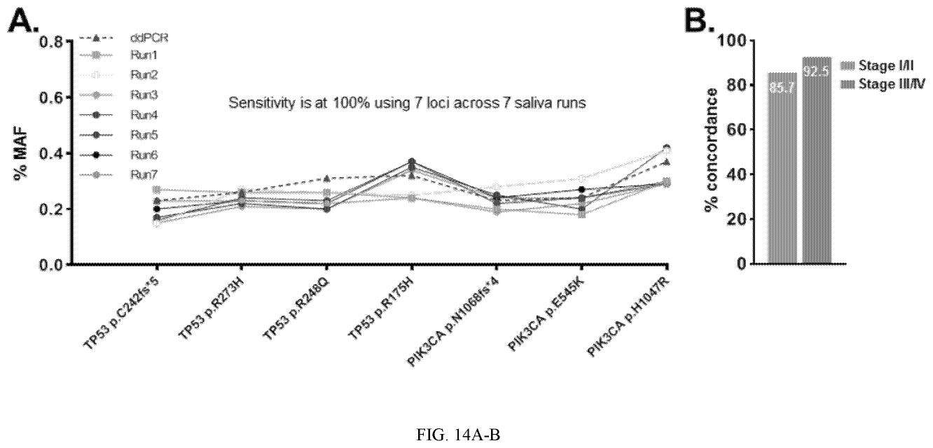

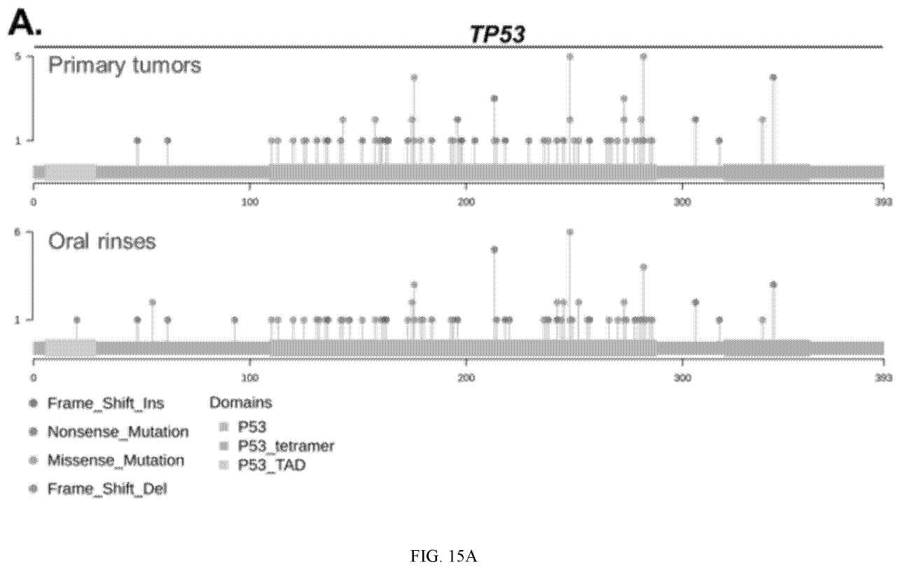

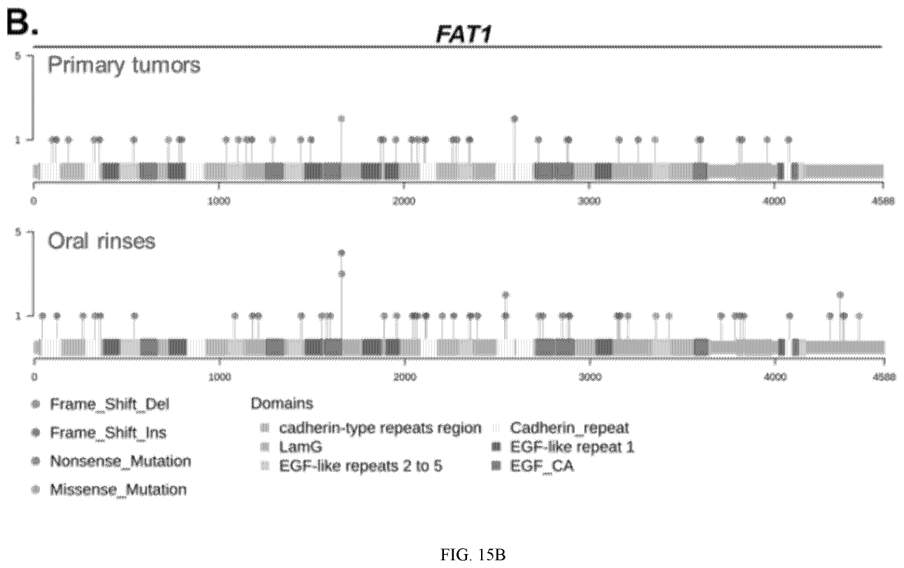

To confirm these proof-of-concept results in an independent cohort of OCSCC specimens, the inventors have applied the same targeted sequencing panel that was used to sequence 92 OCSCC tumors described in FIG. 10, on DNA extracted from the matched preoperative saliva collected from these 92 patients. As DNA isolated from saliva is frequently degraded due to the presence of high-DNase activity, tagged samples were 12-plexed and sequenced on Illumina NextSeq sequencer generating ˜67 million reads per sample, resulting in an average depth of 27,700× across all of the saliva tested (ranging from 11,915× to 42,101×). Based on the inventors' previous published experience with detecting tumor associated mutations in bodily fluids, the cut-off for variants calling in saliva was set at 0.1% (with minimum 10,000× coverage). Using these criteria, a 0.1% minor allele frequency represents a mutant allele read count of at least 10, suggesting that the detected mutations are not a sequencing artifact. High-quality sequence reads were selected on the basis of quality scores generated by the sequencing instrument, to indicate the probability a base was called in error. To further ensure that a variant is not an error resulting in a false positive call, the inventors performed a vigorous multistep analytical validation of the saliva sequencing method: (i) independent re-sequencing of 24 saliva samples confirmed 94% of the detected variants, supporting the reproducibility of this approach; (ii) no functionally relevant somatic variants were detected in saliva samples collected from 13 subjects without a visible oral cavity lesion and without a history of tobacco and alcohol usage, supporting the specificity of the assay; (iii) the inventors further performed analytical validation of the sequencing method using a panel of 7 synthetic loci across 7 independent saliva sequencing runs (FIG. 14A), which further confirmed that the targeted NGS approach is reproducible and provides sensitivity for mutation detection which is on par with that of droplet digital PCR (ddPCR) assay (FIG. 14A), a gold-standard method for detecting low prevalence tumor-associated mutations. Notably, mutations identified in primary tumors were also detected in 90.9% (80 of 88) of matched saliva samples collected from same patients, with only a minor decrease in detection frequency seen in patients with early stage disease (FIG. 14B). No mutations were detected in four saliva specimens collected from patients who displayed no mutations in their primary tumors. FIG. 15 highlights the high concordance in mutation distribution across the tested genes between primary tumors and saliva specimens. These preliminary data highlight the feasibility of somatic mutation identification in driver genes in saliva samples collected at the time of OCSCC diagnosis.

To examine the potential of using salivary biomarkers to assess the risk of malignant transformation, the inventors used the same 7 driver gene panel to sequence DNA extracted from 12 patients with high grade oral dysplasia and matched saliva specimens. Mutations in tumor driver genes were detected in 10 (83.3%) of the dysplastic lesions, with 3 (25%) carrying mutations in more than one gene. These observations suggest that driving clonal events occur early in progression, and that concomitant disruption of several driver genes may favor malignant transformation. As expected from the inventors' hypothesis, at 0.1% allelic frequency the ultra-deep targeted sequencing was able to detect dysplasia-associated mutations in 8 (80%) of the 10 saliva specimens with identified somatic mutations, whereas no mutations were detected in 2 saliva specimens collected from patients that displayed no mutations in the dysplastic lesions. The inventors do anticipate some loss in sensitivity for the detection of somatic mutations in saliva vs. tissue samples in premalignancies which is consistent with progressive decrease in sensitivity throughout the OCSCC continuum from advanced stage OCSCC (92.5%) to early stage OCSCC (85.7%) to high-grade dysplasia (80%). Nevertheless, this small loss in sensitivity will only minimally affect the statistical efficiency. While the preliminary data provide proof of concept that driver mutations associated with oral premalignancy can be detected in paired saliva specimens even before they invade and acquire malignant potential, it is currently unknown how far prior to cancer diagnosis somatic mutations are detectable in saliva from patients with oral dysplasia (i.e. sensitivity), if somatic mutations can be detected in individuals who did not develop cancer (i.e. specificity based on prevalence in controls), and the positive- and negative-predictive values for the presence of somatic mutations and future incidence of head and neck cancers. The proposed study will seek to address these questions. While in the preliminary data, the inventors have explored several targeted sequencing panels (presented across the preliminary results and unpublished data), the revolving high frequency and driver mutations have always emerged across multiple experiments, leading to the inventors' current inclusive and informed panel proposed in Aim 1.

Aim 1—To define the presence of somatic mutations in key driver genes in dysplastic and control oral tissues. Building on previous work, the inventors propose to define the driver mutational landscape of oral dysplastic lesions by targeted sequencing of the most commonly altered OCSCC genes in oral premalignant lesions with a histologic diagnosis of mild dysplasia, moderate dysplasia, severe dysplasia (as defined by the 2017 WHO Classification of Head and Neck Tumors), and reactive hyperplasia (control group) 68,72-74. The inventors hypothesize that the prevalence of somatic mutations in individual genes, the total number of mutated genes as well as combinations of genes will be significantly higher in the dysplastic lesions when compared to the controls. The inventors further hypothesize that the proportion of dysplastic cases with detectable somatic mutations will be greater with increasing histologic grade. These analyses are expected to identify dysplasia-specific molecular changes that underlie the pathogenesis of OCSCC that could be developed into prognostic and screening biomarkers.

Design: The inventors will utilize a cross-sectional study design to compare the prevalence of somatic mutations across the continuum of carcinogenesis. They will use existing archival de-identified, coded diagnostic specimens from the University of Chicago previously collected by Dr. Mark Lingen (PI). Cases will include 200 mild, 200 moderate, and 200 severe dysplasias. Control tissue will include a random sampling of 100 reactive oral lesions or hyperplasia frequency matched to the combined case group (mild+moderate+severe dysplasias) by 5-year age group, gender, and smoking (never, former, current) at a 1:2 ratio to cases. DNA will be isolated from formalin-fixed paraffin embedded (FFPE) samples and evaluated for the presence of somatic mutations in 19 key driver genes that have been found to be significantly mutated in the HNSCC TCGA project (CDKN2A, FAT1, TP53, CASP8, AJUBA, PIK3CA, NOTCH1, KMT2D, NSD1, HLA-A, TGFBR2, HRAS, FBXW7, RB1, PIK3R1, TRAF3, NFE2L2, CUL3, and PTEN). The inventors propose to use a larger gene panel at this stage and will narrow the panel to decrease costs and increase sequencing coverage as described in the preliminary data with the goal of achieving reliable scalability and high throughput. Analyses will be conducted for each of the individual genes, the number of genes per patient with detectable somatic mutations, and combinations of genes with detectable somatic mutations.

Targeted sequencing and identification of mutations: The inventors will employ an approach that is similar to what they have previously used to identify genetic changes in OCSCC and other cancers (68). The specimens will be selected based on purity and quality, ensuring sensitive detection of genomic alterations, critical parameters for the success of high throughput DNA sequencing. The inventors will perform an independent review of all tissue and only samples meeting stringent criteria will be included. In brief, FFPE tissue will be carefully reviewed and independently confirmed by two pathologists. If there is lack of concordance between two pathologists, a third pathologist will be consulted to confirm the status. Tissues will be microdissected as needed to confirm 1) the diagnosis, 2) that the representative section contains predominantly dysplastic tissue, and 3) that there are no pathological signs of invasive SCC within the specimen. DNA from dysplastic or control tissue will be purified and used to prepare fragment libraries suitable for targeted sequencing approaches. The 19 genes with the greatest mutational frequency in the TCGA dataset, as listed above, will be targeted for sequencing using an amplicon-based panel with subsequent massively parallel sequencing of at least 5,000×depth coverage on an Illumina NovaSeq instrument at the Clinical Genomics Laboratory (Dr. Jeremy Segal; please see letter of support). The inventors have chosen to pursue the targeted sequencing in a clinical laboratory setting to facilitate clinical validation and translation. Sequencing data will be analyzed using custom bioinformatic approaches. Briefly, amplicon assay data will be pre-processed using a custom quality and on-target filter, aligned to the hg (38) reference human genome using Novoalign. Variant calling will be performed using a University of Chicago developed variant caller (Variant Inspector) and Amplicon Indel Hunter for detection of indels greater than 5 bp, and final variants will be annotated using Alamut Batch software (84).

Statistical analyses: The inventors will determine the prevalence of each of the 19 genes and the number of genes with somatic mutations in individuals. Results will be presented as percentages, with 95% exact binomial confidence intervals. The inventors will compare the prevalence of somatic mutations in each of the 19 genes across disease states (normal vs. mild, moderate, or severe dysplasia as well as across dysplasia grades) using multivariable binary or multinomial logistic regression models. The inventors will also consider multiplicative statistical interactions of somatic mutations with gender and will perform analyses stratified by gender, as appropriate. To identify other possible interactions, the inventors will evaluate combinations of genes across the disease states using unweighted classification and regression tree (CART) analysis, with 10-fold cross-validation. These analyses will be adjusted for age, gender, and smoking. Given the multiple statistical testing across 19 genes, to reduce the probability of false-positive associations, the inventors will utilize a Bonferroni-corrected threshold of P<0.003. To be less conservative the inventors will also consider using a False-Discovery Rate criterion of 5%. Also, because somatic mutations represent causal intermediate states for the carcinogenic association of tobacco use, the key OCSCC risk factor, the inventors will consider analyses stratified by smoking in lieu of model-based adjustment.

Power calculations: In Table 4, the inventors present the precision, as measured by the exact binomial 95% confidence intervals (CI) around a range of somatic mutation prevalence. Given these sample sizes, this study is adequately powered to rule out somatic mutation prevalence greater than 3.6% in control tissues, 1.8% in mild, moderate, or severe dysplasia, and 0.9% in tissues with any grade of dysplasia. In Table 5, the inventors present the minimum detectable odds ratios across a range of prevalence estimates of somatic mutations in single genes for control vs. dysplasia grades and for comparisons across dysplasia grades. The minimum detectable odds ratios for control vs. any dysplasia account for multiple statistical testing through the use of a Bonferronicorrected type I error rate of 0.003 (=0.05/19).

TABLE 4
Precision around prevalence estimates for somatic mutations
in normal/hyperplasia and dysplasia grades

| | | Hypothetical | |
|---|---|---|---|
| | | prevalence | |
| Hypothetical | | of somatic | |
| prevalence | | mutations in | |
| of somatic | | one of 19 | |
| mutations in | | genes in mild | |
| one of 19 genes | | dysplasia, | |
| in normal/ | | moderate, or | |
| hyperplasia | | severe dysplasia | |
| (n = 100) | 95% CI | (n = 200) | 95% CI |
| 1% | 0.02-5.5 | 1% | 0.1-3.6 |
| 2% | 0.2-7.0 | 2% | 0.6-5.0 |
| 5% | 1.6-11.3 | 5% | 2.4-9.0 |
| 10% | 4.9-17.6 | 10% | 6.2-15.0 |
| 25% | 16.9-34.7 | 25% | 19.2-31.6 |
| 50% | 39.8-60.2 | 50% | 42.9-57.1 |

TABLE 5
Minimum detectable odds ratios for comparisons of the prevalence
of somatic mutations by histopathologic categories

| Hypothetical | | | Minimum detectable |
|---|---|---|---|
| prevalence | | Hypothetical | odds ratios for |
| of somatic | Minimum detectable | prevalence | comparisons across |
| mutations in | odds ratios for | of somatic | any two grades |
| one of 19 | normal (n = 100) | mutations in | of dysplasia |
| genes in | vs. any grade of | one of 19 | (n = 200 in |
| normal/ | dysplasia (n = 200) | genes in any | each group) |
| hyperplasia | Alpha = 0.003 and | dysplasia | Alpha = 0.05 and |
| (n = 100) | power = 80% | grade (n = 200) | power = 80% |
| 1% | 16.05 | 1% | 10.32 |
| 2% | 9.34 | 2% | 6.39 |
| 5% | 5.16 | 5% | 3.85 |
| 10% | 3.66 | 10% | 2.89 |
| 25% | 2.72 | 25% | 2.26 |
| 50% | 2.61 | 50% | 2.19 |

Expected Outcomes, Potential Pitfalls and Alternative Strategies: The inventors expect the dysplastic oral lesions to harbor mutations in a subset of the 19 genes. A working hypothesis is that premalignant lesions with two or more mutations (regardless of the specific gene mutation) are more likely to undergo malignant transformation (i.e. they are high-risk lesions). The major concern of this aim could be adequacy of dysplastic samples. However, the samples have already been collected and annotated. The inventors do not foresee any potential problems with targeted sequencing from FFPE samples as this is fairly routine and the inventors have performed sequencing from similar samples in the preliminary studies and in numerous published reports. Another concern maybe the lack of matched normal for variant calls. To overcome this limitation, the inventors will employ an approach previously reported in the inventors' published projects, in which the inventors exclude variants present in public databases as SNPs at greater than 1% frequency in the population and only include variants that are likely pathogenic or pathogenic based on the presence of a particular variant in somatic cancer databases, predicted function, and expected variant allele frequency. Molecular mechanisms that initiate premalignancy and drive progression are complex and involve multiple processes. The inventors have focused on targeted sequencing of select driver genes identified in OCSCC rather than a strategy that includes WES, whole genome sequencing, copy number analysis, epigenetic changes, expression analysis, and/or proteomics. While each of these classes of alterations play a critical role in carcinogenesis, the goal is to understand driver molecular changes at the DNA level that could be used to develop highly specific and easily reproducible diagnostic and screening platforms that could be widely used in the clinical setting. WES would have significant limitations in the depth of coverage that could be achieved (˜150× for WES versus greater than 5,000× for targeted sequencing). In addition, the major driver mutations that have previously been clearly identified in OCSCC and are included in the targeted sequencing panel are driving tumorigenesis. Whereas some of the other mutations that would potentially be identified by WES may represent passenger mutations occurring passively during tumorigenesis and of unknown biologic and clinical significance.

Aim 2. To validate the mutations in a retrospective cohort of dysplastic tissues with known clinical outcomes. The inventors have characterized the OCSCC mutational landscape. However, the oral premalignancy mutational landscape is unknown and is predicted to be a subset of the OCSCC landscape. In addition, these genetic alterations have not been interrogated for their ability to stratify premalignant lesions into low- and high-risk categories. The inventors will perform targeted sequencing on an independent and existing retrospective cohort of oral dysplasia biopsies with and without progression to cancer to investigate their potential as tissue-based prognostic biomarkers.

Aim 2.1. To characterize the mutations of key driver genes in dysplastic oral mucosa in patients that do and do not progress to OCSCC. The inventors hypothesize that the prevalence of somatic mutations in individual genes, the total number of mutated genes, as well as the combinations of mutated genes will be significantly higher in dysplastic tissues from patients that progress to OCSCC.

Aim 2.2. To investigate whether the presence of somatic mutations in key driver genes in dysplastic oral mucosa can predict the time frame of cancer development (<1 years, 1-2 years, 2-3 years, 3-4 years, and 5+years). The inventors hypothesize that somatic mutations will be detected in multiple driver genes in dysplastic tissues collected several years prior to cancer diagnosis. Additionally, the inventors hypothesize that the proportion of dysplasias with detectable somatic mutations will be highest in biopsies closest to OCSCC diagnosis.

Design: The inventors will utilize a case-cohort design to compare somatic mutations in premalignant lesions that progressed to OCSCC vs. lesions that did not progress. The sampling frame for this study will be a cohort of dysplasia patients with available archived FFPE tissue from dysplasia biopsies, demographic and risk factor information, and complete data on progression to cancer. Cases will include 230 dysplasias that progressed to OCSCC at least 6 months after dysplasia diagnosis. Controls will include a stratified random sample cohort of n=460 with stratification by 5-year age group, gender, smoking status, and dysplasia grade (mild dysplasia, moderate dysplasia, and severe dysplasia).

Targeted sequencing and identification of mutations: Specimens will be selected based on stringent criteria to ensure sensitive detection of genomic alterations. In brief, the tissue will be reviewed by two pathologists to confirm 1) the diagnosis and 2) that the representative section contains predominantly dysplasia or neoplastic tissue (for those that progress). The DNA derived from these samples will be used to prepare sequencing libraries using an amplicon-based panel and will be sequenced with over 5,000×depth coverage (given that the gene panel is likely to consist of less than 19 genes) on an Illumina NovaSeq instrument at the Clinical Genomics Laboratory (Dr. Jeremy Segal; please see letter of support). Again, the inventors have chosen to pursue the targeted sequencing in a clinical laboratory setting to facilitate translation to patients. Sequencing data will be analyzed using the same bioinformatic approaches outlined in Aim 1 to identify the somatic mutations. While not central to the proposed research, the inventors strongly believe sequencing the invasive SCC counterpart from the 230 dysplasias that progressed will provide indisputable confirmation of the biological relevance of the mutations identified in the dysplastic lesions.

Statistical analyses: All analyses will account for the case-cohort sampling design through the use of weights—the weights for progressing cases will be 1.0 given the 100% selection, while the weights for the subcohort will be the inverse of the selection probability into the subcohort (85-87). The inventors will compare the prevalence of somatic mutations in each of the 19 genes as well as the number of mutated genes between cases and the subcohort using weighted Cox proportional hazards regression models. The inventors will investigate interactions across the combinations of genes in weighted Cox regression analyses. Additionally, the inventors will compare combinations of genes between cases and the subcohort using unweighted CART analysis, with 10-fold cross-validation. These analyses will be adjusted for age, gender, smoking, and grade of dysplasia. The inventors will also consider multiplicative statistical interactions of somatic mutations with gender or with smoking, and will perform analyses stratified by gender or smoking, as appropriate. Given the multiple statistical testing across 19 genes, to reduce the probability of false-positive associations, the inventors will utilize a Bonferroni-corrected threshold of P<0.003. To be less conservative the inventors will also consider using a False-Discovery Rate criterion of 5%. Also, because somatic mutations represent causal intermediate states for the carcinogenic association of tobacco use, the key OCSCC risk factor, the inventors will consider analyses stratified by smoking in lieu of model-based adjustment. Analyses will be conducted overall and stratified by time between dysplasia diagnosis and cancer diagnosis (<1 year, 1-2 years, 2-3 years, 3-4 years, and 5+ years). Results will be summarized as hazard ratios, sensitivity (proportion of oral cancers with detectable somatic mutations in preceding dysplasias), specificity (1-prevalence of somatic mutations in the subcohort), positive predictive values (incidence of oral cancer given the presence of somatic mutations), and the complement of the negative predictive value (incidence of oral cancer in the absence of somatic mutations in preceding dysplasias). All analyses will account for the sampling design through the use of inverse probability weighting. In Table 6 below, the inventors present minimum detectable prevalence of somatic mutations in progressors and odds ratios for comparisons of somatic mutation prevalence in single genes between cases and the subcohort. These minimum detectable odds ratios account for sampling/weighting through the use of a design effect of 1.3 as well as for multiple statistical testing through the use of a Bonferroni-corrected alpha of 0.003

TABLE 6
Minimum detectable Odds Ratios for the comparison of somatic
mutations in progressing and non-progressing lesions

| | | Minimum detectable |
|---|---|---|
| | | odds ratios |
| | | in progressing vs. |
| Hypothetical prevalence | Minimum detectable | non-progressing |
| of somatic mutations | prevalence in | dysplasias |
| in one of 19 genes | progressing | Alpha = 0.003 |
| in non-progressing | dysplasias | power = 80% design |
| dysplasias (n = 460) | (n = 230) | effect due sampling = 1.3 |
| 1% | 4.5% | 8.73 |
| 2% | 6.3% | 5.84 |
| 5% | 11.0% | 3.86 |
| 10% | 17.7% | 3.06 |
| 25% | 35.3% | 2.51 |
| 50% | 61.2% | 2.44 |

Expected Outcomes, Potential Pitfalls and Alternative Strategies: The inventors expect that somatic mutations will be detected in driver genes in dysplastic tissues. In addition, the inventors expect that the prevalence of mutations will be higher in dysplastic tissues from patients that progress to OCSCC and that the proportion of dysplasias with detectable mutations will be highest in biopsies closest to cancer diagnosis. A potential concern, similar to Aim 1, is sample acquisition. However, the samples have already been identified and annotated. Another potential concern may be that Aims 1 and 2 are interdependent. In reality, the cohorts from the three Aims are entirely independent. The 19 genes identified by the TCGA, which include most of the genes identified by other large unbiased studies, is a reasonable starting point. A targeted gene panel is developed and applied for practical purposes to contain costs, maintain the highest depth coverage, facilitate the scalability of the assay, and its translation to clinical use. If a single bona fide mutation is identified in one of the 19 genes in Aim 1, the inventors will include it in the subsequent panel so there is purposefully very low stringency for inclusion. An alternative strategy is to perform targeted sequencing of all 19 genes or develop a different panel of genes altogether.

Aim 3: To investigate the presence of dysplasia-specific somatic mutations in key driver genes in saliva collected prior to the diagnosis of OCSCC. The inventors have shown that released tumor DNA can be identified in the saliva of OCSCC patients, with a median fraction of mutant DNA in the saliva of 0.65%, using highly sensitive and specific assays (68). Moreover, the preliminary data provide proof of concept that genetic alterations associated with dysplastic lesions can be detected in saliva even before they invade and acquire malignant potential. To further extend these studies to oral premalignancy, in collaboration with Drs. Anil Chaturvedi and Hormuzd Katki (National Cancer Institute), the inventors propose to conduct a case-cohort study using saliva of 177 OCSCC and 354 controls within five population-based US cohorts—the control arm of the Prostate, Lung, Colorectal, and Ovarian (PLCO) Cancer Screening Trial, the National Institutes of Health-American Association of Retired Persons Diet and Health Study (NIH-AARP), the Agricultural Health Study, the Cancer Prevention Study-II (CPS-II), and the Southern Community Cohort Study (SCCS). The inventors will investigate the association of mutations in key driver genes with the prospective risk of developing OCSCC. Additionally, within OCSCC cases, the inventors will investigate the time-course of the detection of mutations prior to OCSCC diagnosis. The work proposed is also highly cost-effective as it leverages multiple population-based cohorts with existing saliva samples, detailed demographic and behavioral data, and high-quality outcome ascertainment over several years of follow-up.

Aim 3.1. To compare the presence of somatic mutations in saliva in key driver genes between OCSCC and controls. The inventors hypothesize that the prevalence of somatic mutations in individual genes, total number of mutated genes, as well as combinations of mutated genes in the gene panel will be significantly higher in saliva from cases that ultimately develop OCSCC when compared to controls.

Aim 3.2. To investigate the presence of somatic mutations in key driver genes in saliva prior to the diagnosis of OCSCC, overall as well as stratified by time between specimen collection and cancer diagnosis (<1 years, 1-2 years, 2-3 years, 3-4 years, and 5+ years). The inventors hypothesize that somatic mutations will be detected in multiple driver genes in the gene panel in saliva samples collected several years prior to OCSCC diagnosis. The inventors also hypothesize that the proportion of dysplasias with detectable somatic mutations will be highest in samples closest to OCSCC diagnosis.

Design: The inventors will conduct a case-cohort study of 177 oral cavity cancers and 344 controls within 5 population-based US cohorts—the control arm of the Prostate, Lung, Colorectal, and Ovarian (PLCO) Cancer Screening Trial, the NIH-AARP Diet and Health Study, the Agricultural Health Study, the Southern Community Cohort Study (SCCS), and the Cancer Prevention Study-II (CPS-II), to investigate whether driver somatic mutations could be identified in saliva/buccal swabs prior to the detection of head and neck cancers. These cohorts represent almost all prospective cohort studies in the United States that have collected pre-diagnostic saliva samples. Cases will include individuals with incident head and neck cancers, including cancers of the oral cavity, oropharynx, hypopharynx, and larynx. Controls will represent a random sample of each cohort, stratified by 5-year age group, gender, and smoking (ever, former, current), and will be matched at a 2:1 ratio to cases. Table 7 shows the number of cases and the subcohort size in each cohort. The inventors recognize that saliva was collected using different protocols across different cohorts (Scope mouthwash using the “swishing” method, saliva collected using Oragene kits, and buccal swabs). The inventors do not anticipate differences in assay performance based on the method of saliva collection. For example, the inventors' study by Wang et al. (68), preliminary data, and unpublished data found minimal differences in DNA yield and fraction of mutant DNA across different methods of specimen collection, in part, because the sequencing approach provides an ultra-high depth of coverage and comprehensive analytic pipeline to overcome the shortcomings from even highly degraded DNA. Importantly, other studies have successfully utilized these cohorts for prospective studies of incident head and neck cancer. The inventors present three specific examples of prior use of oral rinses/saliva from these cohorts for pooled case-cohort or nested case-control analyses: 1) A study conducted using oral samples from PLCO and CPS-II to investigate the association of oral human papillomavirus (HPV) infection with risk of head and neck cancer (88); 2) a study conducted using oral samples from PLCO and CPS-II to investigate the association of the microbiome and risk of head and neck cancer (89); and 3) a case-cohort study conducted within four of the five cohorts included in the study (PLCO, NIH-AARP, Agricultural Health Study, and CPS-II) to investigate the association of the oral microbiome with risk of lung cancer, esophageal cancer, and gastric cancer (personal communication from Dr. Anil Chaturvedi, NCI, the epidemiologist collaborator on this grant proposal). Collectively, these prior studies underscore successful utilization of the oral samples from the proposed cohorts for cancer epidemiologic studies.

Mutation detection in saliva with Safe-SeqS: The sensitive Safe-SeqS error-reduction technology for detection of low frequency mutations will be used (FIG. 13). One of the challenges with using massively parallel sequencing for the detection of infrequent events is the high error rate, which ranges from 0.05%-1%. From previous studies, it is known that circulating or released tDNA is commonly present at frequencies less than 1%. Safe-SeqS has three key components to circumvent this problem: 1) template DNA molecules are assigned a unique identifier (UID) that can be tracked73, 2) PCR amplification of each UID generates UID families and 3) redundant sequencing of PCR products allows for high fidelity mutation detection, using the idea of “supermutants” (79). Supermutants are defined as an UID sequence family in which >90% of the family members have an identical mutation. The only way in which a supermutant can be generated is if the alteration was present in the original template molecule and not the result of an artifact or error generated during the amplification or sequencing process (FIG. 13). High quality sequence reads will be selected based on quality scores, which will be generated by the sequencing instrument to indicate the probability a base was called in error. The template-specific portion of the reads will be matched to reference sequences. Reads from a common template molecule will then be grouped based on the UIDs that are incorporated as molecular barcodes. Artifactual mutations introduced during the sample preparation or sequencing steps will be reduced by requiring a mutation to be present in >90% of reads in each UID family or supermutant. Each sample will be run in triplicate, with the mutant allele fractions defined as the total number of supermutants divided by the total number of UIDs. DNA from archived normal individuals will be used as a control, using at least five independent assays per queried mutation. Only saliva samples in which the mutant allele fractions significantly exceed their frequencies in control DNA (i.e., P-value <0.05) will be scored as positive.

Statistical analyses: For Aim 3.1, the inventors will quantify the presence of somatic mutations in oral cavity cancer cases prior to cancer diagnosis. Specifically, for each oral cavity cancer patient, the inventors will note the presence/absence of somatic mutations in each of the evaluated genes. Analyses will be conducted overall for all head and neck cancers, and by time between saliva sampling and cancer diagnosis (<1 year, 1-2 years, 2-3 years, 3-4 years, and 5+ years), as well as by parent cohort. The inventors will also conduct analyses stratified by key OCSCC risk factors—smoking status and alcohol consumption. These analyses will be conducted at the level of each gene, the total number of genes with detectable somatic mutations, and combinations of genes with somatic mutations. For Aim 3.2, the inventors will compare the prevalence of somatic mutations between cases and the subcohort. The inventors will incorporate the stratified random sampling of the subcohort by the use of inverse-probability weights. The inventors will utilize weighted Cox proportional hazards regression models to estimate hazard ratios and absolute risks for the incidence of OCSCC according to the presence of somatic mutations in each of the genes and the total number of genes with detectable somatic mutations. The inventors will investigate combinations of genes between cases and the subcohort using unweighted CART analysis, with 10-fold cross-validation. The weighted Cox regression models will be adjusted for age, gender, and study. Because somatic mutations represent causal intermediate states for the carcinogenic association of tobacco and alcohol, the key OCSCC risk factors, the inventors will consider analyses stratified by these risk factors in lieu of model-based adjustment. These analyses will be conducted overall and by time between saliva sampling and cancer diagnosis (<1 year, 1-2 years, 2-3 years, 3-4 years, and 5+ years). Given the multiple statistical testing across genes, to reduce the probability of false-positive associations, the inventors will utilize a Bonferroni-corrected threshold of P <0.003. To be less conservative the inventors will also consider using a False-Discovery Rate criterion of 5%. The primary analyses will be combined across the five cohorts; however, the inventors will conduct exploratory analyses stratified by the parent cohort. To the extent that results are heterogeneous across cohorts, the inventors will utilize random-effects metaanalysis methods to pool results from each cohort. The inventors will validate the findings using leave-one-cohort-out cross-validation to strengthen the inferences. The inventors will also summarize the analyses conducted for Aim 3.1 and Aim 3.2 as sensitivity (proportion of OCSCC with detectable somatic mutations in saliva prior to cancer diagnosis), specificity (1—prevalence of somatic mutations in the subcohort), positive predictive values (incidence of OCSCC given the presence of somatic mutations), and the complement of the negative predictive value (incidence of OCSCC in the absence of somatic mutations in saliva). Table 8 below shows the precision around the detection of somatic mutations for a gene at varying levels of prevalence in cases (Aim 3.1) as well as the minimum detectable odds ratios at varying levels of detection of mutations in controls (Aim 3.2 at alpha=0.003 and 80% power). These minimum detectable odds ratios account for sampling/weighting through the use of a design effect of 1.3 as well as for multiple statistical testing through the use of a Bonferroni-corrected alpha of 0.003. The inventors believe the odds ratios noted below are biologically meaningful, given that the exposures represent somatic mutations in key driver genes involved in HNSCC. Further, odds ratios estimates of gross chromosomal abnormalities, such as LOH at 3p, 9p, and 17p, in oral lesions have ranged from 12-52 in prior studies (24), suggesting that the minimum detectable odds ratios are plausible.

Expected Outcomes, Potential Pitfalls and Alternative Strategies: The identification of rare variants is technically challenging and could involve sequencing errors and artifacts. The inventors will incorporate three levels of quality-control. First, the inventors will only include samples that have high-quality sequence reads based on quality scores generated by the sequencing instrument for the probability of error in base-calling (68). Second, the inventors will conduct Safe-SeqS in three independent runs and will note the mutant allele frequencies as an average of three runs (68). Third, in addition to positive and negative controls, the inventors plan to incorporate 10% of specimens as blinded duplicates to assess assay reproducibility. Given the multiple statistical testing across genes, to reduce the probability of false-positive associations for the case-control comparisons, the inventors will utilize a Bonferroni-corrected threshold of P<0.003. The inventors recognize that samples have been collected using different protocols across the cohorts but prior successful use of oral rinses/saliva from these cohorts is cited. Moreover, the inventors do not anticipate differences in assay performance based and this heterogeneity actually proves the robustness and potential broad adaption of the approach across diverse conditions as would be necessary for a clinical test to be viable. Again, as stated above, from the inventors' previous work (68), preliminary data, and unpublished analyses the inventors found minimal differences in DNA yield and fraction of mutant DNA across different methods of specimen collection. The inventors' primary analyses will be combined across the five cohorts. However, it is possible that there will be differences between cohorts. Therefore, the inventors will conduct exploratory analyses stratified by the parent cohort.

Scientific Rigor and Reproducibility. The data derived from the assays are expected to be scientifically sound. The inventors will follow stringent methods for choosing biospecimen samples. To ascertain that only histologically valid sections of high quality and purity from a tissue block are used, laser capture microdissection will be performed as needed. All assays will be conducted in triplicate and controls have been built into the power calculations. For bioinformatic analyses of the sequencing data, because the inventors will target previously established genetic markers of HNSCC in a large number of samples with high sequencing depth, it will be of sufficient statistical power (as demonstrated in the Research Design). The inventors will also include rigorous false positive and false negative controls in the analyses, and use both internal and external, publicly available datasets to assess the performance of the assays, identify potential artifacts and test presumptions. Strict quality control procedures and metrics have been and will continue to be a focus of the inventors' research team.

### VI. References for Example 2

The following references, to the extent that they provide exemplary procedural or other details supplementary to those set forth herein, are specifically incorporated herein by reference.
1. Poling, J. S. et al. Human papillomavirus (HPV) status of non-tobacco related squamous cell carcinomas of the lateral tongue. Oral Oncol 50, 306-310, oi:10.1016/j.oraloncology 0.2014.01.006 (2014).
2. Castellsague, X. et al. HPV Involvement in Head and Neck Cancers: Comprehensive Assessment of Biomarkers in 3680 Patients. J Natl Cancer Inst 108, djv403, doi:10.1093/jnci/djv403 (2016).
3. Lingen, M. W. et al. Low etiologic fraction for high-risk human papillomavirus in oral cavity squamous cell carcinomas. Oral Oncol 49, 1-8, doi:10.1016/j.oraloncology.2012.07.002 (2013).
4. Zafereo, M. E. et al. Squamous cell carcinoma of the oral cavity often overexpresses p16 but is rarely driven by human papillomavirus. Oral Oncol 56, 47-53, doi:10.1016/j.oraloncology.2016.03.003 (2016).
5. Axell, T., Pindborg, J. J., Smith, C. J. & van der Waal, I. Oral white lesions with special reference to precancerous and tobacco-related lesions: conclusions of an international symposium held in Uppsala, Sweden, May 18-21 1994. International Collaborative Group on Oral White Lesions. J Oral Pathol Med 25, 49-54 (1996).
6. Forastiere, A., Koch, W., Trotti, A. & Sidransky, D. Head and neck cancer. N Engl J Med 345, 1890-1900, doi:10.1056/NEJMra001375 (2001).
7. Rhodus, N. L. Oral cancer: leukoplakia and squamous cell carcinoma. Dent Clin North Am 49, 143-165, ix, doi:10.1016/j.cden.2004.07.003 (2005).
8. Shiboski, C. H., Shiboski, S. C. & Silverman, S., Jr. Trends in oral cancer rates in the United States, 1973-1996. Community Dent Oral Epidemiol 28, 249-256 (2000).
9. Siegel, R. L., Miller, K. D. & Jemal, A. Cancer Statistics, 2017. CA: a cancer journal for clinicians 67, 7-30, doi:10.3322/caac.21387 (2017).
10. Warnakulasuriya, S. Global epidemiology of oral and oropharyngeal cancer. Oral Oncol 45, 309-316, doi:10.1016/j.oraloncology.2008.06.002 (2009).
11. Petersen, P. E. Strengthening the prevention of oral cancer: the WHO perspective. Community Dent Oral Epidemiol 33, 397-399, doi:10.1111/j.1600-0528.2005.00251.x (2005).
12. Bouquot, J. E. Common oral lesions found during a mass screening examination. J Am Dent Assoc 112, 50-57 (1986).
13. Jiang, W. W., Fujii, H., Shirai, T., Mega, H. & Takagi, M. Accumulative increase of loss of heterozygosity from leukoplakia to foci of early cancerization in leukoplakia of the oral cavity. Cancer 92, 2349-2356 (2001).
14. Mao, L. et al. Frequent microsatellite alterations at chromosomes 9p21 and 3p14 in oral premalignant lesions and their value in cancer risk assessment. Nat Med 2, 682-685 (1996).
15. Partridge, M. et al. Detection of minimal residual cancer to investigate why oral tumors recur despite seemingly adequate treatment. Clin Cancer Res 6, 2718-2725 (2000).
16. Thomson, P. J. Field change and oral cancer: new evidence for widespread carcinogenesis? Int J Oral Maxillofac Surg 31, 262-266, doi:10.1054/ijom.2002.0220 (2002).
17. Lingen, M. W., Kalmar, J. R., Karrison, T. & Speight, P. M. Critical evaluation of diagnostic aids for the detection of oral cancer. Oral Oncol 44, 10-22, doi:10.1016/j.oraloncology.2007.06.011 (2008).
18. Patton, L. L., Epstein, J. B. & Kerr, A. R. Adjunctive techniques for oral cancer examination and lesion diagnosis: a systematic review of the literature. J Am Dent Assoc 139, 896-905; quiz 993-894 (2008).
19. Rethman, M. P. et al. Evidence-based clinical recommendations regarding screening for oral squamous cell carcinomas. J Am Dent Assoc 141, 509-520 (2010).
20. Macey, R. et al. Diagnostic tests for oral cancer and potentially malignant disorders in patients presenting with clinically evident lesions. The Cochrane database of systematic reviews, Cd010276, doi:10.1002/14651858.CD010276.pub2 (2015).
21. Walsh, T. et al. Clinical assessment to screen for the detection of oral cavity cancer and potentially malignant disorders in apparently healthy adults. The Cochrane database of systematic reviews, Cd010173, doi:10.1002/14651858.CD010173.pub2 (2013).
22. Lingen, M. W. et al. Evidence-based clinical practice guideline for the evaluation of potentially malignant disorders in the oral cavity: A report of the American Dental Association. J Am Dent Assoc 148, 712-727.e710, doi:10.1016/j.adaj.2017.07.032 (2017).
23. Lingen, M. W. et al. Adjuncts for the evaluation of potentially malignant disorders in the oral cavity: Diagnostic test accuracy systematic review and meta-analysis-a report of the American Dental Association. J Am Dent Assoc 148, 797-813.e752, doi:10.1016/j.adaj.2017.08.045 (2017).
24. Karabulut, A. et al. Observer variability in the histologic assessment of oral premalignant lesions. J Oral Pathol Med 24, 198-200 (1995).
25. Nankivell, P. et al. The binary oral dysplasia grading system: validity testing and suggested improvement. Oral surgery, oral medicine, oral pathology and oral radiology 115, 87-94, doi:10.1016/j.0000.2012.10.015 (2013).
26. Dost, F., Le Cao, K., Ford, P. J., Ades, C. & Farah, C. S. Malignant transformation of oral epithelial dysplasia: a real-world evaluation of histopathologic grading. Oral surgery, oral medicine, oral pathology and oral radiology 117, 343-352, doi:10.1016/j.0000.2013.09.017 (2014).
27. Speight, P. M. et al. Interobserver agreement in dysplasia grading: toward an enhanced gold standard for clinical pathology trials. Oral surgery, oral medicine, oral pathology and oral radiology 120, 474-482.e472, doi:10.1016/j.0000.2015.05.023 (2015).
28. Krishnan, L. et al. Inter- and intra-observer variability in three grading systems for oral epithelial dysplasia. Journal of oral and maxillofacial pathology: JOMFP 20, 261-268, doi:10.4103/0973-029×.185928 (2016).
29. R, S. A. et al. Inter- and Intra-Observer Variability in Diagnosis of Oral Dysplasia. Asian Pacific journal of cancer prevention: APJCP 18, 3251-3254, doi:10.22034/apjcp.2017.18.12.3251 (2017).
30. Warnakulasuriya, S., Reibel, J., Bouquot, J. & Dabelsteen, E. Oral epithelial dysplasia classification systems: predictive value, utility, weaknesses and scope for improvement. J Oral Pathol Med 37, 127-133, doi:10.1111/j.1600-0714.2007.00584.x (2008).
31. Kujan, O. et al. Why oral histopathology suffers inter-observer variability on grading oral epithelial dysplasia: an attempt to understand the sources of variation. Oral Oncol 43, 224-231, doi:10.1016/j.oraloncology.2006.03 0.009 (2007).
32. Abbey, L. M. et al. Intraexaminer and interexaminer reliability in the diagnosis of oral epithelial dysplasia. Oral Surg Oral Med Oral Pathol Oral Radiol Endod 80, 188-191 (1995).
33. Mincer, H. H., Coleman, S. A. & Hopkins, K. P. Observations on the clinical characteristics of oral lesions showing histologic epithelial dysplasia. Oral surgery, oral medicine, and oral pathology 33, 389-399 (1972).
34. Arduino, P. G. et al. Outcome of oral dysplasia: a retrospective hospital-based study of 207 patients with a long follow-up. J Oral Pathol Med 38, 540-544, doi:10.1111/j.1600-0714.2009.00782.x (2009).
35. Mehanna, H. M., Rattay, T., Smith, J. & McConkey, C. C. Treatment and follow-up of oral dysplasia—a systematic review and meta-analysis. Head Neck 31, 1600-1609, doi:10.1002/hed.21131 (2009).
36. Zhang, L. & Rosin, M. P. Loss of heterozygosity: a potential tool in management of oral premalignant lesions? J Oral Pathol Med 30, 513-520 (2001).
37. Zhang, L. et al. Loss of heterozygosity (LOH) profiles-validated risk predictors for progression to oral cancer. Cancer Prev Res (Phila) 5, 1081-1089, doi:10.1158/1940-6207.capr-12-0173 (2012).
38. Yang, Y. et al. Progress risk assessment of oral premalignant lesions with saliva miRNA analysis. BMC cancer 13, 129, doi:10.1186/1471-2407-13-129 (2013).
39. Xiao, W. et al. Upregulation of miR-31* is negatively associated with recurrent/newly formed oral leukoplakia. PLoS One 7, e38648, doi: 10.1371/j ournal.pone.0038648 (2012).
40. Towle, R. et al. Global analysis of DNA methylation changes during progression of oral cancer. Oral Oncol 49, 1033-1042, doi:10.1016/j.oraloncology.2013.08.005 (2013).
41. Torres-Rendon, A., Stewart, R., Craig, G. T., Wells, M. & Speight, P. M. DNA ploidy analysis by image cytometry helps to identify oral epithelial dysplasias with a high risk of malignant progression. Oral Oncol 468-473, doi:10.1016/j.oraloncology.2008.07.006 (2009).
42. Takeshima, M. et al. High frequency of hypermethylation of p14, p15 and p16 in oral pre-cancerous lesions associated with betel-quid chewing in Sri Lanka. J Oral Pathol Med 37, 475-479, doi:10.1111/j.1600-0714.2008.00644.x (2008).
43. Sperandio, M. et al. Predictive value of dysplasia grading and DNA ploidy in malignant transformation of oral potentially malignant disorders. Cancer Prev Res (Phila) 6, 822-831, doi:10.1158/1940-6207.capr-13-0001 (2013).
44. Shridhar, K. et al. DNA methylation markers for oral pre-cancer progression: A critical review. Oral Oncol 53, 1-9, doi:10.1016/j.oraloncology.2015.11.012 (2016).
45. Saito, T. et al. Flow cytometric analysis of nuclear DNA content in oral leukoplakia: relation to clinicopathologic findings. Int J Oral Maxillofac Surg 24, 44-47 (1995).
46. Philipone, E. et al. MicroRNAs-208b-3p, 204-5p, 129-2-3p and 3065-5p as predictive markers of oral leukoplakia that progress to cancer. American journal of cancer research 6, 1537-1546 (2016).
47. Pentenero, M. et al. DNA aneuploidy and dysplasia in oral potentially malignant disorders: association with cigarette smoking and site. Oral Oncol 45, 887-890, doi:10.1016/j.oraloncology.2009.03.008 (2009).
48. Partridge, M. et al. Allelic imbalance at chromosomal loci implicated in the pathogenesis of oral precancer, cumulative loss and its relationship with progression to cancer. Oral Oncol 34, 77-83 (1998).
49. Maimaiti, A., Abudoukeremu, K., Tie, L., Pan, Y. & Li, X. MicroRNA expression profiling and functional annotation analysis of their targets associated with the malignant transformation of oral leukoplakia. Gene 558, 271-277, doi:10.1016/j.gene.2015.01.004 (2015).
50. Maclellan, S. A. et al. Differential expression of miRNAs in the serum of patients with high-risk oral lesions. Cancer medicine 1, 268-274, doi:10.1002/cam4.17 (2012).
51. Kresty, L. A. et al. Alterations of p16(INK4a) and p14(ARF) in patients with severe oral epithelial dysplasia. Cancer Res 62, 5295-5300 (2002).
52. Hung, K. F. et al. MicroRNA-31 upregulation predicts increased risk of progression of oral potentially malignant disorder. Oral Oncol 53, 42-47, doi:10.1016/j.oraloncology.2015.11.017 (2016).
53. Hall, G. L. et al. p16 Promoter methylation is a potential predictor of malignant transformation in oral epithelial dysplasia. Cancer epidemiology, biomarkers & prevention: a publication of the American Association for Cancer Research, cosponsored by the American Society of Preventive Oncology 17, 2174-2179, doi:10.1158/1055-9965.epi-07-2867 (2008).
54. Donadini, A. et al. Oral cancer genesis and progression: DNA near-diploid aneuploidization and endoreduplication by high resolution flow cytometry. Cellular oncology: the official journal of the International Society for Cellular Oncology 32, 373-383, doi:10.3233/clo-2010-0525 (2010).
55. Diwakar, N., Sperandio, M., Sherriff, M., Brown, A. & Odell, E. W. Heterogeneity, histological features and DNA ploidy in oral carcinoma by image-based analysis. Oral Oncol 41, 416-422, doi:10.1016/j.oraloncology.2004.10.009 (2005).
56. D'Souza, W. & Saranath, D. Clinical implications of epigenetic regulation in oral cancer. Oral Oncol 51, 1061-1068, doi:10.1016/j.oraloncology.2015.09.006 (2015).
57. Clague, J. et al. Genetic variation in MicroRNA genes and risk of oral premalignant lesions. Molecular carcinogenesis 49, 183-189, doi:10.1002/mc.20588 (2010).
58. Cervigne, N. K. et al. Identification of a microRNA signature associated with progression of leukoplakia to oral carcinoma. Human molecular genetics 18, 4818-4829, doi:10.1093/hmg/ddp446 (2009).
59. Cao, J. et al. Methylation of p16 CpG island associated with malignant progression of oral epithelial dysplasia: a prospective cohort study. Clin Cancer Res 15, 5178-5183, doi:10.1158/1078-0432.ccr-09-0580 (2009).
60. Bremmer, J. F. et al. Prognostic value of DNA ploidy status in patients with oral leukoplakia. Oral Oncol 47, 956-960, doi:10.1016/j.oraloncology.2011.07.025 (2011).
61. Bremmer, J. F. et al. A noninvasive genetic screening test to detect oral preneoplastic lesions. Laboratory investigation; a journal of technical methods and pathology 85, 1481-1488, doi:10.1038/1abinvest.3700342 (2005).
62. Bremmer, J. F. et al. Comparative evaluation of genetic assays to identify oral pre-cancerous fields. J Oral Pathol Med 37, 599-606, doi:10.1111/j.1600-0714.2008.00682.x (2008).
63. Bradley, G. et al. Abnormal DNA content in oral epithelial dysplasia is associated with increased risk of progression to carcinoma. British journal of cancer 103, 1432-1442, doi:10.1038/sj.bj c.6605905 (2010).
64. Abdulmajeed, A. A. & Farah, C. S. Gene expression profiling for the purposes of biomarker discovery in oral potentially malignant lesions: a systematic review. Clinical Medicine Insights. Oncology 7, 279-290, doi:10.4137/cmo.s12950 (2013).
65. Koch, W. M. et al. p53 mutation and locoregional treatment failure in head and neck squamous cell carcinoma. J Natl Cancer Inst 88, 1580-1586 (1996).
66. Nawroz, H., Koch, W., Anker, P., Stroun, M. & Sidransky, D. Microsatellite alterations in serum DNA of head and neck cancer patients. Nat Med 2, 1035-1037 (1996).
67. Bettegowda, C. et al. Detection of circulating tumor DNA in early- and late-stage human malignancies. Sci Transl Med 6, 224ra224, doi:10.1126/scitranslmed.3007094 (2014).
68. Wang, Y. et al. Detection of somatic mutations and HPV in the saliva and plasma of patients with head and neck squamous cell carcinomas. Sci Transl Med 7, 293ra104, doi:10.1126/scitranslmed.aaa8507 (2015).
69. Diehl, F. et al. Analysis of mutations in DNA isolated from plasma and stool of colorectal cancer patients. Gastroenterology 135, 489-498, doi:10.1053/j.gastro.2008.05.039 (2008).
70. Kinde, I. et al. Evaluation of DNA from the Papanicolaou test to detect ovarian and endometrial cancers. Sci Transl Med 5, 167ra164, doi:10.1126/scitranslmed.3004952 (2013).
71. Wang, Y. et al. Detection of tumor-derived DNA in cerebrospinal fluid of patients with primary tumors of the brain and spinal cord. Proc Natl Acad Sci USA 112, 9704-9709, doi:10.1073/pnas.1511694112 (2015).
72. Agrawal, N. et al. Exome sequencing of head and neck squamous cell carcinoma reveals inactivating mutations in NOTCH1. Science 333, 1154-1157, doi:10.1126/science.1206923 (2011).
73. Stransky, N. et al. The mutational landscape of head and neck squamous cell carcinoma. Science 333, 1157-1160, doi:10.1126/science.1208130 (2011).
74. Comprehensive genomic characterization of head and neck squamous cell carcinomas. Nature 517, 576-582, doi:10.1038/nature14129 (2015).
75. Anglim, P. P. et al. Identification of a panel of sensitive and specific DNA methylation markers for squamous cell lung cancer. Mol Cancer 7, 62, doi:10.1186/1476-4598-7-62 (2008).
76. Schiffman, J. D. et al. Oncogenic BRAF mutation with CDKN2A inactivation is characteristic of a subset of pediatric malignant astrocytomas. Cancer Res 70, 512-519, doi:10.1158/0008-5472.CAN-09-1851 (2010).
77. Diehl, F. et al. Detection and quantification of mutations in the plasma of patients with colorectal tumors. Proc Natl Acad Sci USA 102, 16368-16373, doi:10.1073/pnas.0507904102 (2005).
78. Diehl, F. et al. Circulating mutant DNA to assess tumor dynamics. Nat Med 14, 985-990, doi:10.1038/nm.1789 (2008). 79. Kinde, I., Wu, J., Papadopoulos, N., Kinzler, K. W. & Vogelstein, B. Detection and quantification of rare mutations with massively parallel sequencing. Proc Natl Acad Sci USA 108, 9530-9535, doi:10.1073/pnas.1105422108 (2011).
80. Vogelstein, B. & Kinzler, K. W. Digital PCR. Proc Natl Acad Sci USA 96, 9236-9241 (1999).
81. Chaturvedi, A. K. et al. Human papillomavirus and rising oropharyngeal cancer incidence in the United States. J Clin Oncol 29, 4294-4301, doi:10.1200/JCO.2011.36.4596 (2011).
82. D'Souza, G. et al. Case-control study of human papillomavirus and oropharyngeal cancer. N Engl J Med 356, 1944-1956, doi:10.1056/NEJMoa065497 (2007).
83. Sausen, M. et al. Integrated genomic analyses identify ARID1A and ARID1B alterations in the childhood cancer neuroblastoma. Nat Genet 45, 12-17, doi:10.1038/ng.2493 (2013).
84. Kadri, S. et al. Clinical Validation of a Next-Generation Sequencing Genomic Oncology Panel via Cross-Platform Benchmarking against Established Amplicon Sequencing Assays. The Journal of molecular diagnostics: JMD 19, 43-56, doi:10.1016/j.jmoldx.2016.07.012 (2017).
85. Langholz, B. & Thomas, D. C. Nested case-control and case-cohort methods of sampling from a cohort: a critical comparison. Am J Epidemiol 131, 169-176, doi:10.1093/oxfordjournals.aje.a115471 (1990).
86. Wacholder, S. Practical considerations in choosing between the case-cohort and nested case-control designs. Epidemiology 2, 155-158, doi:10.1097/00001648-199103000-00013 (1991).
87. Mark, S. D. & Katki, H. A. Specifying and Implementing Nonparametric and Semiparametric Survival Estimators in Two-Stage (Nested) Cohort Studies With Missing Case Data. Journal of the American Statistical Association 101, 460-471, doi:10.1198/016214505000000952 (2006).
88. Agalliu, I. et al. Associations of Oral alpha-, beta-, and gamma-Human Papillomavirus Types With Risk of Incident Head and Neck Cancer. JAMA Oncol 2, 599-606, doi:10.1001/jamaonco1.2015.5504 (2016).
89. Hayes, R. B. et al. Association of Oral Microbiome With Risk for Incident Head and Neck Squamous Cell Cancer. JAMA Oncol 4, 358-365, doi:10.1001/jamaonco1.2017.4777 (2018).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for evaluating a subject comprising detecting genetic mutation(s) in the DNA sequence of one or more oral cavity squamous cell carcinoma (OCSCC) biomarker(s) in a biological sample from the subject, wherein the biological sample consists of an oral rinse sample comprising saliva DNA, wherein the OCSCC biomarker(s) consist of TP53, CDKN2A, FAT1, CASP8, NOTCH1, PIK3CA, and HRAS, and wherein the genetic mutations are detected by next generation sequencing (NGS).

2. A method for evaluating a subject comprising detecting genetic mutation(s) in the DNA sequence of one or more head and neck cancer or oral cavity squamous cell carcinoma (OCSCC) biomarker(s) in a biological sample from the subject comprising DNA, wherein the biomarker(s) comprise TP53, CDKN2A, FAT1, CASP8, NOTCH1, PIK3CA, and/or HRAS.

3. The method of claim 2, wherein the biological sample comprises saliva DNA.

4. The method of claim 3, wherein the biological sample comprises an oral rinse sample.

5. The method of any one of claims 2-4, wherein the biological sample comprises cells or an extract thereof.

6. The method of any one of claims 2-5, wherein the biological sample excludes serum or plasma.

7. The method of any one of claims 2-6, wherein the method excludes detecting genetic mutations in the DNA sequence of one or more biomarkers in serum or plasma.

8. The method of any one of claims 3-7, wherein the method excludes detecting genetic mutation(s) or analysis of DNA in a non-saliva sample.

9. The method of any one of claims 2-8, wherein the method excludes centrifugation of the biological sample from the subject.

10. The method of any one of claims 2-8, wherein the method excludes centrifugation of the biological sample from the subject prior to DNA isolation.

11. The method of claim 7-10, wherein the method further comprises isolating DNA from a cellular fraction of the biological sample.

12. The method of any one of claims 2-11, wherein the method further comprises ligation of an adaptor to the DNA.

13. The method of claim 12, wherein the adaptor comprises at least one barcode.

14. The method of claim 12 or 13 wherein the adaptor comprises a 5′ and/or 3′ primer binding site.

15. The method of any one of claims 2-14, wherein the method further comprises enrichment of the DNA in the biological sample for the biomarker genes.

16. The method of claim 15, wherein enrichment comprises contacting the sample with a nucleic acid probe complimentary to the biomarker gene under conditions that allow for the hybridization of the probe and DNA in the biological sample that is at least partially complimentary to the probe.

17. The method of claim 16, wherein the enrichment further comprises isolating the DNA hybridized to the probe.

18. The method of claim 17, wherein the method further comprises sequencing the DNA hybridized to the probe.

19. The method of any one of claims 2-18, wherein the method further comprises sequencing DNA comprising all or part of the biomarker genes to provide the sequence of all or part of the biomarker genes.

20. The method of claim 19, wherein sequencing comprising contacting the biomarker gene with a polymerase and primer(s)s that hybridize to the biomarker gene or adjacent regions and using polymerase chain reaction (PCR) to amplify DNA sequences comprising the gene.

21. The method of claim 19 or 20, wherein sequencing comprises next generation sequencing.

22. The method of any one of claims 19-21, wherein the coding exon regions of the biomarker gene are sequenced.

23. The method of claim 22, wherein all of the coding exon regions of the biomarker gene are sequenced.

24. The method of any one of claims 19-23, wherein the method further comprises comparing the sequence of the biomarker genes to a control.

25. The method of claim 24, wherein the control comprises the wild-type sequence of the gene.

26. The method of any one of claims 2-25, wherein the number of biomarkers evaluated in the biological sample is 1-7 biomarkers.

27. The method of any one of claims 2-26, wherein the biomarker comprises TP53.

28. The method of any one of claims 2-27, wherein the biomarker comprises CDKN2A.

29. The method of any one of claims 2-28, wherein the biomarker comprises FAT1.

30. The method of any one of claims 2-29, wherein the biomarker comprises CASP8.

31. The method of any one of claims 2-30, wherein the biomarker comprises NOTCH1.

32. The method of any one of claims 2-31, wherein the biomarker comprises HRAS.

33. The method of any one of claims 2-32, wherein the biomarker comprises PIK3CA.

34. The method of any one of claims 2-26, wherein the biomarkers comprise TP53, CDKN2A, FAT1, CASP8, and Notch1.

35. The method of claim 34, wherein the biomarkers comprise TP53, CDKN2A, FAT1, CASP8, Notch1, PIK3CA, and HRAS.

36. The method of claim 34, wherein the biomarkers consist of TP53, CDKN2A, FAT1, CASP8, Notch1, PIK3CA, and HRAS.

37. The method of any one of claims 2-36, wherein at least one genetic mutation was detected.

38. The method of claim 37, wherein the method further comprises performing one or more diagnostic tests for head and neck cancer or OCSCC.

39. The method of claim 38, wherein the diagnostic test comprises a conventional visual and tactile exam, tissue biopsy, and/or histological evaluation of a tissue biopsy.

40. The method of any one of claims 37-39, wherein the method further comprises treating the subject for head and neck cancer or OCSCC.

41. The method of claim 40, wherein the treatment comprises surgery, chemotherapy, radiation, or combinations thereof.

42. The method of claim 41, wherein the treatment comprises chemotherapy and wherein the chemotherapy comprises cisplatin.

43. The method of any one of claims 2-42, wherein no genetic mutations were detected.

44. The method of claim 43, wherein the method excludes performing one or more diagnostic tests for head and neck cancer or OCSCC.

45. The method of any one of claims 2-43, wherein the subject is a human subject.

46. The method of claim 45, wherein the subject is greater than 50 years old.

47. The method of any one of claims 2-46, wherein the subject does not have any symptoms of head and neck cancer or OCSCC.

48. The method of any one of claims 2-46, wherein the subject has one or more symptoms of head and neck cancer or OCSCC.

49. The method of any one of claims 2-48, wherein the method excludes whole exome sequencing methods.

50. The method of any one of claims 2-49, wherein the method excludes droplet digital PCR.

51. The method of any one of claims 2-50, wherein the OCSCC comprises HPV-negative OCSCC.

52. The method of any one of claims 2-51, wherein the mutation is further defined as a somatic mutation.

53. The method of any one of claims 2-52, wherein the variant allele frequency (VAF) of the mutation is less than 1%.

54. The method of any one of claims 2-53, wherein the DNA excludes cfDNA.

55. The method of any one of claims 2-54, wherein the subject has not been treated with therapeutic levels of chemotherapy or radiation.

56. The method of any one of claims 2-55, wherein the method further comprises diagnosing the subject with head and neck cancer or OCSCC based on the evaluation.

57. The method of claim 56, wherein the OCSCC comprises carcinoma of the tongue, buccal mucosa, alveolus, gingivobuccal sulcus, hard palate, lip, retromolar trigone, maxilla, or gum.

58. The method of claim 56, wherein the subject is diagnosed with premalignant lesion, stage I, II, III, or IV based on the evaluation.

59. The method of any one of claims 2-58, wherein the subject is a non-smoker.

60. The method of any one of claims 2-58, wherein the subject is a smoker.

61. A method for treating a subject with head and neck cancer or OCSCC or premalignant lesion, the method comprising administering a treatment for head and neck cancer or OCSCC to a subject that has, or has been determined to have, at least one genetic mutation in the DNA sequence of one or more head and neck cancer or OCSCC biomarker(s) in a biological sample from the subject comprising DNA, wherein the biomarker(s) comprise TP53, CDKN2A, FAT1, CASP8, NOTCH1, PIK3CA, and/or HRAS.

62. The method of claim 61, wherein the biological sample comprises saliva DNA.

63. The method of claim 62, wherein the biological sample comprises an oral rinse sample.

64. The method of any one of claims 61-63, wherein the biological sample comprises cells or an extract thereof.

65. The method of any one of claims 61-64, wherein the biological sample excludes serum or plasma.

66. The method of any one of claims 61-65, wherein the method is for treating OCSCC.

67. The method of claim 66, wherein the method excludes treatment of head and neck cancer and/or subjects having head and neck cancer.

68. The method of any one of claims 61-67, wherein the subject excludes one that has had detection of genetic mutations in the DNA sequence of one or more biomarkers in serum or plasma.

69. The method of any one of claims 61-68, wherein the wherein the subject excludes one that has had detection of genetic mutation(s) or analysis of DNA in a non-saliva sample.

70. The method of any one of claims 61-69, wherein the wherein the subject excludes one that has had centrifugation of the biological sample.

71. The method of any one of claims 61-70, wherein the wherein the subject excludes one that has had centrifugation of the biological sample prior to DNA isolation.

72. The method of claim 71, wherein DNA from a cellular fraction of the biological sample was evaluated for genetic mutations in the biomarker genes.

73. The method of claim 72, wherein the evaluation comprised ligation of an adaptor to the DNA.

74. The method of claim 73, wherein the adaptor comprised at least one barcode.

75. The method of claim 73 or 74 wherein the adaptor comprised a 5′ and/or 3′ primer binding site.

76. The method of any one of claims 72-75, wherein the evaluation further comprised enrichment of the DNA in the biological sample for the biomarker genes.

77. The method of claim 76, wherein enrichment comprised contacting the sample with a nucleic acid probe complimentary to the biomarker gene under conditions that allow for the hybridization of the probe and DNA in the biological sample that is at least partially complimentary to the probe.

78. The method of claim 77, wherein the enrichment further comprised isolating the DNA hybridized to the probe.

79. The method of claim 78, wherein the evaluation further comprised sequencing the DNA hybridized to the probe.

80. The method of any one of claims 72-79, wherein the evaluation further comprised sequencing DNA comprising all or part of the biomarker genes to provide the sequence of all or part of the biomarker genes.

81. The method of claim 80, wherein sequencing comprised contacting the biomarker gene with a polymerase and primer(s)s that hybridize to the biomarker gene or adjacent regions and using polymerase chain reaction (PCR) to amplify DNA sequences comprising the OCSCC gene.

82. The method of claim 80 or 81, wherein sequencing comprises next generation sequencing.

83. The method of any one of claims 80-82, wherein the coding exon regions of the gene were sequenced.

84. The method of claim 83, wherein all of the coding exon regions of the gene were sequenced.

85. The method of any one of claims 80-84, wherein the method further comprised comparing the sequence of the biomarker genes to a control.

86. The method of claim 85, wherein the control comprised the wild-type sequence of the gene.

87. The method of any one of claims 61-86, wherein the number of biomarkers evaluated in the biological sample was 1-7 biomarkers.

88. The method of any one of claims 61-87, wherein the biomarker comprised TP53.

89. The method of any one of claims 61-88, wherein the biomarker comprised CDKN2A.

90. The method of any one of claims 61-89, wherein the biomarker comprised FAT1.

91. The method of any one of claims 61-90, wherein the biomarker comprised CASP8.

92. The method of any one of claims 61-91, wherein the biomarker comprised NOTCH1.

93. The method of any one of claims 61-92, wherein the biomarker comprised HRAS.

94. The method of any one of claims 61-93, wherein the biomarker comprised PIK3CA.

95. The method of any one of claims 61-87, wherein the biomarkers comprised TP53, CDKN2A, FAT1, CASP8, and Notch1.

96. The method of claim 95, wherein the biomarkers comprised TP53, CDKN2A, FAT1, CASP8, Notch1, PIK3CA, and HRAS.

97. The method of claim 95, wherein the biomarkers consisted of TP53, CDKN2A, FAT1, CASP8, Notch1, PIK3CA, and HRAS.

98. The method of any one of claims 61-97, wherein the method further comprises performing one or more diagnostic tests for head and neck cancer or OCSCC.

99. The method of claim 98, wherein the diagnostic test comprises a conventional visual and tactile exam, tissue biopsy, and/or histological evaluation of a tissue biopsy.

100. The method of any one of claims 61-99, wherein the treatment comprises surgical excision of a tumor, neck dissection, radiation therapy, and/or chemotherapy.

101. The method of any one of claims 61-100, wherein the subject is a human subject.

102. The method of claim 101, wherein the subject is greater than 50 years old.

103. The method of any one of claims 61-102, wherein the subject does not have any symptoms of head and neck cancer or OCSCC.

104. The method of any one of claims 61-102, wherein the subject has one or more symptoms of head and neck cancer or OCSCC.

105. The method of any one of claims 72-104, wherein the evaluation excludes whole exome sequencing methods.

106. The method of any one of claims 72-105, wherein the evaluation excludes droplet digital PCR.

107. The method of any one of claims 61-106, wherein the OCSCC comprises HPV-negative OCSCC.

108. The method of any one of claims 61-107, wherein the variant allele frequency (VAF) of the mutation is less than 1%

109. The method of any one of claims 61-108, wherein the subject has not been previously treated with therapeutic levels of chemotherapy or radiation.

110. The method of any one of claims 61-108, wherein the OCSCC comprises carcinoma of the tongue, buccal mucosa, alveolus, gingivobuccal sulcus, hard palate, lip, retromolar trigone, maxilla, or gum.

111. The method of any one of claims 61-110, wherein the subject is a non-smoker.

112. The method of any one of claims 61-110, wherein the subject is a smoker.

113. A method of diagnosing or screening a subject for head and neck cancer or OCSCC or pre-malignant comprising
a) detecting genetic mutations in the DNA sequence of one or more biomarker(s) in a biological sample from the subject comprising DNA, wherein the biomarker(s) comprise TP53, CDKN2A, FAT1, CASP8, NOTCH1, PIK3CA, and/or HRAS;
b) determining that the subject has or is at high risk of having head and neck cancer or OCSCC when at least one genetic mutation in a biomarker gene is detected or determining that the subject does not have or is at low risk of having when no genetic mutation in a biomarker gene is detected.

114. The method of claim 113, wherein the biological sample comprises saliva DNA.

115. The method of claim 114, wherein the biological sample comprises an oral rinse sample.

116. The method of any one of claims 113-115, wherein the biological sample comprises cells or an extract thereof.

117. The method of claim 116, wherein the method further comprises isolating DNA from a cellular fraction of the biological sample.

118. The method of any one of claims 113-117, wherein the biological sample excludes serum or plasma.

119. The method of any one of claims 113-118, wherein the method excludes detecting genetic mutations in the DNA sequence of one or more biomarkers in serum or plasma.

120. The method of any one of claims 113-119, wherein the method excludes detecting genetic mutation(s) or analysis of DNA in a non-saliva sample.

121. The method of any one of claims 61-65, wherein the method is for diagnosing or screening subjects for OCSCC.

122. The method of claim 66, wherein the method excludes diagnosing or screening subjects for head and neck cancer and/or subjects having head and neck cancer.

123. The method of any one of claims 113-122, wherein the method excludes centrifugation of the biological sample from the subject.

124. The method of any one of claims 113-123, wherein the method excludes centrifugation of the biological sample from the subject prior to DNA isolation.

125. The method of any one of claims 113-124, wherein the method further comprises ligation of an adaptor to the DNA.

126. The method of claim 125, wherein the adaptor comprises at least one barcode.

127. The method of claim 125 or 126 wherein the adaptor comprises a 5′ and/or 3′ primer binding site.

128. The method of any one of claims 113-127, wherein the method further comprises enrichment of the DNA in the biological sample for the biomarker genes.

129. The method of claim 128, wherein enrichment comprises contacting the sample with a nucleic acid probe complimentary to the biomarker gene under conditions that allow for the hybridization of the probe and DNA in the biological sample that is at least partially complimentary to the probe.

130. The method of claim 129, wherein the enrichment further comprises isolating the DNA hybridized to the probe.

131. The method of claim 130, wherein the method further comprises sequencing the DNA hybridized to the probe.

132. The method of any one of claims 113-131, wherein the method further comprises sequencing DNA comprising all or part of the biomarker genes to provide the sequence of all or part of the biomarker genes.

133. The method of claim 132, wherein sequencing comprising contacting the biomarker gene with a polymerase and primer(s)s that hybridize to the biomarker gene or adjacent regions and using polymerase chain reaction (PCR) to amplify DNA sequences comprising the biomarker gene.

134. The method of claim 132 or 133, wherein sequencing comprises next generation sequencing.

135. The method of claim 132 or 133, wherein the coding exon regions of the biomarker gene are sequenced.

136. The method of claim 135, wherein all of the coding exon regions of the biomarker gene are sequenced.

137. The method of any one of claims 132-136, wherein the method further comprises comparing the sequence of the biomarker genes to a control.

138. The method of claim 137, wherein the control comprises the wild-type sequence of the gene.

139. The method of any one of claims 113-138, wherein the number of biomarkers evaluated in the biological sample is 1-7 biomarkers.

140. The method of any one of claims 113-139, wherein the biomarker comprises TP53.

141. The method of any one of claims 113-140, wherein the biomarker comprises CDKN2A.

142. The method of any one of claims 113-141, wherein the biomarker comprises FAT1.

143. The method of any one of claims 113-142, wherein the biomarker comprises CASP8.

144. The method of any one of claims 113-143, wherein the biomarker comprises NOTCH1.

145. The method of any one of claims 113-144, wherein the biomarker comprises HRAS.

146. The method of any one of claims 113-145, wherein the biomarker comprises PIK3CA.

147. The method of any one of claims 113-139, wherein the biomarkers comprise TP53, CDKN2A, FAT1, CASP8, and Notch1.

148. The method of claim 147, wherein the biomarkers comprise TP53, CDKN2A, FAT1, CASP8, Notch1, PIK3CA, and HRAS.

149. The method of claim 147, wherein the biomarkers consist of TP53, CDKN2A, FAT1, CASP8, Notch1, PIK3CA, and HRAS.

150. The method of any one of claims 113-149, wherein the method further comprises performing one or more diagnostic tests for head and neck cancer or OCSCC.

151. The method of claim 150, wherein the diagnostic test comprises a conventional visual and tactile exam, tissue biopsy, and/or histological evaluation of a tissue biopsy.

152. The method of any one of claims 113-151, wherein the method further comprises treating the subject determined to have or be at high risk for head and neck cancer or OCSCC.

153. The method of claim 152, wherein the treatment comprises surgical excision of a OCSCC tumor, neck dissection, radiation therapy, and/or chemotherapy.

154. The method of any one of claims 113-150, wherein the method excludes performing one or more diagnostic tests for head and neck cancer or OCSCC on the subject determined to not have or be at low risk for having head and neck cancer or OCSCC.

155. The method of any one of claims 113-154, wherein the subject is a human subject.

156. The method of claim 155, wherein the subject is greater than 50 years old.

157. The method of any one of claims 113-156, wherein the subject does not have any symptoms of head and neck cancer or OCSCC.

158. The method of any one of claims 113-156, wherein the subject has one or more symptoms of head and neck cancer or OCSCC.

159. The method of any one of claims 113-158, wherein the method excludes whole exome sequencing methods.

160. The method of any one of claims 113-159, wherein the method excludes droplet digital PCR.

161. The method of any one of claims 113-160, wherein the OCSCC comprises HPV-negative OCSCC.

162. The method of any one of claims 113-161, wherein the variant allele frequency (VAF) of the mutation is less than 1%

163. The method of any one of claims 113-162, wherein the DNA excludes cfDNA.

164. The method of any one of claims 113-163, wherein the subject has not been treated with therapeutic levels of chemotherapy or radiation.

165. The method of any one of claims 113-164, wherein the OCSCC comprises carcinoma of the tongue, buccal mucosa, alveolus, gingivobuccal sulcus, hard palate, lip, retromolar trigone, maxilla, or gum.

166. The method of any one of claims 113-164, wherein the head and neck cancer or OCSCC is pre-malignant, stage I, II, III, or IV cancer.

167. The method of any one of claims 113-166, wherein the subject is a non-smoker.

168. The method of any one of claims 113-166, wherein the subject is a smoker.

169. A kit comprising primers or probes for sequencing one or more biomarker(s), wherein the biomarker(s) comprise TP53, CDKN2A, FAT1, CASP8, NOTCH1, PIK3CA, and/or HRAS.

170. The kit of claim 169, wherein the kit further comprises saliva collection vessels.

171. The kit of claim 169 or 170, wherein the kit further comprises DNA adaptors comprising a barcode.

172. The kit of any one of claims 169-171, wherein the DNA adaptors further comprise a 5′ and/or 3′ primer binding site.

173. The kit of any one of claims 169-172, wherein the kit further comprises one or more nucleic acid probes complimentary to the biomarker gene.

174. The kit of claim 173, wherein the probes are attached to a capture moiety.

175. The kit of claim 174, wherein the capture moiety comprises biotin.

176. The kit of claim 175, wherein the kit further comprises streptavidin bound to a solid support.

177. The kit of any one of claims 172-176, wherein the kit further comprises primers that hybridize with the adaptor.

178. The kit of any one of claims 169-177, wherein the kit further comprises one or more negative or positive control samples.

179. A method comprising: (i) isolating saliva DNA from an oral rinse sample from a subject; and (ii) sequencing TP53, CDKN2A, FAT1, CASP8, NOTCH1, PIK3CA, and HRAS genes in the DNA isolated from (i).

180. A method of making a nucleic acid comprising: isolating saliva DNA from an oral rinse sample from a subject; annealing primers to the isolated DNA, wherein the primers amplify and/or sequence the TP53, CDKN2A, FAT1, CASP8, NOTCH1, PIK3CA, and HRAS genes in the isolated DNA.
